(19) United States
(12) Patent Application Publication
(10) Pub. No.: US 2025/0041446 A1
(43) Pub. Date: Feb. 6, 2025

(54) A-REPEAT MINIGENE COMPOSITIONS FOR TARGETED REPRESSION OF SELECTED CHROMOSOMAL REGIONS AND METHODS OF USE THEREOF

(71) Applicant: UNIVERSITY OF MASSACHUSETTS, Boston, MA (US)

(72) Inventors: Jeanne Bentley LAWRENCE, Worcester, MA (US); Melvys VALLEDOR CEBALLOS, Worcester, MA (US); Meg BYRON, Sterling, MA (US)

(21) Appl. No.: 18/717,595

(22) PCT Filed: Dec. 9, 2022
(86) PCT No.: PCT/US2022/052431

§ 371 Date: Jun. 7, 2024

Related U.S. Application Data
(60) Provisional application No. 63/287,711, filed on Dec. 9, 2021.

(51) Int. Cl. A61K 48/00; A61P 25/28; C12N 9/22; C12N 15/11; C12N 15/90

(52) U.S. Cl. CPC A61K 48/005; A61P 25/28; C12N 9/22; C12N 15/111; C12N 15/907; C12N 2310/20

(57) ABSTRACT

This invention relates to compositions and methods for modulating gene expression, e.g., allele-specific gene expression, and to DNA sequences that can be integrated into targeted genomic locations (e.g., introns, exons, non-coding regions) within or near one or more alleles and confer reduced expression of said allele(s). Targeted alleles include, but are not limited to, gene sequences, translocated sequences, fully or partially duplicated sequences, and integrated viral-derived sequences.

### CLAIM OF PRIORITY

This application claims the benefit of U.S. Provisional Patent Application Ser. No. 63/287,711, filed on Dec. 9, 2021. The entire contents of the foregoing are hereby incorporated by reference.

### STATEMENT REGARDING FEDERALLY SPONSORED RESEARCH OR DEVELOPMENT

This invention was made with government support under Grants Nos. HD091357 and GM122597 awarded by the National Institutes of Health. The government has certain rights in the invention.

### TECHNICAL FIELD

This invention relates to compositions and methods for repressing genes within a small region on one homologous chromosome to modulate allele-specific gene expression, and more particularly to nucleotide sequences encoding an XIST A-Repeat domain or minigene as described herein, and fusion nucleotide sequences comprising a promoter and nucleotide sequence encoding an XIST A-Repeat domain or minigene as described herein. The said fusion nucleotide sequences can be targeted to integrate into the genome at a target site, e.g., a deleterious locus or other region of interest, which may be a SNP within an intron, or other sequence that is uniquely present (or absen) on one allele, and the RNA transcribed from the fusion nucleotide sequence is sufficient to mediate silencing of neighboring genes whose promoters are located 20 kb-5 mb from the target site. Target sites include, but are not limited to, non-coding or coding sequences in or near specific gene sequences, translocated sequences and duplicated sequences.

### BACKGROUND

In many circumstances in biomedicine it would be desirable to modulate expression of one or more genes in part of a chromosome without impacting genes throughout the whole chromosome. About 0.6-0.7% of live births ( 1/140 in the United States) are impacted by a chromosomal abnormality that causes a duplication or deletion of chromosomal material (Czerminski and Lawrence, Dev Cell. 2020 Feb. 10; 52(3): 294-308.e3; Malani, “Genetics, Chromosome Abnormalities,” 2021 (statpearls.com/articlelibrary/viewarticle/32619/)), and the fraction of known cases is increasing as better ways to detect smaller changes are implemented (G. Logsdon with E. Eichler, Nat Reviews Genetics, 2020). Down Syndrome (˜ 1/750 live births) is the most common sub-category of these disorders and is caused by trisomy for chromosome 21. Other chromosomal imbalances are individually much rarer, but collectively are more frequent than DS, and many involve duplication or deletion of small parts of a chromosome, rather than the whole chromosome. Chromosomal abnormalties and pathogenic copy number variations (CNVs) are a major part of the human genetic burden that is not addressed by current progress on single-gene disorders, nor has the extent of this burden been fully identified. The ability to modulate expression of multiple genes in a limited chromosomal region would have wide applicability not only as a tool for research but as a potential therapeutic strategy applicable to a broad array of collectively common conditions. The X-linked XIST gene encodes a long non-coding RNA that spreads across the nuclear chromosome structure and silences genes throughout one whole female X-chromosome, but targeted insertion of XIST can comprehensively silence genes on an autosome, as shown for chromosome 21. There is no known way to limit the spread of XIST RNA on the chromosome in cis, and the extreme length of the 14-19 kb XIST cDNA presents technical obstacles to manipulation and in vivo delivery of XIST as a therapeutic agent.

### SUMMARY

Described herein are methods for targeting an epigenetic mechanism (XIST A-repeat minigenes) to regulate the expression of closely-linked genes within a small chromosomal region, without impacting genes across the whole chromosome. For example, described herein are methods and compositions to use an XIST A-repeat domain minigene targeted to a chromosomal region, e.g., a deleterious locus, including a duplicated locus, to repress expression of genes in that region. More specifically, we have shown in trisomy 21 stem cells that a minigene containing the small (450 bp) “A-repeat” fragment of the large (14 kb) XIST cDNA can be targeted into an intron of one Chromosome 21 gene and reduce to normal disomic levels expression of genes in the “Down Syndrome Critical Region”. A-repeat minigenes lack most natural sequences required for the RNA and silencing to spread across the chromosome, and the smaller size of the minigene is advantageous for in vivo delivery techniques. For many genetic conditions the repression of one or more genes, e.g., deleterious genes, clustered in a small chromosomal region is desirable, whereas broader transcriptional repression of genes throughout the chromosome would be harmful. A-repeat minigenes produce RNA that can repress multiple endogenous genes within a limited region up to ˜10 Mb centered on the insertion site (so up to about 5 Mb from the insertion on either side), but specifically avoid the chromosome-wide spread that is a defining characteristic of natural XIST RNA. We have inserted the A-repeat into two genes important in Down Syndrome pathology, DYRK1A and APP. For DYRK1A, we show that the A-repeat silences from within the intron. There is no suitable common SNP in the APP or DYRK1A coding regions to enable allele-specific gene targeting, but the present approach can work by targeting into a SNP in an intron or adjacent intergenic sequence. Therefore, A-repeat minigenes also provide a solution to allow allele specific silencing for many genes in which there is no SNP in the coding region to create and indel to disrupt function. This approach could have broad potential applications for biomedical research and therapeutics, requiring only changing the targeting site of the same XIST A repeat transgene. In addition, methods and compositions defined here have important therapeutic potential for the approximately 300,000 people in the U.S. with Down Syndrome, almost all of whom will be afflicted with Alzheimer's dementia (AD) 20-30 years before the non-DS population, and may benefit from sustained repression of one of three APP genes on the trisomic Chr21.

The present methods and compositions have a number of advantages, including in some embodiments: The A-repeat miningene does not spread, providing local control over silencing; and the A-repeat minigene deletes most XIST domains to reduce the 14-17 kb full-length to no more than 5 kb (which fits into AAV delivery vectors). The discovery that the tiny A-repeat fragment alone is functional makes it feasible to build small transgenes with additional properties by “addition” to the A-repeat fragment.

Provided here are methods for silencing one or more alleles of a target gene, e.g., an endogenous gene, in a cell, the method comprising inserting a silencing sequence comprising a promoter sequence and an XIST A-repeat minigene comprising about eight or nine, and up to 50, preferably 6-20, XIST A-repeats comprising a sequence as described herein into the genome of the cell, wherein the silencing sequence is inserted at a site that is up to 5 Mb, e.g., 100-500 kb, away from the target gene promoter. Also provided are methods of silencing one or more alleles of a target gene, e.g., APP or DYRK1A, in a cell, the method comprising inserting an A-repeat minigene silencing sequence of up to 5 kB comprising a promoter sequence and at least eight or nine A-repeats, and up to 50 A-repeats, preferably 6-20, or 20-50 or 30-50 A-repeats, wherein each A-repeat comprises a sequence that is at least 80%, 85%, 90%, 95%, or is 100% identical to GCCCA[T/A]CGGGG[C/T]N[G/T/A][C/T]GGATA[C/T]CTG, wherein N is any nucleotide, and preferably forms hairpin loops, optionally with T-rich flanking regions in between each repeat, into the genome of the cell, wherein the silencing sequence is inserted up to 5 Mb, e.g., 100-500 kb, away from the target gene promoter. Exemplary A-repeat and silencing sequences are described herein. In some embodiments, a local chromosome region comprising a number of genes is silenced, up to 10 Mb (i.e., 5 Mb on either side of the insertion site, with the strongest repression 2 Mb on either side of insertion site). In some embodiments, the methods are used for silencing of the Down Syndrome Critical Region, in which the DYRK1A gene resides. In some embodiments, the A-repeat minigenes comprise up to 450 bp, 500 bp, 1 kb, 2 kb, 2.5 kB, 3 kB, or 4 kB of XIST, either contiguous sequence or domains as described herein, optionally linked with peptide linkers. In some embodiments, the method can be used for, e.g., results in, silencing of a plurality of genes that have promoters within up to 5 Mb, preferably up to 100-500 kb, of the insertion site. Also provided are the A-repeat minigenes themselves, as well as vectors comprising the A-repeat minigenes, for use in silencing one or more target genes that have promoters within up to 5 Mb, preferably up to 100-500 kb, of the insertion site.

In some embodiments, the silenced genes are endogenous genes. In preferred embodiments, the silencing site is inserted at a specific site, e.g., inserted at an intended site, not randomly into the genome.

In some embodiments, genomic insertion of the silencing sequence is directed using a method such as zinc-finger nucleases or TALENs or zinc fingers (ZFs) that specifically target the genomic insertion site. In some embodiments, genomic insertion of the nucleotide sequence is directed by Cas9 complexed with a guide RNA that specifically target the genomic insertion site.

In some embodiments, the XIST A-repeat domain is inserted at a copy number variation or single-nucleotide polymorphism (SNP) located within a 5′ UTR, intron, or exon of one or more alleles of the target gene.

In some embodiments, the XIST A-repeat domain is inserted at a sequence that is present on just one homologous chromosome, optionally a single-nucleotide polymorphism (SNP) or copy number variation (CNV), that is present within a 5′ UTR, intron, or exon of one allele of the target gene but absent in other alleles of the target gene.

In some embodiments, the target gene is present in two or more copies in the cell, and the presence of two or more copies of the target gene is associated with a disease.

In some embodiments, the disease is selected from the group of Down Syndrome, Alzheimer's disease, Chromosomal imbalance disorders, and microduplication disorders.

In some embodiments, the disease is Down Syndrome or Alzheimer's Disease and the target gene is amyloid precursor protein (APP), DYRK1A, DSCR3 (VPS26C), TTC3, PIGP, HLCS, RCAN1, CBR1, DONSON, ETS2, PSMG1, MX1, BACE2, IFNAR1, IFNGR2, IFNAR2, and/or IL1.

In some embodiments, the cell is a cell in a living subject, e.g., a mammal, e.g., a human who has a disease, e.g., selected from the group of Down Syndrome, Alzheimer's disease, Chromosomal imbalance disorders, and microduplication disorders.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

FIGS. 1A-F: XIST RNA compacts a highly distended chromosome while heterochromatic hallmarks are sequentially accumulated. DAPI was used to stain DNA. A) Active (Xa) and inactive (Xi) X-Chromosomes in somatic nucleus labeled with X-Chromosome paint. Originally red and blue channels separated at right, with DAPI dense Barr body indicated (arrow). B) Same chromosome paint in pluripotent nucleus. Originally green channel with nuclear outline at right and inverted black & White far right. C) Immunofluorescence (IF) alone or with XIST RNA FISH for 4 classic heterochromatin hallmarks after 1 week of XIST expression in pluripotent or differentiated iPSCs. D-E) Enrichment of H4K27me3 & H2AK119ub by IF, after 1-7 days (D) or 2-24 hours (E) of XIST expression. F) Enrichment of H4K20me & macroH2A by IF, after 1-7 days of XIST expression.

FIGS. 2A-L. XIST RNA spreads broadly at low density within hours and alters chromatin differently when at high and low density. DAPI DNA is blue (F-L). A-D) XIST RNA (FISH) in nuclei over time-course of XIST expression. Black & white image shows RNA signal with outline of nucleus. Heatmap of XIST RNA signal intensity at center and illustration showing sparse and dense XIST RNA (dots) zones in nucleus at right. E) Xist RNA (FISH) territories over time-course in differentiating mouse ES cells containing an inducible Xist transgene integrated on Chr11. Black & white image shows RNA signal with outline of nucleus. F) A field of cells after 4 hours XIST induction. Originally green channel is separated below with edge of XIST sparse-zone signal-threshold outlined in white. Select foci are shown as heatmap (inserts) to illustrate density changes. G) Tautomycin treated human Tig-1 fibroblasts release XIST RNA. Green channel is separated at right. H-J) IF of H2AK119ub (H-I) and H3K27me3 (J) with XIST RNA FISH. Originally red & green channels separated below, with edges of signal-threshold outlined at bottom. K) Linescans of representative nuclei showing IF labeling across the XIST RNA territory (boxed region) for H2AK119ub and H3K27me3. L) DAPI condensation in dense XIST RNA zone. Separated channels and representative intensity heatmaps of the XIST RNA region in close-up, below.

FIGS. 3A-H. Formation of Barr body architecture occurs days before most gene silencing. DAPI DNA is blue (B-D & G). A) Cot-1 RNA hole formation (RNA FISH) over time-course of XIST expression in pluripotent iPSCs. B-C) CoT-1 and XIST RNA FISH with representative linescans below. Edges of XIST RNA signal indicated by black box. D) XIST and APP RNA FISH in differentiating iPSCs. Inserts: single channel close-up of APP gene signals (arrows). E) Gene silencing (loss of transcription focus) for four genes over time-course of XIST expression in pluripotent iPSCs. Ideogram of gene location on Chr21 below. F) CXADR and XIST RNA FISH. Outline of nucleus in white and threshold of XIST RNA signal also outlined. G) XIST and APP RNA FISH. DAPI channel separated below, with APP transcription focus relative to XIST RNA/Barr body indicated (arrows). Inset: APP and XIST RNA with originally blue channel removed for clarity. H) XIST RNA and CXADR DNA FISH. DAPI channel separated below, with location of CXADR gene relative to XIST RNA/Barr body indicated (arrows). Inset: CXADR gene (DNA) and XIST RNA with blue channel removed for clarity.

FIGS. 4A-H. XIST RNA impacts the scaffold early but chromosomal movement to nuclear periphery is late and requires differentiation. DAPI was used to stain DNA (A-C & F). A) SAF-A IF in pluripotent iPSC nuclei. Separated channels at right. B) CIZ1 (IF) and XIST RNA (FISH) in induced and un-induced neighboring nuclei. Separated channels below with nuclei outlined in white. C) CIZ1 (IF) and XIST RNA (FISH). Close-up of XIST RNA region with separated channels at right. D) CIZ1 mRNA levels in pluripotent and differentiated iPSCs (Endothelial & Neural progenitor cells). E) Scoring nuclei with XIST RNA (FISH) and CIZ1 or H2AK119ub enrichment (IF) after XIST induction. F) Simultaneous CIZ1 and H2AK119ub IF. Close-up of XIST RNA region with separated channels at right. G) Scoring number of pluripotent and differentiated cells with transgenic Chr21 located at nuclear periphery. H) Illustration showing timing of human chromosome inactivation hallmarks. Within hours XIST RNA spreads across the chromosome territory (located predominantly near the nucleolus) at low density but doesn't silence most genes. The low-density XIST RNA triggers H2AK119Ub, while the dense XIST RNA domain begins compacting the Barr body (delineated by a Cot-1 RNA depletion), which accumulates H3K27me3. After 3 days, distal coding genes still producing transcription foci are drawn towards the Barr body, where they are silenced and accumulate H4K20me. The chromosome remains at the nucleolus, and free of macroH2A unless it's differentiated and moves into the peripheral heterochromatic compartment.

FIGS. 5A-J. Expression of A-repeat transgene silences RFP reporter and nearby endogenous genes. DAPI DNA is blue (all images). A) A-repeat transgene map and insertion site on Chr21. B) RFP and DYRK1A RNA FISH in un-induced cells, with co-localization of RFP and DYRK1A RNA at transgene target site (insert). Originally red channel separated below, with no reduction in linked DYRK1A TF (arrow). C) RNA FISH for indicated probes, with average RNA territory size indicated in image and in graph. D) RFP in transgenic iPS cell cultures with and without dox induction of A-repeat. Close-up of representative Dox (+) colony (far right) indicates not all cells induce A-repeat. E-F) RNA FISH of indicated probes. Separated channels for Chr21-linked gene RNA below and at right. Locus with A-repeat transgene indicated (arrow). G) Quantification was performed from z-stacks of RNA FISH images. Frequency of un-linked alleles versus those linked to A-repeat RNA. “Trace” signals for DYRK1A was considered silenced due to read-through from transgene (See also FIGS. 13A-K for more details). H) APP and A-repeat RNA FISH. I) Quantification of repressed DYRK1A transcription focus associated with A-repeat using FISH images. J) A-repeat RNA FISH (induced and un-induced iPSC population).

FIGS. 6A-I. De-acetylation is essential for gene silencing but may require high density of A-repeat. DAPI DNA is blue (C & F). A-B) Repression of DYRK1A transcription focus associated to A-repeat (A) or flXIST (B) by RNA FISH (Two-way ANOVA for significance). C) Representative FISH images quantified in A. Three color image (left) and green channel removed for clarity (right). (See also FIGS. 14A-G for more details). D) CoT-1 RNA (left) in neighboring iPSCs induced and un-induced for A-repeat expression (right). E) H3K27ac (IF) and A-repeat RNA (FISH) in neighboring induced and un-induced iPSCs (green channel separated at right), with quantification of signal intensity (below), and cells lacking A-repeat RNA indicated (red circle: graph and arrows: images). F) APP and A-repeat RNA FISH. Red and green channels separated at right. G-I) H3K27ac and H2AK119ub (IF). Two-color images (right) and originally green channel alone (left). Linescans (far right) of originally two-color images (with white line), with edge of H2AK119ub signal indicated by black box. Close-up of originally green channel in black and white (H: insert) with H2AK119ub depletion indicated (arrows). J) Quantification of repressed DYRK1A transcription focus associated to flXIST RNA using FISH images.

FIGS. 7A-E. A. Map of full length XIST RNA coding sequence is shown with conserved repeat sequences indicated below. Boxes indicate sequences included in three A-repeat minigenes: the smallest has just the A-repeat (450 bp), and the 1 kb and 2.5 kb minigenes add other XIST sequences (to the A-repeat), including portions of the conserved F, E, and B repeats. B. Fusion construct with A-repeat minigenes designed to promote targeted integration into the DYRK1A locus in the Down Syndrome Critical Region of Chr21. All three A-repeat minigenes were cloned into a donor plasmid under an inducible promoter with homology arms to target DYRK1A intron. Donor plasmid was integrated by transfection with zinc finger nucleases that cut the target intron in DYRK1A. C-E. RNA FISH to cells expressing A-repeat minigenes. All three A-repeat minigenes show a single small dot-like accumulation in contrast to the larger accumulation of the full-length XIST RNA which spreads across the whole nuclear chromosome territory (shown in inset in FIG. 7C, and FIGS. 5A-I and FIGS. 13A-K).

FIG. 8. Bulk RNAseq data shows two A-repeat minigenes (450 bp and 2.5 Kb) repress expression of numerous genes near the minigene insertion site (in DYRK1A intron, pink line), in Down Syndrome derived iPSCs. Shown is ˜28 Mb of Chr21. The most effectively repressed genes are limited to a region of ˜5 Mb, as indicated by genes that decrease with higher statistical significance (black dots). Polynomial regression curves shows some trend of decrease in an 8 Mb region (repA, for 450 bp minigene and miniXIST, 2.5 Kb minigene), with shaded confidence intervals. The 0.00 line marks the reference of uncorrected trisomic transcription levels (no dox), while the lines are from cultures induced to express A-repeat minigenes. Dotted dark grey line indicates theoretical ⅓ reduction if all cells were fully silenced one of the three alleles. For technical reasons, a subset of cells is typically not induced by doxycycline to express the minigene RNA, yet the strong trend of repression of multiple genes in the target region is evident. Vertical grey shaded area highlights 10 Mb segment centered on insertion site, beyond which repression does not extend, as illustrated by APP and PRMT2. (Note: Quantifying DYRK1a expression by RNAseq is complicated by any read-through from minigene promoter into DYRK1A sequences).

FIGS. 9A-H: XIST RNA compacts an initially distended chromosome and heterochromatic hallmarks are largely similar between pluripotent and differentiated cells. DAPI was used to stain DNA (A, F-H). A) Chr21 library DNA in Down syndrome iPSC showing 3-chr21, with XIST RNA FISH indicating compacted transgenic chromosome. Originally green channel separated below. B-E) The timing of chromatin hallmarks scored in pluripotent and differentiating iPSCs during 7 days of XIST expression. Only macroH2A (E) shows a significant difference between differentiating and pluripotent cultures. F-H) MacroH2A enrichment was only observed upon differentiation in human iPSCs (F & G) and ES cells(Hoffman et al., 2005) under older growth and maintenance protocols using inactivated feeders. However, using modern iPSC feeder-free culture conditions we observe macroH2A enrichment beginning on day 3 in pluripotent cells (H), suggesting modern culture methods may change epigenetic plasticity of these cells. Red and green channels separated below main images.

FIGS. 10A-E: Low level spread of XIST RNA is seen early in the process and may often be missed but they impact chromatin. DAPI was used to stain DNA (all images). A-B) A field of iPSC at 4 hours (A) and 8 days (B) show the change in the XIST RNA territory over time. The originally green channel is separated at right with threshold edges of the 4-hour XIST territory outlined. Inserts show two representative XIST RNA signals (arrows) with a 6-color heat map of pixel intensity showing sparse and dense zones. Note: FIG. 2 in main text shows region of same 4 hr field. Due to the low intensity of the sparse XIST RNA and the dynamic range between that and the transcription focus, the initial sparse spread of XIST RNA may often be missed, particularly if cells are only observed on a computer screen (with poor dynamic range) rather than by eye under a microscope, or if images are processed too much. C) During X-inactivation in very early mouse embryos, endogenous Xist-RNA also exhibits a large sparse dispersal rather than a small compact cloud (surrounding trophectoderm cells are not included in the image). The originally green channel is separated for select Xist RNA territories in inserts. Originally blue channel for entire cell mass separated at right. D) Field of 4 hr induced cells with H2AK119ub (IF) enrichment under XIST RNA (FISH) territories. Note, not all cells in this field responded to induction and expressed XIST. A 6-color heat map of XIST RNA and H2AK119ub IF pixel intensity is separated below. E) H2AK1129Ub enrichment is seen across the entire XIST RNA sparse zone, while H3K27me3 is only seen over the center dense XIST RNA zone. Originally red and green channels separated below, and Illustration of the threshold-edge of the signals in insert.

FIGS. 11A-F: Cot-1 RNA “hole”/Barr body formation over the inactivating chromosome. DAPI was used to stain DNA (all images). A) DAPI dense Barr bodies (BB) are not easy to detect in all cell preps (particularly pluripotent cells), making the presence of a Cot-1 RNA hole the most reliable way of detecting the BB. Originally red and blue channels separated at right with location of inactive chromosome (with XIST RNA expression) indicated (arrows). B-E) Reduction of Cot-1 RNA over XIST RNA territory in 4 hr, 8 hr, 3-day and 10-day nuclei. Linescans across regions delineated in 3-color images (white lines) are at right. Edges of XIST RNA territory indicated by black boxes. F) APP and CoT-1 RNA FISH show APP transcription focus at edge of CoT-1 RNA hole prior to silencing in iPSC. Linescan across region (white line) at right. Closeup of region, with originally blue channel removed, in insert.

FIGS. 12A-C: XIST RNA impacts the scaffold early but chromosomal movement to nuclear periphery is late and requires differentiation. A) Detection of CIZ1 and H2Ak119ub accumulation before and after XIST expression. To determine whether CIZ1 or H2Ak119ub appears first, simultaneous staining for both proteins was done at 2 hrs of XIST induction (RNA hybridization was not included to optimize detection of both antibodies). In most cells, both proteins were detected and co-localized in a single bright cloud, presumed to be the XIST transcription focus. But a small fraction of cells at 2 hours contained an enriched focus of just one signal (CIZ1 or H2AK119ub). Because some non-induced cells already contained H2AK119ub foci, we can conclude that a small subset of cells are enriched for CIZ1 without H2AK119ub modification. B-C) Nuclear location of the precociously inactivated Xi in several pluripotent human ES cell lines (B) and in the H9 hESC line after differentiation (C). Illustrations of each type of chromosome locations scored is at left.

FIGS. 13A-K: High density focal A-repeat RNA silences nearby genes while low levels of A-repeat RNA distribute broadly but remain in the nucleus. DAPI DNA is blue (B-G). A) Diagram of Chr21 gene loci examined for silencing by A-repeat RNA. B) Field of induced cells with DYRK1A and A-repeat RNA FISH. DSCR3 (also known as VPS26) (C), TTC3 (D), PIGP (E), HLCS (F) & APP (G)) RNA foci were scored in relation to DYRK1A RNA foci to ascertain hybridization frequency in un-induced cells. These were then compared to induced samples to determine silencing frequency by A-repeat. D) TTC3 & DYRK1 RNA FISH in uninduced cells (left) and TCC3 & A-repeat RNA FISH in induced cells (right). Separated channels in black & white as indicated. Silenced allele indicated (arrow). E) PIGP & DYRK1 RNA FISH in uninduced cells (left) and PIGP & A-repeat RNA FISH in induced cells (right). Separated channels in black & white as indicated. Silenced allele (grey arrows) and expressed alleles (white arrows) indicated. F) HLCS & DYRK1 RNA FISH in uninduced cells (left) and HLCS & A-repeat RNA FISH in induced cells (right). Reduced hybridization efficiency resulted in some DYRK1 foci not having a corresponding HLCS focus (white arrow). Silenced allele (grey arrow) and expressed alleles (white arrows) also indicated. G) Because the APP gene is 11 MB away from the DRYRK1 locus (where the A-repeat is targeted), they can be far apart in some nuclei (arrows), but three foci were apparent in all cells whether induced or uninduced. H) Example illustrating that all genes examined were not monoallelically expressed in cells, (except those on the Xi(Clemson, Hall, Byron, McNeil, & Lawrence, 2006)) I-J) A single channel image of A-repeat and RFP RNA (FISH) I a field of cells shows a bright RNA focus and a lower density dispersed nucleoplasmic RNA signal filling individual nuclei. A-repeat RNA is restricted to the cytoplasm, while RFP mRNA is transported to the cytoplasm for translation. K) A-repeat transcription foci are gone after 30 min of transcriptional inhibition (left) leaving only dispersed signal delineating nuclei, and another 30 min is required for complete loss of signal.

FIGS. 14A-G: Nuclear periphery is not involved in gene silencing but TSA treatment during silencing reveals an HDAC-dependent and HDAC-independent silencing state. DAPI DNA is blue (B-G). A) Nuclear localization of A-repeat RNA focus (on transgenic Chr21) compared to DYRK1A alleles on non-transgenic Chr21s in pluripotent and endothelial differentiated iPSCs. B-C) H3K27ac (IF) in cells treated with TSA or DMSO for 4 hours. D-E) Representative example of A-repeat and DYRK1A RNA FISH images used in FIG. 6A quantification. TSA treatment (or DMSO alone) following gene silencing (D) or during gene silencing (E). F-G) Representative example of flXIST and DYRK1A/APP RNA FISH images used in FIG. 6B quantification. TSA treatment (or DMSO alone) following gene silencing (F) or during gene silencing (G). DYRK1A was used for short-term TSA treatment during flXIST mediated chromosome silencing, since APP took days to silence.

FIG. 15. Tagman RT-qPCR assay showing relative to TcMAC21 (normalized as 1), repression of human chr21 genes in TcMAC21/A-repeat transgenic mice in different tissues such as the brain, heart, and kidney.

### DETAILED DESCRIPTION

The commonality of the numerous but rare chromosomal disorders or pathogenic copy number variations (CNVs) is that they are caused by too many (or few) copies of genes within a specific chromosomal region. However currently there is no known way to repress or otherwise modulate expression of multiple genes within a specific chromosomal region. In certain medical conditions it may be desirable to regulate multiple genes clustered in a chromosomal region, such as the interferon receptor gene cluster on Chr21 or major histocompatibility genes clustered on Chr6. Numerous genome editing methods and compositions are known that can direct insertions, deletions, or substitutions of DNA within a specified target exon, e.g., an exon that has a sequence that is present on one allele, e.g., a CNV or single nucleotide polymorphism (SNP), including zinc finger nucleases (ZFNs; Cathomen et al. (2008). Zinc-finger Nucleases: The Next Generation Emerges. Molecular Therapy 16, 1200-1207), transcription activator-like effector nucleases (TALENs; Joung et al. (2013). TALENs: a widely applicable technology for targeted genome editing. Nature Reviews Molecular Cell Biology 14, 49-55), and CRISPR-Cas9 (Hsu et al. (2014). Development and applications of CRISPR-Cas9 for Genome Engineering. Cell 157, 1262-1278; Sander et al. (2014). CRISPR-Cas systems for editing, regulating and targeting genomes. Nature Biotechnology 32, 347-355), as well as others. Thus, it is of great interest for therapeutics, diagnostics, reagents, and biological assays to be able to modulate gene expression, e.g., in an allele-specific manner to reduce expression of one allele without affecting expression of other allele(s), and to silence multiple genes in a small chromosomal region.

In some embodiments, the present methods use targeted insertion of a single silencing sequence at a specific site to repress the expression of multiple endogenous genes within a specific small chromosomal region of interest, and, importantly, preserve full expression of most genes across the chromosome in cis. By deleting most of the long XIST cDNA sequence, this prevents chromosome-wide spread of silencing, which is desirable for many applications in biology, for repression of specific chromosomal loci. In addition, the smaller A-repeat minigenes thus created are more amenable to in vivo delivery techniques, such as using AAV vectors. In addition, the approach allows genes from only one homologous chromosome to be modulated by targeting the minigene into a common SNP anywhere within the desired chromsomal region. As the example illustrates, A-repeat minigenes can function from within an intron of a gene, and introns more frequently have common SNPs that can be used for targeting discrimination of different homologous chromosome. Despite advances in genome editing, known methods for introducing an indel into an exon to disrupt gene function are unable to reduce expression of a specific target allele that lacks an exonic SNP, nor do they repress neighboring genes. SNPs are more common in introns but most genes lack common SNPs in the exon coding regions, as is the case for DYRK1A and APP. In contrast, the A-repeat domain minigene can be targeted to a SNP in an intron and can silence the promoter of that gene and closely-linked loci. Finally, known compositions are also unable to simultaneously reduce expression of genes within and across a desired target locus, whereas the present methods allow repression of promoters of other genes in the silencing region (up to ˜10 Mb centered on the insertion site, so up to about 5 Mb away) surrounding the integration site of a single nucleotide sequence, without affecting expression of synthetic genes outside this region. Thus, there is an unmet need for new compositions that reduce expression of either a desired target allele or multiple alleles in a desired target locus by integrating a single nucleotide sequence into a chromosomal region, and also provide wide flexibility to target common SNPs prevalent in introns in order to repress a particular allele on a particular homologous chromosome. It is known that many or most genes within the genome are not dosage-sensitive, although it is not clearly known what fraction of genes is dosage sensitive. Therefore, in circumstances in which silencing of one gene allele (e.g., one deleterious allele) is beneficial, it will often be the case that repression of one or multiple neighboring genes (on that one homologous chromosome) will have no deleterious effect, because normal expression of those genes from other chromosomes will be maintained.

With full length XIST, it is possible to insert one gene and silence a whole chromosome, which is ideal for a whole chromosome disorder, like trisomy 21 (Down Syndrome) (see, e.g., U.S. Pat. Nos. 10,004,765; 9,914,936; 9,681,646; 9,297,023; 8,574,900; and 8,212,019). XIST RNA is a 14-19 kb long non-coding RNA, much of which is not conserved in primary sequence, but it contains several areas of small tandem repeats that are relatively conserved in primary sequence (Brown et al., Cell 1992) and are thought to have conserved secondary structures. Natural XIST RNA is transcribed from just one X chromosome and the RNA accumulates and spreads across that chromosome to trigger X-chromosome inactivation in cis in female cells. A hallmark property of the long XIST RNA transcripts is that it spreads across the whole chromosome, and it has been shown that this X-chromosome gene can be inserted into an autosome, specifically chromosome 21, and comprehensively silence that autosome. Thus the full-length XIST molecule has the ability to silence a few hundred genes across a chromosome, but it cannot be used to silence selective genes or small gene cluster or region of a chromosome because it will spread and silence all genes on that chromosome. While the spreading property of XIST RNA may be beneficial for chromosomal abnormalities, such as in Down Syndrome, it could not be applied more broadly for selective gene silencing nor for the large number of smaller chromosomal imbalances that are an unaddressed part of the human genetic burden. In addition, the size of the full-length XIST transcript prohibits its delivery by current methods, such as by AAV delivery.

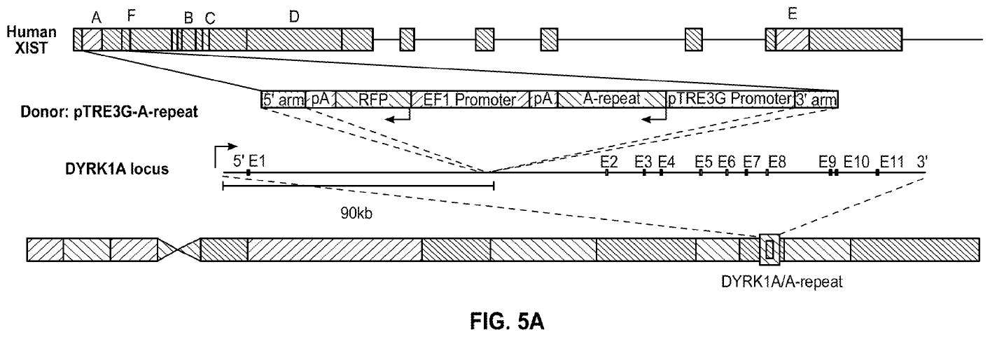

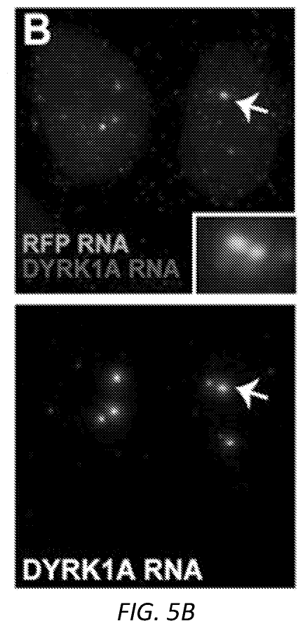

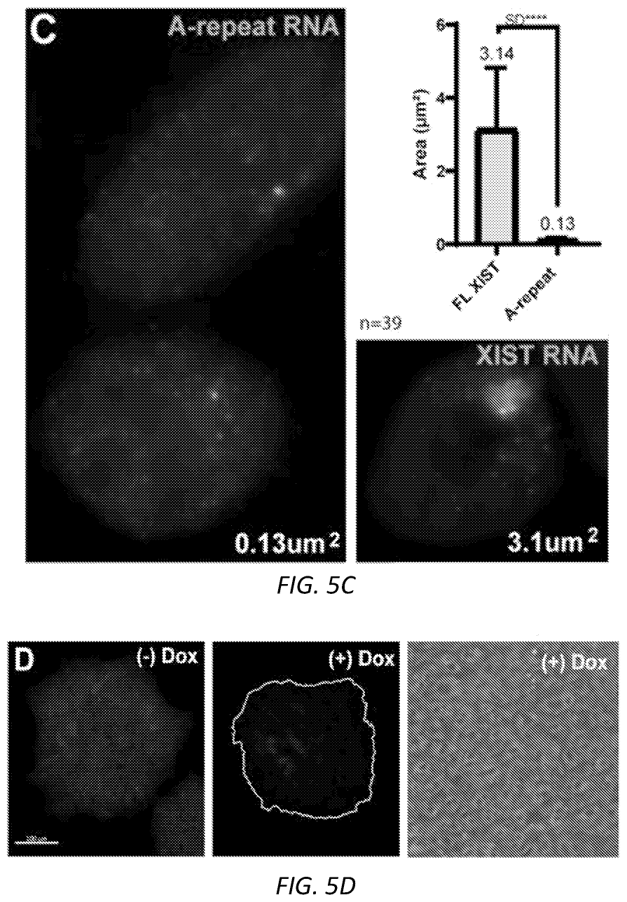

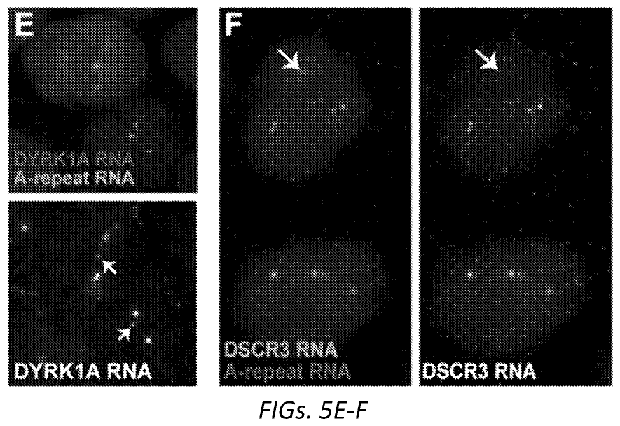

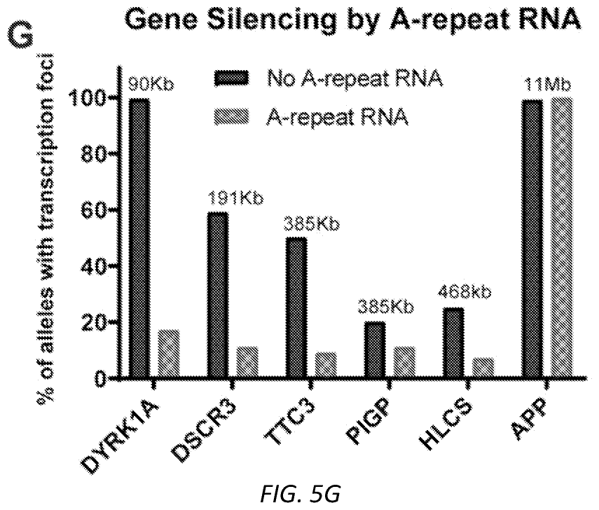

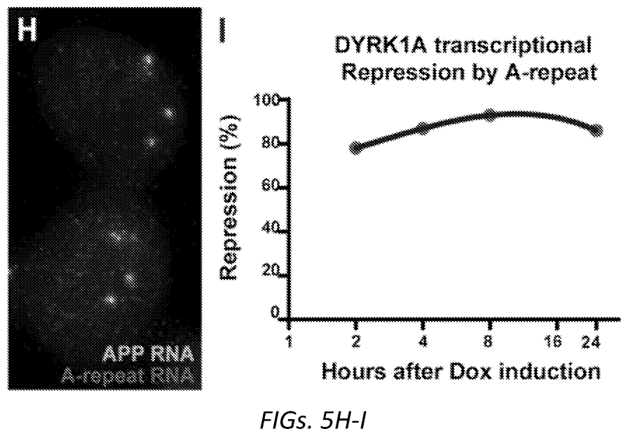

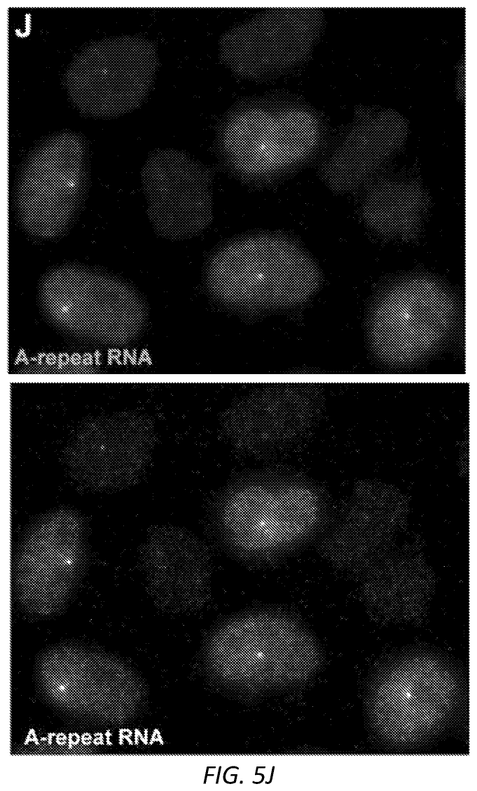

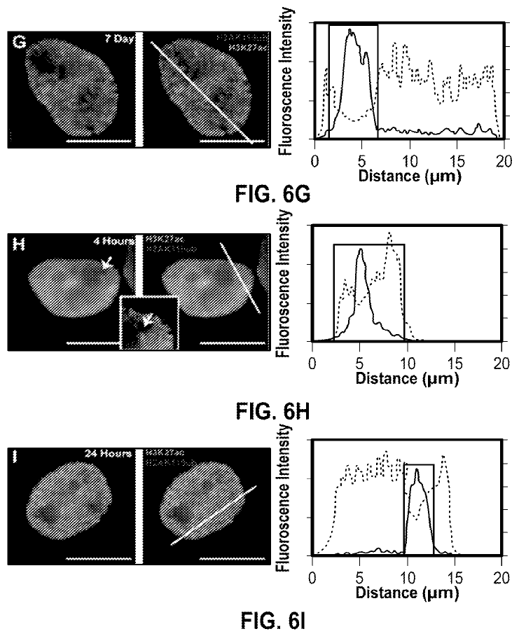

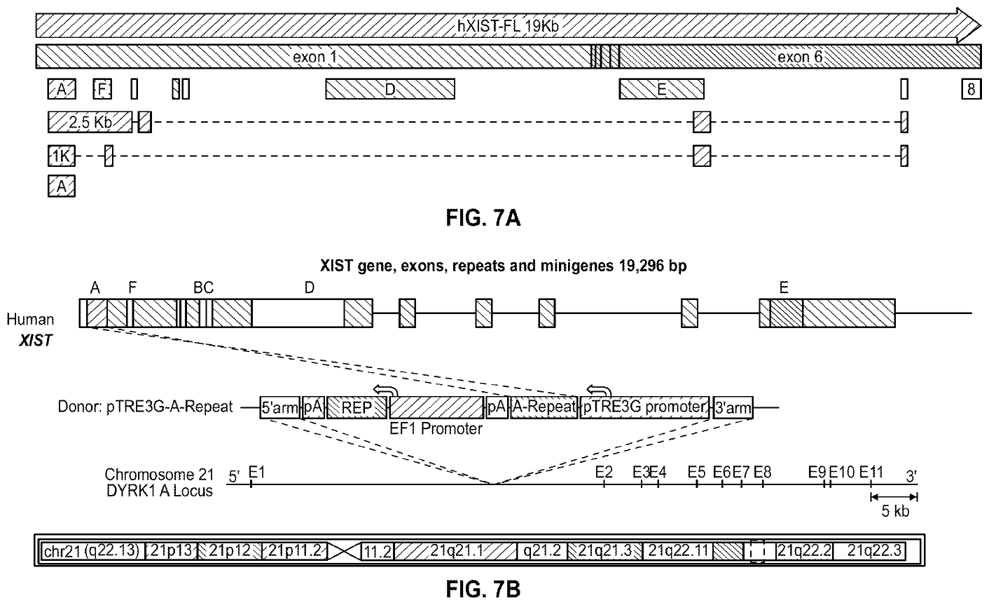

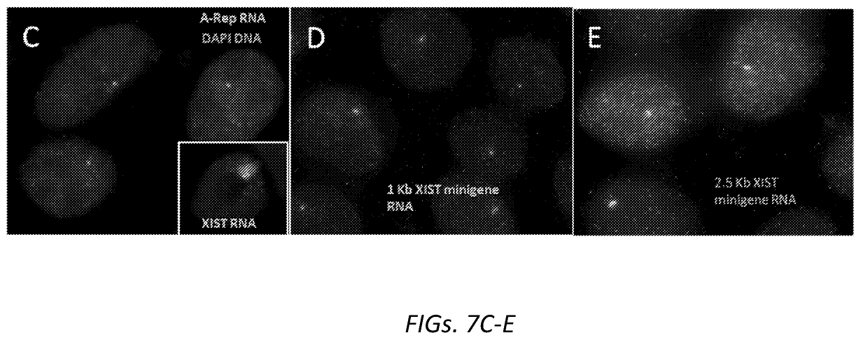

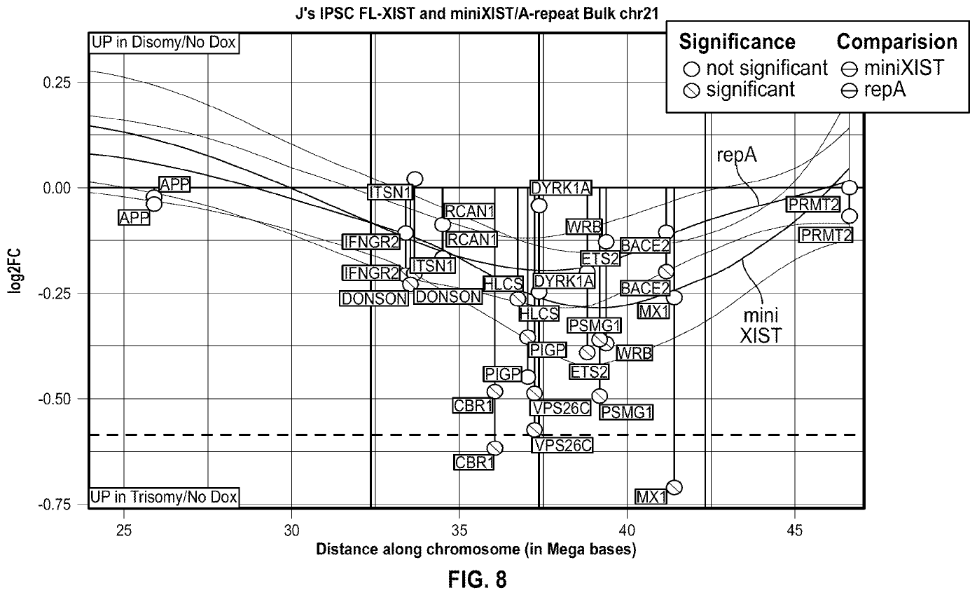

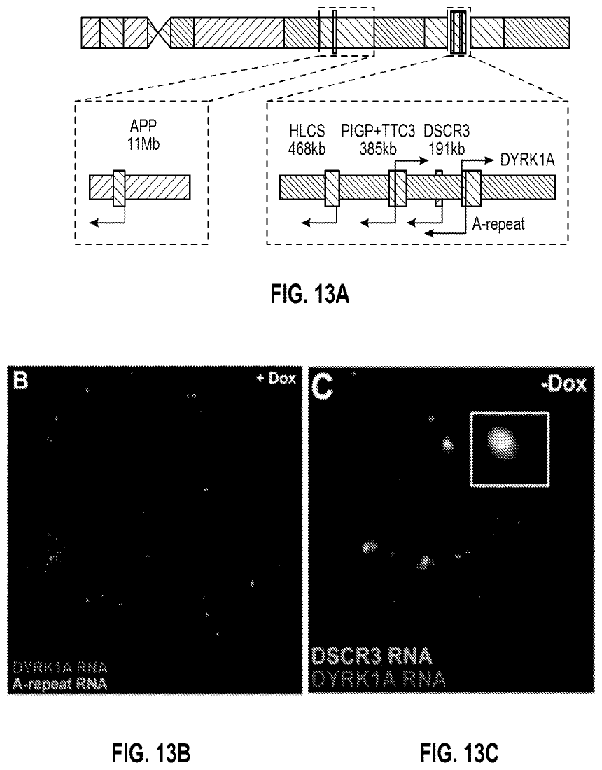

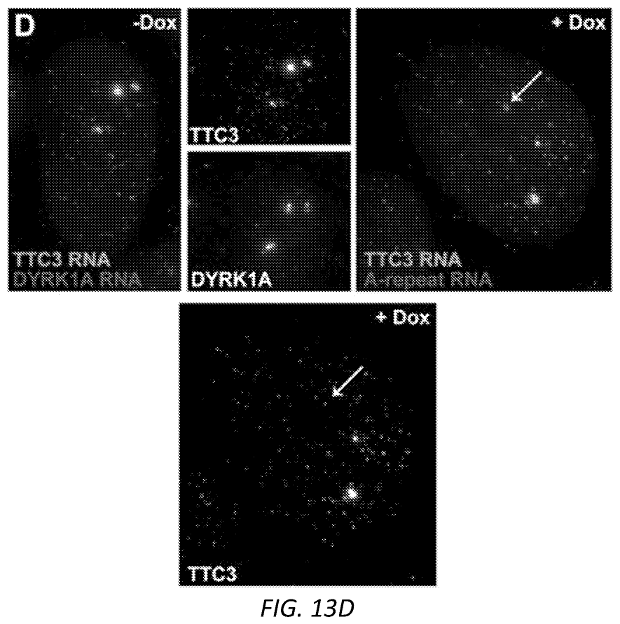

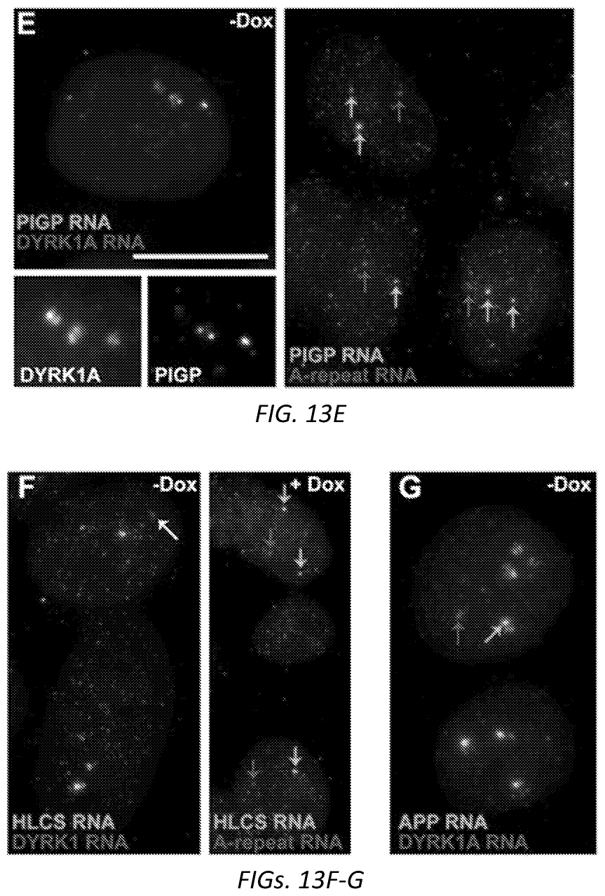

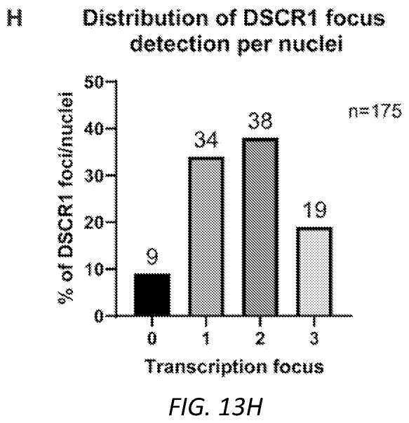

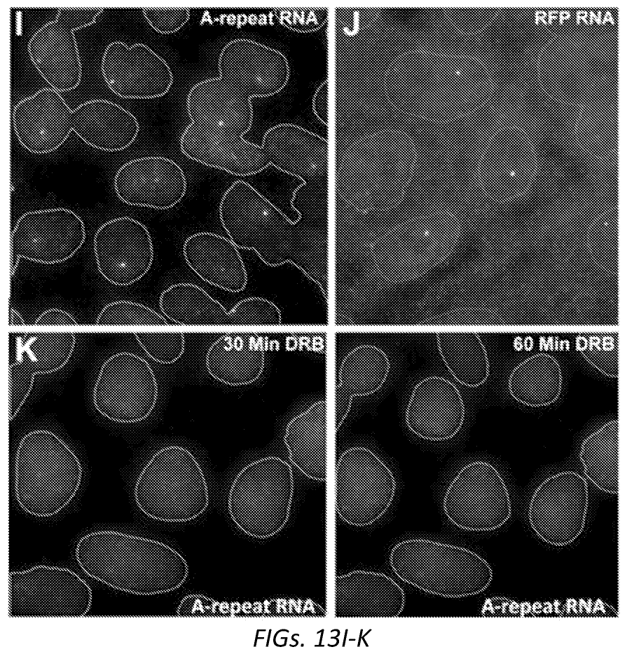

Described herein are methods using an XIST A-repeat mini gene of up to about 5 Kb; these smaller trans genes are not only more readily “deliverable” (e.g. by AAV vectors etc.), but can also be used to repress a duplicated chromosomal region without spreading broadly and silencing normal genes across a whole chromosome, to provide more local repression. These compositions and methods that make use of XIST ‘minigenes”, truncated and patch-work versions of the XIST gene with properties distinct from the full-length XIST RNA, can be utilized in distinct ways. As shown herein, the small (450 bp) segment of Xist that contains the “A-repeat domain” has the capability to silence locally one or very few genes at the chromosome integration site, without spreading across the chromosome (See FIGS. 5A-I, FIGS. 13A-K, FIGS. 7A-E, and FIG. 8). The A-repeat minigene is also of an advantageous size that can be readily delivered into cells in vivo (e.g., using AAV vectors or other current delivery methods) and can be more easily manipulated and inserted into a chromosomal target site. In addition, because the Xist A-repeat minigene RNA gene silencing does not depend on generation of an indel (to disrupt the coding sequence or mRNA), it can be inserted anywhere within a gene, such as in an intron. This makes it especially value for any circumstance in which it is advantageous to silence just one allele of a given gene which requires specific targeting to a polymorphism (such as a SNP) within that gene. The A-repeat minigene also shows the ability to repress expression of tightly spaced adjacent genes to the integration site, and hence could repress over-expression of small duplications of a few adjacent genes, as occurs in conditions relating to gene copy number variations (see, e.g., Vulto-van Silfhout et al., Hum Mutat. 2013 December; 34(12):1679-87; Lupski, Environ Mol Mutagen. 2015 June; 56(5):419-36; Harel and Lupski, Clin Genet. 2018 March; 93(3):439-449.

As is known in the literature and described in the examples, full-length XIST RNA triggers recruitment of numerous chromatin-modifying enzymes that induce many changes to the chromomosome, including numerous histone and non-histone modifications; examples of these include ubiquitination of histone H2A, methylation of H3K27, substitution of macroH2A, deacetylation of histone H3 and of H4, binding/recruitment of CIZ-1 matrix protein, enrichment of SAF-A, recruitment of SMCHD1 and several other RNA-binding proteins reported to lead phase separation (Pandya-Jones et al, Nature 587, 145-151 (2020)). It has been widely held that these numerous changes work cooperatively to silence genes on the chromosome, and studies seek to understand which parts of the 17 kb XIST transcript are responsible by deleting small parts from the long transcripts. In mice, deletion of the small (˜450 nt) XIST A-repeat domain (containing 9-50 nt repeats) from the long XIST transcript results in loss of XIST RNA's chromosome silencing activity (Wutz et al., Nat Genet 2002, 30:167-174), and other studies have confirmed that deletion of the A-repeat domain impairs the function of the long XIST transcript. However, the A-repeat is only ˜4% of the XIST RNA and thus was assumed that other domains of XIST RNA are required for its silencing function. Hence, XIST RNA function has been studied by deleting certain fragments from the 17 kb transcript, but generally not by testing individual fragments separately, which were assumed to lack function alone. Prior studies investigating whether the A-repeat domain alone was sufficient for transcriptional silencing of endogenous genes, using non-targeted insertion of constructs into random chromosomal sites (mediated by randomly integrated FRT sites), concludied that “Additional sequences are required for the spread of silencing to endogenous genes on the chromosome.” (Minks et al., 2013). The repression of immediately flanking reporter inserted on the same plasmid in Minks et al. may well occur by transcriptional interference, which is mechanistically different than the epigenetic (chromatin modification) mechanism by which A-repeat sequences repress endogenous genes in the chromosomal region. Transcriptional interference impacts expression of two tightly-juxtaposed loci, and is known to occur in a variety of biological contexts, including effects in studies of transgenes. As summarized by Eszterhas et al., Mol Cell Biol. 2002 January; 22(2):469-79 (2002), “transcriptional interference is the influence, generally suppressive, of one active transcriptional unit on another unit linked in cis”. Hence, the repression of a linked reporter by induction of an adjacent strong promoter (on XIST transgene) would frequently involve transcriptional interference. This contrasts with the repression of endogenous genes up to several megabases distant from the transgene achieved using the present methods (see FIG. 8), which occurs via epigenetic chromatin modification. With transcriptional interference, the repression of gene B by induced expression of gene A is not due to repression by the RNA from gene A. In contrast, we show that the A-repeat RNA repression of gene promoters 100 kb or more away requires histone de-acetylase activity, in keeping with other evidence that the A-repeat sequence is required for full-length XIST RNA to recruit the repressor Spen involved in deacetylation of histone H3 and H4. While it was unanticipated that this fragment could still retain function outside the context of ˜96% of the XIST transcript, our findings indicate it does, and further define specific utility for this function.

In contrast to expectations, the present results revealed that the human A-repeat devoid of other XIST sequences does support silencing of endogenous loci that are about 50 or 100 kb up to about 0.5, 1, 2, 3, 4, or 5 Mb away (i.e., within up to a 10 Mb segment centered on insertion site, referred to herein as the silencing region). The present study tested this in a cell system that provides a better assessment of the function of the A-repeat domain; in the developmentally correct cell system used herein, the full-length XIST RNA showed full chromosome silencing function.

Importantly, the present results show that the A-repeat minigene RNA forms a focal accumulation at that chromosomal region (a region of up to about 10 Mb) but does not spread further across the chromosome, hence in a limited region near the A-minigene transcription site other genes are repressed locallyin the silencing region across the chromosome. Furthermore, we showed the A-repeat minigene can function if inserted into the intron of a gene, and hence can provide allele-specific silencing of the many genes that lack common SNPs in coding sequences.

Thus, the present invention includes use of genomic engineering methods (such as CRISPR/Cas, ZF, TALEN, HDR, or other gene editing method), to insert an “A-repeat domain” minigene to silence a desired region, e.g., a deleterious locus. The XIST A-repeat sequence is inserted into a chromosome, where it will silence the gene into which it is inserted, and adjacent endogenous genes within the silencing region. As shown herein, the A-repeat sequence can be inserted into the intron of a gene and effectively silence the promoter of that gene up to about 5 Mb away. This is important because for many genes, such as APP (which is important in Alzheimer's Disease), there are no common SNPs in coding regions that could be used to create an indel or for specific gene targeting and the A repeat could work from any SNP to silence the gene. In some embodiments, a local chromosome region comprising a number of genes is silenced, up to 10 Mb (i.e., 5 Mb on either side of the insertion site, with the strongest repression 2 Mb on either side of insertion site). In some embodiments, the methods are used for silencing of the Down Syndrome Critical Region, in which the DYRK1A gene resides.

In addition, the present methods can be used as an experimental tool to suppress any gene cluster of interest, not just deleterious genes. Examples of clustered genes might include: homeobox genes, globin genes, major histocompatibility genes, histone genes, olfactory receptor genes, and interferon receptor genes. In addition, any genes with CNVs (genes in copy number variations) can be targeted to test for functional effects of the CNV to determine whether they may be/are pathogenic.

### XIST A-Repeat Minigene Silencing Sequences and Constructs

In the present application, the “A-repeat Minigene” refers to a transgene containing ˜9 and up to about 50, e.g., 6-20, 20-50, 30-50, 6-40, or 6-30 tandem copies of an A repeat as described herein, e.g., comprising a GC-rich core sequence and a T-rich spacer sequence in between, e.g., an about 50 bp A-repeat sequence taken from the 5′ end of the Xist gene regardless of the origin of the sequence, or whether more tandem copies of the 50 bp sequence are present. For example, the present compositions can include, and the present methods can be carried out with, an Xist gene encoding an Xist RNA from humans or another mammal (e.g., a rodent such as a mouse, dog, cat, cow, horse, sheep, goat, or another mammalian or non-mammalian animal). The scientific literature has adopted a loose convention whereby the term is fully capitalized (XIST) when referring to a human sequence but not fully capitalized (Xist) when referring to the murine sequence. That convention is not used here, and either human or non-human sequences may be used as described herein.

The silencing sequences described herein are DNA polypeptides comprising fragments of the A repeat of XIST and in some cases, further comprise consensus motifs for proteins that direct genome structure—e.g. CTCF motif of C-C-(A/T)-(C/G)-(C/T)-A-G-(G/A)-(G/T)-G-G-(C/A)-(G/A)-(C/G) (Kim et al. (2007) Cell, 128(6):P1231-1245) or YY1 consensus motif of G-G-C-G-C-C-A-T-N-T-T or of C-C-G-C-C-A-T-N-T-T (Kim and Kim. (2009) Genomics, 93:152-158). In some embodiments, the silencing sequence comprises a sequence shown herein, e.g., in the Examples below.

An exemplary sequence for an A repeat domain full sequence is as follows:

GAATTTTTCTTTGGAATCATTTTTGGTTGACATCTCTGTTTTTTGTGGA
TCAGTTTTTTACTCTTCCACTCTCTTTTCTATATTTTGCCCATCGGGGC
TGCGGATACCTGGTTTTATTATTTTTTCTTTGCCCAACGGGGCCGTGGA
TACCTGCCTTTTAATTCTTTTTTATTCGCCCATCGGGGCCGCGGATACC
TGCTTTTTATTTTTTTTTCCTTAGCCCATCGGGGTATCGGATACCTGCT
GATTCCCTTCCCCTCTGAACCCCCAACACTCTGGCCCATCGGGGTGACG
GATATCTGCTTTTTAAAAATTTTCTTTTTTTGGCCCATCGGGGCTTCGG
ATACCTGCTTTTTTTTTTTTTATTTTTCCTTGCCCATCGGGGCCTCGGA
TACCTGCTTTAATTTTTGTTTTTCTGGCCCATCGGGGCCGCGGATACCT
GCTTTGATTTTTTTTTTTCATCGCCCATCGGTGCTTTTTATGGATGAAA
AAATGTT

The human A repeat region is composed of 8.5 repeats with high conservation on GC palindromic repeats that can form stems within the repeat unit and can also pair with other repeats. These conserved repeats are flanked by a T rich spacer of different nucleotide range length (see the Clustal analysis below). As shown in the Clustal analysis, there is variation within the units, but they are all functional. For simplification purpose we show a consensus sequence extracted from these repeats using the Benson repeat finder below. In addition, Crooks 2004 conservation motifs (Crooks et al., Genome Res. 2004; 14(6):1188-1190) are shown below and they are more explicit in that they show the degree of representation for each nucleotide. This software only admits analysis of sequences of the same length, therefore here we present the motif for the GC palindromic region and another one where all the repeats were arbitrarily trimmed to 43 nt.

Clustal analysis of pre-defined repeats, length: 494

| | | |
|---|---|---|
| 5 | -CCCCAACACTCTGGCCCATCGGGGTGACGGATATCTGCTTTTTAAAAA----------- | 48 |
| 4 | -TTTTTTTTCCTTAGCCCATCGGGGTATCGGATACCTGCTGATTCCCTTCCCCTCTGAAC | 59 |
| 9 | -TTTTTTTTTCATCGCCCATCGGTGC---------------------------------- | 25 |
| 2 | -TTATTTTTTCTTTGCCCAACGGGGCCGTGGATACCTGCCTTTTAA-------------- | 45 |
| 3 | -TTCTTTTTTATTCGCCCATCGGGGCCGCGGATACCTGCTTTTTAT-------------- | 45 |
| 1 | -TTTTCTATATTTTGCCCATCGGGGCTGCGGATACCTGGTTTTA---------------- | 43 |
| 7 | TTTTATTTTTCCTTGCCCATCGGGGCCTCGGATACCTGCTTTAA---------------- | 44 |
| 6 | -TTTTCTTTTTTTGGCCCATCGGGGCTTCGGATACCTGCTTTTTTTTT------------ | 47 |
| 8 | TTTTTGTTTTTCTGGCCCATCGGGGCCGCGGATACCTGCTTTGATT-------------- | 46 |
| | * ***** *** * | |

### Benson Consensus (Repeat Finder):

TTCGCCCATCGGGGCCGCGGATACCTGCTTTTTATTTTTTTTTC

2. Crooks, 2004 Analysis of 43 nt Repeat Units Including Some T-Rich Sequence, and Consensus Logo

In some embodiments, the XIST A-repeats comprise a sequence that is at least 80%, 85%, 90%, 95%, or 100% identical to GCCCA[T/A]CGGGG[C/T]N[G/T/A][C/T]GGATA [C/T]CTG, wherein N is any nucleotide, and which retain the ability to form hair-pin loops. Sequence properties of the A-repeats allow it to form structures termed “hairpin loops”, formed by short palindromic sequences that can hybridize to form a double-stranded section of the RNA, which then creates a single-stranded loop of non-complementary sequences. An earlier study that showed that silencing ability of the full-length ˜14 kb mouse Xist transcript is reduced by deletion of the ˜450 bp A-repeat domain also provided some evidence that regions which form hair-pin loops are involved (Wutz with Jaenisch, 2002). For various RNAs, these hair-pin loop structures have been commonly shown to bind proteins, such as Spen, which binds A-repeat RNA and recruits the histone deacetylases that repress gene transcription. Hence, the primary sequence for a ncRNA can vary provided certain aspects of structure are kept. As indicated in the sequence information below, A-repeats units vary slightly in length but are ˜46 bp and have small changes in the natural sequence, such that each tandem repeat is not identical. However, there is a core sequence feature, characterized by palindromic G and C rich motifs that can form two highly stable hair pin structures; as shown in the figure these well conserved and likely important nucleotides for function. The stem loops can form either by hybridization of complementary sequences within the same repeat or between the tandem repeats. Also, the natural number of repeat units can vary slightly but is generally ˜8.5 (one unit is only partially present). Hence, for the invention described here, it is key that the non-coding RNA sequence preserves these structural properties of the A-repeat RNA to enable its function to recruit repressive factors, particularly histone deacetylases, to chromatin, which represses gene expression. Even when the A-repeat RNA recruits Spen or other chromatin factors that repress transcription of nearby genes, a key feature is that A-repeat RNA does not repress its own transcription, by mechanisms that are not understood.

Calculations of sequence similarity or sequence identity between sequences (the terms are used interchangeably herein) can be performed as follows. To determine the percent identity of two amino acid sequences, or of two nucleic acid sequences, the sequences can be aligned for optimal comparison purposes (e.g., gaps can be introduced in one or both of a first and a second amino acid or nucleic acid sequence for optimal alignment and non-homologous sequences can be disregarded for comparison purposes. The comparison of sequences and determination of percent identity between two sequences can be accomplished using a mathematical algorithm. In some embodiments, the percent identity between two amino acid sequences is determined using the Needleman and Wunsch, (1970, J. Mol. Biol. 48: 444-453) algorithm which has been incorporated into the GAP program in the GCG software package, using either a BLOSUM 62 matrix or a PAM250 matrix, and a gap weight of 16, 14, 12, 10, 8, 6, or 4 and a length weight of 1, 2, 3, 4, 5, or 6.0), using a PAM120 weight residue table, a gap length penalty of 12 and a gap penalty of 4.

### Additional Domains

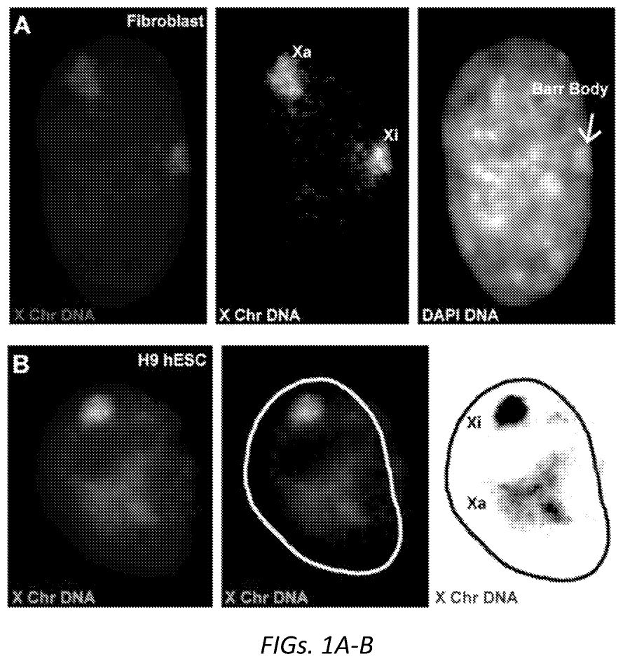

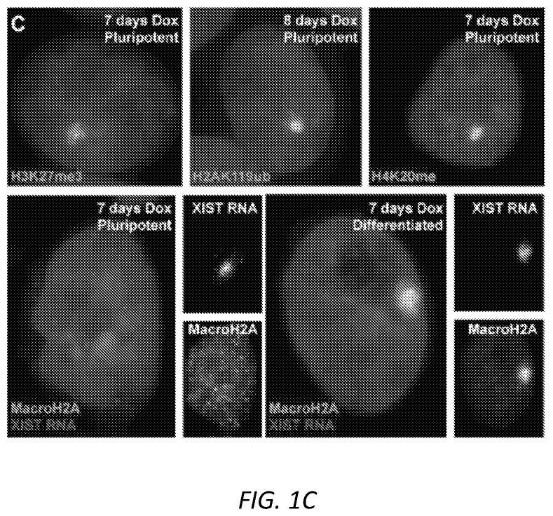

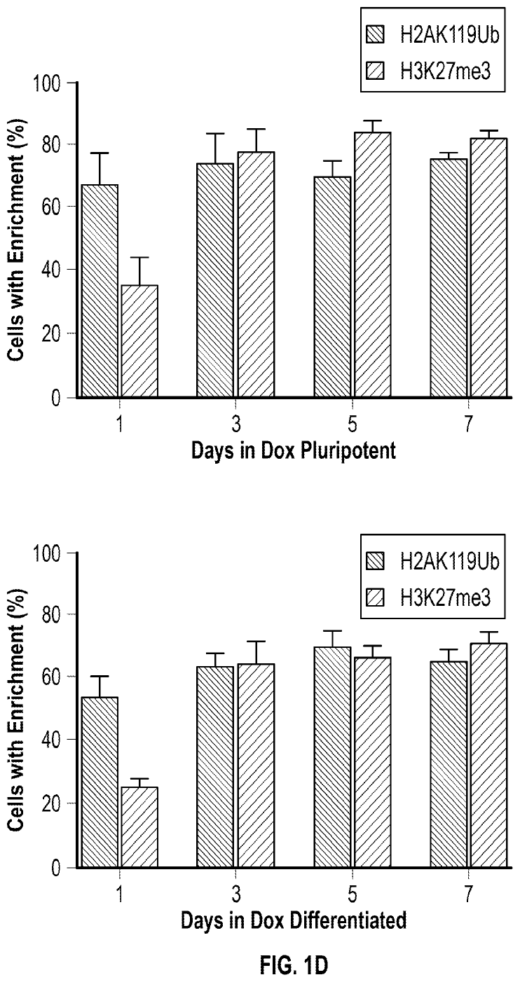

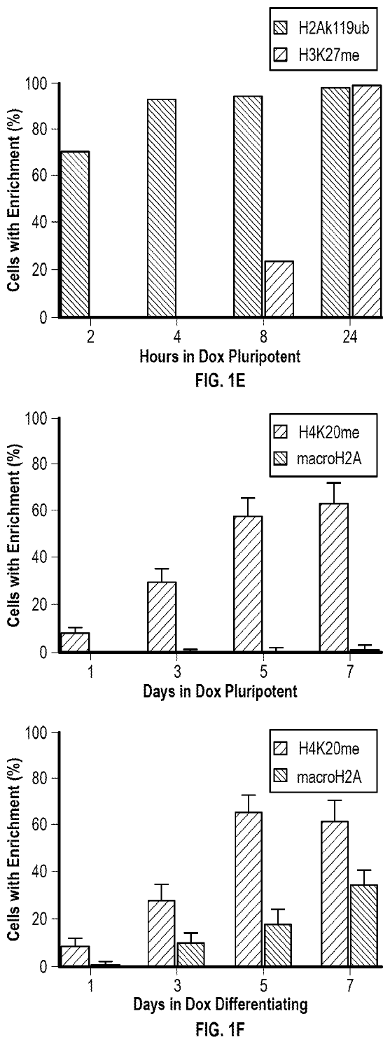

In some embodiments, other portions of XIST can also be included, e.g., one or more of the F, B, C, and/or D repeats, without compromising the localized nature of the silencing to the specific local region of interest. As shown in FIG. 7A, we have generated modifications of the 450 bp A-repeat minigene, all targeted to the DYRK1A intron site in the Down Syndrome Critical Region of Chr21, and RNA from all three minigenes is localized to a small focal region of the nuclear chromosome, rather than spreading across a larger nuclear territory, as does full-length XIST RNA (See FIGS. 1A-F, 2A-J, 9A-H and 10A-E). FIG. 8 shows RNA seq data demonstrating that numerous genes in a small chromosomal region are repressed, with the most significantly repressed genes in a 5 Mb region of the Down syndrome critical region. These results suggest that the additional 2.5 kb minigene containing additional XIST fragments behaves similarly to the 450 bp A-repeat minigene; repressive function may be enhanced to some degree. Other results suggest that doubling the number of A-repeat monomers from 9 to about 18 may also enhance the level or breadth of silencing in the local region. Hence, addition of other sequence elements to the minimal A-repeat minigene may be used to modulate desirable properties, such as epigenetic alterations (e.g., H3K27 methylation) rendering the silent state less readily reversible by triggering secondary chromatin modifications at the targeted chromosomal locus.

In some embodiments, no other portions of XIST can also be included, e.g., none of the F, B, C, and/or D repeats.

In the nucleic acid constructs described herein the silencing sequences can be linked to at least one regulatory sequence (i.e., a regulatory sequence that promotes expression of the silencing RNA, and a regulatory sequence that promotes expression of a selectable marker, if any). More specifically, the regulatory sequence can include a promoter, which may be constitutively active, inducible, tissue-specific, or a developmental stage-specific promoter. For example, the transgene can use an endogenous promoter if it is targeted to the 5′ UTR, or can include its own promoter if targeted to an intron. The promoter can be chosen depending of the cell type of interest. Enhancers and polyadenylation sequences can also be included.

The construct elements as described here may be variants of naturally occurring DNA sequences. Preferably, any construct element (e.g., a silencing sequence, other non-coding, silencing RNA, or a targeting element) includes a nucleotide sequence that is at least 80% identical to its corresponding naturally occurring sequence (its reference sequence, e.g., an Xist coding region, a human Chr 21 sequence, or any duplicated or translocated genomic sequence). More preferably, the silencing sequence or the sequence of a targeting element is at least 90%, 91%, 92%, 93%, 94%, 95%, 96%, 97%, 98%, 99% identical to its reference sequence.

As used herein, “% identity” of two nucleic acid sequences is determined using the algorithm of Karlin and Altschul (Proc. Natl. Acad. Sci. USA, 87:2264-2268, 1990), modified as in Karlin and Altschul, Proc. Natl. Acad. Sci. USA, 90:5873-5877, 1993). Such an algorithm is incorporated into the NBLAST and XBLAST programs of Altschul et al. (J. Mol. Biol. 215:403-410, 1990). BLAST nucleotide searches are performed with the NBLAST program, score=100, wordlength=12. BLAST protein searches are performed with the XBLAST program, score=50, wordlength=3. To obtain gapped alignment for comparison purposes GappedBLAST is utilized as described in Altschul et al. (Nucl. Acids Res., 25:3389-3402, 1997). When utilizing BLAST and GappedBLAST programs the default parameters of the respective programs (e.g., XBLAST and NBLAST) are used to obtain nucleotide sequences homologous to a nucleic acid molecule as described herein.

### Integration of the Targeting Constructs

In some embodiments, the present methods can include the use of targeting constructs including a sequence that enhances or facilitates non-homologous end joining or homologous recombination—e.g., a zinc finger nuclease, TALEN, or CRISPR/Cas—to promote the insertion of a silencing sequence as described herein into the genome of a cell at a desired location. In addition to zinc fingers, TALENs, and CRISPR/Cas, other methods can be used to promote site-specific integration of a minigene as described herein into the genome of a cell. Such methods can include ObLiGaRe nonhomologous end-joining in vivo capture (Yamamoto et al., G3 (Bethesda). 2015 September; 5(9): 1843-1847); prime editing (Anzolone et al., Nature. 2019 December; 576(7785): 149-157); twin prime editing (Anzolone et al., Nat Biotechnol. 2022 May; 40(5): 731-740); Find and cut-and-transfer (FiCAT) mammalian genome engineering (Pallares-Masmitji et al., Nature Communications volume 12, Article number: 7071 (2021)); transposons (Ding et al., Cell. 2005 Aug. 12; 122(3):473-83); RNA-guided retargeting of Sleeping Beauty transposition (Kovac et al., (2020) eLife 9:e53868); Cre-Lox and FLP/FRT recombinases (Branda and Dymecki, Dev Cell. 2004 January; 6(1):7-28); homology-independent targeted insertion (HITI) (Suzuki and Belmonte, Journal of Human Genetics 63: 157-164 (2018)); programmable addition via site-specific targeting elements (PASTE) (Yamall et al., Nat Biotechnol (2022). doi.org/10.1038/s41587-022-01527-4).

In some embodiments, the sequence is inserted into the genome at a SNP or other sequence (e.g., CNV) that is present on one allele, i.e., on an allele at a point in the genome that is within the silencing region (i.e., about 50 or 100 kb up to about 0.5, 1, 2, 3, 4, or 5 MB away) from the promoter of a target gene to be silenced.

As would be understood in the art, the term “recombination” is used to indicate the process by which genetic material at a given locus is modified as a consequence of an interaction with other genetic material. Homologous recombination indicates that recombination has occurred as a consequence of interaction between segments of genetic material that are homologous or identical. In contrast, “non-homologous” recombination indicates a recombination occurring as a consequence of the interaction between segments of genetic material that are not homologous (and therefore not identical). Non-homologous end joining (NHEJ) is an example of non-homologous recombination.

The nucleic acid constructs described herein can include targeting sequences or elements (the terms are used interchangeably herein) that promote sequence specific integration of an Xist minigene into a specific genomic region (e.g., by homologous recombination). Methods for achieving site-specific integration by ends-in or ends-out targeting are known in the art and in the nucleic acid constructs of this invention, the targeting elements are selected and oriented with respect to the silencing sequence according to whether ends-in or ends-out targeting is desired. In certain embodiments, two targeting elements flank the silencing sequence.

A targeting sequence or element may vary in size. In certain embodiments, a targeting element may be at least or about 50, 100, 200, 300, 400, 500, 600, 700, 800, 900, 1000, 2000 bp in length (or any integer value in between, or any range with these specific values as endpoints, e.g., 50-500 or 50-1000). In certain embodiments, a targeting element is homologous to a sequence that occurs naturally in a trisomic and/or translocated chromosomal region, including a polymorphic sequence which may be present on just one of the homologous chromosomes.

### Zinc Finger Nuclease- and TALE-Dependent Targeting

Zinc finger domains and TALENs can recognize and target highly specific chromosomal sequences to facilitate targeted integration of the transgene. In some embodiments, targeting the present silencing constructs to a specific locus can be facilitated by introducing a chimeric zinc finger nuclease (ZFN), i.e., a DNA-cleavage domain (nuclease) operatively linked to a DNA-binding domain including at least one zinc finger, into a cell. Typically the DNA-binding domain is at the N-terminus of the chimeric protein molecule, and the DNA-cleavage domain is located at the C-terminus of the molecule. These nucleases exploit endogenous cellular mechanisms for homologous recombination and repair of double stranded breaks in genetic material. ZFNs can be used to target a wide variety of endogenous nucleic acid sequences in a cell or organism. The present compositions can include cleavage vectors that target a ZFN to a target region, and the methods include transfection or transformation of a host cell or organism by introducing a cleavage vector encoding a ZFN (e.g., a chimeric ZFN), or by introducing directly into the cell the mRNA that encodes the recombinant zinc finger nuclease, or the protein for the ZFN itself. One can then identify a resulting cell or organism in which a selected endogenous DNA sequence is cleaved and exhibits a mutation or DNA break at a specific site, into which the transgene will become integrated.

The ZFN can include multiple (e.g., at least three (e.g., 3, 4, 5, 6, 7, 8, 9 or more)) zinc fingers in order to improve its target specificity. The zinc finger domain can be derived from any class or type of zinc finger. For example, the zinc finger domain can include the Cys2His2 type of zinc finger that is very generally represented, for example, by the zinc finger transcription factors TFIIIA or Sp1. In a preferred embodiment, the zinc finger domain comprises three Cys2His2 type zinc fingers.

To target genetic recombination or mutation, two 9 bp zinc finger DNA recognition sequences are identified in the host DNA. These recognition sites will be in an inverted orientation with respect to one another and separated by about 6 bp of DNA. ZFNs are then generated by designing and producing zinc finger combinations that bind DNA specifically at the target locus, and then linking the zinc fingers to a cleavage domain of a Type II restriction enzyme.

A silencing sequence flanked by sequences (typically 400 bp-5 kb in length) homologous to the desired site of integration can be inserted (e.g., by homologous recombination) into the site cleaved by the endonuclease, thereby achieving a targeted insertion. The silencing sequence may be referred to as “donor” nucleic acid or DNA.

In some embodiments, the cleavage vector includes a transcription activator-like effector nuclease (TALEN). TALENs function in a manner somewhat similar to ZFNs, in that they can be used to induce sequence-specific cleavage; see, e.g., Miller et al., Nat Biotechnol. 2011 February; 29(2):143-8. Hockemeyer et al., Nat Biotechnol. 29(8):731-4 (2011); Moscou et al., 2009, Science 326:1501; Boch et al., 2009, Science 326:1509-1512. Methods are known in the art for designing TALENs, see, e.g., Rayon et al., Nature Biotechnology 30:460-465 (2012).

### CRIPR Cas9-Mediated Targeting

The present methods include the delivery of nucleic acids encoding a CRISPR gene editing complex. The gene editing complex includes a Cas9 editing enzyme and one or more guide RNAs directing the editing enzyme to a specific genomic locus/loci.

### Guide RNAs Directing the Editing Enzyme to a Specific Genomic Locus/Loci

The gene editing complex also includes guide RNAs directing the editing enzyme to a specific genomic locus, i.e., comprising a sequence that is complementary to the sequence of a nucleic acid encoding the specific genomic locus, and that include a PAM sequence that is targetable by the co-administered Cas9 editing enzyme. Exemplary loci are described herein, see, e.g., Table 1.

### Cas9 Editing Enzymes

The methods include the delivery of Cas9 editing enzymes to the cells. The editing enzymes can include one or more of Streptococcus thermophilus (ST) Cas9 (StCas9); Treponema denticola (TD) (TdCas9); Streptococcus pyogenes (SP) (SpCas9); Staphylococcus aureus (SA) Cas9 (SaCas9); or Neisseria haracteriza (NM) Cas9 (NmCas9), as well as variants thereof that are at least 80%, 85%, 90%, 95%, 99% or 100% identical thereto that retain at least one function of the parent protein, e.g., the ability to complex with a gRNA, bind to target DNA specified by the gRNA, and alter the sequence of the target DNA. Variants include the SpCas9 D1135E variant; SpCas9 VRER variant; SpCas9 EQR variant; the SpRY variant; and the SpCas9 VQR variant, among others.

The sequences of the Cas9s are known in the art; see, e.g., Kleinstiver et al., Nature. 2015 Jul. 23; 523(7561): 481-485; WO 2016/141224; U.S. Pat. No. 9,512,446; US-2014-0295557; WO 2014/204578; and WO 2014/144761. The methods can also include the use of the other previously described variants of the SpCas9 platform (e.g., truncated sgRNAs (Tsai et al., Nat Biotechnol 33, 187-197 (2015); Fu et al., Nat Biotechnol 32, 279-284 (2014)), nickase mutations (Mali et al., Nat Biotechnol 31, 833-838 (2013); Ran et al., Cell 154, 1380-1389 (2013)), FokI-dCas9 fusions (Guilinger et al., Nat Biotechnol 32, 577-582 (2014); Tsai et al., Nat Biotechnol 32, 569-576 (2014); WO2014144288).

See also Hou, Z. et al. Efficient genome engineering in human pluripotent stem cells using Cas9 from Neisseria haracteriza. Proc Natl Acad Sci USA (2013); Fonfara, I. et al. Phylogeny of Cas9 determines functional exchangeability of dual-RNA and Cas9 among orthologous type II CRISPR-Cas systems. Nucleic Acids Res 42, 2577-2590 (2014); Esvelt, K. M. et al. Orthogonal Cas9 proteins for RNA-guided gene regulation and editing. Nat Methods 10, 1116-1121 (2013); Cong, L. et al. Multiplex genome engineering using CRISPR/Cas systems. Science 339, 819-823 (2013); Horvath, P. et al. Diversity, activity, and evolution of CRISPR loci in Streptococcus thermophilus. J Bacteriol 190, 1401-1412 (2008).

The Cas9 can be delivered as a purified protein (e.g., a recombinantly produced purified protein, prefolded and optionally complexed with the sgRNA, e.g., as a ribonucleoprotein (RNP)), or as a nucleic acid encoding the Cas9, e.g., an expression construct (e.g., DNA or RNA). Purified Cas9 proteins can be produced using methods known in the art, e.g., expressed in prokaryotic or eukaryotic cells and purified using standard methodology. For example, the methods can include delivering the Cas9 protein and guide RNA together, e.g., as a complex. For example, the Cas9 and gRNA can be can be overexpressed in a host cell and purified, then complexed with the guide RNA (e.g., in a test tube) to form a ribonucleoprotein (RNP), and delivered to cells. In some embodiments, the Cas9 can be expressed in and purified from bacteria through the use of bacterial Cas9 expression plasmids. For example, His-tagged Cas9 proteins can be expressed in bacterial cells and then purified using nickel affinity chromatography. The RNPs can be delivered to the cells in vivo or in vitro, e.g., using lipid-mediated transfection or electroporation. See, e.g., Liang et al., Journal of biotechnology 208 (2015): 44-53; Zuris et al. Nature biotechnology 33.1 (2015): 73-80; Kim et al. Genome research 24.6 (2014): 1012-1019. Efficiency of protein delivery can be enhanced, e.g., using electroporation (see, e.g., Wang et al., Journal of Genetics and Genomics 43(5):319-327 (2016)); cationic or lipophilic carriers (see, e.g., Yu et al., Biotechnol Lett. 2016; 38: 919-929; Zuris et al., Nat Biotechnol. 33(1):73-80 (2015)); PNA/DNA-containing NPs (see Ricciardi et al., Nat Commun 9, 2481 (2018); or even lentiviral packaging particles (see, e.g., Choi et al., Gene Therapy 23, 627-633 (2016)). Methods of delivering nucleic acids encoding Cas9 are known in the art and described herein.

### Selection Markers

In addition, the nucleic acids may contain a marker for the selection of transfected cells (for instance, a drug resistance gene for selection by a drug such as neomycin, hygromycin, and G418). Such vectors include pMAM, pDR2, pBK-RSV, pBK-CMV, pOPRSV, pOP13, and so on. More generally, the term “marker” refers to a gene or sequence whose presence or absence conveys a detectable phenotype to the host cell or organism. Various types of markers include, but are not limited to, selection markers, screening markers, and molecular markers. Selection markers are usually genes that can be expressed to convey a phenotype that makes an organism resistant or susceptible to a specific set of environmental conditions. Screening markers can also convey a phenotype that is a readily observable and distinguishable trait, such as green fluorescent protein (GFP), GUS or β-galactosidase. Molecular markers are, for example, sequence features that can be uniquely identified by oligonucleotide probing, for example RFLP (restriction fragment length polymorphism), or SSR markers (simple sequence repeat). To amplify the gene copies in host cell lines, the expression vector may include an aminoglycoside transferase (APH) gene, thymidine kinase (TK) gene, E. coli xanthine guanine phosphoribosyl transferase (Ecogpt) gene, dihydrofolate reductase (dhfr) gene, and such as a selective marker.

Expression of the selection marker can be driven by the same regulatory elements (e.g., promoters) as the silencing sequence, or can be driven by a separate regulatory element.

### Vectors

The various sequences, including the silencing sequence and the targeting construct (e.g., ZFN, TALE, or CRISPR-CAS/gRNA), can be introduced into a host cell on one or more expression vectors (e.g., on separate vectors or separate types of vectors at the same time or sequentially), or can be introduced as naked nucleic acids (e.g., silencing sequence DNA and mRNA transcripts and RNA guide RNA), or as protein/nucleic acid complexes (e.g., Cas/gRNA ribonucleoproteins and separate silencing sequence DNA). Methods for introducing the various nucleic acids, constructs, and vectors are discussed further below and are well known in the art.

Retrovirus vectors and adeno-associated virus vectors can be used as a recombinant gene delivery system for the transfer of exogenous genes. These vectors provide efficient delivery of genes into cells, and the transferred nucleic acids are stably integrated into the chromosomal DNA of the host cell. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, Blood 76:271 (1990)). A replication defective retrovirus can be packaged into virions, which can be used to infect a target cell through the use of a helper virus by standard techniques. Protocols for producing recombinant retroviruses and for infecting cells in vitro with such viruses can be found in Ausubel, et al., eds., Current Protocols in Molecular Biology, Greene Publishing Associates, (1989), Sections 9.10-9.14, and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include ΨCrip, ΨCre, Ψ2 and ΨAm. Retroviruses have been used to introduce a variety of genes into many different cell types, including epithelial cells, in vitro (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. Nos. 4,868,116; 4,980,286; PCT Application WO 89/07136; PCT Application WO 89/02468; PCT Application WO 89/05345; and PCT Application WO 92/07573).

Other viral vectors may be employed as expression constructs in the present invention. Vectors derived from, for example, vaccinia virus, adeno-associated virus (AAV, e.g., MV), or herpes virus may be employed. Extensive literature is available regarding the construction and use of viral vectors. For example, see Miller et al. (Nature Biotechnol. 24:1022-1026, 2006) for information regarding adeno associated viruses. The AAV can be any AAV serotype, including any derivative or pseudotype (e.g., AAV1, 2, 3, 4, 5, 6, 7, 8, 9, 10, 2/1, 2/5, 2/8, 2/9, 3/1, 3/5, 3/8, or 3/9). As used herein, the serotype of an rAAV vector or an rAAV particle refers to the serotype of the capsid proteins of the recombinant virus. In some embodiments, the rAAV particle is rAAV5. In some embodiments, the rAAV particle is rAAV9 or a derivative thereof such as AAV-PHP.B or AAV-PHP.eB. Non-limiting examples of derivatives and pseudotypes include AAVrh.10, rAAV2/1, rAAV2/5, rAAV2/8, rAAV2/9, AAV2-AAV3 hybrid, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y73IF), AAV2.5T, AAV-HAE1/2, AAV clone 32/83, AAVShH10, AAV2 (Y→F), AAV8 (Y733F), AAV2.15, AAV2.4, AAVM41, and AAVr3.45. AAV serotypes and derivatives/pseudotypes, and methods of producing such are known in the art (see, e.g., Mol Ther. 2012 April; 20(4):699-708). In some embodiments, the rAAV particle is a pseudotyped rAAV particle, which comprises (a) an rAAV vector comprising ITRs from one serotype (e.g., AAV2, AAV3) and (b) a capsid comprised of capsid proteins derived from another serotype (e.g., AAV1, AAV2, AAV3, AAV4, AAV5, AAV6, AAV7, AAV8, AAV9, or AAV10). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

Defective hepatitis B viruses can also be used for transformation of host cells. In vitro studies show that the virus can retain the ability for helper-dependent packaging and reverse transcription despite the deletion of up to 80% of its genome. Potentially large portions of the viral genome can be replaced with foreign genetic material. The hepatotropism and persistence (integration) are particularly attractive properties for liver-directed gene transfer. The chloramphenicol acetyltransferase (CAT) gene has been successfully introduced into duck hepatitis B virus genome in the place of the viral polymerase, surface, and pre-surface coding sequences. The defective virus was cotransfected with wild-type virus into an avian hepatoma cell line, and culture media containing high titers of the recombinant virus were used to infect primary duckling hepatocytes. Stable CAT gene expression was subsequently detected.

Expression constructs can be administered in any effective carrier, e.g., any formulation or composition capable of effectively delivering the component gene to cells. Approaches include insertion of the gene in viral vectors, including recombinant retroviruses, adenovirus, adeno-associated virus, lentivirus, and herpes simplex virus-1, or recombinant bacterial or eukaryotic plasmids. Viral vectors transfect cells directly; plasmid DNA can be delivered naked or with the help of, for example, nanoparticles (e.g., using PBAE (poly(β-amino ester), C320 (see, e.g., Eltoukhy et al., Biomaterials 33, 3594-3603 (2012); zugates et al., Mol Ther. 2007 July; 15(7):1306-12), cationic liposomes (lipofectamine) or derivatized (e.g., antibody conjugated), polylysine conjugates, artificial viral envelopes or other such intracellular carriers, as well as direct injection of the gene construct or CaPO₄ precipitation.

In certain embodiments, the oligo- or polynucleotides and/or expression vectors containing silencing sequences and/or ZFN, TALE, CRISPR-CAS/gRNA may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. Also contemplated are cationic lipid-nucleic acid complexes, such as lipofectamine-nucleic acid complexes. Lipids and liposomes suitable for use in delivering the present constructs and vectors can be obtained from commercial sources or made by methods known in the art.

### Transformation

Transformation can be carried out by a variety of known techniques that depend on the particular requirements of each cell or organism. Such techniques have been worked out for a number of organisms and cells and are readily adaptable. Stable transformation involves DNA entry into cells and into the cell nucleus. For example, transformation can be carried out in culture, followed by selection for transformants and regeneration of the transformants. Methods often used for transferring DNA or RNA into cells include forming DNA or RNA complexes with cationic lipids, liposomes or other carrier materials, micro-injection, particle gun bombardment, electroporation, and incorporating transforming DNA or RNA into virus vectors.

A preferred approach for introduction of nucleic acid into a cell is by use of a viral vector containing nucleic acid, e.g., a cDNA. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells that have taken up viral vector nucleic acid.

Direct microinjection of DNA into various cells, including egg or embryo cells, has also been employed effectively for transforming many species. In the mouse, the existence of pluripotent embryonic stem (ES) cells that can be cultured in vitro has been exploited to generate transformed mice. The ES cells can be transformed in culture, then micro-injected into mouse blastocysts, where they integrate into the developing embryo and ultimately generate germline chimeras. By interbreeding heterozygous siblings, homozygous animals carrying the desired gene can be obtained.

### Pharmaceutical Compositions, RNAs, and Cells

Also provided herein are compositions (e.g., pharmaceutically acceptable compositions) that include the proteins, nucleic acids, constructs or vectors described herein. Various combinations of the proteins, nucleic acids, constructs and vectors described herein can be formulated as pharmaceutical compositions.

Also within the scope of the present disclosure are RNAs and proteins encoded by the vector and compositions that include them (e.g., lyophilized preparations or solutions, including pharmaceutically acceptable solutions or other pharmaceutical formulations), and methods of use thereof.

In another embodiment, described herein are cells that include the nucleic acid constructs, vectors (e.g., an adeno associated vector), and compositions described herein. The cell can be isolated in the sense that it can be a cell within an environment other than that in which it normally resides (e.g., the cell can be one that is removed from the organism in which it originated). The cell can be a germ cell, a stem cell (e.g., an embryonic stem cell, an adult stem cell, or an induced pluripotent stem cell (iPS cell or IPSC)), or a precursor cell. Where adult stem cells are used, the cell can be a hematopoietic stem cell, a cardiac muscle stem cell, a mesenchymal stem cell, or a neural stem cell (e.g., a neural progenitor cell). The cell can also be a differentiated cell (e.g., a fibroblast or neuron).

### Methods of Treatment

The present methods can be used to silence one or more alleles to produce a therapeutic effect, in any circumstance in which the long-term silencing of an allele or small gene cluster is desirable, in some cases without disrupting expression and normal function of the other allele. The methods can include obtaining sequence of a subject's genome within the silencing region of (i.e., about 50 or 100 kb up to about 0.5, 1, 2, 3, or 4 MB away) from a promoter of one or more alleles of a target gene in a subject. In some embodiments, the methods include identifying a SNP or other unique sequence (e.g., a junction site in the case of a duplication or transversion) associated with only one of the alleles of the target gene (in cases where only one allele is desired to be silenced) or a common sequence in all of the alleles of the target gene (in cases where all of the alleles are desired to be silenced). The methods include contacting cells of the subject with a silencing sequence and a targeting construct that directs insertion of the silencing sequence into the SNP or common sequence. Insertion of the silencing sequence then results in downregulation or cessation of expression of the target gene and other genes in the silencing region.

For example, Down Syndrome (DS), or Trisomy 21, is the most common chromosomal disorder in newborns and is the leading genetic cause of intellectual disability in children, affecting approximately 300,000 people (and their families) in the U.S. and millions worldwide. In addition to consistent intellectual disability, autism, and common speech deficits, individuals with DS also have high risk of congenital cardiac defects, leukemia and other medical challenges. Unfortunately, as the average lifespan of DS patients has increased to 60 years, it became clear that Trisomy 21 is a form of early-onset Alzheimer's Disease (AD). All DS individuals develop amyloid plaques as early as adolescence and ˜80% develop clinical AD dementia by age 60 (Mann and Esiri, 1989; Wisniewski et al., 1985a; Zigman et al., 1996). It is widely accepted that this is due primarily to trisomy for the APP gene on Chr21, as patients with APP gene duplication but without trisomy 21 also develop early-onset AD (Cabrejo et al., 2006; Kasuga et al., 2009; Rovelet-Lecrux et al., 2006, 2007; Sleegers et al., 2006). APP is an essential component of all Alzheimer pathogenesis, and its triplication causes amyloid plaques to form in the brains of essentially all individuals with DS at a very early age and Alzheimer dementia to develop in over 80%, 20-30 years earlier than the non DS population. Hence, there is a compelling need to find a solution for people, including those with DS or APP gene duplication, to avoid the onset of AD.

However, eliminating expression of all of the APP genes in an individual is not desirable, so allele-specific silencing is required. Since there is no common SNP in a coding region of the APP gene that can be targeted to create an indel and frame-shift, the methods described herein can be used to reduce the APP locus to disomy (normal two copies), by inserting a silencing sequence described herein at a SNP within the silencing region, i.e., about 50 or 100 kb up to about 0.5, 1, 2, 3, or 4 MB away from the promoter of one of the APP alleles. It is known that silencing one APP allele would greatly reduce the risk or slow the development of AD in most of the 300,000 individuals living with DS in the U.S. (and six million worldwide).

In addition, since APP is expressed early in development and highly in neural tissue, it is possible that reducing APP to normal levels could have beneficial effects on cognitive disabilities of individuals with Down Syndrome, who often score as more severely impacted as adults, suggesting progressive cognitive decline after childhood. Previous studies have shown that expression of full-length XIST fully corrects trisomy 21 dosage in neural cells, and the treated neural cells retain epigenetic plasticity to initiate chromosome-wide repression; dosage correction by XIST was also shown to promote (delayed) differentiation of trisomic NSCs to neurons (Czerminski and Lawrence, Dev Cell. 2020 Feb. 10; 52(3): 294-308.e3).

Furthermore, Trisomy 21 confers hematopoietic complications including a 500-fold greater incidence of acute megakaryocytic leukemia (AMKL) and a ˜20-fold greater risk for acute lymphoblastic leukemia (ALL). Subjects with DS have increased susceptibility to viral infections and chronic inflammation that may contribute to cognitive impairment and decline. Trisomy 21 promotes an excess CD43+ progenitors, but not the earlier CD34+ hemogenic endothelium population. Bone marrow transplantation of genetically modified hematopoietic stem cells (HSC) has been actively pursued for clinical applications, and cord blood could serve as an accessible source of HSCs for all DS newborns. Silencing one of the chr21 from pluripotency using a full length XIST targeted to chr21 prevents development of DS hematopoietic cell pathologies in vitro, including the over-production of megakaryocytes and erythrocytes. See Chiang et al., Nat Commun. 2018 Dec. 5; 9(1):5180. Since the present A-repeat minigenes are shown to have silencing ability for important regions of chromosome 21 and are small enough to fit in current delivery vectors, the present methods can also be used to silence clusters of genes most strongly implicated for DS phenotypes, including the APP gene; DYRK1A and nearby genes (e.g., DYRK1A, DSCR3 (VPS26C), TTC3, PIGP, HLCS, RCAN1, CBR1, DONSON, ETS2, PSMG1, and MX1, and optionally BACE2, IFNAR1, IFNGR2, IFNAR2, and IL1) in the Down syndrome critical region; and the interferon gene cluster (Sullivan et al., Elife. 2016 Jul. 29; 5:e16220).

This approach may also have relevance to AD in the general population. Reducing the APP gene expression and “amyloid load” that is central to developing AD could be beneficial to many in the aging human population more generally, particularly those at higher risk for AD (e.g. such as with APOE4 risk allele). It is a reasonable possibility that sustained repression of one APP allele in aging individuals may be beneficial to the non-DS population, 20-25% of whom will get Alzheimer's dementia if they live into their 80s and 90s.

Current strategies to achieve sustained reduction in expression of a desired protein often rely on creating an indel in the coding region of the gene using CRISPR/Cas9. However, using this approach in the APP gene generated many trisomy 21 cells in which all three alleles of APP were disrupted, resulting in no APP protein. Sequence analysis showed that indels of different sizes occurred at all three alleles, creating a deleterious monosomy. In some cells, the indels deleted more of the exon or the whole exon creating an aberrant truncated protein, whereas a deletion in an intronic sequence does not pose the same risk. Thus it is advantageous that A-repeat minigenes can be designed to target into a SNP in an intron that is heterozygous in the cells to be targeted, as shown here for the APP gene. Common SNPs that are present in a large fraction of the population are far more prevalent in non-coding sequences and many genes lack common SNPs in the coding region, as is the case for APP. To overcome this, Table 1 provides a list of common SNPS in APP non-coding regions that would enable allele-specific insertion of the transgene.

TABLE 1
SNPs in APP on human Chr21

| Chrom Start | Chrom End | name | Ref NCBI | Ref UCSC | Observed | class | func | Loc Type | alleles |
|---|---|---|---|---|---|---|---|---|---|
| 26136136 | 26136137 | rs416524 | G | G | A/G | single | intron | Exact | A, G, |
| 26131209 | 26131210 | rs8131895 | C | C | A/C | single | intron | Exact | A, C, |
| 26130150 | 26130151 | rs2830076 | T | T | C/T | single | intron | Exact | C, T, |
| 26119685 | 26119685 | rs5843212 | — | — | —/AC | Insertion | intron | Between | —, AC, |
| 26123864 | 26123865 | rs2234990 | T | T | A/T | single | intron | exact | A, T, |
| 26121884 | 26121885 | rs2830066 | C | C | C/T | single | intron | exact | C, T, |
| 26130124 | 26130125 | rs2830075 | C | C | C/T | single | intron | exact | C, T |
| 26126977 | 26126977 | rs34453423 | — | — | —/A | Insertion | intron | Between | —, A, |
| 26124349 | 26124350 | rs2830068 | T | T | C/T | single | intron | exact | C, T, |
| 26135331 | 26135332 | rs2830081 | A | A | A/C | single | intron | exact | A, C |
| 26134651 | 26134652 | rs13046704 | G | G | C/G | single | intron | exact | C, G, |
| 26128824 | 26128825 | rs2830073 | T | T | C/T | single | intron | exact | C, T, |
| 26132523 | 26132524 | rs11356038 | A | A | —/A | deletion | intron | exact | —, A, |
| 26135741 | 26135742 | rs432766 | T | T | C/T | single | intron | exact | C, T |
| 26130405 | 26130406 | rs2830077 | C | C | A/C | single | intron | exact | A, C |
| 26127199 | 26127200 | rs35029493 | A | A | —/A | deletion | intron | exact | —, A, |
| 26128342 | 26128343 | rs2830072 | T | T | G/T | single | intron | exact | G, T, |
| 26129210 | 26129211 | rs11910723 | G | G | A/C | single | intron | exact | A, G, |
| 26118884 | 26118885 | rs5843211 | A | A | —/A | deletion | intron | exact | —, A, |

Other conditions that can be treated with these methods include chromosomal imbalance disorders, such as translocations that produce partial chromosome trisomy such as 9p syndrome (the third most common trisomy at birth) and microduplication disorders such as Charcot-marie Tooth, duplications associated with intellectual or other deficits including autism (such as Ch 22q11 duplication syndrome (22q11.2 dup), Potocki-Lupski Syndrome (17p11.2 dup), and others (see Lupski, Genome Med. 2009 Apr. 24; 1(4):42)). For example, genomic regions of interest can include, but are not limited to, 1q21 microduplication (which is associated with risk of mental retardation and autism spectrum disorder); 2p15p16 microduplication (which is associated with mental retardation); 3q29 microduplication (which is associated with mold to moderate mental retardation); 15q13.1 microduplication (which is associated with mental retardation, schizophrenia, and autism), 15q24 microduplication (which is associated with developmental delay), and others, including 22q11.2 duplication syndrome (1.5 to 3 Mb in length, 1 in 850 low-risk pregnancies); 17p11.2 duplication syndrome (also known as Potocki-Lupski Syndrome, 3.7-Mb); 7q11.23 duplication (1.5-Mb); 16p11.2 duplication syndrome (593 kB), see Goldenberg, Pediatr Ann. 2018 May 1; 47(5):e198-e203.

Single nucleotide polymorphisms (SNPs) or other unique sequences located in the selected genomic region (e.g., in 5′ UTR, intron, or exon of a target gene) can be identified, e.g., from publically available databases (e.g. NCBI Short Genetic Variations database (dbSNP) available at ncbi.nlm.nih.gov/projects/SNP/index.html) or from quantification of alleles (frequency and sequence) present in a population (e.g., subset of patients or population of cells) (see, e.g., Aggeli et al. (2018). Nucleic Acids Res 46(7): e42) or from sequencing of the relevant region of a subject to be treated. If the former, the sequence of the genomic loci in the subject should be determined and heterozygosity confirmed in the case of allele-specific targeting or homozygosity in the case of pan-allelic targeting.

In some embodiments, the following method is used to identify SNPs/Unique sequences:
1. Look for common SNP in data bases like UCSC Genome Browser.
2. SNPs could be in any site of the gene, including an intron or at the 5′end of the non coding region or in neighboring intergenic region.
3. SNPs that are sufficiently common to maximize the chances of heterozygosity in a patient are key. The maximum likelihood of heterozygosity in a given patient is estimated to be for alleles with frequency closest to 50%. This increases the frequency of heterozygosity such that both SNPs are in a patient, and one out of the 3 chromosomes will have a different SNP. For example, a SNP locus #1 with 2 alleles, each with frequency of 0.5, in a patient with 3 chromosomes, then the chance of heterozygosity in a patient would be 75% at SNP locus #1. If a second locus is added with similarly two common alleles, the probability of finding heterozygosity at least one of these two SNP loci would be about 94%. (calculated as 1-(0.75×0.75)).
4. Of SNPs that fit the above criteria for likelihood of heterozygosity, SNPs that would be advantageous for allele specific targeting are prioritized. While SNPs with a single nucleotide change can work, if the SNP involves a two nt change, or there are two SNPs close together (in same haplotype), this would facilitate highly specific targeting reagents.
5. Identify unique sgRNAs where the SNP is in the PAM or seed sequence and prioritize by predicted efficiency.

Guide RNAs can be designed according to known methods in the prior art (e.g., Akcakaya et al. (2018). Nature 561: 416-419; Tycko et al. (2016). 4; 63(3): 355-370). Selected guide RNAs can be synthesized (e.g., by a commercial source such as Sigma) and screened by methods known in the art to select sgRNAs:Cas9 complexes that efficiently and specifically cut the targeted SNP sequence and do not cut the sequence of the other allele.

### XIST A-Repeat Minigenes as Experimental Tool and List of Examples of Duplication/Deletion Syndromes

In addition to potential therapeutic applications, A-repeat minigenes provide an experimental tool to manipulate the expression of genes clustered in a small chromosomal region, which is of interest for many questions in biology. For example, we have made a DS pluripotent stem cell system with an inducible A-repeat minigene that represses genes in the several Mb “Down Syndrome Critical Region” of Chr21, and are using this system to investigate how repressing the extra copy of this region impacts cell pathologies Down syndrome and identify underlying genome-wide expression pathways. Similarly, we will target A-repeat minigenes into one allele of the cluster of four interferon receptor genes (on Chr21) as a recent hypothesis in the field postulates that DS is essentially an interferonopathy, causing many major co-morbidities of Trisomy 21. For other conditions unrelated to DS, such as autoimmune disorders or organ transplant rejection, there is high interest in regulated expression of the clustered interferon receptor genes or the major histocompatibility complex gene clustered closely on Chr6.

The A-repeat minigene invention can be readily applied to essentially any region of any chromosome for research or therapeutic purposes, by simply changing the sequences that target insertion of the A-repeat minigenes to a specific site. In addition to fundamental biology, such as investigating potential functions of non-coding and highly repetitive regions of chromosomes, A-repeat minigenes can address a strong need for a way to investigate which genes or chromosomal regions are dosage sensitive, and to investigate how an expanding plethora of small structural variations impacts cells to cause a variety of developmental and other medical disorders. This experimental approach is applicable to deletion syndromes as well as duplications, because the inducible A-repeat minigene can be targeted to silence in normal cells that is deleted in patient cells, thereby providing a stem cell model of that deletion disorder.

The field has little understanding of what fraction of genes in the genome is dosage sensitive, nor which genes have an effect if present in an extra copy or just one copy. One in 140 births in the USA have an identified chromosomal imbalance, typically recognized because it causes a pathology. In prenatal diagnostic testing, such as amniocentesis, small (˜10 Mb) chromosomal deletions or duplications can be identified by cytogenetics, but the clinician has little way to predict whether or not that change will cause a phenotype or a severe outcome, unless that same region has been previously reported in other patients with a known syndrome. Hence, an investigative tool to modulate expression dosage from specific chromosomal regions could determine if there is impact on genome-wide pathways and development and differentiation of human stem cells in vitro.

A significant genetic cause of autism serves to illustrate that duplication or deletion of the same chromosomal region (Chr16q11.2) can cause the same neurodevelopmental disorder, although the particularly aspects of the syndrome may differ. A-repeat minigenes can be designed for insertion into this region and then used to either repress the duplicated sequences in duplication-patient cells, or, to repress the region in normal cells to mimic the dosage imbalance of deletion patients. Some of the many other examples of duplication or deletion syndromes for which this experimental tool would be valuable are listed in the Table below. Note the size of the regions involved are well within the range which A-repeat minigenes can regulate.

| Syndrome | Frequency | Size of duplication/deletion |
|---|---|---|
| 22q11.2 duplication | 1 in 850 low-risk | 1.5 to 3 Mb in length |
| syndrome | pregnancies | |
| 17p11.2 duplication | | 3.7-Mb |
| syndrome Potocki-Lupski | | |
| syndrome | | |
| 7q11.23 duplication | | 1.5-Mb |
| 16p11.2 duplication | | 593 KB |
| syndrome | | |
| 22q11.2 deletion syndrome | 1 in 1,000 low-risk | 1.5-3 Mb |
| (DiGeorge syndrome, | pregnancies | |
| velocardiofacial syndrome) | | |
| 7q11.23 deletion (Williams | 1 in 10,000 people | 1.5 to 1.78 Mb |
| syndrome) | | |
| 17p11.2 deletion syndrome | 1 in 25,000 | 3.5-Mb deletion (95%) |
| (Smith-Magenis syndrome) | | |
| 16p11.2 deletion syndrome | 3 in 10,000 people, and in | 593-kilobase (kB) deletion |
| | approximately 1% of people | |
| | with a diagnosis of autism | |

### EXAMPLES

The invention is further described in the following examples, which do not limit the scope of the invention described in the claims.

### Example 1

To investigate the interrelationships between spread of XIST RNA and changes to overall architecture, histone modifications, and transcriptional silencing, we examined RNA, DNA and proteins on individual inactivating chromosomes in human iPS cells using molecular cytology.

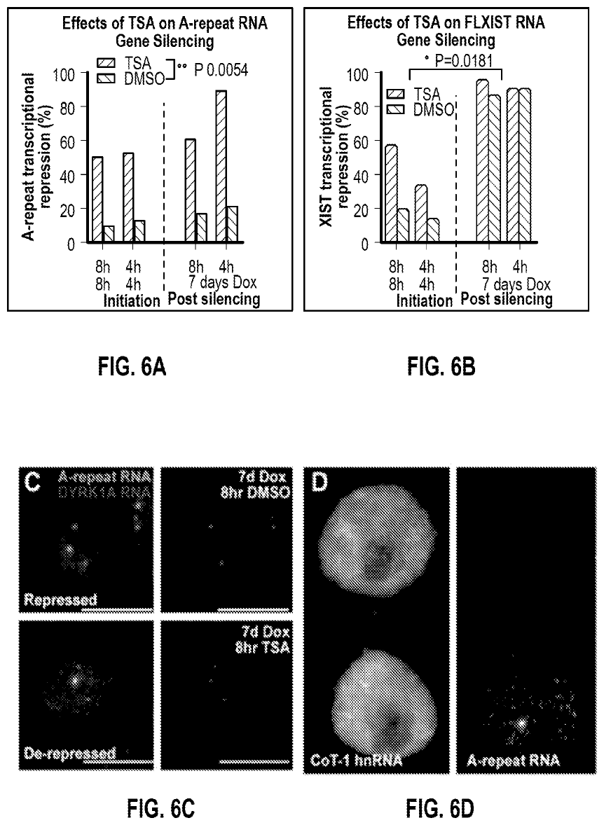

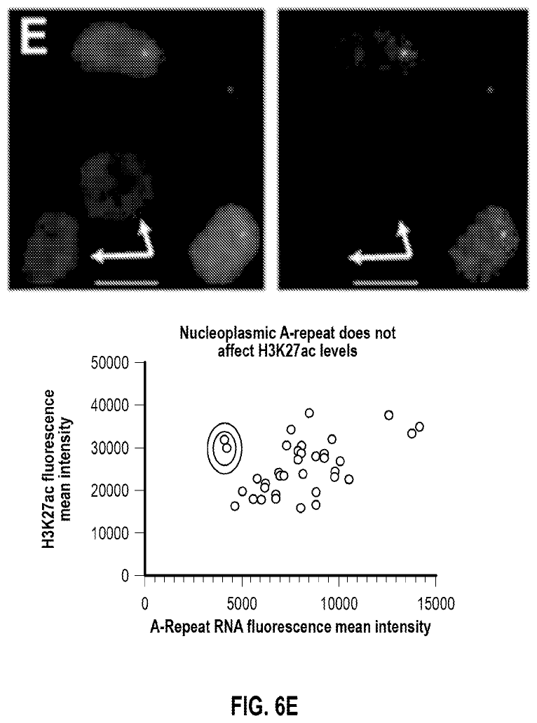

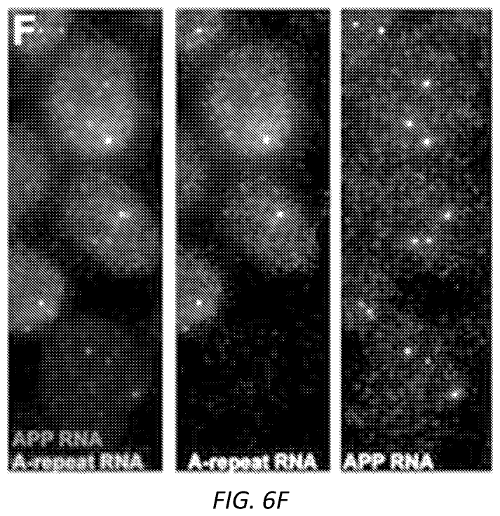

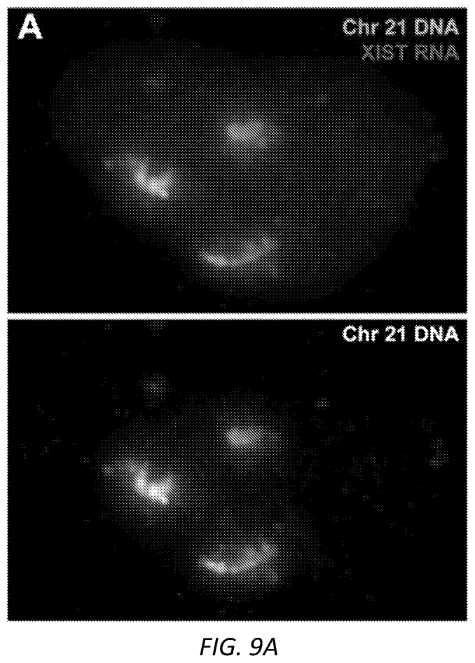

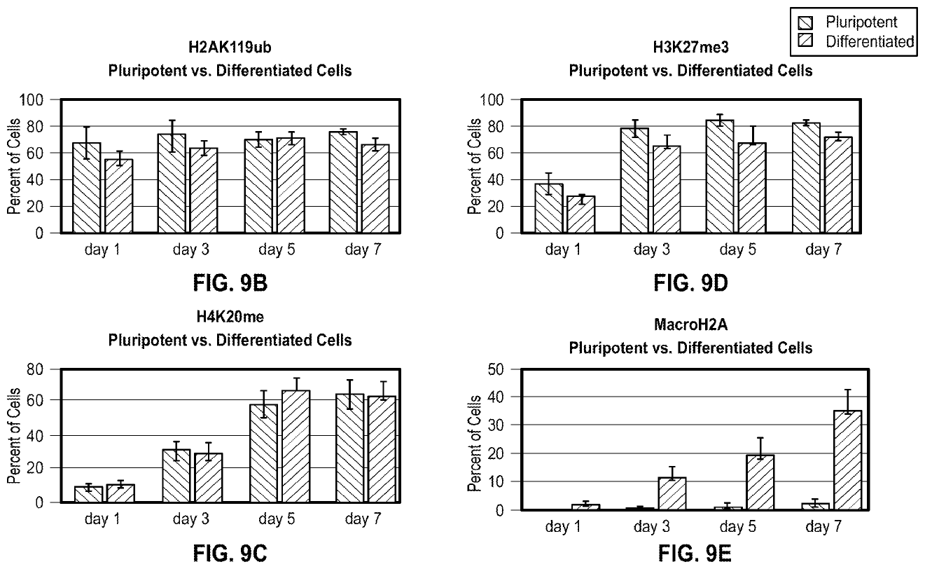

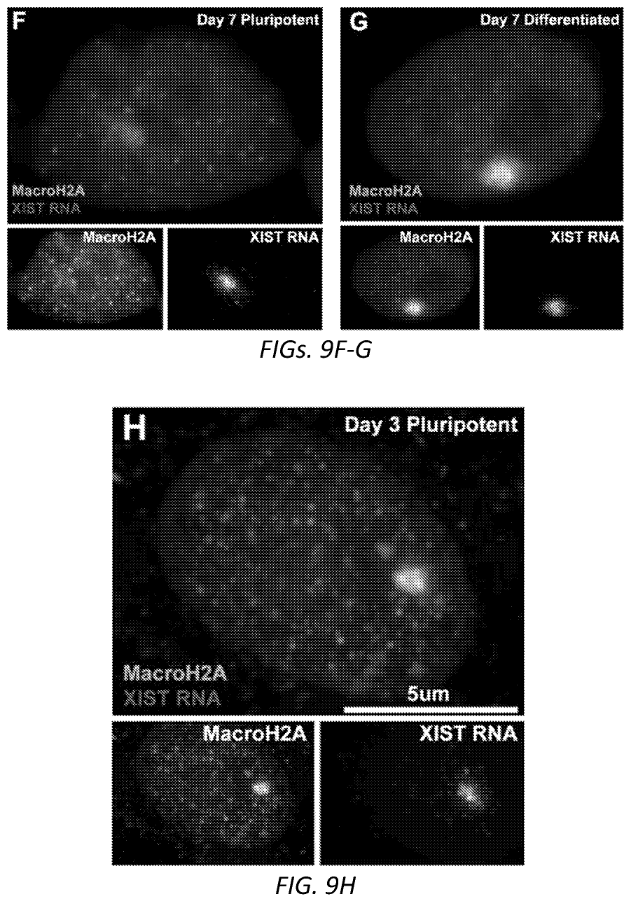

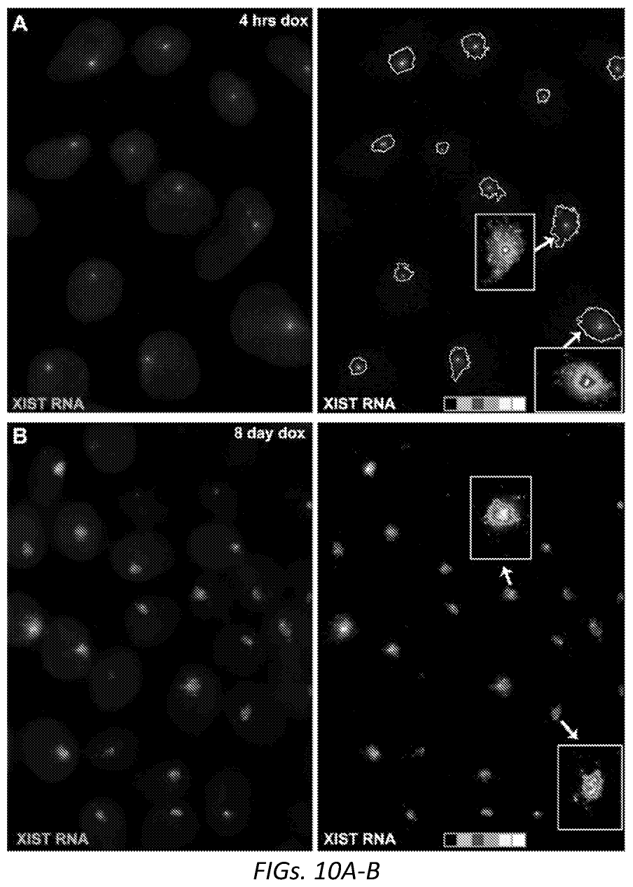

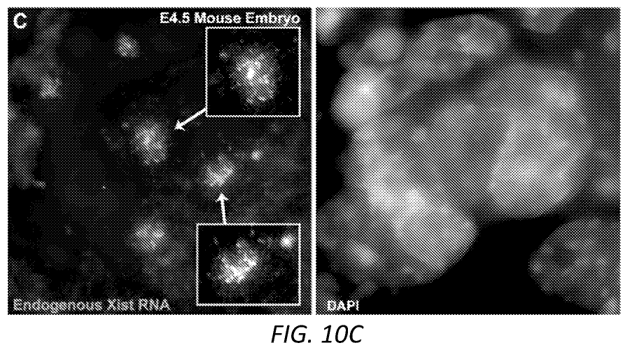

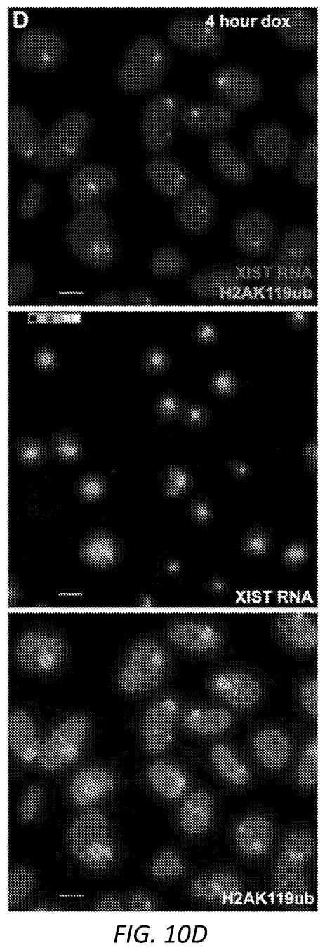

FIGS. 1-3 herein describe the chromosome-wide spread of the full-length XIST RNA, a long transcript that induces many chromatin modifications that collectively result in silencing of genes throughout the whole chromosome. This contrasts with the properties of the much smaller A-repeat mingene, which lacks most XIST sequences, most importantly those needed for broad spread of RNA across the chromosome; as shown in FIGS. 5-6, RNA from this single XIST fragment is itself able to repress gene expression of a very small chromosomal region, near the insertion site, repression is restricted locally, without the chromosome-wide spread of natural XIST RNA. FIG. 9A shows what a chromosome 21 territory is and spread of full-length XIST RNA, and FIGS. 10A-E emphasize the key property of XIST RNA is how much it spreads across extended chromosome territory, unlike A-repeat minigenes.

The importance of understanding the RNAs relationship to chromosome architecture is impacted by the magnitude of overall architectural condensation induced by XIST RNA in early development. Although the Xi DNA territory in somatic cells is typically only about two-times smaller than the Xa-territory (visualized with a whole X-chromosome DNA library) (FIG. 1A), the true scale of chromosome compaction enacted by XIST RNA needs to be understood in relation to pluripotent cells, which are the cell type where XIST RNA expression/function begins and generally have much more decondensed chromatin. For example, in human H9 ES cells, which contain a precociously inactivated X-chromosome (Hall et al., 2008)), there is a dramatic difference in size between a highly distended Xa-chromosome territory and the compacted Xi-territory (FIG. 1B). This contrast with somatic cells emphasizes the extent to which the initiation process requires not only that XIST RNA repress the transcription of genes, but this unique long ncRNA must function across broad physical space to enact large-scale structural transformation. This point provides perspective for other observations below.

### Materials and Methods

The following materials and methods were used in the Example set forth herein.

KEY RESOURCES TABLE

| | | |
|---|---|---|
| Reagent or | | |
| Resource | Source | Identifier |

Antibodies

| | | |
|---|---|---|
| Anti-H3K27me3 | Millipore | Cat# 07-449; |
| | | RRID:AB_310624 |
| Anti-UbH2A | Cell Signaling | Cat# 8240; |
| | | RRID: AB_10891618 |
| Anti-H4K20me | Abcam | ab9051 |
| Anti-macroH2A | Millipore | 07-219 |
| Anti-hnRNP-U | Abcam | ab20666 |
| Anti-CIZ1 | Santa Cruz | sc-393021 |
| Anti-H3K27ac | Diagenode | C15200184 |

Chromosome libraries

| | | |
|---|---|---|
| X chromosome Paint | ID Labs Biotechnology | IDR7023-5 |
| Chromosome 21 | Meta Systems, Newton MA | D-0321-100-FI |
| Paint | | |

FISH probes or amplicons used to make probes

| | | |
|---|---|---|
| hXIST | Addgene | G1A, 24690 |
| | Biosearch Technologies | Stellaris, SMF- |
| | | 2038-1 |
| mXIST | Carolyn Brown | plasmid XIST-MC2 |
| DYRK1A | BACPACResources Center (BPRC) | BAC-RP11-777J19 |
| APP | BPRC | BAC RP11-910G8, |
| USP25 | BPRC | BAC RP11-840D8 |
| CXADR | BPRC | BAC RP11-1150\|14, |
| COL18A1 | BPRC | BAC RP11-867018. |
| A-repeat | Carolyn Brown | p5′ XIST |
| RFP | System Biosciences | HR700PA-RFP |
| hCot1 | Roche | 11581074001 |
| Topo-DSCR3 | This study | MV1060, MV1062 |
| Topo-HLCS | This study | MV1069-MV1071 |
| Topo-TTC3 | This study | MV1072-MV1076 |
| Topo-PIGP | This study | MV1077 |

Experimental Models: Cell lines

| | | |
|---|---|---|
| Parental DS iPSC | G. Q. Daley (Children's Hospital Boston) | DS1-iPS4(Park et |
| clone | | al., 2008) |
| Parental A clone | | (Jiang et al., 2013) |
| (isogenic) | | |
| XIST transgenic | | (Jiang et al., 2013) |
| clone 1 (isogenic) | | |
| XIST transgenic | | (Jiang et al., 2013) |
| clone 5 (isogenic) | | |
| A-repeat | this study | pTRE3G-A-Repeat- |
| transgenic (isogenic) | | EF1a-RFP::DYRK1A |
| H9 hESC | WiCell | Cat# WA09 |
| mESC | Anton Wutz's lab | (Wutz, Rasmussen, |
| | | & Jaenisch, 2002) |
| Tig1 (Female normal | Coriell | Cat# AG06173 |
| human lung primary | | |
| fibroblast) | | |

Fixed cells

| | | |
|---|---|---|
| HES1,2&3 | Carol Ware, Univ of Washington, Stem cell core | (Hall et al., 2008) |
| E4.5 mouse Embryo | Ingolf Bach lab | (Shin et al., 2010) |

Primer list for pTRE3G-A-Repeat-EF1a-RFP :: DYRK1A plasmid

| Primer names | sequence | Plasmid template |
|---|---|---|
| Vector For | GGAAGATCTTCATGTCTGCGGCTCTAGAGCT | pTRE3G-XIST |
| Vector Rev | AAAGAAAAATTCTCTGCAGAATTCCACCACACTGGA | pTRE3G-XIST |
| A-Repeat For | AATTCTGCAGAGAATTTTTCTTTGGAATCATTTTTGG | pTRE3G-XIST |
| | TTGACA | |
| A-Repeat Rev | CCGATCGAAACATTTTTTCATCCATAAAAAGCACCG | pTRE3G-XIST |
| | A | |
| SV40 Poly A For | GGATGAAAAAATGTTTCGATCGGCCGGATATCAC | pTRE3G-XIST |
| SV40 Poly A Rev | GCTGTCCCTCTAAGATACATTGATGAGTTTGGACAA | pTRE3G-XIST |
| | ACCAC | |
| EF1 + RFP For | TGTATCTTAGAGGGACAGCCCCCCCCCAAA | HR700PA-RFP |
| EF1 + RFP Rev | AGGATCCTCAAGTACTTCCAGCGCCTGTG | HR700PA-RFP |
| BGH Poly A For | AGGCGCTGGAAGTACTTGAGGATCCTGATCGAG | HR700PA-RFP |
| BGH Poly A Rev | ACATGAAGATCTTCCCCAGCATGCCTGCTATT | HR700PA-RFP |

Plasmids used to build pTRE3G-A-Repeat-EF1a-RFP::DYRK1A and for transfection

| | | |
|---|---|---|
| pTRE3G-XIST | Addgene | (Jiang et al., 2013) |
| DYRK1A ZFN1&2 | | (Jiang et al., 2013) |
| rtTA/puro | | (Jiang et al., 2013) |
| AAVS1 ZFN | | (DeKelver et al., |
| | | 2010) |

Chemicals

| | | |
|---|---|---|
| PBAE (poly(β-amino | Anderson Lab, MIT | (Eltoukhy et al., |
| ester), C320) | | 2012; Zugates et |
| | | al., 2007) |
| Rho-associated | Calbiochem | Cat# Y27632 |
| protein kinases | | |
| (ROCK) inhibitor | | |
| Essential 8 medium | Gibco | Cat# A15169 |
| irradiated Mouse | Should we put this on the cell lines R&D | Cat# PSC001 |
| Embryonic | Systems | |
| Fibroblasts (iMEFs) | | |
| DMEM/F12 | Invitrogen | Cat# 12660-012 |
| Knockout Serum | Invitrogen | Cat# 10828-028 |
| Replacement | | |
| GlutaMAX | Invitrogen | Cat# 35050-061 |
| Non-essential amino | Invitrogen | Cat# 11140-050 |
| acids | | |
| β-mercaptoethanol | Sigma | Cat# M3148 |
| FGF-β | Invitrogen | Cat# PHG0024 |
| Collagenase type IV | Invitrogen | Cat# 17104-019 |
| Vitronectin (VTN-N) | ThermoFisher | Cat# A14700 |
| Recombinant | | |
| UltraPure 0.5 M | Thermo Fisher Scientific | Cat# 15575020 |
| EDTA, pH 8.0 | | |
| MEM | Invitrogen | Cat# 11095-080 |
| FBS | Invitrogen (Gibco) | Cat# 16000044 |
| CHIR99021 | Tocris | Cat# 4423 |
| CD34 MicroBead Kit | Miltenyi Biotec | Cat# 130-100-453 |
| EGM2 | Lonza | Cat# CC-3162 |
| Y-27632 | Peprotech | Cat# 1293823 |
| | Millipore (Calbiochem) | 688000 |
| 5,6- | Sigma | Cat# D1916 |
| Dichlorobenzimidazole | | |
| 1-β-D- | | |
| ribofuranoside | | |
| (DRB) | | |
| 5-10 uM trichostatin- | Sigma | Cat# T1952 |
| A (TSA) | | |
| Tautomycin | Millipore Sigma | Cat# 580551 |
| Doxycycline | Clonetech Labs | Cat# 631311 |
| Triton X-100 | Roche | Cat# 11332481001 |
| Paraformaldehyde | Ted Pella | Cat# 18505 |
| Phosphate-buffered | Fisher | Cat# BP3994 |
| saline (PBS) | | |
| Ethanol | Decon | Cat# 04355223 |
| Biotin-11-dUTP | Roche | Cat# 11093070910 |
| Digoxigenin-16- | Roche | Cat# 11093088910 |
| dUTP (Dig) | | |
| Formaldehyde | Sigma | Cat# 221198 |
| RNasin Plus RNase | Promega | Cat# N261B |
| inhibitor | | |
| FitcAnti-Dig | Roche | Cat# 11207741910 |
| Alexa Flour 594 | ThermoFisher | Cat# S32356 |
| Streptavidin | | |
| Alexa Fluor 488 | ThermoFisher | Cat# S32354 |
| Streptavidin | | |
| DAPI | Sigma | Cat# D8417 |
| XL-2-TOPO | ThermoFisher | Cat# K8050 |
| NEBuilder HiFi DNA | New England Biolabs | Cat# E2621 |
| Assembly Master | | |
| Mix | | |

pTRE3G-A-Repeat-EF1a-RFP::DYRK1A plasmid. A-Repeat, and backbone with arms to DYRK1A, were PCR amplified from pTRE3G-XIST(Jiang et al., 2013). The EF1RFP was amplified from plasmid HR700PA-RFP (System Biosciences). The five PCR products were GIBSON assembled. Primer sequences are listed in table.

Inducible A-repeat cell line. The inducible A-repeat transgene was targeted to the first intron of the DYRK1A locus in chromosome 21 and the transactivators were targeted to chromosome 19 AAV site in Down Syndrome iPS cells as described in (Jiang et al., 2013), but using PBAE (poly(β-amino ester), C320 (generously provided by the Anderson Lab, MIT(Eltoukhy et al., 2012; Zugates et al., 2007)). Briefly, Down's syndrome iPS cell parental line provided by G. Q. Daley (Children's Hospital Boston)(Park et al., 2008) were grown to exponential phase and cultured in 10 mM of Rho-associated protein kinases (ROCK) inhibitor (Calbiochem; Y27632) 24 h before transfection. 55 mg DNA including five plasmids (pTRE3G-A-Repeat-EF1a-RFP, DYRK1A ZFN1, DYRK1A ZFN2, rtTA/puro and AAVS1 ZFN) with 6:1 ratio of A-repeat:rtTA/puro were mixed with 1:20 ratio of PBAE Polymer and incubated with cells for four hours. Cells were washed with media and kept overnight with Essential 8 medium and rock inhibitor. Next day, cells were selected for puromycin resistance. Red clones were isolated. Expression of the A-repeat was induced with doxycycline. Clones that lost the red fluorescence upon dox induction were used for this study. Expression of A-repeat was validated by RNA FISH and proper targeting by colocalization of the A-repeat and DYRK1A RNA transcription foci by RNA FISH. RFP and DYRK1 RNA were usually detected in separate but adjacent transcription foci. However, we noticed that upon dox induction, some A-repeat transcripts also contained downstream sequences for RFP and DYRK1A in a colocalizing focus, but this co-localized RFP/DYRK1A signal was restricted to the A-repeat transcription focus, and appeared only in the presence of dox, suggesting read-through. Although this read-though RFP/DYRK1 RNA signal persisted in the presence of dox, the RFP protein was no longer present, indicating gene silencing. Thus, no functional mRNA for RFP or DYRK1A was expressed from this locus upon dox induction and gene silencing.

Cells kept in the presence of puromycin selection expressed the A-repeat transgene in almost 100% of cells. The frequency of cells expressing A-repeat dropped over time when grown in the absence of puromycin due to stochastic silencing of the tet-activator. These non-inducing cells were used as internal “non-expressing” controls for many experiments.

Cell culture. Human Down's syndrome iPS cell lines with XIST transgenes, isogenic lines and H9 hESC were maintained on irradiated mouse embryonic fibroblasts (iMEFs) (R&D Systems, PSC001) in hiPSC medium containing DMEM/F12 supplemented with 20% Knockout Serum Replacement, 1 mM glutamine, 100 mM non-essential amino acids, 100 mM b-mercaptoethanol and 10 ng/ml FGF-β. Cultures were passaged every 5-7 days with 1 mg/ml of collagenase type IV. In later studies, cells were grown in Essential 8 medium on plates coated in vitronectin 0.5 ug/cm2. Cells were passed when reached 80% confluency by detaching TIG-1 Female normal human lung primary fibroblast line were cultured in MEM 15% FBS.

Expression of XIST and the A-repeat was induced with doxycycline (500 ng/ml) while maintained as pluripotent, or directly upon differentiation. Random differentiation was achieved by removing iPS cells from feeder layer and feeding them DMEM/F12, 4% Knockout Serum Replacement, 100 mM Non-essential amino acids, 1 mM L-glutamine, 100 mM β-mercaptoethanol. iPS cells were differentiated into endothelial cells with Gsk3 inhibitor (as in (Bao, Lian, & Palecek, 2016) and Moon, in preparation) in LaSR basal media (formulated from Bao 2016 (Bao et al., 2016)) with 6 μM CHIR99021 for the first two days. Endothelial precursor cells were purified using CD34 MicroBead Kit (Miltenyi Biotec, cat #130-100-453); and maintained in EGM2 (Lonza, cat #CC-3162) (with 5 μM Y-27632 for the first day) on vitronectin coated plates. NPC differentiation was performed as (Czerminski & Lawrence, 2020).

For transcriptional, HDAC and protein phosphatase 1 inhibition, cells in coverslips were incubated with 50 ug/ul 5,6-Dichlorobenzimidazole 1-β-D-ribofuranoside (DRB), with 5-10 uM trichostatin-A (TSA) and with 3 uM Tautomycin respectively for the indicated time. Cells were then fixed as indicated below for RNA FISH.

Male mouse J1 ES cells containing a doxycycline-inducible Xist cDNA transgene integrated on Chr-11 (clone #65)(Wutz et al., 2002) were maintained in DMEM (GIBCO), 15% fetal calf serum (FCS, Hyclone), and no supplemental antibiotics. They were grown on mitomycin inactivated (10 ug/ml mitomycin C for 2 hours at 37C) STO fibroblast feeder cells (SNL76) that produce LIF from an ectopic transgene. mES cells were differentiated by removing colonies from feeders (through two two-hour sequential separations of single cell suspension onto gelatinized flasks) and distributing them as a single cell monolayer on gelatinized (0.1% porcine skin gelatin) flasks in the presence of 100 nM all-trans-retinoic acid. Xist RNA expression was induced with 1 μg/ml doxycycline at the same time. Time points were taken by trypsinizing the cells and plating them as a monolayer onto coverslips coated with CellTak (BD) (following the protocol that comes with the CellTak solution) for 1 hour before fixation.

DNA and RNA FISH and immunostaining. These protocols were carried out as previously described (Byron, Hall, & Lawrence, 2013; Clemson, McNeil, Willard, & Lawrence, 1996). Cells were fixed for RNA in situ hybridization as described in (Byron et al., 2013). Briefly, cells cultured on coverslips were extracted with triton X-100 for 3 min and fixed in 4% paraformaldehyde in phosphate-buffered saline (PBS) for 10 min. Cells were then dehydrated in 100% cold ethanol for 10 min and air-dried. Cells were then hybridized with biotin-11-dUTP or digoxigenin-16-dUTP (Dig) labeled Nick translated DNA probes. DSCR3, TTC3, PIG3, HLCS DNA probes were obtained by amplifying ˜10 Kb gene regions from the DS iPS genomic DNA and cloned into TOPO vector A cold TOPO vector was added to the hybridization mixture of TOPO constructions to decrease background.

For hybridizations, 50 ng of labeled probes and CoT-1 competitor were resuspended in 100% formaldehyde, followed by denaturation in 80° C. for 10 min. Hybridizations were performed in 1:1 mixture of denatured probes and 50% formamide hybridization buffer supplemented with 2 U/μl of RNasin Plus RNase inhibitor for 3 h or overnight at 37° C. Cells were then washed three times for 20 min each, followed by detection with fluorescently conjugated secondary antibody anti-dig or streptavidin. DNA was stained with DAPI. In simultaneous DNA/RNA FISH (interphase targeting assay), cellular DNA was denatured and hybridization was performed without eliminating RNA and also treated with 2 U/ml of RNasin Plus RNase inhibitor. For immunostaining with RNA FISH, cells were immunostained first with primary antibodies containing RNasin Plus and fixed in 4% paraformaldehyde after detection, before RNA FISH.

Most antibodies were diluted at 1:500 ratio. X chromosome (ID Labs Biotechnology) was detected with whole chromosome paint probe, following manufacturers instructions.

Image analysis. Cells were imaged in a Zeiss AxioObserver 7, equipped with a 100× Plan-Apochromat oil objective (NA 1.4) and Chroma multi-bandpass dichroic and emission filter sets (Brattleboro, VT), with a Flash 4.0 LT CMOS camera (Hamamatsu). Z stacks were taken for each field to evaluate detectable transcription foci. To evaluate if the A-repeat silenced nearby genes, we compared the frequency that a gene's transcription focus was in close proximity to DYRK1A or RFP RNA foci in the absence of doxycycline, to the frequency the gene's transcription focus was in close proximity to the A-repeat RNA focus in the presence of doxycycline. Images show a plane from the z stack or a MIP (indicated). Most experiments were carried out a minimum of 3 times, with typically 100-300 cells scored in each experiment. Key results were confirmed by at least two independent investigators. Images were minimally enhanced for brightness and contrast to resemble what was seen by eye through the microscope. Some line scans were done in Image J, and some using Profile function from ZEN 3.1 software and plotted in Prism. Heat maps were created with Image J (fuji)

Transcriptomic data was generated for a different study (Moon et al. in prep). Briefly, data was originated from 4 transgenic lines. NPC were achieved as previously described (Czerminski & Lawrence, 2020; Czerminski 2020) and collected for sequencing on diff day 14 (dox at diff day 0) while endothelial cells were differentiated with Gsk3 inhibitor as in (Bao et al., 2016) and collected for sequencing on diff day 12. RNA seq analysis was performed using EdgeR (McCarthy, Chen, & Smyth, 2012), using normalized cpm values. Figure uses log 2 values.

Human XIST RNA Triggers UbH2A within Two Hours Followed by H3K27Me3, H4K20Me, and macroH2A

To examine steps in the initiation of human chromosome silencing with high temporal resolution we used our XIST-transgenic trisomic iPSC system to synchronously induce XIST RNA for different time periods. Using this system, we previously showed that XIST RNA comprehensively silences the ˜400 genes across chromosome 21 in cis by 7 days (Czerminski & Lawrence, 2020; Jiang et al., 2013), and compacts an initially distended Chr 21 territory (FIG. 9A). We began by examining the appearance of four canonical heterochromatin hallmarks after induction of XIST for 1-7 days. Immunofluorescence assays for H3K27me3, H2AK119ub, H4K20me and macroH2A each produce a bright signal against the darker nuclear background (FIG. 1C), allowing sensitive visualization of these marks and XIST RNA on the same chromosome. Since XIST RNA expression begins in pluripotent cells just prior to differentiation, we compared the process in cells maintained as pluripotent or in those switched to differentiation media after dox induction, which would reveal if timing of any of these modifications are differentiation-dependent.

Enrichment for H4K20me and macroH2A appear days after H2AK119ub and H3K27me3 (FIG. 1D&F). Interestingly, all marks appeared with similar kinetics in pluripotent versus differentiating cells except for macroH2A, which generally accumulated after the switch to differentiation conditions (see also: FIGS. 9B-G). Even in differentiating cultures, macroH2A lagged H4K20me by generally two days indicating macroH2A occurs later and is more differentiation dependent (FIG. 1F); we note that some variability in the timing of macroH2A was seen and may reflect methods of iPSC culture and maintenance (see Methods & FIG. 9H).

Both H2AK119ub and H3K27me3 accumulate on the inactivating chromosome in many cells by Day 1 and reached maximum by Day 3, independent of differentiation (FIG. 1D). It is important to know which of these marks are recruited first by human XIST RNA, since earlier reports in mouse suggested Xist RNA recruits PRC2 first (for H3K27me3), followed by PRC1 (for H2AK119ub)(Zhao, Sun, Erwin, Song, & Lee, 2008), reflecting their canonical relationship, while subsequent reports suggest initial deposition of H2AK119ub on the Xi occurs before H3K27me3 (Almeida et al., 2017; Zylicz et al., 2019). We therefore examined cells just 2, 4, and 8 hours after adding doxycycline, and scored H2AK119ub and H3K27me3 enrichment in XIST expressing cells (on parallel slides in the same experiment). Results demonstrate a clear temporal difference, with H2AK119ub remarkably quick, and enriched in 71%, 93% and 98% of XIST RNA-positive cells at just 2, 4, and 8 hours, respectively (FIG. 1E). In contrast, in parallel samples only ˜24% of cells accumulated H3K27me3 by 8 hours, and similar results in multiple experiments affirmed this order. We conclude that in human cells XIST RNA triggers strong H2A119ub modification by PRC1 several hours before H3K27me3.

The appearance of H2AK119ub at the earliest time, just two hours after adding doxycycline, shows extremely close temporal connection with the initial onset of XIST RNA expression.

### Broad Territory of Sparse XIST RNA Triggers H2AK119Ub Whereas H3K27Me3 Localizes to Dense Zone

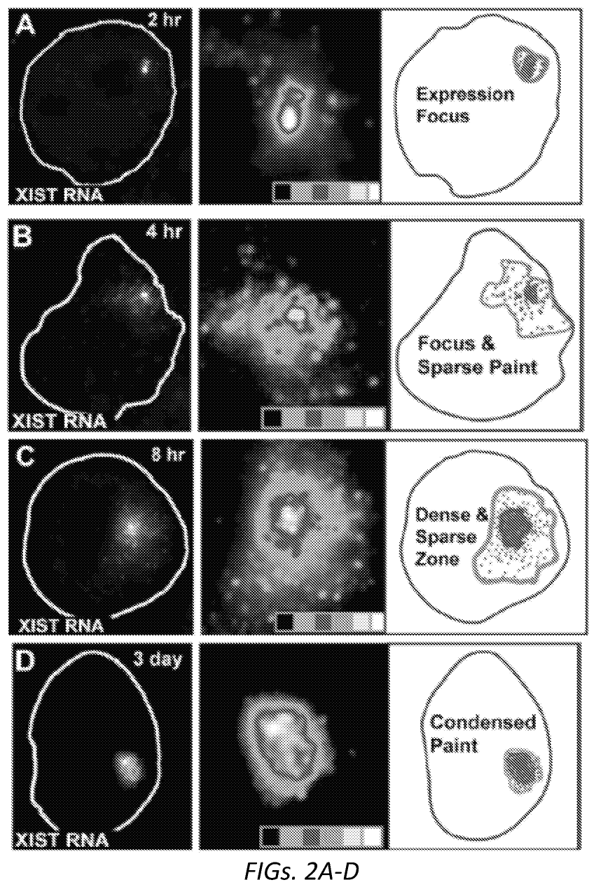

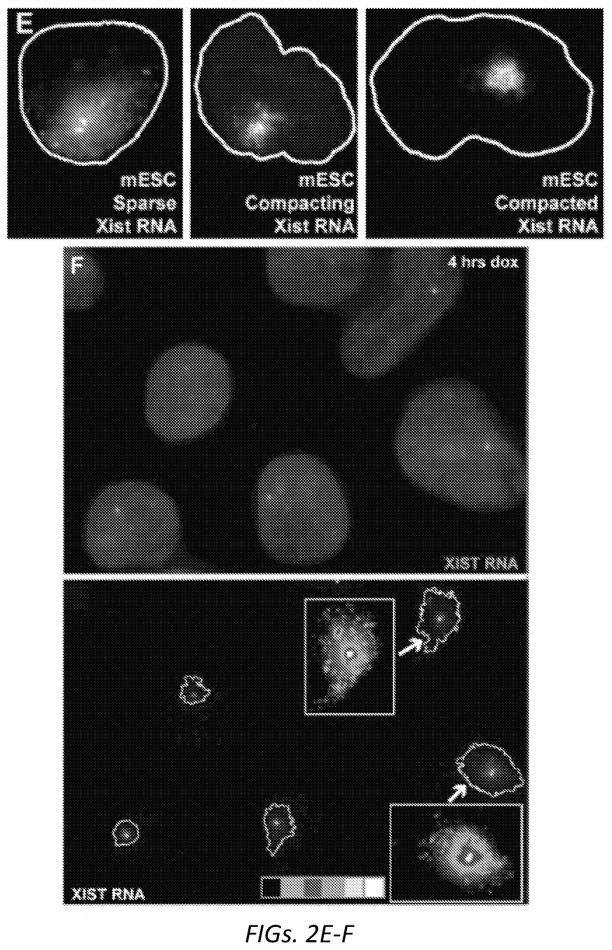

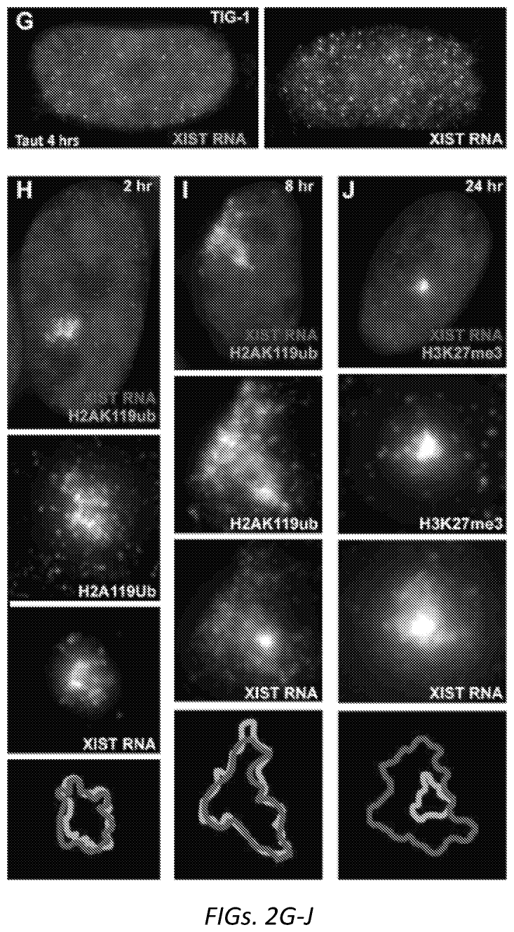

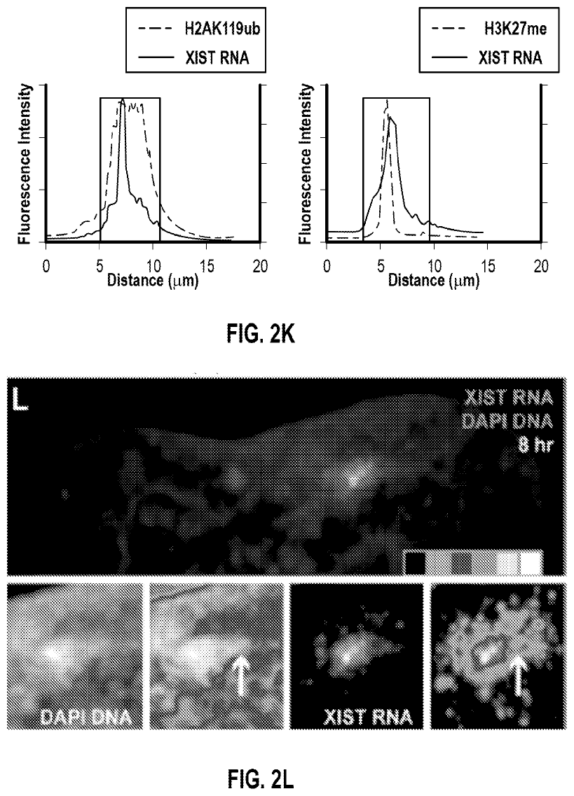

Since H2AK119ub and H3K27me3 enrichment both appear early, we examined their distribution relative to XIST RNA on individual chromosomes. The tight temporal connection between XIST RNA and H2AK119ub is further reflected in their relative distributions. Notably, H2AK119ub is elevated throughout the whole XIST RNA territory including the large sparse-zone (FIGS. 2H-I & K and FIG. 10D). Even adjust two hours when we can see a very low level of XIST transcripts in the sparse-zone, this is coincident with clear, often bright, enrichment for H2AK119ub.

The approach taken here allows direct visualization of XIST RNA spreading across the inactivating chromosome territory relative to the temporal and spatial distribution of H2AK119ub and H3K27me3, at very early time points. XIST RNA first forms a small bright transcription focus (FIG. 2A), but sensitive RNA FISH analysis also consistently detects very low levels of XIST transcripts that spread much further within hours, although they remain within a discrete but large nuclear territory (FIGS. 2B & F and FIG. 10A). As explained under Methods, these low-level transcripts are visible through the microscope by eye, but may be missed if hybridization conditions (or digital imaging) are not optimal. By 8 hours many cells show this sparse punctate distribution of XIST transcripts in a larger region surrounding a smaller more intense focal center of high-density RNA (FIG. 2C). We will refer to these two regions of differing XIST RNA density as the “sparse-zone” and “dense-zone”. Importantly, we detect the same Xist-RNA dense- and sparse-zone distribution during the early stages of chromosome silencing in Xist-transgenic mouse ES cells (FIG. 2E) and in very early mouse embryos during X-inactivation (FIG. 10C). This low-level regional spread of XIST RNA is distinct from complete dispersal of XIST RNA throughout the entire nucleus, as illustrated when XIST RNA is released to drift from the interphase chromosome by a brief (4 hour) treatment with tautomycin (Hall, Byron, Pageau, & Lawrence, 2009) (FIG. 2G).

In contrast, H3K27me3 is incorporated not only later (shown above) but is much more restricted to the smaller dense XIST RNA zone (FIGS. 2J & K). Thus, H2AK119ub staining mirrors XIST RNA distribution largely independent of density, while H3K27me3 enrichment is limited to the dense-zone. If RNA hybridization is omitted (to rule out any impact of hybridization procedures), H2AK119ub clearly and consistently marks a region larger than that of H3K27me3 (FIG. 10E).

Importantly, this indicates that the low levels of sparsely distributed XIST RNA shown here are not noise or inconsequential “drift”, but transcripts functionally interacting with chromatin which triggers H2AK119ub histone modification by PRC1. Moreover, these results indicate that XIST transcript density is a factor that influences its functional effects, and that distinct histone modifications may differ in their requirements for transcript density.

Between ˜1-3 days following XIST induction the dense RNA zone expands and encompasses the progressively smaller sparse-zone. Ultimately the more compact uniformly dense XIST RNA territory is formed (e.g. FIG. 2D & FIG. 10B), as is typical of the XIST RNA coated Barr body of somatic cells. The small dense XIST RNA zone in early timepoints, which eventually overlaps H3K27me3, often coincides with the most distinct focal increase in DNA condensation (FIG. 2L), indicating an early stage in nucleation of the Barr body. Less frequently a slight DAPI-DNA density was also seen under XIST RNA in the larger sparse-zone (FIG. 2L arrow) but was only discernible using optical sectioning and deconvolution for high-resolution maximum image projections. In any case, XIST RNA is initially very sparsely distributed across a highly distended chromosome and as local transcript density increases, they cluster into dense collections that further aggregate, coincident with compaction of the chromosome.

### XIST RNA Acts Early to Modify Architecture Before Most Gene Silencing

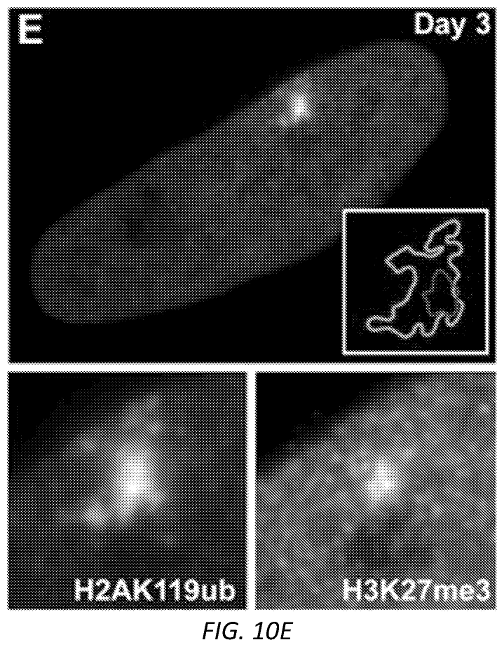

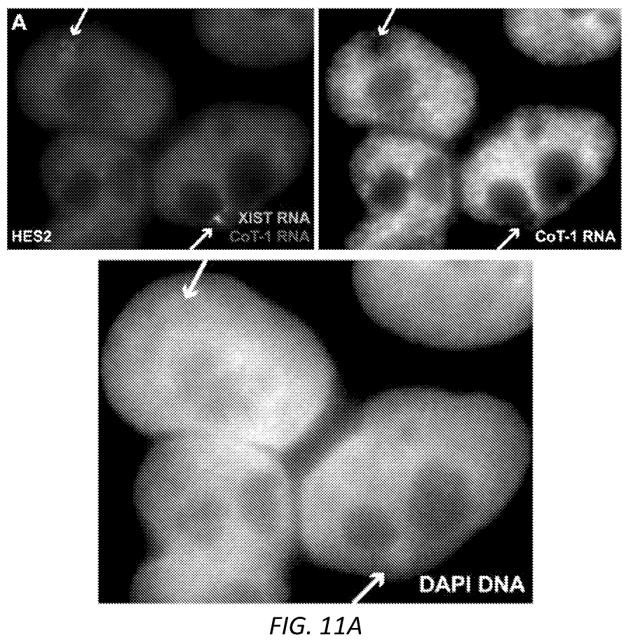

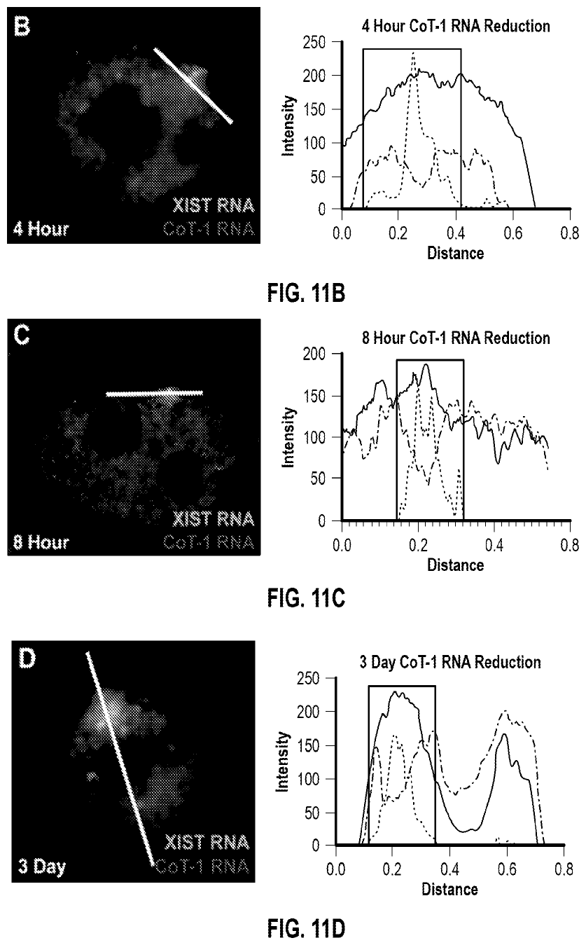

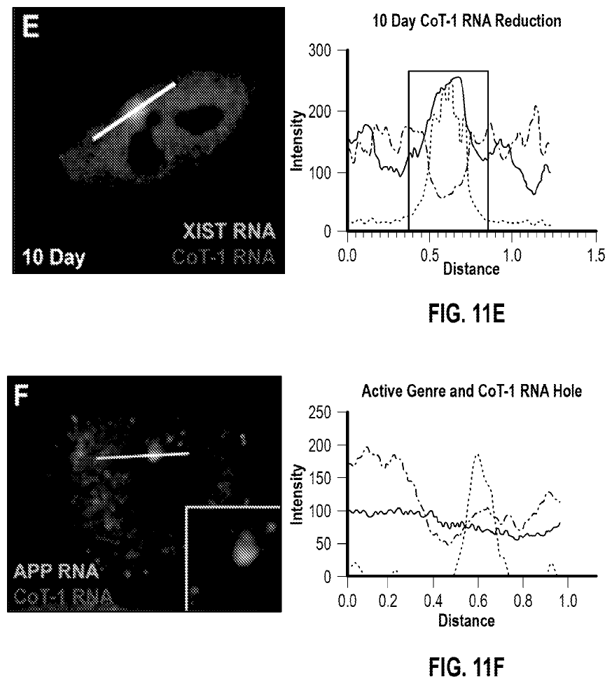

The mature Barr body of somatic cells is also marked by a void of repeat-rich hnRNA, detected by hybridization to CoT-1 RNA (Clemson, Hall, Byron, McNeil, & Lawrence, 2006; Hall et al., 2002), which more reliably delineates the Barr body in human cells (particularly pluripotent cells; e.g. FIG. 11A) as well as mouse cells (in which a dense Barr body is particularly difficult to see with DNA stains)(Chaumeil, Le Baccon, Wutz, & Heard, 2006). Hence, we examined CoT-1 RNA as a hallmark for architecture, but also to compare formation of this “silent domain” to temporal silencing of canonical genes. The Barr body was long thought to comprise the whole Xi, presumed to be condensed due to gene silencing. However, we previously showed that the Barr body is a dense chromosome core of repeat-rich DNA with all of 14 genes examined distributed at the periphery (irrespective of silencing) and just outside the DAPI-dense Barr body (Clemson et al., 2006). Others have shown that even genes on active chromosome territories mostly localize within a peripheral zone (Bickmore, 2013; Bickmore & Teague, 2002; Clemson et al., 2006; Mahy, Perry, & Bickmore, 2002), and this looser organizational pattern becomes more tightly defined on a condensed inactive chromosome (Hall & Lawrence, 2010).

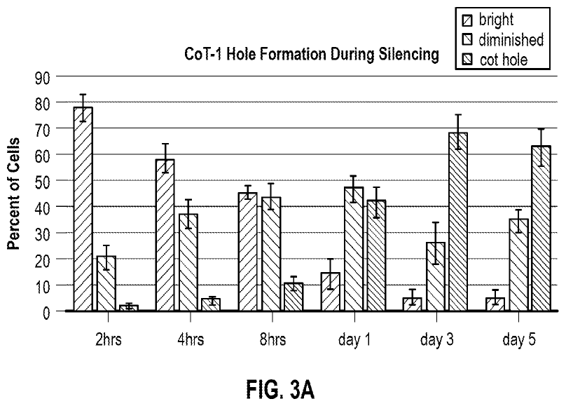

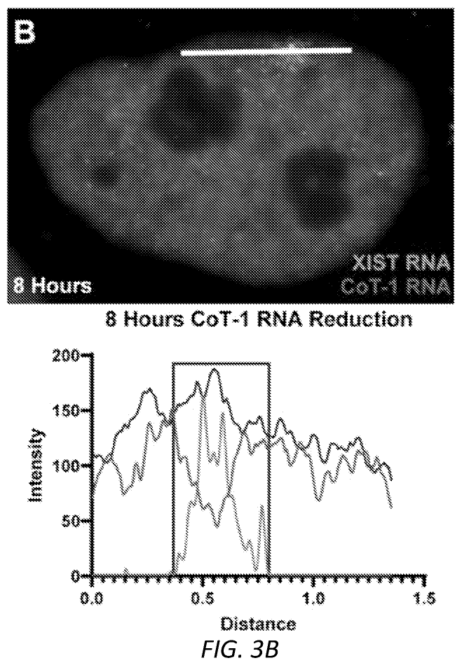

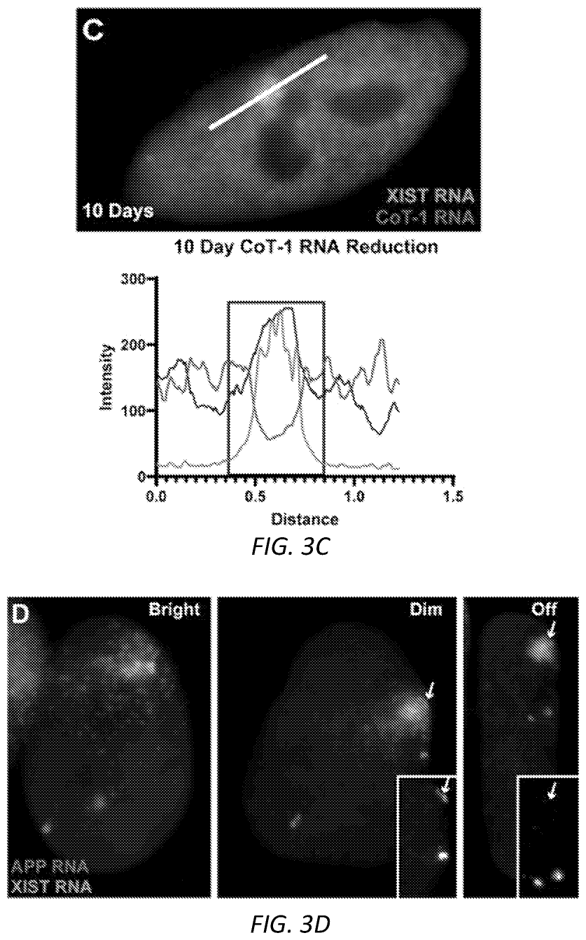

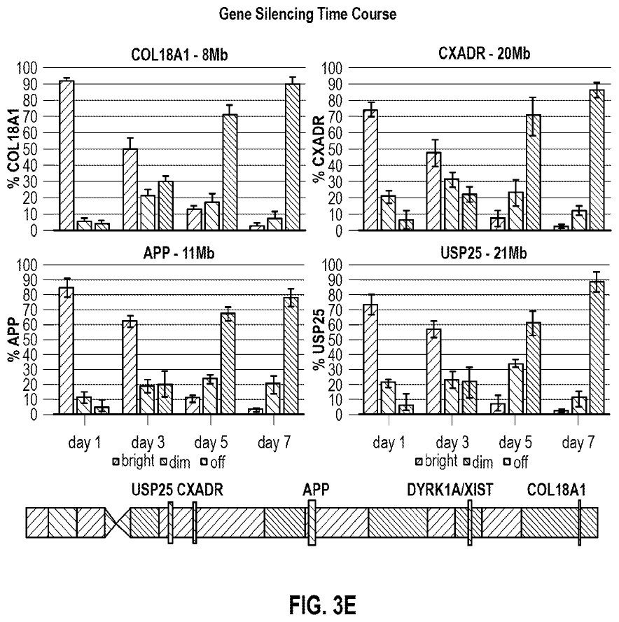

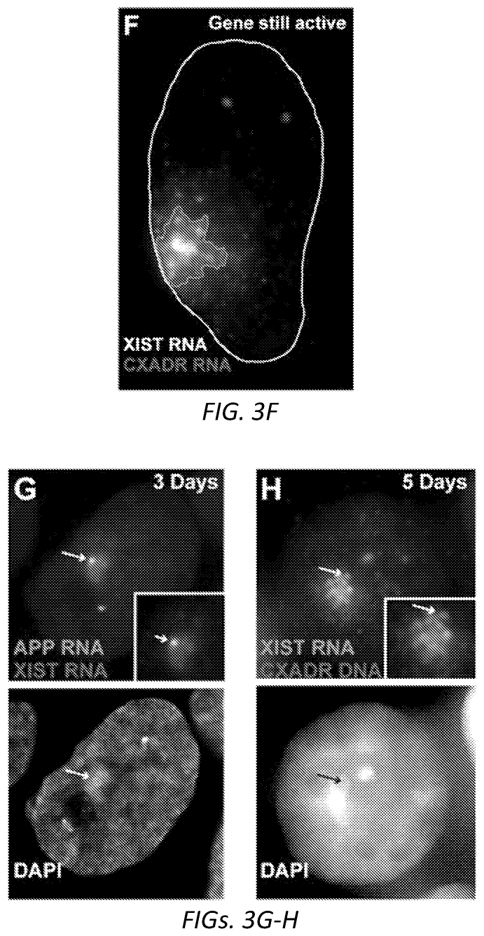

RNA FISH allows analysis of the temporal and spatial relationships of CoT-1 RNA and gene silencing on the inactivating chromosome. Depletion of CoT-1 hnRNA was generally seen by day 1, therefore we examined shorter time-points (FIG. 3A-B). A modest depletion of CoT-1 RNA could be seen in some cells at two hours and this becomes more evident at 4 and 8 hours (FIG. 3B & FIGS. 11B-C). The initial loss of CoT-1 RNA was often clearest at the small dense-zone of bright XIST RNA concentration, with much lower levels of repression over the sparse-zone, which is reflected in the “V” shape of the linescan (FIG. 3B). By 24 hours a more clearly defined larger region of decreased CoT-1 RNA is seen, which eventually encompasses most of the chromosome territory by Day 3 (FIG. 11D), and a fully formed “CoT-1 hole” by the end of the week.

As we previously showed (Clemson et al., 2006; Xing, Johnson, Dobner, & Lawrence, 1993) in situ detection of transcription foci provides a direct read-out of allele-specific gene silencing on the XIST RNA coated chromosome. Hence, we identified genomic probes that detect with high efficiency pre-mRNA foci for four genes which map widely across the chromosome (8-21 MB from XIST). We quantified silencing at days 1, 3, 5, and 7, with CoT-1 RNA examined in parallel. While a CoT-1 RNA depleted domain was apparent in most cells by Day 1 (e.g. FIG. 3A), at this time point none of the four genes showed reduced intensity of transcription from the XIST associated allele compared to the other two alleles in the same cell (FIG. 3D-E). Thus, transcription foci for all four genes continue to be synthesized in these rapidly dividing cells, with significant silencing not observed until Day 3 of XIST expression, and was not maximal until Day 7, in either pluripotent or differentiated cells.

FIG. 3F further illustrates that transcription foci for these genes are expressed in the larger sparse-zone of the XIST RNA territory, outside the more dense XIST RNA dense zone. In keeping with the organization shown for numerous Xi genes in human fibroblasts(Clemson et al., 2006) and differentiating mouse cells(Chaumeil et al., 2006), by Day 5 or 7 silenced genes come “inward” to distribute primarily in the peripheral rim of the condensed chromosome (FIG. 3G-H). Thus, the large DAPI dense domain lacking Cot-1 RNA (Barr Body) is essentially formed about two days before long-range gene silencing occurs.

XIST Rapidly Impacts CIZ-1 Architectural Protein and does so Well Before Peripheral Chromosome Movement

Most studies of XIST RNA function have focused on the RNA as a trigger for a cascade of histone modifications, which are known to impact chromatin structure at the nucleosomal level, linked to transcription. Larger-scale chromosome condensation, a hallmark of the process, is commonly thought to reflect additive effects of local histone modifications and gene silencing. However, the above findings demonstrate that XIST RNA acts to modify cytological-scale architecture well before most gene silencing, and before most histone modifications. Hence, a fundamentally distinct and important possibility is that XIST RNA impacts elements of larger-scale architecture more directly.

In earlier work demonstrating that XIST RNA paints the Xi DNA territory, we showed that after DNase digestion XIST RNA remains with the classically defined nuclear matrix (Clemson, McNeil, Willard, & Lawrence, 1996). Subsequently, two matrix proteins, SAF-A (Helbig & Fackelmayer, 2003) and CIZ-1 (Ridings-Figueroa et al., 2017; H. Sunwoo, D. Colognori, J. E. Froberg, Y. Jeon, & J. T. Lee, 2017) have been shown enriched on Xi and thought to function as tethers for XIST RNA on the chromosome so that it can act strictly in cis to trigger histone modifications. Both SAF-A and CIZ-1 are thought to be recruited to chromatin by XIST RNA and are necessary to maintain XIST RNA localization in some cell-types (Hasegawa et al., 2010; Kolpa, Fackelmayer, & Lawrence, 2016; Ridings-Figueroa et al., 2017; Hongjae Sunwoo, David Colognori, John E. Froberg, Yesu Jeon, & Jeannie T. Lee, 2017).

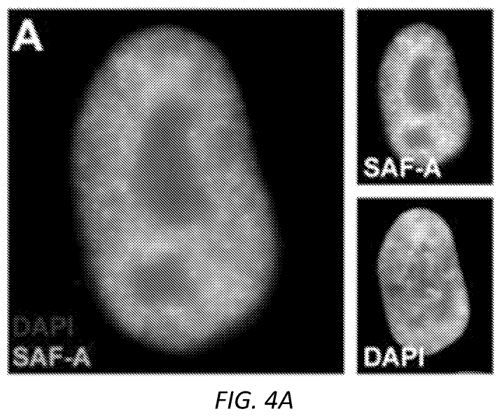

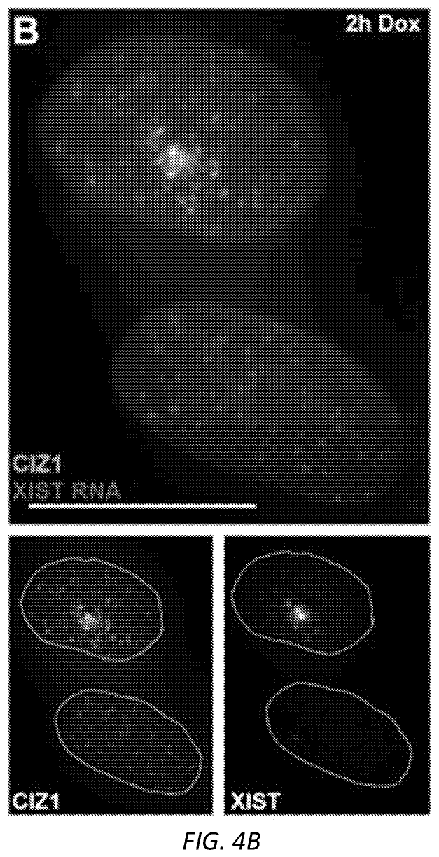

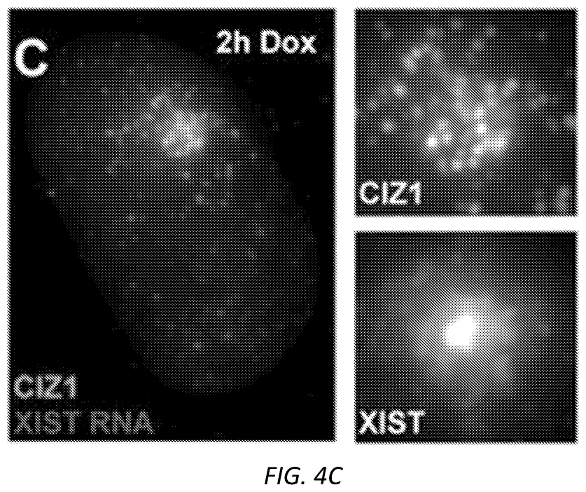

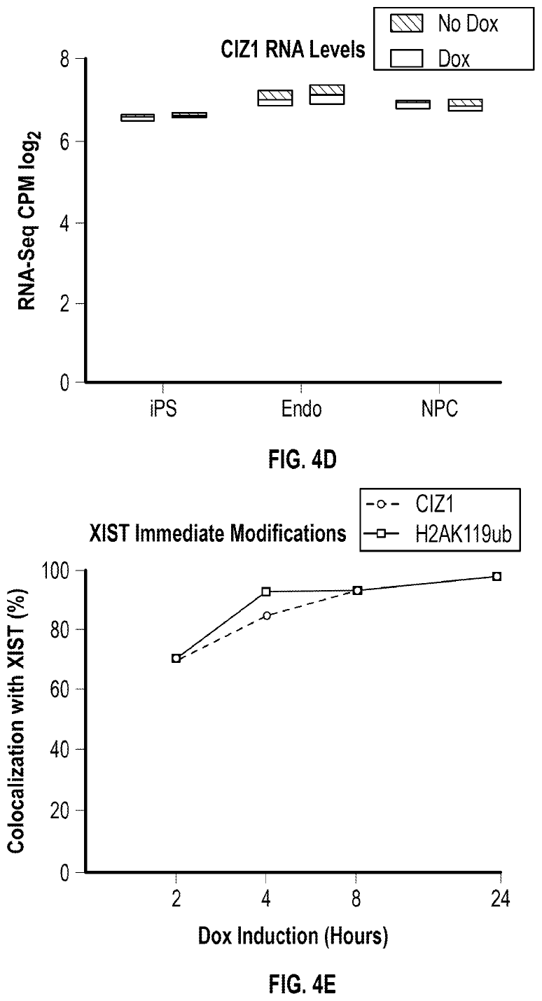

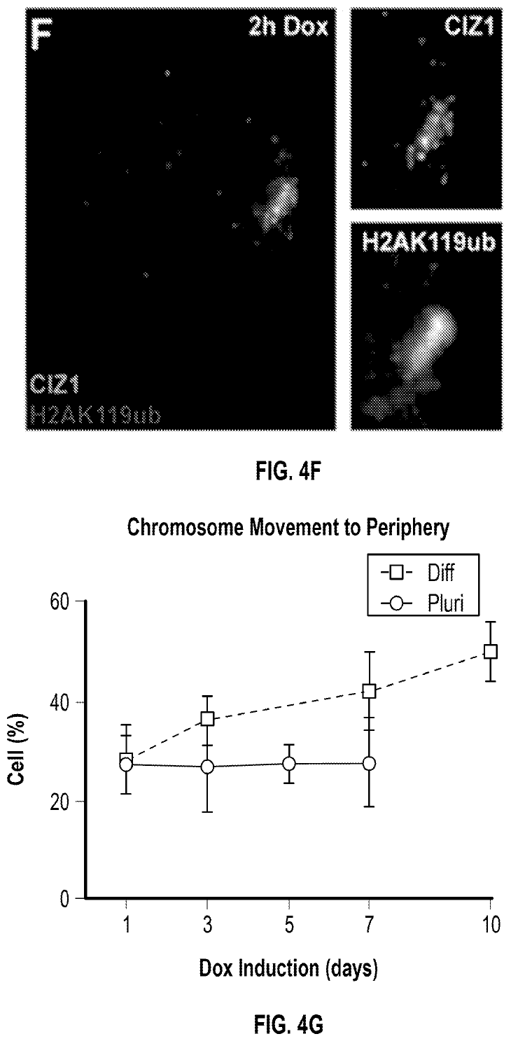

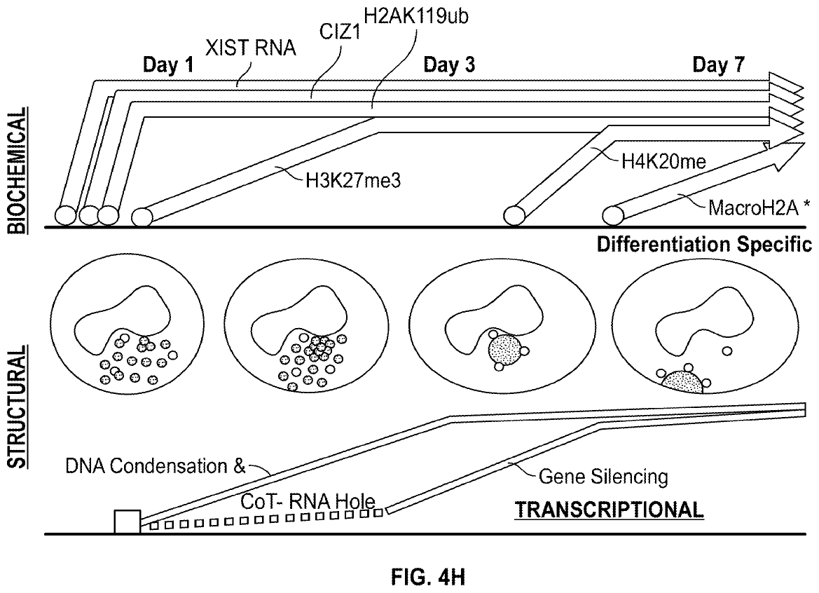

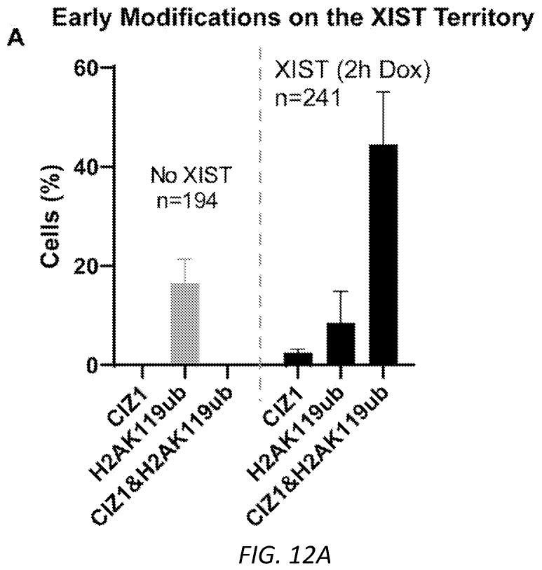

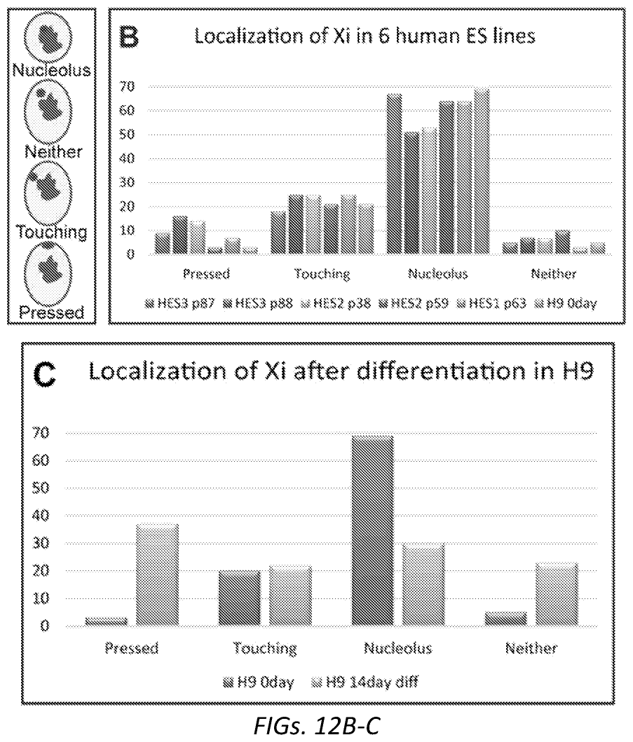

As shown in FIG. 4A, immunofluorescence for SAF-A shows broad chromatin distribution in pluripotent cells (before any XIST expression), consistent with earlier observations in human fibroblast and HEK293 cells. Hence SAF-A is present on chromatin independent of XIST RNA but appears to be enriched with XIST RNA present, although this enrichment by IF is only visible after DNA removal in a matrix preparation (or antigen retrieval procedures)(Helbig & Fackelmayer, 2003; Kolpa et al., 2016) (FIG. 12A). In contrast, CIZ1 staining is essentially negative in iPSCs prior to XIST induction, with at most a few tiny puncta visible against a dark background (FIG. 4B). However, in cells expressing XIST RNA, a very bright territory of CIZ1 overlaps the XIST RNA territory in an otherwise empty nucleoplasm (FIG. 4B). Because robust CIZ1 signal was seen with XIST RNA at Day 1, we examined earlier time points and found many cells with bright CIZ1 had formed within just two hours of adding doxycycline (FIG. 4B-C). At all early time points CIZ1 is strongly detected in both the sparse- and dense-zones of XIST RNA, mirroring the distribution of XIST RNA. Given the lack of CIZ1 staining in pluripotent cells (nuclei or cytoplasm) before induction, it was surprising that such a large, robust accumulation of CIZ1 appears so quickly, with no change in the minimal nucleoplasmic fluorescence. This very short-time frame seems difficult to reconcile with XIST RNA inducing CIZ1 expression and subsequently recruiting newly synthesized protein. In support of this, RNAseq data (Methods) from iPSCs and endothelial cells shows CIZ1 mRNA clearly expressed in iPSCs irrespective of XIST induction and only modestly higher post-differentiation (FIG. 4D). Rather than XIST RNA recruiting CIZ1 to the chromosome, these results suggest that CIZ1 is already there but the epitope, detected by a monoclonal antibody is masked in pluripotent cells, except when interacting with XIST RNA. Indeed, earlier studies of CIZ1's role in DNA replication showed that it is present broadly in nuclei but only detectable by IF (with two antibodies) after chromatin removal in a matrix protocol, hence it was concluded that the CIZ1 epitope is masked by interaction with DNA (Swarts, Stewart, Higgins, & Coverley, 2018). Interestingly, CIZ1 is known to bind DNA (Warder & Keherly, 2003) and the monoclonal antibody we used targets the zinc finger region. Hence, results here strongly indicate that XIST RNA interaction similarly “unmasks” a CIZ1 epitope to reveal CIZ1 that was already present, likely involving a conformational change and/or change in DNA interaction that is triggered by XIST RNA.

Ubiquitination of H2AK119 also occurs very rapidly, likely because the PRC1 enzyme responsible is already present (Chu et al., 2015; Nesterova et al., 2019; Zylicz et al., 2019). Within two hours CIZ1 and H2AK119ub visibly accumulate in 70% of XIST expressing cells, with ˜100% by 24 hours (FIG. 4E-F). We used simultaneous staining for both proteins in an attempt to determine which appears first at the earliest timepoint, but it was inconclusive (FIG. 12A & legend). However, it's clear that changes to both CIZ1 and H2A occur very rapidly, essentially concurrent, and are induced throughout the sparse XIST RNA zone. Importantly, these results strongly support that XIST RNA functions to trigger histone modification(s) but also modifies the structural relationships of a specific non-histone nuclear matrix protein as one of the earliest “first” events.

The lamin proteins are also architectural proteins of the nuclear matrix, and the Xi is known to preferentially associate with the lamina at the nuclear periphery, as seen in ˜80% of normal (TIG-1) human fibroblasts. This repositioning to the lamina may be mediated by XIST interaction with the lamin-B receptor (LBR)(Chen et al., 2016). This study also reported that peripheral movement and lamina association was required for gene silencing, however we find in human pluripotent cells Chr21 genes are silenced without movement of the chromosome to the nuclear periphery (FIG. 4G & FIG. 1C). The silenced chromosome does relocate to the nuclear periphery in many cells upon differentiation (FIG. 4G), but not to the extend seen in fibroblasts (50% vs 80%). To address the possibility that an autosome (carrying rDNA genes) might behave differently, we also examined several pluripotent female human ES cell lines bearing a precociously inactivated X-chromosome (Hall et al., 2008), and again, only upon differentiation did the Xi become more peripheral (FIGS. 12B-C). Thus, XIST RNA impacts chromosome interaction with lamina architecture, but this change occurs later after various histone modifications and requires one or more factors expressed in differentiated cells, such as lamin A/C(Butler, Hall, Smith, & Lawrence, 2009) or possibly macroH2A, or SMCHD1 (Wang, Jegu, Chu, Oh, & Lee, 2018).

FIG. 4H summarizes our findings regarding biochemical, architectural and transcriptional changes triggered by full-length human XIST RNA during initiation of human chromosome silencing. Our collective findings all point to a larger theme: that within two hours XIST RNA spreads widely at low levels to immediately impact certain histone and non-histone chromosomal proteins prior to remodeling overall architecture, essentially all of which occurs days before most transcriptional silencing of genes.

RNA from Just the XIST A-Repeat can Silence Transcription of Local Endogenous Genes

Numerous studies have affirmed that a mutant of mouse Xist lacking the A-repeat domain can no longer transcriptionally silence genes even though the RNA still spreads widely across the chromosome (Brockdorff, 2018; Colognori, Sunwoo, Wang, Wang, & Lee, 2020; Engreitz et al., 2013; Ha et al., 2018; Wutz, Rasmussen, & Jaenisch, 2002). Hence it is well established that the A-repeat is required for silencing, but here we investigate the reciprocal question: whether the tiny (450 bp) A-repeat might itself be sufficient to transcriptionally repress endogenous loci in cis. A previous study examined this question in human HT1080 fibrosarcoma cells using qRT-PCR and found A-repeat RNA could partially repress the GFP reporter gene integrated on the same plasmid (7 kb separation)(Minks, Baldry, Yang, Cotton, & Brown, 2013), but, importantly, could not significantly repress even immediately adjacent endogenous loci (100 kb-3 Mb away). Hence, it was concluded that sequences within the missing 96% of full-length XIST RNA are required to support the function of A-repeat sequences in gene silencing.

However, since XIST RNA mediated silencing is strongly compromised in Ht1080 cells (Hall et al., 2002; Minks et al., 2013), we investigated this question further in human pluripotent cells, where XIST RNA function is optimal. As shown in FIG. 5A, we employed the same inducible promoter, insertion site, editing methodology (ZFNs) and iPS cells as was used for full-length (14 kb) XIST (flXIST) (Jiang et al., 2013) to engineer cells for inducible expression of the tiny (about 450 bp) A-repeat “nanogene” (lacking 96% of the 14 kb XIST transgene). A red fluorescent protein (RFP) gene under a constitutive promoter (EF1α) was included downstream of the A-repeat and correct targeted insertion of the transgene into the DYRK1A locus was confirmed by two-color RNA FISH in uninduced cells (FIG. 5B).

Since it has not been examined previously, the distribution of A-repeat RNA was of interest. The A-repeat produced a much smaller but intense focal RNA accumulation, after dox induction, in clear contrast to the large flXIST RNA territory (FIG. 5C). Microfluorimetric measurements indicate A-repeat RNA foci occupy an area ˜4-5% of the flXIST RNA territory, but the bright focal signal indicates substantial density of this small sequence at that site. Apart from this small focal accumulation, A-repeat RNA did not spread and localize substantially on the chromosome territory. Induction of A-repeat RNA was able to silence the RFP reporter gene integrated with the same plasmid under a separate promoter (1.7 kb away), as iPS cell colonies began losing the red fluorescence (FIG. 5D), supporting results of (Minks et al., 2013). In most experiments, we used a subset of cells that failed to induce A-repeat RNA expression (due to stochastic silencing of the tet-activator, see Methods)(FIGS. 5D & J) as a negative internal control for direct comparison of cells with and without A-repeat RNA (see below).

RFP and A-repeat transgenes are directly adjacent on the same plasmid, but a distinct question is whether the 450 bp A-repeat transcript, expressed from an intron of a large gene (DYRK1A), can impact expression of that gene's endogenous promoter (90 kb away), and potentially other nearby endogenous loci (FIG. 13A). To evaluate A-repeat effects on transcription, we used RNA/RNA FISH with gene-specific genomic probes to directly visualize transcription foci, which allows allele-specific analysis in single cells. Non-dox induced cultures show three clear DYRK1A RNA foci in essentially all cells, due to the high detection efficiency for this probe/RNA (FIG. 5B). After dox-induction for eight days, transcription foci (TF) from the DYRK1A allele in cis with the A-repeat were essentially silenced (83% of cells) (FIGS. 5E & G and FIG. 13B), whereas normal bright TFs were maintained at the other two loci. Generally, TFs at A-repeat expressing loci were entirely absent or a barely visible trace (which other observations indicate is read-through from XIST into the DYRK1a intron, see Methods). Thus, A-repeat transcripts can indeed repress transcription of the endogenous promoter of an active gene 90 kb away from the site of A-repeat transcription. For an extremely close tandem reporter gene (RFP in this study or GFP in (Minks et al., 2013)) it is harder to rule out that A-repeat effects are via steric hindrance, but the DYRK1A promoter is 90 kb away. Furthermore, uninduced cells expressing bright RFP transcription foci had no repressive effect on the nearby DYRK1A promoter (only 8% showed smaller DYRK1A RNA foci, consistent with random variation) (FIG. 5B). These results provided the first evidence that just the small A-repeat sequence itself retains gene silencing function and thus can repress a nearby endogenous locus.

Therefore, we next examined two other nearby genes that map significantly further from the integration site, DSCR3 (191 kb away) and TTC3 (385 kb away) (FIG. 13), which prior microarray results indicated are expressed in these iPSCs (Jiang et al., 2013), and for which we could generate appropriate genomic probes. Since the strength of TF signals will vary for a given gene based on size, intron content, and expression level, three transcription foci are not as consistently detected in each cell as they are for DYRK1A. Hence, we first quantified detection efficiency of TFs for these two genes in uninduced cells, using simultaneous detection of DYRK1A RNA foci for comparison (which also confirms the specific locus) (FIGS. 13C-D). Detection frequencies of TFs for DSCR3 and TTC3 at each allele was 59% and 50%, respectively (FIGS. 5F-G). While not our focus here, it is significant to note that the detection of TFs at two or all three alleles in many cells argues against single-cell seq analysis interpreted to show that most genes express from just one allele, even in trisomy 21 (e.g. Stamoulis et al., 2019) (see FIG. 13H and legend). Analysis of parallel dox-induced samples clearly showed silencing of the A-repeat associated allele in most cells (FIG. 5G and FIG. 13B-D), with the frequency of transcription foci detected dropping by 82% for DSCR3 and 83% for TTC3. This clearly demonstrates that A-repeat RNA effectively repressed transcription of genes a few hundred kb away.

Given these surprising results, we worked to evaluate two other nearby expressed genes, PIG1 (385 kb away) and HLCS (468 kb away), for which transcription foci were detected at lower but significant frequencies (20% and 25%, respectively) (FIGS. 13E-F). Nonetheless, frequency of TFs at the A-repeat allele dropped to 11% and 7% for PIG1 and HLCS, respectively (FIG. 5G). While the efficiency of A-repeat RNA silencing appears to be diminished over the genes within the 400 kb interval, silencing still occurs in many cells (˜46-74%) for loci as much as 438 kb away. We then also examined the more distal APP gene (11 Mb away), for which transcription foci are detected with high efficiency. Three APP transcription foci were always detected even after inducing the A-repeat, with no reduction in size or intensity of RNA from the allele most closely associated with the A-repeat (FIG. 5G-H and FIG. 13G)(7% appeared smaller, consistent with modest stochastic variation).

We conclude that, in the appropriate developmental cell context, just this small A-repeat fragment alone can silence transcription of endogenous genes. Importantly, this is limited to the “local chromosomal neighborhood” shown here for a region 400-450 kb from the transcription site. Consistent with its failure to spread and localize across the chromosome, gene silencing by the A-repeat appeared to drop off outside this range and had no effect on transcription of the APP locus several mega-bases away. Surprisingly, this 450 bp fragment retains this functionality outside the context of 96% of the XIST transcript. Since the small A-repeat transcripts accumulate in bright foci without spreading along the chromosome, this local concentration may increase rapidly. To test this and determine how long it takes the A-repeat RNA foci to silence local gene transcription, we induced cells for just two hours and examined levels of A-repeat and DYRK1A RNA. Within two hours of adding doxycycline dense foci of A-repeat transcripts had formed in many cells, and in parallel had quickly repressed DYRK1A transcription foci from that allele (FIG. 51). Thus, this dense focal concentration of A-repeat RNA can very quickly silence nearby gene transcription.

In addition to the concentrated A-repeat RNA focus, A-repeat transcripts are also seen dispersed uniformly throughout the nucleoplasm at lower levels (FIG. 5J) but are not found in the cytoplasm, as is RFP mRNA (FIGS. 131-J). Similar nucleus-wide dispersal of flXIST RNA is only seen when it is released from the chromosome by manipulation of chromatin phosphorylation (FIG. 2G). Full-length XIST RNA is also highly stable, with a half-life of about five hours (Clemson, Chow, Brown, & Lawrence, 1998; Clemson et al., 1996), whereas we find the A-repeat RNA focus dissipates after 30 minutes of transcriptional inhibition, and nucleoplasmic A-repeat RNA after about an hour (FIG. 13K). Hence, the A-repeat transcript accumulates locally to silence nearby genes but is released from chromatin to disperse. And although it is much less stable than flXIST, it is not immediately degraded and can populate the nucleoplasm, as will be further considered below.

In addition, experiments using RNAseq further showed repression by two A-repeat minigenes (450 bp and 2.5 Kb). The 2.5 Kb minigene includes additional XIST sequences as shown in FIG. 7A (see the Examples), and represses genes in a similar limited region.

### Effective Deacetylation to Initiate Gene Silencing Requires High Density of A-Repeat/XIST Transcripts

Results above show that flXIST RNA spreads rapidly across the chromosome and that A-repeat RNA itself can silence genes, and does so rapidly. Since flXIST transcripts contain the A-repeat and spread across the chromosome territory within hours, why didn't flXIST RNA induce long-range gene silencing more quickly? The widely distributed flXIST RNA is clearly sufficient to trigger robust UbH2A and CIZ1 staining within just two hours, yet it took several days to silence several randomly selected genes, and this occurred only after coalescence of the chromosome and XIST territory.

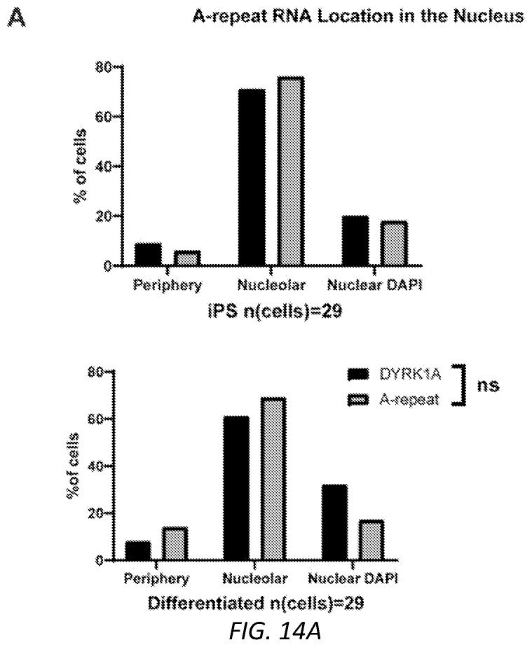

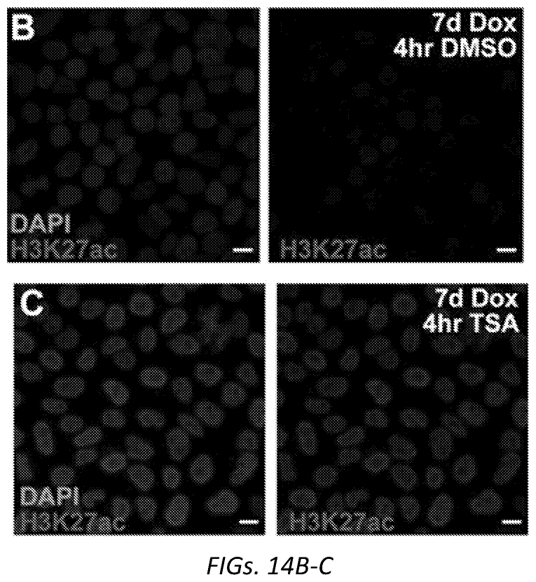

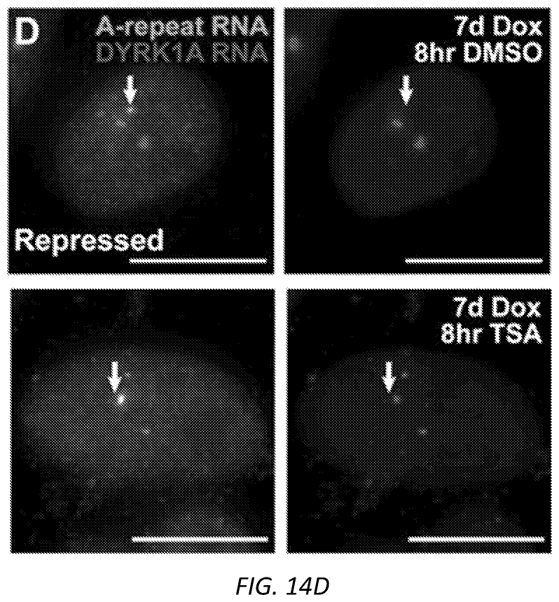

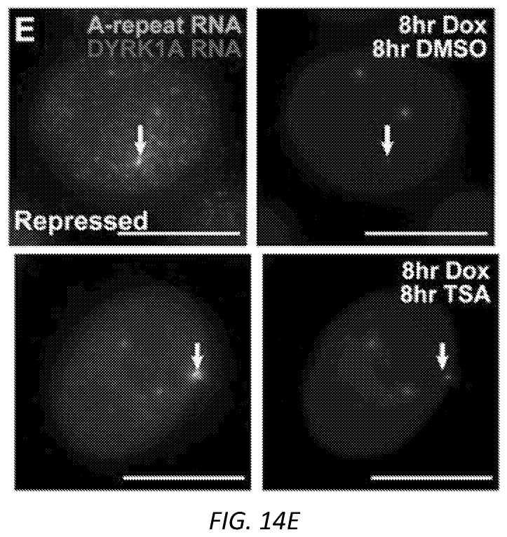

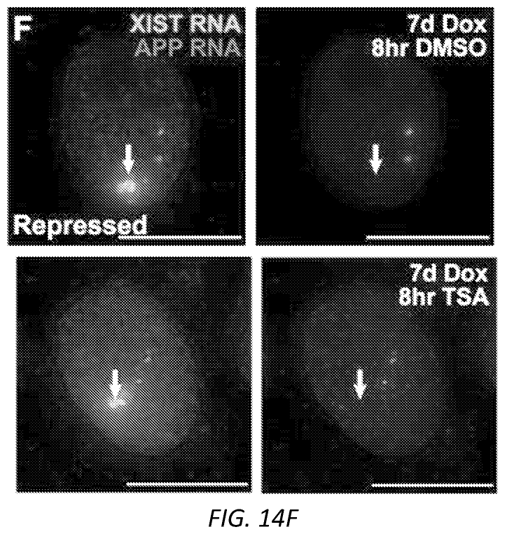

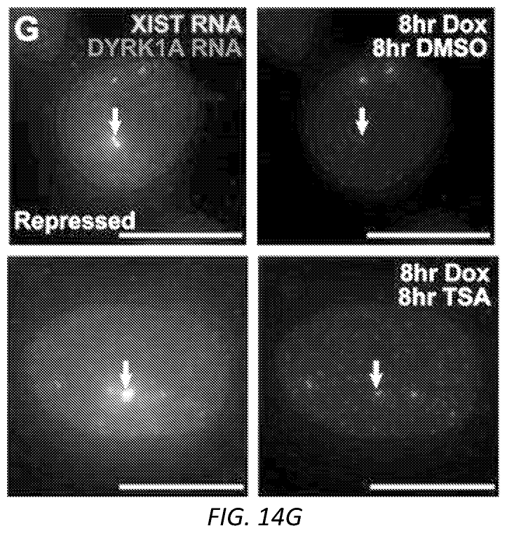

To gain insight into this we further considered how the A-repeat functions, since this sequence is required for the gene silencing process. This has been previously studied by deletion of the A-repeat, whereas here we examine effects of A-repeat alone in terms of two main functions that have been implicated: histone deacetylation and chromosome organization with the nuclear lamina. Evidence indicates the A-repeat domain is required to recruit HDACs for H3/H4 deacetylation (via SPEN) which is important in the chromosome silencing process (Brockdorff, Bowness, & Wei, 2020; Chu et al., 2015; McHugh et al., 2015; Nesterova et al., 2019; Zylicz et al., 2019). In addition, the A-repeat has been shown to bind the lamin B receptor (LBR)(McHugh et al., 2015) and the consequent tethering of the chromosome to the lamina (at the peripheral heterochromatin compartment) was reportedly required for gene silencing (Chen et al., 2016). However, as shown above, our results with flXIST RNA do not support the requirement of peripheral lamina association for gene/chromosome silencing, although our results suggest this could be related to maintaining the XIST-independent heterochromatic state that occurs post-differentiation (when we see peripheral movement). Likewise, A-repeat RNA foci do not localize to the nuclear periphery (in pluripotent or differentiated cells, FIG. 14A), yet are still able to silence genes locally (e.g. FIG. 5G).

Hence, we investigated whether the small 450 bp A-repeat RNA still acts via deacetylation to block transcription when separated from the larger XIST transcript. A 4-hour TSA treatment (or 8-hour at lower concentration) was sufficient to inhibit histone deacetylation, increasing H3K27ac across the nucleoplasm (FIGS. 14B-C), and was short enough to avoid secondary toxic effects. Gene silencing by either the A-repeat RNA or flXIST RNA drops markedly if histone deacetylation is blocked concomitant with dox-induction (FIGS. 6A-C & FIGS. 14E&G), demonstrating that the small A-repeat transcript retains similar function independent of the long XIST transcript; both rely on histone deacetylation to induce initial gene silencing. However, an important difference is seen if deacetylase inhibition follows dox-induction by several days, when gene silencing has already occurred. For the A-repeat RNA, TSA treatment results in re-appearance of transcription foci (FIG. 6A-C & FIG. 14D), indicating that that ongoing HDAC recruitment/activity is required, defining a reversible “HDAC-dependent” state. In contrast, the gene silencing induced by flXIST RNA is not reversed but has become “HDAC-independent” (FIG. 6A-C & FIG. 14F). Hence other domains of XIST RNA are required for modifications, such as H3K27 methylation, that likely block reacetylation and stabilize gene repression.

Unlike more stable “epigenetic” changes, histone deacetylation has a broad role in gene regulation that involves an ongoing dynamic balance between deacetylation (HDAC) and acetylation (HAT). Hence, efficient transcriptional repression by A-repeat RNA may require HDAC density sufficient to compete with HAT activity in active chromatin regions, in order to shift the balance towards repression. As indicated above, in addition to the dense A-repeat RNA foci, many cells contain substantial but lower levels of A-repeat RNA throughout the nucleoplasm. To determine if these lower levels of A-repeat transcripts have any detectable impact on transcription we examined H3K27ac levels, hnRNA levels or specific gene transcription in these cells, in comparison to neighboring cells with no A-repeat transgene expression. Cells with substantial nucleoplasmic A-repeat RNA showed no reduction in hnRNA (as detected by CoT-1 RNA)(FIG. 6D) nor in H3K27ac levels (FIG. 6E) compared to neighboring non-expressing cells. Similarly, the TFs for all genes studied above were only repressed when in cis with the dense A-repeat RNA foci with no difference for alleles within the nucleoplasm containing substantial A-repeat RNA signal, when compared to nearby cells lacking A-repeat expression (e.g. FIG. 6F).

The above results suggest that effective deacetylation by A-repeat sequences within the full-length XIST RNA may be density dependent, which led us to examine H3K27ac staining during silencing by the flXIST transcript. To examine acetylation across the time-course on individual inactivating chromosomes, we used H2AK119ub as a proxy for XIST RNA to optimize detection of H3K27ac (to eliminate RNA hybridization that can weaken IF). FIG. 6G shows that in cells expressing flXIST RNA for seven days, when the process is essentially complete, there is a marked “acetylation void” over the whole inactivate chromosome 21, as labeled by H2AK119ub enrichment. Hence, we examined the extent to which deacetylation was seen at earlier time points. Unlike H2AK119ub enrichment which coincides with XIST RNA spread from the earliest time points, any decrease in histone acetylation staining is barely discernible at early hours (e.g. FIG. 6H), and only becomes more clearly evidenced at ˜1 day (FIG. 6I), although not to the level seen for the fully silenced chromosome (Day 7). In some cells at early timepoints a dip in the acetylase staining can be seen in the smaller dense zone (FIG. 6H: insert), and consistent with that, we found DYRK1A gene silencing at the epicenter of XIST expression is silenced more rapidly (FIG. 6J). Nonetheless, for most of the chromosome the spread of histone deacetylation and gene silencing lags substantially behind H2AK119ub, CIZ1, and formation of the CoT-1 RNA void, and is subsequent to the overall architectural condensation that builds the dense territory of XIST RNA.

Thus the collective results here indicate that the HDAC activity of the A-repeat element is necessary and sufficient for initiation of gene silencing, but this necessary first step also requires greater transcript density, which comes once the flXIST RNA territory coalesces on the condensing chromosome territory. Thus, much of the long flXIST transcript functions to spread RNA across the chromosome and architecturally compact the chromosome territory to increase flXIST transcript/A-repeat/HDAC density to effectively silence genes (HDAC-dependent state) and then “lock-down” the silent state (HDAC-independent), which is later stabilized during differentiation (XIST-independent).

### Example 2. Generalized Method to Reduce Expression of One Allele by Integrating the A-Repeat Construct into 5′ UTR, Intron, or Exon of One Allele

This example describes a generalized method for reducing expression of one allele by integrating the A-repeat construct into or near a SNP or other unique sequence located in proximity to the allele (e.g., 5′ UTR, intron, or exon) was developed.

In brief, the gene or genomic region of interest would be selected. Exemplary examples of genomic regions of interest include, but are not limited to 1q21 microduplication, 2p15p16 microduplication, 3q29 microduplication, 15q13.1 microduplication, and, 15q24 microduplication. Single nucleotide polymorphisms (SNPs) or other allele-specific unique sequences located in the selected genomic region (e.g. if gene in 5′ UTR, introns, or exons) are identified either from publically available databases (e.g. NCBI Short Genetic Variations database (dbSNP) available at ncbi.nlm.nih.gov/projects/SNP/index.html) or from quantification of alleles (frequency and sequence) present in a population (e.g. subset of patients or population of cells) (Aggeli et al. (2018). Diff-seq: A high throughput sequencing-based mismatch detection assay for DNA variant enrichment and discovery. Nucleic Acids Res 46(7): e42). The SNPs or other allele-specific unique sequences identified are rank ordered based on those with allele frequencies closest to 50 percent and those with higher numbers of nucleotide differences between the two allele sequences. This rank ordering prioritizes frequency of heterozygosity such that both alleles are present in the cell being targeted and prioritizes SNPs for which highly specific targeting reagents (e.g. guide RNA design if targeting accomplished by CRISPR-Cas9) can be designed. Guide RNAs are designed according to known methods in the prior art (e.g., Akcakaya et al. (2018). In vivo CRISPR editing with no detectable genome-wide off-target mutations. Nature 561: 416-419; Tycko et al. (2016). Method for optimizing CRISPR-Cas9 Genome Editing Specificity. 4; 63(3): 355-370). The guide RNAs are synthesized by vendors (e.g. Sigma) and screened by methods known in the art (e.g. TE71 assay, Surveyor assay; Bell et al. (2014) A high-throughput screening strategy for detecting CRISPR-Cas9 induced mutations using next-generation sequencing. BMC Genomics 15:1002) to select sgRNAs:Cas9 complexes that efficiently and specifically cut the targeted SNP sequence and do not cut the sequence of the other allele.

### Example 3. Insertion of an A-Repeat Construct into a Mouse Model of Down Syndrome

TcMAC21 is a newly developed DS mouse model that carries the long arm of the human chr21 (Kazuki et al., eLife 9:e56223 (2020)). These mice express the green fluorescent protein (GFP) and express >90% human chr21 genes. They recapitulate several phenotypes seen in human DS individuals such as smaller cerebellum, heart defects, and learning and memory deficits.

Using an rAAV donor and CRISPR/Cas9, we targeted the A-repeat into human chr21 into the human DYRK1a locus in TcMAC21 mouse zygotes and generated transgenic mice. RNA fluorescent in situ hybridization (FISH) in mouse tail tip fibroblasts was used to confirm insertion of the A-repeat fragment into the human chr21.

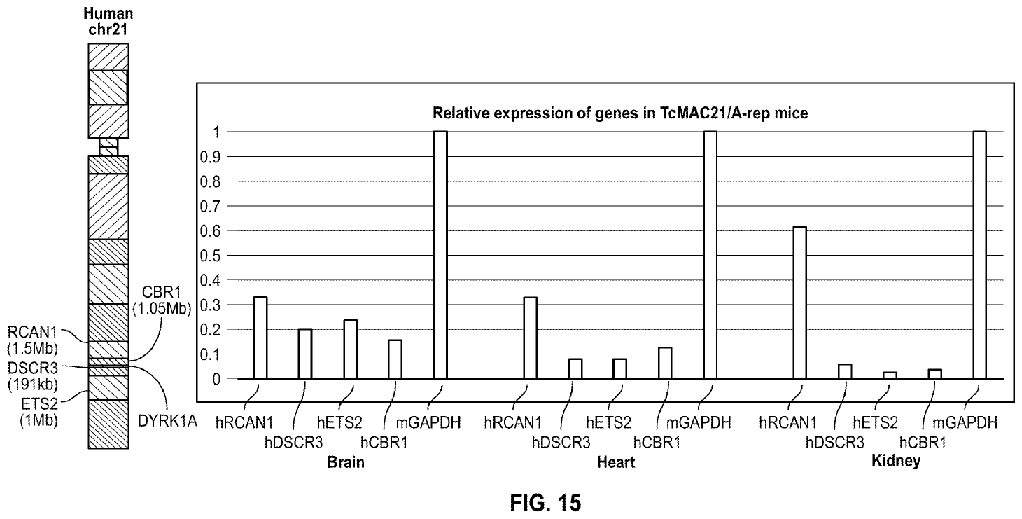

To further determine whether the A-repeat repressed expression of human chr21 genes in the DSCR in vivo, we performed an RT-qPCR assay. This allowed us to quantitatively examine the relative levels of several chr21 genes near the A-repeat insertion site in the TcMAC21/A-repeat mice that were normalized to the TcMAC21 mice. We observed about 70% repression of genes examined near the site of insertion of the A-fragment in different mouse tissues brain, heart, and kidney in 15 day old mice (FIG. 15). By repressing the DSCR (“DS Critical Region”), we were able to reduce the dosage of several transcription factors (ETS2, ERG) that are speculated to be involved in hematopoiesis.

### EXEMPLARY SEQUENCES AND CONSTRUCTS

In some embodiments, the sequence of a protein or nucleic acid used in a composition or method described herein is at least 80%, 85%, 90%, 95%, 97%, 98%, or 99% identical to a reference sequence set forth herein. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 80% of the length of the reference sequence, and in some embodiments is at least 90% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can be determined using the Needleman and Wunsch ((1970) J. Mol. Biol. 48:444-453) algorithm which has been incorporated into the GAP program in the GCG software package (available on the world wide web at gcg.com), using the default parameters, e.g., a Blossum 62 scoring matrix with a gap penalty of 12, a gap extend penalty of 4, and a frameshift gap penalty of 5.

A repeat full sequence (in exemplary transgenes)
GAATTTTTCTTTGGAATCATTTTTGGTTGACATCTCTGTTTTTTGTGGATCAGTTTTTTACT
CTTCCACTCTCTTTTCTATATTTTGCCCATCGGGGCTGCGGATACCTGGTTTTATTATTTTT
TCTTTGCCCAACGGGGCCGTGGATACCTGCCTTTTAATTCTTTTTTATTCGCCCATCGGGGC
CGCGGATACCTGCTTTTTATTTTTTTTTCCTTAGCCCATCGGGGTATCGGATACCTGCTGAT
TCCCTTCCCCTCTGAACCCCCAACACTCTGGCCCATCGGGGTGACGGATATCTGCTTTTTAA
AAATTTTCTTTTTTTGGCCCATCGGGGCTTCGGATACCTGCTTTTTTTTTTTTTATTTTTCC
TTGCCCATCGGGGCCTCGGATACCTGCTTTAATTTTTGTTTTTCTGGCCCATCGGGGCCGCG
GATACCTGCTTTGATTTTTTTTTTTCATCGCCCATCGGTGCTTTTTATGGATGAAAAAATGT
T
A repeat consensus
Core sequence missing spacer T rich
GCCCATCGGGGCCGCGGATACCTG
Benson consensus (repeat finder)
TTCGCCCATCGGGGCCGCGGATACCTGCTTTTTATTTTTTTTTC
-Clustal analysis of pre-defined repeats

| | | |
|---|---|---|
| 5 | -CCCCAACACTCTGGCCCATCGGGGTGACGGATATCTGCTTTTTAAAAA----------- | 48 |
| 4 | -TTTTTTTTCCTTAGCCCATCGGGGTATCGGATACCTGCTGATTCCCTTCCCCTCTGAAC | 59 |
| 9 | -TTTTTTTTTCATCGCCCATCGGTGC---------------------------------- | 25 |
| 2 | -TTATTTTTTCTTTGCCCAACGGGGCCGTGGATACCTGCCTTTTAA-------------- | 45 |
| 3 | -TTCTTTTTTATTCGCCCATCGGGGCCGCGGATACCTGCTTTTTAT-------------- | 45 |
| 1 | -TTTTCTATATTTTGCCCATCGGGGCTGCGGATACCTGGTTTTA---------------- | 43 |
| 7 | TTTTATTTTTCCTTGCCCATCGGGGCCTCGGATACCTGCTTTAA---------------- | 44 |
| 6 | -TTTTCTTTTTTTGGCCCATCGGGGCTTCGGATACCTGCTTTTTTTTT------------ | 47 |
| 8 | TTTTTGTTTTTCTGGCCCATCGGGGCCGCGGATACCTGCTTTGATT-------------- | 46 |

* ***** *** *
Query: 1 Query ID: lcl|Query_547181 Length: 494
-Blast analysis of full A-repeat (this is one possible outcome, many others)
Query range 2: 61 to 120

| | | | |
|---|---|---|---|
| Query | 57 | AGGTATCCGCGGCCCCGATGGGC--AGAAAAACAAAAATT-----AAA--GCAGGTATCC | 107 |
| Query_547183 | 57 | AGGTATCCGCGGCCCCGATGGGC--AGAAAAACAAAAATT-----AAA--GCAGGTATCC | 107 |
| Query_547183 | 299 | AGGTATCCGCGGCCCCGATGGGC--GAATAAAAAAGAATT-----AAAAGGCAGGTATCC | 351 |
| Query_547183 | 344 | AGGTATCCACGGCCCCGTTGGGC--AAAGAAAAAATAATA-----AAA--CCAGGTATCC | 394 |
| Query_547183 | 254 | AGGTATCCGATACCCCGATGGGCTAAGGAAAAAAAAATAA-----AAA--GCAGGTATCC | 306 |
| Query_547183 | 147 | AGGTATCCGAAGCCCCGATGGGC--CAAAAAAAGAAAATTTTTAAAAA--GCAGATATCC | 202 |

F repeats 1206-1561, repeat = 355 nts , 40 nt each
GACATTGCGGCATTGCTCAGCATGGCGGGCTGTGCTTTGTTAGGTTGTCCAAAATGGCGGAT
CCAGTTCTGTCGCAGTGTTCAAGTGGCGGGAAGGCCACATCATGATGGGCGAGGCTTTGTTA
AGTGGTTAGCATGGTGGTGGACATGTGCGGTCACACAGGAAAAGATGGCGGCTGAAGGTCTT
GCCGCAGTGTAAAACATGGCGGGCCTCTTTGTCTTTGCTGTGTGCTTTTCGTGTTGGGTTTT
GCCGCAGGGACAATATGGCAGGCGTTGTCATATGTATATCATGGCTTTTGTCACGTGGACAT
CATGGCGGGCTTGCCGCATTGTTAAAGATGGCGGGTTTTGCCGCCT
F1: 1206 GACATTGCGGCATTGCTCAGCATGGCGGGCTGTGCTTTTG 1245
F2: 1386 GGTCTTGCCGCAGTGTAAAACATGGCGGGCCTCTTTGTCT 1425
F3: 1522 GGGCTTGCCGCATTGTTAAAGATGGCGGGTTTTGCCGCCT 1561
Consensus: ggncTTGCcGCAtTGttaAacATGGCGGGctttgctgtct
Distance between repeats :
F1-F2 = 141 nt
F2-F3 = 97 nt
Repeat Bh (tandem 8 mer repeats, 94 nts region) NTS 1975-2069:
ATACCTCCCCCCCCACCCCCCAACCCCCCCAACTCCCCACCCCCACCCCCCACCCCCCACCT
CCCCACCCCCCTACCCCCCTACCCCCCTACCCCCC

| | | | |
|---|---|---|---|
| B1 | 1975 | TACCTCCC | 1982 |
| B2 | 1987 | CACCCCCC | 1994 |
| B3 | 1995 | AACCCCCC | 2002 |
| B4 | 2004 | AACTCCCC | 2012 |
| B5 | 2012 | CACCCCCA | 2019 |
| B6 | 2017 | CACCCCCC | 2024 |
| B7 | 2025 | CACCCCCC | 2032 |
| B8 | 2030 | CACCTCCC | 2037 |
| B9 | 2038 | CACCCCCC | 2045 |
| B10 | 2046 | TACCCCCC | 2053 |
| B11 | 2054 | TACCCCCC | 2061 |
| B12 | 2062 | TACCCCCC | 2069 |

Consensus: ACCCCCC
B Repeat 119 bp (2809-2927) in human separated from Bh, but not
in mouse
CCTCCCCAGCCCTGCTCCCAGCAAACCCCTAGTCTAGCCCCAGCCCTACTCCCACCCCGCCC
CAGCCCTGCCCCAGCCCCAGTCCCCTAACCCCCCAGCCCTAGCCCCAGTCCCAGTCC
1. TCCCCa
2. ACCCCT
3. GCCCCA
4. ACCCCG
5. GCCCCA
6. GCCCCA
7. GCCCCA
8. TCCCCT
9. ACCCCCCA
10. GCCCCA
C repeat (only one in humans)
TGCTCAAAATAAGTTGTCCATTGCTTATCCTATTATACTGGGATATTCCGTTTACCCTTGGC
ATTGCTGATCTTCAGTACTGACTCCTTGACCATTTTCAGTTAATGCATACAATCCCAT
D repeat (5962-8595) = 2634 nt. Tandem 10x279-305 nt
CCTTTTGAAGGACAGCATGGTTGGTGACACCTAAGGCCCCATTTCTTGGCCTCCCAATATGT
GTGATTGTATTTGTCGAGGTTGCTATGCACTAGAGAAGGAAAGTGCTCCCCTCATCCCCACT
TTTCCCTTCCAGCAGGAAGTGCCCACCCCATAAGACCCTTTTATTTGGAGAGTCTAGGTGCA
CAATTGTAAGTGACCACAAGCATGCATCTTGGACATTTATGTGCGTAATCGCACACTGCTCA
TTCCATGTGAATAAGGTCCTACTCTCCGACCCCTTTTGCAATACAGAAGGGTTGCTGATAAC
GCAGTCCCCTTTTCTTGGCATGTTGTGTGTGATTATAATCGTCTGGGATCCTATGCACTAGA
AAAGGAGGGTCCTCTCCACATACCTCAGTCTCACCTTTCCCTTCCAGCAGGGAGTGCCCACT
CCATAAGACTCTCACATTTGGACAGTCAAGGTGCGTAATTGTTAAGTGAACACAACCATGCA
CCTTAGACATGGATTTGCATAACTACACACAGCTCAACCTATCTGAATAAAATCCTACTCTC
AGACCCCTTTTGCAGTACAGCAGGGGTGCTGATCACCAAGGCCCTTTTTCCTGGCCTGGTAT
GCGTGTGATTATGTTTGTCCCGGTTCCTGTGTATTAGACATGGAAGCCTCCCCTGCCACACT
CCACCCCCAATCTTCCTTTCCCTTCCGGCAGGGAGTGCCCTCTCCATAAGACGCTTACGTTT
GGACAATCAAGGTGCACAGTTGTAAGTGACCACAGGCATACACCTTGGACATTAATGTGCAT
AACCACTTTGCCCATTCCATCTGAATAAGGTCCTACTCTCAGACCCCTTTTGCAGTACAGCA
GGGGTGCTGATCACCAAGGCCCCTTTTCTTGGCCTGTTATGTGCGTGATTATATTTGTCTGG
GTTCCTGTGTATTAGACAAGGAAGCCTTCCCCCCGCCCCCACCCCCACTCCCAGTCTTCCTT
TCCCTTCCAGCAGGGAGTGCCCCCTCCATAAGATCATTACATTTGGACAATCAAGGTGCACA
ATTATAAGTGACCACAGCCATGCACCTTGGACATTATTGGACATTAATGTGCGTAACTGCAC
ATGGCCCATCCCATCTGAATAAGGTCCTACTCTCAGATGCCCTTTGCAGTACAGCAGGGGTA
CTGAATCACCAAGGCCCTTTTTCTTGGCCTGTTATGTGTGTGATTATATTTATCCCAGTTTC
TGTGTAATAGACATGAAAGCCTCCCCTGCCACACCCCACCTCCAATCTTCCTTTCCCTTCCA
CCAGGGAGTGTCCACTCCATATACCCTTACATTTGGACAATCAAGGTGCACAATTGTAAGTG
AGCATAGGCACTCACCTTGGACATGAATGTGCATAACTGCACATGGCCCATCCCATCTGAAT
AAGGTCCTACTCTCAGACCCTTTTTGCAGTACAGCAGGGGTGCTGATCACCAAGGCCCCTTT
TCCTGGCCTGTTATGTGTGTGATTATATTTGTTCCAGTTCCTGTGTAATAGACATGGAAGCC
TCCCCTGCCACACTCCACCCCCAATCTTCCTTTCCCTTCTGGCAGGAAGTACCCGCTCCATA
AGACCCTTACATTTGGACAGTCAAGGTGCACAATTGTATGTGACCACAACCATGCACCTTGG
ACATAAATGTGTGTAACTGCACATGGCCCATCCCATCTGAATAAGGTCCTACTCTCAGACCC
CTTTTGCAGTACAGTAGGTGTGCTGATAACCAAGGCCCCTCTTCCTGGCCTGTTAACGTATG
TGATTATATTTGTCTGGGTTCCAGTGTATAAGACATGGAAGCCTCCCCTGCCCCACCCCACC
CTCAATCTTCCTTTCCCTTCTGGCAGGGAGTGCCAGCTCCATAAGAACCTTACATTTGGACA
GTCAAGGTGCACAATTCTAAGTGACCGCAGCCATGCACCTTGGTCAATAATGTGTGTAACTG
CACACGGCCTATCTCATCTGAATAAGGCCTTACTCTCAGACCCCTTTTGCAGTACAGCAGGG
GTGCTGATAACCAAGGCCCATTTTCCTGGCCTGTTATGTGTGTGATTATATTTGTCCAGGTT
TCTGTGTACTAGACAAGGAAGCCTCCTCTGCCCCATCCCATCTACGCATAATCTTTCTTTTC
CTCCCAGCAGGGAGTGCTCACTCCATAAGACCCTTACATTTGGACAATCAAGGTGCACAATT
GTAAGTGACCACAACCATGCATCTTGGAAATTTATGTGCATAACTGCACATGGCTTATCCTA
TTTGAATAAAGTCCTACTCTCAGACCCCCTTTGCAGTATAGCTGGGGTGCTGATCACTGAGG
CCTCTTTGCTTGGCTTGTCTATATTCTTGTGTACTAGATAAGGGCACCTTCTCATGGACTCC
CTTTGCTTTTCAACAAGGAGTACCCACTACTTTTTAAGATTCTTATATTTGTCCAAAGTACA
TGGTTTTAATTGACCACAACAATGTCCCTTGGACATTAATGTATGTAATCACCACATGGTTC
ATCCTAATTAAACAAAGTTCTACCTTCTCACCCTCCATTTGCAGTATACCAGGGTTGCTGAC
CCCCTAAGTCCCCTTTTCTTGGCTTGTTGA
>D1
CCTTTTGAAGGACAGCATGGTTGGTGACACCTAAGGCCCCATTTCTTGGCCTCCCAATATGT
GTGATTGTATTTGTCGAGGTTGCTATGCACTAGAGAAGGAAAGTGCTCCCCTCATCCCCACT
TTTCCCTTCCAGCAGGAAGTGCCCACCCCATAAGACCCTTTTATTTGGAGAGTCTAGGTGCA
CAATTGTAAGTGACCACAAGCATGCATCTTGGACATTTATGTGCGTAATCGCACACTGCTCA
TTCCATGTGAATAAGGTCCTACTCTCCGACC
>D2
CCTTTTGCAATACAGAAGGGTTGCTGATAACGCAGTCCCCTTTTCTTGGCATGTTGTGTGTG
ATTATAATCGTCTGGGATCCTATGCACTAGAAAAGGAGGGTCCTCTCCACATACCTCAGTCT
CACCTTTCCCTTCCAGCAGGGAGTGCCCACTCCATAAGACTCTCACATTTGGACAGTCAAGG
TGCGTAATTGTTAAGTGAACACAACCATGCACCTTAGACATGGATTTGCATAACTACACACA
GCTCAACCTATCTGAATAAAATCCTACTCTCAGACC
>D3
CCTTTTGCAGTACAGCAGGGGTGCTGATCACCAAGGCCCTTTTTCCTGGCCTGGTATGCGTG
TGATTATGTTTGTCCCGGTTCCTGTGTATTAGACATGGAAGCCTCCCCTGCCACACTCCACC
CCCAATCTTCCTTTCCCTTCCGGCAGGGAGTGCCCTCTCCATAAGACGCTTACGTTTGGACA
ATCAAGGTGCACAGTTGTAAGTGACCACAGGCATACACCTTGGACATTAATGTGCATAACCA
CTTTGCCCATTCCATCTGAATAAGGTCCTACTCTCAGACC
>D4
CCTTTTGCAGTACAGCAGGGGTGCTGATCACCAAGGCCCCTTTTCTTGGCCTGTTATGTGCG
TGATTATATTTGTCTGGGTTCCTGTGTATTAGACAAGGAAGCCTTCCCCCCGCCCCCACCCC
CACTCCCAGTCTTCCTTTCCCTTCCAGCAGGGAGTGCCCCCTCCATAAGATCATTACATTTG
GACAATCAAGGTGCACAATTATAAGTGACCACAGCCATGCACCTTGGACATTATTGGACATT
AATGTGCGTAACTGCACATGGCCCATCCCATCTGAATAAGGTCCTACTCTCAGATGC
>D5
CCTTTGCAGTACAGCAGGGGTACTGAATCACCAAGGCCCTTTTTCTTGGCCTGTTATGTGTG
TGATTATATTTATCCCAGTTTCTGTGTAATAGACATGAAAGCCTCCCCTGCCACACCCCACC
TCCAATCTTCCTTTCCCTTCCACCAGGGAGTGTCCACTCCATATACCCTTACATTTGGACAA
TCAAGGTGCACAATTGTAAGTGAGCATAGGCACTCACCTTGGACATGAATGTGCATAACTGC
ACATGGCCCATCCCATCTGAATAAGGTCCTACTCTCAGACC
>D6
CTTTTTGCAGTACAGCAGGGGTGCTGATCACCAAGGCCCCTTTTCCTGGCCTGTTATGTGTG
TGATTATATTTGTTCCAGTTCCTGTGTAATAGACATGGAAGCCTCCCCTGCCACACTCCACC
CCCAATCTTCCTTTCCCTTCTGGCAGGAAGTACCCGCTCCATAAGACCCTTACATTTGGACA
GTCAAGGTGCACAATTGTATGTGACCACAACCATGCACCTTGGACATAAATGTGTGTAACTG
CACATGGCCCATCCCATCTGAATAAGGTCCTACTCTCAGACC
>D7
CCTTTTGCAGTACAGTAGGTGTGCTGATAACCAAGGCCCCTCTTCCTGGCCTGTTAACGTAT
GTGATTATATTTGTCTGGGTTCCAGTGTATAAGACATGGAAGCCTCCCCTGCCCCACCCCAC
CCTCAATCTTCCTTTCCCTTCTGGCAGGGAGTGCCAGCTCCATAAGAACCTTACATTTGGAC
AGTCAAGGTGCACAATTCTAAGTGACCGCAGCCATGCACCTTGGTCAATAATGTGTGTAACT
GCACACGGCCTATCTCATCTGAATAAGGCCTTACTCTCAGACC
>D8
CCTTTTGCAGTACAGCAGGGGTGCTGATAACCAAGGCCCATTTTCCTGGCCTGTTATGTGTG
TGATTATATTTGTCCAGGTTTCTGTGTACTAGACAAGGAAGCCTCCTCTGCCCCATCCCATC
TACGCATAATCTTTCTTTTCCTCCCAGCAGGGAGTGCTCACTCCATAAGACCCTTACATTTG
GACAATCAAGGTGCACAATTGTAAGTGACCACAACCATGCATCTTGGAAATTTATGTGCATA
ACTGCACATGGCTTATCCTATTTGAATAAAGTCCTACTCTCAGACC
>D9
CCCTTTGCAGTATAGCTGGGGTGCTGATCACTGAGGCCTCTTTGCTTGGCTTGTCTATATTC
TTGTGTACTAGATAAGGGCACCTTCTCATGGACTCCCTTTGCTTTTCAACAAGGAGTACCCA
CTACTTTTTAAGATTCTTATATTTGTCCAAAGTACATGGTTTTAATTGACCACAACAATGTC
CCTTGGACATTAATGTATGTAATCACCACATGGTTCATCCTAATTAAACAAAGTTCTACCTT
CTCACCCTCCATTTGCAGTATACCAGGGTTGCTGACCCCCTAAGTCC
>D10
CCTTTTCTTGGCTTGTTGA
A repeat +F + B repeat
A -9.5x50 nt
F = 3x40 nt
Bh = 12x8 nt
B = 10x4 nt
A repeat full sequence
GAATTTTTCTTTGGAATCATTTTTGGTTGACATCTCTGTTTTTTGTGGATCAGTTTTTTACT
CTTCCACTCTCTTTTCTATATTTTGCCCATCGGGGCTGCGGATACCTGGTTTTATTATTTTT
TCTTTGCCCAACGGGGCCGTGGATACCTGCCTTTTAATTCTTTTTTATTCGCCCATCGGGGC
CGCGGATACCTGCTTTTTATTTTTTTTTCCTTAGCCCATCGGGGTATCGGATACCTGCTGAT
TCCCTTCCCCTCTGAACCCCCAACACTCTGGCCCATCGGGGTGACGGATATCTGCTTTTTAA
AAATTTTCTTTTTTTGGCCCATCGGGGCTTCGGATACCTGCTTTTTTTTTTTTTATTTTTCC
TTGCCCATCGGGGCCTCGGATACCTGCTTTAATTTTTGTTTTTCTGGCCCATCGGGGCCGCG
GATACCTGCTTTGATTTTTTTTTTTCATCGCCCATCGGTGCTTTTTATGGATGAAAAAATGT
T
F repeats
GACATTGCGGCATTGCTCAGCATGGCGGGCTGTGCTTTGTTAGGTTGTCCAAAATGGCGGAT
CCAGTTCTGTCGCAGTGTTCAAGTGGCGGGAAGGCCACATCATGATGGGCGAGGCTTTGTTA
AGTGGTTAGCATGGTGGTGGACATGTGCGGTCACACAGGAAAAGATGGCGGCTGAAGGTCTT
GCCGCAGTGTAAAACATGGCGGGCCTCTTTGTCTTTGCTGTGTGCTTTTCGTGTTGGGTTTT
GCCGCAGGGACAATATGGCAGGCGTTGTCATATGTATATCATGGCTTTTGTCACGTGGACAT
CATGGCGGGCTTGCCGCATTGTTAAAGATGGCGGGTTTTGCCGCCT
Repeat Bh
ATACCTCCCCCCCCACCCCCCAACCCCCCCAACTCCCCACCCCCACCCCCCACCCCCCACCT
CCCCACCCCCCTACCCCCCTACCCCCCTACCCCCC
B Repeat
CCTCCCCAGCCCTGCTCCCAGCAAACCCCTAGTCTAGCCCCAGCCCTACTCCCACCCCGCCC
CAGCCCTGCCCCAGCCCCAGTCCCCTAACCCCCCAGCCCTAGCCCCAGTCCCAGTCC
C repeat
TGCTCAAAATAAGTTGTCCATTGCTTATCCTATTATACTGGGATATTCCGTTTACCCTTGGC
ATTGCTGATCTTCAGTACTGACTCCTTGACCATTTTCAGTTAATGCATACAATCCCAT
D repeat
CCTTTTGAAGGACAGCATGGTTGGTGACACCTAAGGCCCCATTTCTTGGCCTCCCAATATGT
GTGATTGTATTTGTCGAGGTTGCTATGCACTAGAGAAGGAAAGTGCTCCCCTCATCCCCACT
TTTCCCTTCCAGCAGGAAGTGCCCACCCCATAAGACCCTTTTATTTGGAGAGTCTAGGTGCA
CAATTGTAAGTGACCACAAGCATGCATCTTGGACATTTATGTGCGTAATCGCACACTGCTCA
TTCCATGTGAATAAGGTCCTACTCTCCGACCCCTTTTGCAATACAGAAGGGTTGCTGATAAC
GCAGTCCCCTTTTCTTGGCATGTTGTGTGTGATTATAATCGTCTGGGATCCTATGCACTAGA
AAAGGAGGGTCCTCTCCACATACCTCAGTCTCACCTTTCCCTTCCAGCAGGGAGTGCCCACT
CCATAAGACTCTCACATTTGGACAGTCAAGGTGCGTAATTGTTAAGTGAACACAACCATGCA
CCTTAGACATGGATTTGCATAACTACACACAGCTCAACCTATCTGAATAAAATCCTACTCTC
AGACCCCTTTTGCAGTACAGCAGGGGTGCTGATCACCAAGGCCCTTTTTCCTGGCCTGGTAT
GCGTGTGATTATGTTTGTCCCGGTTCCTGTGTATTAGACATGGAAGCCTCCCCTGCCACACT
CCACCCCCAATCTTCCTTTCCCTTCCGGCAGGGAGTGCCCTCTCCATAAGACGCTTACGTTT
GGACAATCAAGGTGCACAGTTGTAAGTGACCACAGGCATACACCTTGGACATTAATGTGCAT
AACCACTTTGCCCATTCCATCTGAATAAGGTCCTACTCTCAGACCCCTTTTGCAGTACAGCA
GGGGTGCTGATCACCAAGGCCCCTTTTCTTGGCCTGTTATGTGCGTGATTATATTTGTCTGG
GTTCCTGTGTATTAGACAAGGAAGCCTTCCCCCCGCCCCCACCCCCACTCCCAGTCTTCCTT
TCCCTTCCAGCAGGGAGTGCCCCCTCCATAAGATCATTACATTTGGACAATCAAGGTGCACA
ATTATAAGTGACCACAGCCATGCACCTTGGACATTATTGGACATTAATGTGCGTAACTGCAC
ATGGCCCATCCCATCTGAATAAGGTCCTACTCTCAGATGCCCTTTGCAGTACAGCAGGGGTA
CTGAATCACCAAGGCCCTTTTTCTTGGCCTGTTATGTGTGTGATTATATTTATCCCAGTTTC
TGTGTAATAGACATGAAAGCCTCCCCTGCCACACCCCACCTCCAATCTTCCTTTCCCTTCCA
CCAGGGAGTGTCCACTCCATATACCCTTACATTTGGACAATCAAGGTGCACAATTGTAAGTG
AGCATAGGCACTCACCTTGGACATGAATGTGCATAACTGCACATGGCCCATCCCATCTGAAT
AAGGTCCTACTCTCAGACCCTTTTTGCAGTACAGCAGGGGTGCTGATCACCAAGGCCCCTTT
TCCTGGCCTGTTATGTGTGTGATTATATTTGTTCCAGTTCCTGTGTAATAGACATGGAAGCC
TCCCCTGCCACACTCCACCCCCAATCTTCCTTTCCCTTCTGGCAGGAAGTACCCGCTCCATA
AGACCCTTACATTTGGACAGTCAAGGTGCACAATTGTATGTGACCACAACCATGCACCTTGG
ACATAAATGTGTGTAACTGCACATGGCCCATCCCATCTGAATAAGGTCCTACTCTCAGACCC
CTTTTGCAGTACAGTAGGTGTGCTGATAACCAAGGCCCCTCTTCCTGGCCTGTTAACGTATG
TGATTATATTTGTCTGGGTTCCAGTGTATAAGACATGGAAGCCTCCCCTGCCCCACCCCACC
CTCAATCTTCCTTTCCCTTCTGGCAGGGAGTGCCAGCTCCATAAGAACCTTACATTTGGACA
GTCAAGGTGCACAATTCTAAGTGACCGCAGCCATGCACCTTGGTCAATAATGTGTGTAACTG
CACACGGCCTATCTCATCTGAATAAGGCCTTACTCTCAGACCCCTTTTGCAGTACAGCAGGG
GTGCTGATAACCAAGGCCCATTTTCCTGGCCTGTTATGTGTGTGATTATATTTGTCCAGGTT
TCTGTGTACTAGACAAGGAAGCCTCCTCTGCCCCATCCCATCTACGCATAATCTTTCTTTTC
CTCCCAGCAGGGAGTGCTCACTCCATAAGACCCTTACATTTGGACAATCAAGGTGCACAATT
GTAAGTGACCACAACCATGCATCTTGGAAATTTATGTGCATAACTGCACATGGCTTATCCTA
TTTGAATAAAGTCCTACTCTCAGACCCCCTTTGCAGTATAGCTGGGGTGCTGATCACTGAGG
CCTCTTTGCTTGGCTTGTCTATATTCTTGTGTACTAGATAAGGGCACCTTCTCATGGACTCC
CTTTGCTTTTCAACAAGGAGTACCCACTACTTTTTAAGATTCTTATATTTGTCCAAAGTACA
TGGTTTTAATTGACCACAACAATGTCCCTTGGACATTAATGTATGTAATCACCACATGGTTC
ATCCTAATTAAACAAAGTTCTACCTTCTCACCCTCCATTTGCAGTATACCAGGGTTGCTGAC
CCCCTAAGTCCCCTTTTCTTGGCTTGTTGA
Exon4 (structured, conserved seq)
ATCTTCCTCAGAAGAATAGGCTTGTTGTTTTACAGTGTTAGTGATCCATTCCCTTTGACGAT
CCCTAGGTGGAGATGGGGCATGAGGATCCTCCAGGGGAAAAGCTCACTACCACTGGGCAACA
ACCCTAGGTCAGGAGGTTCTGTCAAGATACTTTCCTGGTCCCAGATAGGAAGATAAAGTCTC
AAAAACAACCACCACACGTCAAG
E repeat = Rich on T, binds heavily to proteins, 11947-13702 = 1756 nt
TGTGTATTTCTTTGTCTCTTTCTTTCTTGTCTTTGCTCTTTGTTCTCTATCTAAAGTGTGTC
TTACCCATTTCCATGTTTCTCTTGCTAATTTCTTTCGTGTGTGCCTTTGCCTCATTTTCTCT
TTTTGTTCACAAGAGTGGTCTGTGTCTTGTCTTAGACATATCTCTCATTTTTCATTTTGTTG
CTATTTCTCTTTGCTCTCCTAGATGTGGCTCTTCTTTCACGCTTTATTTCATGTCTCCTTTT
TGGGTCACATGCTGTGTGCTTTTTGTCCTTTTCTTGTTCTGTCTACCTCTCCTTTCTCTGCC
TACCTCTCTTTTCTCTTTGTGAACTGTGATTATTTGTTACCCCTTCCCCTTCTCGTTCGTTT
TAAATTTCACCTTTTTTCTGAGTCTGGCCTCCTTTCTGCTGTTTCTACTTTTTATCTCACAT
TTCTCATTTCTGCATTTCCTTTCTGCCTCTCTTGGGCTATTCTCTCTCTCCTCCCCTGCGTG
CCTCAGCATCTCTTGCTGTTTGTGATTTTCTATTTCAGTATTAATCTCTGTTGGCTTGTATT
TGTTCTCTGCTTCTTCCCTTTCTACTCACCTTTGAGTATTTCAGCCTCTTCATGAATCTATC
TCCCTCTCTTTGATTTCATGTAATCTCTCCTTAAATATTTCTTTGCATATGTGGGCAAGTGT
ACGTGTGTGTGTGTCATGTGTGGCAGAGGGGCTTCCTAACCCCTGCCTGATAGGTGCAGAAC
GTCGGCTATCAGAGCAAGCATTGTGGAGCGGTTCCTTATGCCAGGCTGCCATGTGAGATGAT
CCAAGACCAAAACAAGGCCCTAGACTGCAGTAAAACCCAGAACTCAAGTAGGGCAGAAGGTG
GAAGGCTCATATGGATAGAAGGCCCAAAGTATAAGACAGATGGTTTGAGACTTGAGACCCGA
GGACTAAGATGGAAAGCCCATGTTCCAAGATAGATAGAAGCCTCAGGCCTGAAACCAACAAA
AGCCTCAAGAGCCAAGAAAACAGAGGGTGGCCTGAATTGGACCGAAGGCCTGAGTTGGATGG
AAGTCTCAAGGCTTGAGTTAGAAGTCTTAAGACCTGGGACAGGACACATGGAAGGCCTAAGA
ACTGAGACTTGTGACACAAGGCCAACGACCTAAGATTAGCCCAGGGTTGTAGCTGGAAGACC
TACAACCCAAGGATGGAAGGCCCCTGTCACAAAGCCTACCTAGATGGATAGAGGACCCAAGC
GAAAAAGGTATCTCAAGACTAACGGCCGGAATCTGGAGGCCCATGACCCAGAACCCAGGAAG
GATAGAAGCTTGAAGACCTGGGGAAATCCCAAGATGAGAACCCTAAACCCTACCTCTTTTCT
ATTGTTTACACTTCTTACTCTTAGATATTTCCAGTTCTCCTGTTTATCTTTAAGCCTGATTC
TTTTGAGATGTACTTTTTGATGTTGCCGGTTACCTTTAGATTGACAGTATTATGCCTGGGCC
AGTCTTGAGCCAGCTTTAAATCACAGCTTTTACCTATTTGTTAGGCTATAGTGTTTTGTAAA
CTTCTGTTTCTATTCACATCTTCTCCACTTGAGAGAGACACCAAAATCCAGTCAGTATCTAA
TCTGGCTTTTGTTAACTTCCCTCAGGAGCAGACATTCATATAGGTGATACTGTATTTCAGTC
CTTTCTTTTGACCCCAGAAGCCCTAGACTGAGAAGATAAAATGGTCAGGTTGTTGGGGAAAA
AAAAGTGCCAGGCTCTCTAG
A repeat + B repeat (713 nt)
GAATTTTTCTTTGGAATCATTTTTGGTTGACATCTCTGTTTTTTGTGGATCAGTTTTTTACT
CTTCCACTCTCTTTTCTATATTTTGCCCATCGGGGCTGCGGATACCTGGTTTTATTATTTTT
TCTTTGCCCAACGGGGCCGTGGATACCTGCCTTTTAATTCTTTTTTATTCGCCCATCGGGGC
CGCGGATACCTGCTTTTTATTTTTTTTTCCTTAGCCCATCGGGGTATCGGATACCTGCTGAT
TCCCTTCCCCTCTGAACCCCCAACACTCTGGCCCATCGGGGTGACGGATATCTGCTTTTTAA
AAATTTTCTTTTTTTGGCCCATCGGGGCTTCGGATACCTGCTTTTTTTTTTTTTATTTTTCC
TTGCCCATCGGGGCCTCGGATACCTGCTTTAATTTTTGTTTTTCTGGCCCATCGGGGCCGCG
GATACCTGCTTTGATTTTTTTTTTTCATCGCCCATCGGTGCTTTTTATGGATGAAAAAATGT
T
ATACCTCCCCCCCCACCCCCCAACCCCCCCAACTCCCCACCCCCACCCCCCACCCCCCACCT
CCCCACCCCCCTACCCCCCTACCCCCCTACCCCCC
CCTCCCCAGCCCTGCTCCCAGCAAACCCCTAGTCTAGCCCCAGCCCTACTCCCACCCCGCCC
CAGCCCTGCCCCAGCCCCAGTCCCCTAACCCCCCAGCCCTAGCCCCAGTCCCAGTCC
A repeat +F + B repeat (1069 nt)
GAATTTTTCTTTGGAATCATTTTTGGTTGACATCTCTGTTTTTTGTGGATCAGTTTTTTACT
CTTCCACTCTCTTTTCTATATTTTGCCCATCGGGGCTGCGGATACCTGGTTTTATTATTTTT
TCTTTGCCCAACGGGGCCGTGGATACCTGCCTTTTAATTCTTTTTTATTCGCCCATCGGGGC
CGCGGATACCTGCTTTTTATTTTTTTTTCCTTAGCCCATCGGGGTATCGGATACCTGCTGAT
TCCCTTCCCCTCTGAACCCCCAACACTCTGGCCCATCGGGGTGACGGATATCTGCTTTTTAA
AAATTTTCTTTTTTTGGCCCATCGGGGCTTCGGATACCTGCTTTTTTTTTTTTTATTTTTCC
TTGCCCATCGGGGCCTCGGATACCTGCTTTAATTTTTGTTTTTCTGGCCCATCGGGGCCGCG
GATACCTGCTTTGATTTTTTTTTTTCATCGCCCATCGGTGCTTTTTATGGATGAAAAAATGT
T
GACATTGCGGCATTGCTCAGCATGGCGGGCTGTGCTTTGTTAGGTTGTCCAAAATGGCGGAT
CCAGTTCTGTCGCAGTGTTCAAGTGGCGGGAAGGCCACATCATGATGGGCGAGGCTTTGTTA
AGTGGTTAGCATGGTGGTGGACATGTGCGGTCACACAGGAAAAGATGGCGGCTGAAGGTCTT
GCCGCAGTGTAAAACATGGCGGGCCTCTTTGTCTTTGCTGTGTGCTTTTCGTGTTGGGTTTT
GCCGCAGGGACAATATGGCAGGCGTTGTCATATGTATATCATGGCTTTTGTCACGTGGACAT
CATGGCGGGCTTGCCGCATTGTTAAAGATGGCGGGTTTTGCCGCCT
ATACCTCCCCCCCCACCCCCCAACCCCCCCAACTCCCCACCCCCACCCCCCACCCCCCACCT
CCCCACCCCCCTACCCCCCTACCCCCCTACCCCCC
CCTCCCCAGCCCTGCTCCCAGCAAACCCCTAGTCTAGCCCCAGCCCTACTCCCACCCCGCCC
CAGCCCTGCCCCAGCCCCAGTCCCCTAACCCCCCAGCCCTAGCCCCAGTCCCAGTCC
A repeat +F + B +C repeat (1189 nt)
GAATTTTTCTTTGGAATCATTTTTGGTTGACATCTCTGTTTTTTGTGGATCAGTTTTTTACT
CTTCCACTCTCTTTTCTATATTTTGCCCATCGGGGCTGCGGATACCTGGTTTTATTATTTTT
TCTTTGCCCAACGGGGCCGTGGATACCTGCCTTTTAATTCTTTTTTATTCGCCCATCGGGGC
CGCGGATACCTGCTTTTTATTTTTTTTTCCTTAGCCCATCGGGGTATCGGATACCTGCTGAT
TCCCTTCCCCTCTGAACCCCCAACACTCTGGCCCATCGGGGTGACGGATATCTGCTTTTTAA
AAATTTTCTTTTTTTGGCCCATCGGGGCTTCGGATACCTGCTTTTTTTTTTTTTATTTTTCC
TTGCCCATCGGGGCCTCGGATACCTGCTTTAATTTTTGTTTTTCTGGCCCATCGGGGCCGCG
GATACCTGCTTTGATTTTTTTTTTTCATCGCCCATCGGTGCTTTTTATGGATGAAAAAATGT
T
GACATTGCGGCATTGCTCAGCATGGCGGGCTGTGCTTTGTTAGGTTGTCCAAAATGGCGGAT
CCAGTTCTGTCGCAGTGTTCAAGTGGCGGGAAGGCCACATCATGATGGGCGAGGCTTTGTTA
AGTGGTTAGCATGGTGGTGGACATGTGCGGTCACACAGGAAAAGATGGCGGCTGAAGGTCTT
GCCGCAGTGTAAAACATGGCGGGCCTCTTTGTCTTTGCTGTGTGCTTTTCGTGTTGGGTTTT
GCCGCAGGGACAATATGGCAGGCGTTGTCATATGTATATCATGGCTTTTGTCACGTGGACAT
CATGGCGGGCTTGCCGCATTGTTAAAGATGGCGGGTTTTGCCGCCT
ATACCTCCCCCCCCACCCCCCAACCCCCCCAACTCCCCACCCCCACCCCCCACCCCCCACCT
CCCCACCCCCCTACCCCCCTACCCCCCTACCCCCC
CCTCCCCAGCCCTGCTCCCAGCAAACCCCTAGTCTAGCCCCAGCCCTACTCCCACCCCGCCC
CAGCCCTGCCCCAGCCCCAGTCCCCTAACCCCCCAGCCCTAGCCCCAGTCCCAGTCC
TGCTCAAAATAAGTTGTCCATTGCTTATCCTATTATACTGGGATATTCCGTTTACCCTTGGC
ATTGCTGATCTTCAGTACTGACTCCTTGACCATTTTCAGTTAATGCATACAATCCCAT
A repeat +F + B +C + D (3823 nt)
GAATTTTTCTTTGGAATCATTTTTGGTTGACATCTCTGTTTTTTGTGGATCAGTTTTTTACT
CTTCCACTCTCTTTTCTATATTTTGCCCATCGGGGCTGCGGATACCTGGTTTTATTATTTTT
TCTTTGCCCAACGGGGCCGTGGATACCTGCCTTTTAATTCTTTTTTATTCGCCCATCGGGGC
CGCGGATACCTGCTTTTTATTTTTTTTTCCTTAGCCCATCGGGGTATCGGATACCTGCTGAT
TCCCTTCCCCTCTGAACCCCCAACACTCTGGCCCATCGGGGTGACGGATATCTGCTTTTTAA
AAATTTTCTTTTTTTGGCCCATCGGGGCTTCGGATACCTGCTTTTTTTTTTTTTATTTTTCC
TTGCCCATCGGGGCCTCGGATACCTGCTTTAATTTTTGTTTTTCTGGCCCATCGGGGCCGCG
GATACCTGCTTTGATTTTTTTTTTTCATCGCCCATCGGTGCTTTTTATGGATGAAAAAATGT
T
GACATTGCGGCATTGCTCAGCATGGCGGGCTGTGCTTTGTTAGGTTGTCCAAAATGGCGGAT
CCAGTTCTGTCGCAGTGTTCAAGTGGCGGGAAGGCCACATCATGATGGGCGAGGCTTTGTTA
AGTGGTTAGCATGGTGGTGGACATGTGCGGTCACACAGGAAAAGATGGCGGCTGAAGGTCTT
GCCGCAGTGTAAAACATGGCGGGCCTCTTTGTCTTTGCTGTGTGCTTTTCGTGTTGGGTTTT
GCCGCAGGGACAATATGGCAGGCGTTGTCATATGTATATCATGGCTTTTGTCACGTGGACAT
CATGGCGGGCTTGCCGCATTGTTAAAGATGGCGGGTTTTGCCGCCT
ATACCTCCCCCCCCACCCCCCAACCCCCCCAACTCCCCACCCCCACCCCCCACCCCCCACCT
CCCCACCCCCCTACCCCCCTACCCCCCTACCCCCC
CCTCCCCAGCCCTGCTCCCAGCAAACCCCTAGTCTAGCCCCAGCCCTACTCCCACCCCGCCC
CAGCCCTGCCCCAGCCCCAGTCCCCTAACCCCCCAGCCCTAGCCCCAGTCCCAGTCC
TGCTCAAAATAAGTTGTCCATTGCTTATCCTATTATACTGGGATATTCCGTTTACCCTTGGC
ATTGCTGATCTTCAGTACTGACTCCTTGACCATTTTCAGTTAATGCATACAATCCCAT
CCTTTTGAAGGACAGCATGGTTGGTGACACCTAAGGCCCCATTTCTTGGCCTCCCAATATGT
GTGATTGTATTTGTCGAGGTTGCTATGCACTAGAGAAGGAAAGTGCTCCCCTCATCCCCACT
TTTCCCTTCCAGCAGGAAGTGCCCACCCCATAAGACCCTTTTATTTGGAGAGTCTAGGTGCA
CAATTGTAAGTGACCACAAGCATGCATCTTGGACATTTATGTGCGTAATCGCACACTGCTCA
TTCCATGTGAATAAGGTCCTACTCTCCGACCCCTTTTGCAATACAGAAGGGTTGCTGATAAC
GCAGTCCCCTTTTCTTGGCATGTTGTGTGTGATTATAATCGTCTGGGATCCTATGCACTAGA
AAAGGAGGGTCCTCTCCACATACCTCAGTCTCACCTTTCCCTTCCAGCAGGGAGTGCCCACT
CCATAAGACTCTCACATTTGGACAGTCAAGGTGCGTAATTGTTAAGTGAACACAACCATGCA
CCTTAGACATGGATTTGCATAACTACACACAGCTCAACCTATCTGAATAAAATCCTACTCTC
AGACCCCTTTTGCAGTACAGCAGGGGTGCTGATCACCAAGGCCCTTTTTCCTGGCCTGGTAT
GCGTGTGATTATGTTTGTCCCGGTTCCTGTGTATTAGACATGGAAGCCTCCCCTGCCACACT
CCACCCCCAATCTTCCTTTCCCTTCCGGCAGGGAGTGCCCTCTCCATAAGACGCTTACGTTT
GGACAATCAAGGTGCACAGTTGTAAGTGACCACAGGCATACACCTTGGACATTAATGTGCAT
AACCACTTTGCCCATTCCATCTGAATAAGGTCCTACTCTCAGACCCCTTTTGCAGTACAGCA
GGGGTGCTGATCACCAAGGCCCCTTTTCTTGGCCTGTTATGTGCGTGATTATATTTGTCTGG
GTTCCTGTGTATTAGACAAGGAAGCCTTCCCCCCGCCCCCACCCCCACTCCCAGTCTTCCTT
TCCCTTCCAGCAGGGAGTGCCCCCTCCATAAGATCATTACATTTGGACAATCAAGGTGCACA
ATTATAAGTGACCACAGCCATGCACCTTGGACATTATTGGACATTAATGTGCGTAACTGCAC
ATGGCCCATCCCATCTGAATAAGGTCCTACTCTCAGATGCCCTTTGCAGTACAGCAGGGGTA
CTGAATCACCAAGGCCCTTTTTCTTGGCCTGTTATGTGTGTGATTATATTTATCCCAGTTTC
TGTGTAATAGACATGAAAGCCTCCCCTGCCACACCCCACCTCCAATCTTCCTTTCCCTTCCA
CCAGGGAGTGTCCACTCCATATACCCTTACATTTGGACAATCAAGGTGCACAATTGTAAGTG
AGCATAGGCACTCACCTTGGACATGAATGTGCATAACTGCACATGGCCCATCCCATCTGAAT
AAGGTCCTACTCTCAGACCCTTTTTGCAGTACAGCAGGGGTGCTGATCACCAAGGCCCCTTT
TCCTGGCCTGTTATGTGTGTGATTATATTTGTTCCAGTTCCTGTGTAATAGACATGGAAGCC
TCCCCTGCCACACTCCACCCCCAATCTTCCTTTCCCTTCTGGCAGGAAGTACCCGCTCCATA
AGACCCTTACATTTGGACAGTCAAGGTGCACAATTGTATGTGACCACAACCATGCACCTTGG
ACATAAATGTGTGTAACTGCACATGGCCCATCCCATCTGAATAAGGTCCTACTCTCAGACCC
CTTTTGCAGTACAGTAGGTGTGCTGATAACCAAGGCCCCTCTTCCTGGCCTGTTAACGTATG
TGATTATATTTGTCTGGGTTCCAGTGTATAAGACATGGAAGCCTCCCCTGCCCCACCCCACC
CTCAATCTTCCTTTCCCTTCTGGCAGGGAGTGCCAGCTCCATAAGAACCTTACATTTGGACA
GTCAAGGTGCACAATTCTAAGTGACCGCAGCCATGCACCTTGGTCAATAATGTGTGTAACTG
CACACGGCCTATCTCATCTGAATAAGGCCTTACTCTCAGACCCCTTTTGCAGTACAGCAGGG
GTGCTGATAACCAAGGCCCATTTTCCTGGCCTGTTATGTGTGTGATTATATTTGTCCAGGTT
TCTGTGTACTAGACAAGGAAGCCTCCTCTGCCCCATCCCATCTACGCATAATCTTTCTTTTC
CTCCCAGCAGGGAGTGCTCACTCCATAAGACCCTTACATTTGGACAATCAAGGTGCACAATT
GTAAGTGACCACAACCATGCATCTTGGAAATTTATGTGCATAACTGCACATGGCTTATCCTA
TTTGAATAAAGTCCTACTCTCAGACCCCCTTTGCAGTATAGCTGGGGTGCTGATCACTGAGG
CCTCTTTGCTTGGCTTGTCTATATTCTTGTGTACTAGATAAGGGCACCTTCTCATGGACTCC
CTTTGCTTTTCAACAAGGAGTACCCACTACTTTTTAAGATTCTTATATTTGTCCAAAGTACA
TGGTTTTAATTGACCACAACAATGTCCCTTGGACATTAATGTATGTAATCACCACATGGTTC
ATCCTAATTAAACAAAGTTCTACCTTCTCACCCTCCATTTGCAGTATACCAGGGTTGCTGAC
CCCCTAAGTCCCCTTTTCTTGGCTTGTTGA
A repeat +F + B +C + E (2945 nt)
GAATTTTTCTTTGGAATCATTTTTGGTTGACATCTCTGTTTTTTGTGGATCAGTTTTTTACT
CTTCCACTCTCTTTTCTATATTTTGCCCATCGGGGCTGCGGATACCTGGTTTTATTATTTTT
TCTTTGCCCAACGGGGCCGTGGATACCTGCCTTTTAATTCTTTTTTATTCGCCCATCGGGGC
CGCGGATACCTGCTTTTTATTTTTTTTTCCTTAGCCCATCGGGGTATCGGATACCTGCTGAT
TCCCTTCCCCTCTGAACCCCCAACACTCTGGCCCATCGGGGTGACGGATATCTGCTTTTTAA
AAATTTTCTTTTTTTGGCCCATCGGGGCTTCGGATACCTGCTTTTTTTTTTTTTATTTTTCC
TTGCCCATCGGGGCCTCGGATACCTGCTTTAATTTTTGTTTTTCTGGCCCATCGGGGCCGCG
GATACCTGCTTTGATTTTTTTTTTTCATCGCCCATCGGTGCTTTTTATGGATGAAAAAATGT
T
GACATTGCGGCATTGCTCAGCATGGCGGGCTGTGCTTTGTTAGGTTGTCCAAAATGGCGGAT
CCAGTTCTGTCGCAGTGTTCAAGTGGCGGGAAGGCCACATCATGATGGGCGAGGCTTTGTTA
AGTGGTTAGCATGGTGGTGGACATGTGCGGTCACACAGGAAAAGATGGCGGCTGAAGGTCTT
GCCGCAGTGTAAAACATGGCGGGCCTCTTTGTCTTTGCTGTGTGCTTTTCGTGTTGGGTTTT
GCCGCAGGGACAATATGGCAGGCGTTGTCATATGTATATCATGGCTTTTGTCACGTGGACAT
CATGGCGGGCTTGCCGCATTGTTAAAGATGGCGGGTTTTGCCGCCT
ATACCTCCCCCCCCACCCCCCAACCCCCCCAACTCCCCACCCCCACCCCCCACCCCCCACCT
CCCCACCCCCCTACCCCCCTACCCCCCTACCCCCC
CCTCCCCAGCCCTGCTCCCAGCAAACCCCTAGTCTAGCCCCAGCCCTACTCCCACCCCGCCC
CAGCCCTGCCCCAGCCCCAGTCCCCTAACCCCCCAGCCCTAGCCCCAGTCCCAGTCC
TGCTCAAAATAAGTTGTCCATTGCTTATCCTATTATACTGGGATATTCCGTTTACCCTTGGC
ATTGCTGATCTTCAGTACTGACTCCTTGACCATTTTCAGTTAATGCATACAATCCCAT
TGTGTATTTCTTTGTCTCTTTCTTTCTTGTCTTTGCTCTTTGTTCTCTATCTAAAGTGTGTC
TTACCCATTTCCATGTTTCTCTTGCTAATTTCTTTCGTGTGTGCCTTTGCCTCATTTTCTCT
TTTTGTTCACAAGAGTGGTCTGTGTCTTGTCTTAGACATATCTCTCATTTTTCATTTTGTTG
CTATTTCTCTTTGCTCTCCTAGATGTGGCTCTTCTTTCACGCTTTATTTCATGTCTCCTTTT
TGGGTCACATGCTGTGTGCTTTTTGTCCTTTTCTTGTTCTGTCTACCTCTCCTTTCTCTGCC
TACCTCTCTTTTCTCTTTGTGAACTGTGATTATTTGTTACCCCTTCCCCTTCTCGTTCGTTT
TAAATTTCACCTTTTTTCTGAGTCTGGCCTCCTTTCTGCTGTTTCTACTTTTTATCTCACAT
TTCTCATTTCTGCATTTCCTTTCTGCCTCTCTTGGGCTATTCTCTCTCTCCTCCCCTGCGTG
CCTCAGCATCTCTTGCTGTTTGTGATTTTCTATTTCAGTATTAATCTCTGTTGGCTTGTATT
TGTTCTCTGCTTCTTCCCTTTCTACTCACCTTTGAGTATTTCAGCCTCTTCATGAATCTATC
TCCCTCTCTTTGATTTCATGTAATCTCTCCTTAAATATTTCTTTGCATATGTGGGCAAGTGT
ACGTGTGTGTGTGTCATGTGTGGCAGAGGGGCTTCCTAACCCCTGCCTGATAGGTGCAGAAC
GTCGGCTATCAGAGCAAGCATTGTGGAGCGGTTCCTTATGCCAGGCTGCCATGTGAGATGAT
CCAAGACCAAAACAAGGCCCTAGACTGCAGTAAAACCCAGAACTCAAGTAGGGCAGAAGGTG
GAAGGCTCATATGGATAGAAGGCCCAAAGTATAAGACAGATGGTTTGAGACTTGAGACCCGA
GGACTAAGATGGAAAGCCCATGTTCCAAGATAGATAGAAGCCTCAGGCCTGAAACCAACAAA
AGCCTCAAGAGCCAAGAAAACAGAGGGTGGCCTGAATTGGACCGAAGGCCTGAGTTGGATGG
AAGTCTCAAGGCTTGAGTTAGAAGTCTTAAGACCTGGGACAGGACACATGGAAGGCCTAAGA
ACTGAGACTTGTGACACAAGGCCAACGACCTAAGATTAGCCCAGGGTTGTAGCTGGAAGACC
TACAACCCAAGGATGGAAGGCCCCTGTCACAAAGCCTACCTAGATGGATAGAGGACCCAAGC
GAAAAAGGTATCTCAAGACTAACGGCCGGAATCTGGAGGCCCATGACCCAGAACCCAGGAAG
GATAGAAGCTTGAAGACCTGGGGAAATCCCAAGATGAGAACCCTAAACCCTACCTCTTTTCT
ATTGTTTACACTTCTTACTCTTAGATATTTCCAGTTCTCCTGTTTATCTTTAAGCCTGATTC
TTTTGAGATGTACTTTTTGATGTTGCCGGTTACCTTTAGATTGACAGTATTATGCCTGGGCC
AGTCTTGAGCCAGCTTTAAATCACAGCTTTTACCTATTTGTTAGGCTATAGTGTTTTGTAAA
CTTCTGTTTCTATTCACATCTTCTCCACTTGAGAGAGACACCAAAATCCAGTCAGTATCTAA
TCTGGCTTTTGTTAACTTCCCTCAGGAGCAGACATTCATATAGGTGATACTGTATTTCAGTC
CTTTCTTTTGACCCCAGAAGCCCTAGACTGAGAAGATAAAATGGTCAGGTTGTTGGGGAAAA
AAAAGTGCCAGGCTCTCTAG
A repeat +F + B repeat + D + E (5459 nt)
GAATTTTTCTTTGGAATCATTTTTGGTTGACATCTCTGTTTTTTGTGGATCAGTTTTTTACT
CTTCCACTCTCTTTTCTATATTTTGCCCATCGGGGCTGCGGATACCTGGTTTTATTATTTTT
TCTTTGCCCAACGGGGCCGTGGATACCTGCCTTTTAATTCTTTTTTATTCGCCCATCGGGGC
CGCGGATACCTGCTTTTTATTTTTTTTTCCTTAGCCCATCGGGGTATCGGATACCTGCTGAT
TCCCTTCCCCTCTGAACCCCCAACACTCTGGCCCATCGGGGTGACGGATATCTGCTTTTTAA
AAATTTTCTTTTTTTGGCCCATCGGGGCTTCGGATACCTGCTTTTTTTTTTTTTATTTTTCC
TTGCCCATCGGGGCCTCGGATACCTGCTTTAATTTTTGTTTTTCTGGCCCATCGGGGCCGCG
GATACCTGCTTTGATTTTTTTTTTTCATCGCCCATCGGTGCTTTTTATGGATGAAAAAATGT
T
GACATTGCGGCATTGCTCAGCATGGCGGGCTGTGCTTTGTTAGGTTGTCCAAAATGGCGGAT
CCAGTTCTGTCGCAGTGTTCAAGTGGCGGGAAGGCCACATCATGATGGGCGAGGCTTTGTTA
AGTGGTTAGCATGGTGGTGGACATGTGCGGTCACACAGGAAAAGATGGCGGCTGAAGGTCTT
GCCGCAGTGTAAAACATGGCGGGCCTCTTTGTCTTTGCTGTGTGCTTTTCGTGTTGGGTTTT
GCCGCAGGGACAATATGGCAGGCGTTGTCATATGTATATCATGGCTTTTGTCACGTGGACAT
CATGGCGGGCTTGCCGCATTGTTAAAGATGGCGGGTTTTGCCGCCT
ATACCTCCCCCCCCACCCCCCAACCCCCCCAACTCCCCACCCCCACCCCCCACCCCCCACCT
CCCCACCCCCCTACCCCCCTACCCCCCTACCCCCC
CCTCCCCAGCCCTGCTCCCAGCAAACCCCTAGTCTAGCCCCAGCCCTACTCCCACCCCGCCC
CAGCCCTGCCCCAGCCCCAGTCCCCTAACCCCCCAGCCCTAGCCCCAGTCCCAGTCC
CCTTTTGAAGGACAGCATGGTTGGTGACACCTAAGGCCCCATTTCTTGGCCTCCCAATATGT
GTGATTGTATTTGTCGAGGTTGCTATGCACTAGAGAAGGAAAGTGCTCCCCTCATCCCCACT
TTTCCCTTCCAGCAGGAAGTGCCCACCCCATAAGACCCTTTTATTTGGAGAGTCTAGGTGCA
CAATTGTAAGTGACCACAAGCATGCATCTTGGACATTTATGTGCGTAATCGCACACTGCTCA
TTCCATGTGAATAAGGTCCTACTCTCCGACCCCTTTTGCAATACAGAAGGGTTGCTGATAAC
GCAGTCCCCTTTTCTTGGCATGTTGTGTGTGATTATAATCGTCTGGGATCCTATGCACTAGA
AAAGGAGGGTCCTCTCCACATACCTCAGTCTCACCTTTCCCTTCCAGCAGGGAGTGCCCACT
CCATAAGACTCTCACATTTGGACAGTCAAGGTGCGTAATTGTTAAGTGAACACAACCATGCA
CCTTAGACATGGATTTGCATAACTACACACAGCTCAACCTATCTGAATAAAATCCTACTCTC
AGACCCCTTTTGCAGTACAGCAGGGGTGCTGATCACCAAGGCCCTTTTTCCTGGCCTGGTAT
GCGTGTGATTATGTTTGTCCCGGTTCCTGTGTATTAGACATGGAAGCCTCCCCTGCCACACT
CCACCCCCAATCTTCCTTTCCCTTCCGGCAGGGAGTGCCCTCTCCATAAGACGCTTACGTTT
GGACAATCAAGGTGCACAGTTGTAAGTGACCACAGGCATACACCTTGGACATTAATGTGCAT
AACCACTTTGCCCATTCCATCTGAATAAGGTCCTACTCTCAGACCCCTTTTGCAGTACAGCA
GGGGTGCTGATCACCAAGGCCCCTTTTCTTGGCCTGTTATGTGCGTGATTATATTTGTCTGG
GTTCCTGTGTATTAGACAAGGAAGCCTTCCCCCCGCCCCCACCCCCACTCCCAGTCTTCCTT
TCCCTTCCAGCAGGGAGTGCCCCCTCCATAAGATCATTACATTTGGACAATCAAGGTGCACA
ATTATAAGTGACCACAGCCATGCACCTTGGACATTATTGGACATTAATGTGCGTAACTGCAC
ATGGCCCATCCCATCTGAATAAGGTCCTACTCTCAGATGCCCTTTGCAGTACAGCAGGGGTA
CTGAATCACCAAGGCCCTTTTTCTTGGCCTGTTATGTGTGTGATTATATTTATCCCAGTTTC
TGTGTAATAGACATGAAAGCCTCCCCTGCCACACCCCACCTCCAATCTTCCTTTCCCTTCCA
CCAGGGAGTGTCCACTCCATATACCCTTACATTTGGACAATCAAGGTGCACAATTGTAAGTG
AGCATAGGCACTCACCTTGGACATGAATGTGCATAACTGCACATGGCCCATCCCATCTGAAT
AAGGTCCTACTCTCAGACCCTTTTTGCAGTACAGCAGGGGTGCTGATCACCAAGGCCCCTTT
TCCTGGCCTGTTATGTGTGTGATTATATTTGTTCCAGTTCCTGTGTAATAGACATGGAAGCC
TCCCCTGCCACACTCCACCCCCAATCTTCCTTTCCCTTCTGGCAGGAAGTACCCGCTCCATA
AGACCCTTACATTTGGACAGTCAAGGTGCACAATTGTATGTGACCACAACCATGCACCTTGG
ACATAAATGTGTGTAACTGCACATGGCCCATCCCATCTGAATAAGGTCCTACTCTCAGACCC
CTTTTGCAGTACAGTAGGTGTGCTGATAACCAAGGCCCCTCTTCCTGGCCTGTTAACGTATG
TGATTATATTTGTCTGGGTTCCAGTGTATAAGACATGGAAGCCTCCCCTGCCCCACCCCACC
CTCAATCTTCCTTTCCCTTCTGGCAGGGAGTGCCAGCTCCATAAGAACCTTACATTTGGACA
GTCAAGGTGCACAATTCTAAGTGACCGCAGCCATGCACCTTGGTCAATAATGTGTGTAACTG
CACACGGCCTATCTCATCTGAATAAGGCCTTACTCTCAGACCCCTTTTGCAGTACAGCAGGG
GTGCTGATAACCAAGGCCCATTTTCCTGGCCTGTTATGTGTGTGATTATATTTGTCCAGGTT
TCTGTGTACTAGACAAGGAAGCCTCCTCTGCCCCATCCCATCTACGCATAATCTTTCTTTTC
CTCCCAGCAGGGAGTGCTCACTCCATAAGACCCTTACATTTGGACAATCAAGGTGCACAATT
GTAAGTGACCACAACCATGCATCTTGGAAATTTATGTGCATAACTGCACATGGCTTATCCTA
TTTGAATAAAGTCCTACTCTCAGACCCCCTTTGCAGTATAGCTGGGGTGCTGATCACTGAGG
CCTCTTTGCTTGGCTTGTCTATATTCTTGTGTACTAGATAAGGGCACCTTCTCATGGACTCC
CTTTGCTTTTCAACAAGGAGTACCCACTACTTTTTAAGATTCTTATATTTGTCCAAAGTACA
TGGTTTTAATTGACCACAACAATGTCCCTTGGACATTAATGTATGTAATCACCACATGGTTC
ATCCTAATTAAACAAAGTTCTACCTTCTCACCCTCCATTTGCAGTATACCAGGGTTGCTGAC
CCCCTAAGTCCCCTTTTCTTGGCTTGTTGA
TGTGTATTTCTTTGTCTCTTTCTTTCTTGTCTTTGCTCTTTGTTCTCTATCTAAAGTGTGTC
TTACCCATTTCCATGTTTCTCTTGCTAATTTCTTTCGTGTGTGCCTTTGCCTCATTTTCTCT
TTTTGTTCACAAGAGTGGTCTGTGTCTTGTCTTAGACATATCTCTCATTTTTCATTTTGTTG
CTATTTCTCTTTGCTCTCCTAGATGTGGCTCTTCTTTCACGCTTTATTTCATGTCTCCTTTT
TGGGTCACATGCTGTGTGCTTTTTGTCCTTTTCTTGTTCTGTCTACCTCTCCTTTCTCTGCC
TACCTCTCTTTTCTCTTTGTGAACTGTGATTATTTGTTACCCCTTCCCCTTCTCGTTCGTTT
TAAATTTCACCTTTTTTCTGAGTCTGGCCTCCTTTCTGCTGTTTCTACTTTTTATCTCACAT
TTCTCATTTCTGCATTTCCTTTCTGCCTCTCTTGGGCTATTCTCTCTCTCCTCCCCTGCGTG
CCTCAGCATCTCTTGCTGTTTGTGATTTTCTATTTCAGTATTAATCTCTGTTGGCTTGTATT
TGTTCTCTGCTTCTTCCCTTTCTACTCACCTTTGAGTATTTCAGCCTCTTCATGAATCTATC
TCCCTCTCTTTGATTTCATGTAATCTCTCCTTAAATATTTCTTTGCATATGTGGGCAAGTGT
ACGTGTGTGTGTGTCATGTGTGGCAGAGGGGCTTCCTAACCCCTGCCTGATAGGTGCAGAAC
GTCGGCTATCAGAGCAAGCATTGTGGAGCGGTTCCTTATGCCAGGCTGCCATGTGAGATGAT
CCAAGACCAAAACAAGGCCCTAGACTGCAGTAAAACCCAGAACTCAAGTAGGGCAGAAGGTG
GAAGGCTCATATGGATAGAAGGCCCAAAGTATAAGACAGATGGTTTGAGACTTGAGACCCGA
GGACTAAGATGGAAAGCCCATGTTCCAAGATAGATAGAAGCCTCAGGCCTGAAACCAACAAA
AGCCTCAAGAGCCAAGAAAACAGAGGGTGGCCTGAATTGGACCGAAGGCCTGAGTTGGATGG
AAGTCTCAAGGCTTGAGTTAGAAGTCTTAAGACCTGGGACAGGACACATGGAAGGCCTAAGA
ACTGAGACTTGTGACACAAGGCCAACGACCTAAGATTAGCCCAGGGTTGTAGCTGGAAGACC
TACAACCCAAGGATGGAAGGCCCCTGTCACAAAGCCTACCTAGATGGATAGAGGACCCAAGC
GAAAAAGGTATCTCAAGACTAACGGCCGGAATCTGGAGGCCCATGACCCAGAACCCAGGAAG
GATAGAAGCTTGAAGACCTGGGGAAATCCCAAGATGAGAACCCTAAACCCTACCTCTTTTCT
ATTGTTTACACTTCTTACTCTTAGATATTTCCAGTTCTCCTGTTTATCTTTAAGCCTGATTC
TTTTGAGATGTACTTTTTGATGTTGCCGGTTACCTTTAGATTGACAGTATTATGCCTGGGCC
AGTCTTGAGCCAGCTTTAAATCACAGCTTTTACCTATTTGTTAGGCTATAGTGTTTTGTAAA
CTTCTGTTTCTATTCACATCTTCTCCACTTGAGAGAGACACCAAAATCCAGTCAGTATCTAA
TCTGGCTTTTGTTAACTTCCCTCAGGAGCAGACATTCATATAGGTGATACTGTATTTCAGTC
CTTTCTTTTGACCCCAGAAGCCCTAGACTGAGAAGATAAAATGGTCAGGTTGTTGGGGAAAA
AAAAGTGCCAGGCTCTCTAG
All repeats (5579 nt)
GAATTTTTCTTTGGAATCATTTTTGGTTGACATCTCTGTTTTTTGTGGATCAGTTTTTTACT
CTTCCACTCTCTTTTCTATATTTTGCCCATCGGGGCTGCGGATACCTGGTTTTATTATTTTT
TCTTTGCCCAACGGGGCCGTGGATACCTGCCTTTTAATTCTTTTTTATTCGCCCATCGGGGC
CGCGGATACCTGCTTTTTATTTTTTTTTCCTTAGCCCATCGGGGTATCGGATACCTGCTGAT
TCCCTTCCCCTCTGAACCCCCAACACTCTGGCCCATCGGGGTGACGGATATCTGCTTTTTAA
AAATTTTCTTTTTTTGGCCCATCGGGGCTTCGGATACCTGCTTTTTTTTTTTTTATTTTTCC
TTGCCCATCGGGGCCTCGGATACCTGCTTTAATTTTTGTTTTTCTGGCCCATCGGGGCCGCG
GATACCTGCTTTGATTTTTTTTTTTCATCGCCCATCGGTGCTTTTTATGGATGAAAAAATGT
T
GACATTGCGGCATTGCTCAGCATGGCGGGCTGTGCTTTGTTAGGTTGTCCAAAATGGCGGAT
CCAGTTCTGTCGCAGTGTTCAAGTGGCGGGAAGGCCACATCATGATGGGCGAGGCTTTGTTA
AGTGGTTAGCATGGTGGTGGACATGTGCGGTCACACAGGAAAAGATGGCGGCTGAAGGTCTT
GCCGCAGTGTAAAACATGGCGGGCCTCTTTGTCTTTGCTGTGTGCTTTTCGTGTTGGGTTTT
GCCGCAGGGACAATATGGCAGGCGTTGTCATATGTATATCATGGCTTTTGTCACGTGGACAT
CATGGCGGGCTTGCCGCATTGTTAAAGATGGCGGGTTTTGCCGCCT
ATACCTCCCCCCCCACCCCCCAACCCCCCCAACTCCCCACCCCCACCCCCCACCCCCCACCT
CCCCACCCCCCTACCCCCCTACCCCCCTACCCCCC
CCTCCCCAGCCCTGCTCCCAGCAAACCCCTAGTCTAGCCCCAGCCCTACTCCCACCCCGCCC
CAGCCCTGCCCCAGCCCCAGTCCCCTAACCCCCCAGCCCTAGCCCCAGTCCCAGTCC
TGCTCAAAATAAGTIGTCCATTGCTTATCCTATTATACTGGGATATTCCGTTTACCCTTGGC
ATTGCTGATCTTCAGTACTGACTCCTTGACCATTTTCAGTTAATGCATACAATCCCAT
CCTTTTGAAGGACAGCATGGTTGGTGACACCTAAGGCCCCATTTCTTGGCCTCCCAATATGT
GTGATTGTATTTGTCGAGGTTGCTATGCACTAGAGAAGGAAAGTGCTCCCCTCATCCCCACT
TTTCCCTTCCAGCAGGAAGTGCCCACCCCATAAGACCCTTTTATTTGGAGAGTCTAGGTGCA
CAATTGTAAGTGACCACAAGCATGCATCTTGGACATTTATGTGCGTAATCGCACACTGCTCA
TTCCATGTGAATAAGGTCCTACTCTCCGACCCCTTTTGCAATACAGAAGGGTTGCTGATAAC
GCAGTCCCCTTTTCTTGGCATGTTGTGTGTGATTATAATCGTCTGGGATCCTATGCACTAGA
AAAGGAGGGTCCTCTCCACATACCTCAGTCTCACCTTTCCCTTCCAGCAGGGAGTGCCCACT
CCATAAGACTCTCACATTTGGACAGTCAAGGTGCGTAATTGTTAAGTGAACACAACCATGCA
CCTTAGACATGGATTTGCATAACTACACACAGCTCAACCTATCTGAATAAAATCCTACTCTC
AGACCCCTTTTGCAGTACAGCAGGGGTGCTGATCACCAAGGCCCTTTTTCCTGGCCTGGTAT
GCGTGTGATTATGTTTGTCCCGGTTCCTGTGTATTAGACATGGAAGCCTCCCCTGCCACACT
CCACCCCCAATCTTCCTTTCCCTTCCGGCAGGGAGTGCCCTCTCCATAAGACGCTTACGTTT
GGACAATCAAGGTGCACAGTTGTAAGTGACCACAGGCATACACCTTGGACATTAATGTGCAT
AACCACTTTGCCCATTCCATCTGAATAAGGTCCTACTCTCAGACCCCTTTTGCAGTACAGCA
GGGGTGCTGATCACCAAGGCCCCTTTTCTTGGCCTGTTATGTGCGTGATTATATTTGTCTGG
GTTCCTGTGTATTAGACAAGGAAGCCTTCCCCCCGCCCCCACCCCCACTCCCAGTCTTCCTT
TCCCTTCCAGCAGGGAGTGCCCCCTCCATAAGATCATTACATTTGGACAATCAAGGTGCACA
ATTATAAGTGACCACAGCCATGCACCTTGGACATTATTGGACATTAATGTGCGTAACTGCAC
ATGGCCCATCCCATCTGAATAAGGTCCTACTCTCAGATGCCCTTTGCAGTACAGCAGGGGTA
CTGAATCACCAAGGCCCTTTTTCTTGGCCTGTTATGTGTGTGATTATATTTATCCCAGTTTC
TGTGTAATAGACATGAAAGCCTCCCCTGCCACACCCCACCTCCAATCTTCCTTTCCCTTCCA
CCAGGGAGTGTCCACTCCATATACCCTTACATTTGGACAATCAAGGTGCACAATTGTAAGTG
AGCATAGGCACTCACCTTGGACATGAATGTGCATAACTGCACATGGCCCATCCCATCTGAAT
AAGGTCCTACTCTCAGACCCTTTTTGCAGTACAGCAGGGGTGCTGATCACCAAGGCCCCTTT
TCCTGGCCTGTTATGTGTGTGATTATATTTGTTCCAGTTCCTGTGTAATAGACATGGAAGCC
TCCCCTGCCACACTCCACCCCCAATCTTCCTTTCCCTTCTGGCAGGAAGTACCCGCTCCATA
AGACCCTTACATTTGGACAGTCAAGGTGCACAATTGTATGTGACCACAACCATGCACCTTGG
ACATAAATGTGTGTAACTGCACATGGCCCATCCCATCTGAATAAGGTCCTACTCTCAGACCC
CTTTTGCAGTACAGTAGGTGTGCTGATAACCAAGGCCCCTCTTCCTGGCCTGTTAACGTATG
TGATTATATTTGTCTGGGTTCCAGTGTATAAGACATGGAAGCCTCCCCTGCCCCACCCCACC
CTCAATCTTCCTTTCCCTTCTGGCAGGGAGTGCCAGCTCCATAAGAACCTTACATTTGGACA
GTCAAGGTGCACAATTCTAAGTGACCGCAGCCATGCACCTTGGTCAATAATGTGTGTAACTG
CACACGGCCTATCTCATCTGAATAAGGCCTTACTCTCAGACCCCTTTTGCAGTACAGCAGGG
GTGCTGATAACCAAGGCCCATTTTCCTGGCCTGTTATGTGTGTGATTATATTTGTCCAGGTT
TCTGTGTACTAGACAAGGAAGCCTCCTCTGCCCCATCCCATCTACGCATAATCTTTCTTTTC
CTCCCAGCAGGGAGTGCTCACTCCATAAGACCCTTACATTTGGACAATCAAGGTGCACAATT
GTAAGTGACCACAACCATGCATCTTGGAAATTTATGTGCATAACTGCACATGGCTTATCCTA
TTTGAATAAAGTCCTACTCTCAGACCCCCTTTGCAGTATAGCTGGGGTGCTGATCACTGAGG
CCTCTTTGCTTGGCTTGTCTATATTCTTGTGTACTAGATAAGGGCACCTTCTCATGGACTCC
CTTTGCTTTTCAACAAGGAGTACCCACTACTTTTTAAGATTCTTATATTTGTCCAAAGTACA
TGGTTTTAATTGACCACAACAATGTCCCTTGGACATTAATGTATGTAATCACCACATGGTTC
ATCCTAATTAAACAAAGTTCTACCTTCTCACCCTCCATTTGCAGTATACCAGGGTTGCTGAC
CCCCTAAGTCCCCTTTTCTTGGCTTGTTGA
TGTGTATTTCTTTGTCTCTTTCTTTCTTGTCTTTGCTCTTTGTTCTCTATCTAAAGTGTGTC
TTACCCATTTCCATGTTTCTCTTGCTAATTTCTTTCGTGTGTGCCTTTGCCTCATTTTCTCT
TTTTGTTCACAAGAGTGGTCTGTGTCTTGTCTTAGACATATCTCTCATTTTTCATTTTGTTG
CTATTTCTCTTTGCTCTCCTAGATGTGGCTCTTCTTTCACGCTTTATTTCATGTCTCCTTTT
TGGGTCACATGCTGTGTGCTTTTTGTCCTTTTCTTGTTCTGTCTACCTCTCCTTTCTCTGCC
TACCTCTCTTTTCTCTTTGTGAACTGTGATTATTTGTTACCCCTTCCCCTTCTCGTTCGTTT
TAAATTTCACCTTTTTTCTGAGTCTGGCCTCCTTTCTGCTGTTTCTACTTTTTATCTCACAT
TTCTCATTTCTGCATTTCCTTTCTGCCTCTCTTGGGCTATTCTCTCTCTCCTCCCCTGCGTG
CCTCAGCATCTCTTGCTGTTTGTGATTTTCTATTTCAGTATTAATCTCTGTTGGCTTGTATT
TGTTCTCTGCTTCTTCCCTTTCTACTCACCTTTGAGTATTTCAGCCTCTTCATGAATCTATC
TCCCTCTCTTTGATTTCATGTAATCTCTCCTTAAATATTTCTTTGCATATGTGGGCAAGTGT
ACGTGTGTGTGTGTCATGTGTGGCAGAGGGGCTTCCTAACCCCTGCCTGATAGGTGCAGAAC
GTCGGCTATCAGAGCAAGCATTGTGGAGCGGTTCCTTATGCCAGGCTGCCATGTGAGATGAT
CCAAGACCAAAACAAGGCCCTAGACTGCAGTAAAACCCAGAACTCAAGTAGGGCAGAAGGTG
GAAGGCTCATATGGATAGAAGGCCCAAAGTATAAGACAGATGGTTTGAGACTTGAGACCCGA
GGACTAAGATGGAAAGCCCATGTTCCAAGATAGATAGAAGCCTCAGGCCTGAAACCAACAAA
AGCCTCAAGAGCCAAGAAAACAGAGGGTGGCCTGAATTGGACCGAAGGCCTGAGTTGGATGG
AAGTCTCAAGGCTTGAGTTAGAAGTCTTAAGACCTGGGACAGGACACATGGAAGGCCTAAGA
ACTGAGACTTGTGACACAAGGCCAACGACCTAAGATTAGCCCAGGGTTGTAGCTGGAAGACC
TACAACCCAAGGATGGAAGGCCCCTGTCACAAAGCCTACCTAGATGGATAGAGGACCCAAGC
GAAAAAGGTATCTCAAGACTAACGGCCGGAATCTGGAGGCCCATGACCCAGAACCCAGGAAG
GATAGAAGCTTGAAGACCTGGGGAAATCCCAAGATGAGAACCCTAAACCCTACCTCTTTTCT
ATTGTTTACACTTCTTACTCTTAGATATTTCCAGTTCTCCTGTTTATCTTTAAGCCTGATTC
TTTTGAGATGTACTTTTTGATGTTGCCGGTTACCTTTAGATTGACAGTATTATGCCTGGGCC
AGTCTTGAGCCAGCTTTAAATCACAGCTTTTACCTATTTGTTAGGCTATAGTGTTTTGTAAA
CTTCTGTTTCTATTCACATCTTCTCCACTTGAGAGAGACACCAAAATCCAGTCAGTATCTAA
TCTGGCTTTTGTTAACTTCCCTCAGGAGCAGACATTCATATAGGTGATACTGTATTTCAGTC
CTTTCTTTTGACCCCAGAAGCCCTAGACTGAGAAGATAAAATGGTCAGGTTGTTGGGGAAAA
AAAAGTGCCAGGCTCTCTAG
A repeat +F + B+C + exon4+ E (3154 nt)
GAATTTTTCTTTGGAATCATTTTTGGTTGACATCTCTGTTTTTTGTGGATCAGTTTTTTACT
CTTCCACTCTCTTTTCTATATTTTGCCCATCGGGGCTGCGGATACCTGGTTTTATTATTTTT
TCTTTGCCCAACGGGGCCGTGGATACCTGCCTTTTAATTCTTTTTTATTCGCCCATCGGGGC
CGCGGATACCTGCTTTTTATTTTTTTTTCCTTAGCCCATCGGGGTATCGGATACCTGCTGAT
TCCCTTCCCCTCTGAACCCCCAACACTCTGGCCCATCGGGGTGACGGATATCTGCTTTTTAA
AAATTTTCTTTTTTTGGCCCATCGGGGCTTCGGATACCTGCTTTTTTTTTTTTTATTTTTCC
TTGCCCATCGGGGCCTCGGATACCTGCTTTAATTTTTGTTTTTCTGGCCCATCGGGGCCGCG
GATACCTGCTTTGATTTTTTTTTTTCATCGCCCATCGGTGCTTTTTATGGATGAAAAAATGT
T
GACATTGCGGCATTGCTCAGCATGGCGGGCTGTGCTTTGTTAGGTTGTCCAAAATGGCGGAT
CCAGTTCTGTCGCAGTGTTCAAGTGGCGGGAAGGCCACATCATGATGGGCGAGGCTTTGTTA
AGTGGTTAGCATGGTGGTGGACATGTGCGGTCACACAGGAAAAGATGGCGGCTGAAGGTCTT
GCCGCAGTGTAAAACATGGCGGGCCTCTTTGTCTTTGCTGTGTGCTTTTCGTGTTGGGTTTT
GCCGCAGGGACAATATGGCAGGCGTTGTCATATGTATATCATGGCTTTTGTCACGTGGACAT
CATGGCGGGCTTGCCGCATTGTTAAAGATGGCGGGTTTTGCCGCCT
ATACCTCCCCCCCCACCCCCCAACCCCCCCAACTCCCCACCCCCACCCCCCACCCCCCACCT
CCCCACCCCCCTACCCCCCTACCCCCCTACCCCCC
CCTCCCCAGCCCTGCTCCCAGCAAACCCCTAGTCTAGCCCCAGCCCTACTCCCACCCCGCCC
CAGCCCTGCCCCAGCCCCAGTCCCCTAACCCCCCAGCCCTAGCCCCAGTCCCAGTCC
TGCTCAAAATAAGTTGTCCATTGCTTATCCTATTATACTGGGATATTCCGTTTACCCTTGGC
ATTGCTGATCTTCAGTACTGACTCCTTGACCATTTTCAGTTAATGCATACAATCCCAT
ATCTTCCTCAGAAGAATAGGCTTGTTGTTTTACAGTGTTAGTGATCCATTCCCTTTGACGAT
CCCTAGGTGGAGATGGGGCATGAGGATCCTCCAGGGGAAAAGCTCACTACCACTGGGCAACA
ACCCTAGGTCAGGAGGTTCTGTCAAGATACTTTCCTGGTCCCAGATAGGAAGATAAAGTCTC
AAAAACAACCACCACACGTCAAG
TGTGTATTTCTTTGTCTCTTTCTTTCTTGTCTTTGCTCTTTGTTCTCTATCTAAAGTGTGTC
TTACCCATTTCCATGTTTCTCTTGCTAATTTCTTTCGTGTGTGCCTTTGCCTCATTTTCTCT
TTTTGTTCACAAGAGTGGTCTGTGTCTTGTCTTAGACATATCTCTCATTTTTCATTTTGTTG
CTATTTCTCTTTGCTCTCCTAGATGTGGCTCTTCTTTCACGCTTTATTTCATGTCTCCTTTT
TGGGTCACATGCTGTGTGCTTTTTGTCCTTTTCTTGTTCTGTCTACCTCTCCTTTCTCTGCC
TACCTCTCTTTTCTCTTTGTGAACTGTGATTATTTGTTACCCCTTCCCCTTCTCGTTCGTTT
TAAATTTCACCTTTTTTCTGAGTCTGGCCTCCTTTCTGCTGTTTCTACTTTTTATCTCACAT
TTCTCATTTCTGCATTTCCTTTCTGCCTCTCTTGGGCTATTCTCTCTCTCCTCCCCTGCGTG
CCTCAGCATCTCTTGCTGTTTGTGATTTTCTATTTCAGTATTAATCTCTGTTGGCTTGTATT
TGTTCTCTGCTTCTTCCCTTTCTACTCACCTTTGAGTATTTCAGCCTCTTCATGAATCTATC
TCCCTCTCTTTGATTTCATGTAATCTCTCCTTAAATATTTCTTTGCATATGTGGGCAAGTGT
ACGTGTGTGTGTGTCATGTGTGGCAGAGGGGCTTCCTAACCCCTGCCTGATAGGTGCAGAAC
GTCGGCTATCAGAGCAAGCATTGTGGAGCGGTTCCTTATGCCAGGCTGCCATGTGAGATGAT
CCAAGACCAAAACAAGGCCCTAGACTGCAGTAAAACCCAGAACTCAAGTAGGGCAGAAGGTG
GAAGGCTCATATGGATAGAAGGCCCAAAGTATAAGACAGATGGTTTGAGACTTGAGACCCGA
GGACTAAGATGGAAAGCCCATGTTCCAAGATAGATAGAAGCCTCAGGCCTGAAACCAACAAA
AGCCTCAAGAGCCAAGAAAACAGAGGGTGGCCTGAATTGGACCGAAGGCCTGAGTTGGATGG
AAGTCTCAAGGCTTGAGTTAGAAGTCTTAAGACCTGGGACAGGACACATGGAAGGCCTAAGA
ACTGAGACTTGTGACACAAGGCCAACGACCTAAGATTAGCCCAGGGTTGTAGCTGGAAGACC
TACAACCCAAGGATGGAAGGCCCCTGTCACAAAGCCTACCTAGATGGATAGAGGACCCAAGC
GAAAAAGGTATCTCAAGACTAACGGCCGGAATCTGGAGGCCCATGACCCAGAACCCAGGAAG
GATAGAAGCTTGAAGACCTGGGGAAATCCCAAGATGAGAACCCTAAACCCTACCTCTTTTCT
ATTGTTTACACTTCTTACTCTTAGATATTTCCAGTTCTCCTGTTTATCTTTAAGCCTGATTC
TTTTGAGATGTACTTTTTGATGTTGCCGGTTACCTTTAGATTGACAGTATTATGCCTGGGCC
AGTCTTGAGCCAGCTTTAAATCACAGCTTTTACCTATTTGTTAGGCTATAGTGTTTTGTAAA
CTTCTGTTTCTATTCACATCTTCTCCACTTGAGAGAGACACCAAAATCCAGTCAGTATCTAA
TCTGGCTTTTGTTAACTTCCCTCAGGAGCAGACATTCATATAGGTGATACTGTATTTCAGTC
CTTTCTTTTGACCCCAGAAGCCCTAGACTGAGAAGATAAAATGGTCAGGTTGTTGGGGAAAA
AAAAGTGCCAGGCTCTCTAG
1 Kb XIST-minigene
GAATTTTTCTTTGGAATCATTTTTGGTGACATCTCTGTTTTTTGTGGATCAGTTTTTTACTC
TTCCACTCTCTTTTCTATATTTTGCCCATCGGGGCTGCGGATACCTGGTTTTATTATTTTTT
CTTTGCCCAACGGGGCCGTGGATACCTGCCTTTTAATTCTTTTTTATTCGCCCATCGGGGCC
GCGGATACCTGCTTTTTATTTTTTTTTCCTTAGCCCATCGGGGTATCGGATACCTGCTGATT
CCCTTCCCCTCTGAACCCCCAACACTCTGGCCCATCGGGGTGACGGATATCTGCTTTTTAAA
AATTTTCTTTTTTTGGCCCATCGGGGCTTCGGATACCTGCTTTTTTTTTTTTTATTTTTCCT
TGCCCATCGGGGCCTCGGATACCTGCTTTAATTTTTGTTTTTCTGGCCCATCGGGGCCGCGG
ATACCTGCTTTGATTTTTTTTTTTCATCGCCCATCGGTGCTTTTTATGGATGAAAAAATGTT
TCTTTGCTGTGTGCTTTTCGTGTTGGGTTTTGCCGCAGGGACAATATGGCAGGCGTTGTCAT
ATGTATATCATGGCTTTTGTCACGTGGACATCATGGCGGGCTTGCCGCATTGTTAAAGATGG
CGGGTTTTGCCGCCTAGTCTATTTGTTAGGCTATAGTGTTTTGTAAACTTCTGTTTCTATTC
ACATCTTCTCCACTTGAGAGAGACACCAAAATCCAGTCAGTATCTAATCTGGCTTTTGTTAA
CTTCCCTCAGGAGCAGACATTCATATAGGTGATACTGTATTTCAGTCCTTTCTTTTGACCCC
AGAAGCCCTAGACTGAGAAGATAAAATGGTCAGGTTGTTGGGGAAAAAAAAAGTGCCAGGCT
CTCTAGAGAAAAATGTGAAGAGATGCTCCAGGCCAATGAGAAGAATTAGACAAGAAATACAC
AGATGTGCCAGACTTCTGAGAAGCACCTGCCAGCAACAGCTTCCTTCTTTGAGCTTAG
2.5 Kb XIST-minigene
GAATTTTTCTTTggaatcatttttggtGACATCTCTGTTTTTTGTGGATCAGTTTTTTACTC
TTCCACTCTCTTTTCTATATTTTGCCCATCGGGGCTGCGGATACCTGGTTTTATTATTTTTT
CTTTGCCCAACGGGGCCGTGGATACCTGCCTTTTAATTCTTTTTTATTCGCCCATCGGGGCC
GCGGATACCTGCTTTTTATTTTTTTTTCCTTAGCCCATCGGGGTATCGGATACCTGCTGATT
CCCTTCCCCTCTGAACCCCCAACACTCTGGCCCATCGGGGTGACGGATATCTGCTTTTTAAA
AATTTTCTTTTTTTGGCCCATCGGGGCTTCGGATACCTGCTTTTTTTTTTTTTATTTTTCCT
TGCCCATCGGGGCCTCGGATACCTGCTTTAATTTTTGTTTTTCTGGCCCATCGGGGCCGCGG
ATACCTGCTTTGATTTTTTTTTTTCATCGCCCATCGGTGCTTTTTATGGATGAAAAAATGTT
GGTTTTGTGGGTTGTTGCACTCTCTGGAATATCTACACTTTTTTTTGCTGCTGATCATTTGG
TGGTGTGTGAGTGTACCTACCGCTTTGGCAGAGAATGACTCTGCAGTTAAGCTAAGGGCGTG
TTCAGATTGTGGAGGAAAAGTGGCCGCCATTTTAACTTGCCGCATAACTCGGCTTAGGGCTA
GTCGTTTGTGCTAAGTTAAACTAGGGAGGCAAGATGGATGATAGCAGGTCAGGCAGAGGAAG
TCATGTGCATTGCATGAGCTAAACCTATCTGAATGAATTGATTTGGGGCTTGTTAGGAGCTT
TGCGTgattgttgtatcgggaggcagtaagaatcatcttttatcagtacaagggactagtta
aaaatggaaggttaggaaagactaaggtgcagggcttaaaatGGCGATTTTGACATTGCGGC
ATTGCTCAGCATGGCGGGCTGTGCTTTGTTAGGTTGTCCAAAATGGCGGATCCAGTTCTGTC
GCAGTGTTCAAGTGGCGGGAAGGCCACATCATGATGGGCGAGGCTTTGTTAAGTGGTTAGCA
TGGTGGTGGACATGTGCGGTCACACAGGAAAAGATGGCGGCTGAAGGTCTTGCCGCAGTGTA
AAACATGGCGGGCCTCTTTGTCTTTGCTGTGTGCTTTTCGTGTTGGGTTTTGCCGCAGGGAC
AATATGGCAGGCGTTGTCATATGTATATCATGGCTTTTGTCACGTGGACATCATGGCGGGCT
TGCCGCATTGTTAAAGATGGCGGGTTTTGCCGCCTAGTGCCACGCAGAGCGGGAGAAAAGGT
GGGATGGACAGTGCTGGATTGCTGCATAACCCAACCAATTAGAAATGGGGGTGGAATTGATC
ACAGCCAATTAGAGCAGAAGATGGAATTAGACTGATGACACACTGTCCAGCTACTCAGCGAA
GACCTGGGTGAATTAGCATGGCACTTCGCAGCTGTCTTTAGCCAGTCAGGAGAAAGAAGTGG
AGGGGCCACGTGTATGTCTCCCAGTGGGCGGTACACCAGGTGTTTTCAAGGTCTTTTCAAGG
ACATTTAGCCTTTCCACCTCTGTCCCCTCTTATTTGTCCCCTCCTGTCCAGTGCTGCCTCTT
GCAGTGCTGGATATCTGGCTGTGTGGTCTGAACCTCCCTCCATTCCTCTGTATTGGTGCCTC
ACCTAAGGCTAAGTATACCTCCCCCCCACCCCCCAACCCCCCCAACTCCCCCTCTGGTCTGC
CCTGCACTGCACTGTTGCCATGGGCAGTGCTCCAGGCCTGCTTGGTGTGGACATGGTGGTGA
GCCGTGGCAAGGACCAGAATGGATCACAGATGATCGTTGGCCAACAGGTGGCAGAAGAGGAA
TTCCTGCCTTCCTCAAGAGGAACACCTACCCCTTGGCTAATGCTGGGGTCGGATTTTGATTT
ATATTTATCTTTTGGATGTCAGTCATacagtctgattttgtggtttgctagtgtttgaattt
aagtcttaagtgactattatagaaatgtactatttgttaggctatagtgttttgtaaacttc
tgtttctattcacatcttctccacttgagagagacaccaaaatccagtcagtatctaatctg
gcttttgttaacttccctcaggagcagacattcatataggtgatactgtatttcagtccttt
cttttgaccccagaagccctagactgagaagataaaatggtcaggttgttggggaaaaaaaa
agtgccaggctctctagagaaaaatgtgaagagatgctccaggccaatgagaagaattagac
aagaaatacacagatgtgccagacttctgagaagcacctgccagcaacagcttccttctttg
agcttag
DYRK1A Left Arm Targeting Sequence 684 bp
gtaaactggcaaaggggtggctgggccaaaagacagaggaattaagtaagaagtccaggaaa
aatgaacttcacatcaaattttagagcacggtagccatgaatcttgtgaatagctcccaaaa
atgtcctgtggaagacaactagaaagcattctacaatcaggcacccacctccacctgcagcc
tcctgtgttgttctcatggggcacctctgggctccagctcctccaaggcacctccacactct
ctcaagtacactcttcactcttccccaaacatgattcccctactgctctgcctaactcccac
ttctctttcaagtagcagcttaaacgtcacctcatatttggctggaaaatagaatatagaca
gaggggtaagttaaggctagaaaggcaggctgggtcaacagaatggcaagctaaaacatggg
attttctaaaacagcctaagagggtgccagataaaagtgtgcaaggagtggcacaactccag
tttcatctttagctatagcaattaacaccataaggagtctggattcaattttgccatttact
agctagctaccaacttctgtgtcgctttgggcaaatcaattaaatccatacctccctttcca
tctgcagaatgggtttataacagtacttaaacctcaaggtactaagaacagtaaagagttaa
tg
DYRK1A Right Arm Targeting sequence 502 bp
aaaccagaaagtattctcagtaatgatagtatggataaagcaggtttctatgaccctttatt
acagaatctgtgagtttttcacaattaaaaagtaataaaaagtagtgacaacattcactgaa
ctcttattctatgccaacttgttccggtatgcccttacacccacaaaagccctatgcataag
gtggcattattccagcatgtattgcattgtacacacaaagaggtcaagcactccaccacggc
cctaagcatggtggctgaggtgggaaggccagaggtaggtgggcccgcgcccttttccactc
tgaaccatgcctccaagataggagggtgggaaagtgctcaagacacattagaaattccccat
aaaagacaagattgttgaacacctgcaagtgaataaagataaactgatctcagaggggaaaa
agacgcagggttaggaaacagcaccctgctcgaggacgttctttccaaacagcctgctcatc
acccgt
494 bp XIST-A-repeat/DYRK1A donor plasmid sequence
Tacgtaaactggcaaaggggtggctgggccaaaagacagaggaattaagtaagaagtccagg
aaaaatgaacttcacatcaaattttagagcacggtagccatgaatcttgtgaatagctccca
aaaatgtcctgtggaagacaactagaaagcattctacaatcaggcacccacctccacctgca
gcctcctgtgttgttctcatggggcacctctgggctccagctcctccaaggcacctccacac
tctctcaagtacactcttcactcttccccaaacatgattcccctactgctctgcctaactcc
cacttctctttcaagtagcagcttaaacgtcacctcatatttggctggaaaatagaatatag
acagaggggtaagttaaggctagaaaggcaggctgggtcaacagaatggcaagctaaaacat
gggattttctaaaacagcctaagagggtgccagataaaagtgtgcaaggagtggcacaactc
cagtttcatctttagctatagcaattaacaccataaggagtctggattcaattttgccattt
actagctagctaccaacttctgtgtcgctttgggcaaatcaattaaatccatacctcccttt
ccatctgcagaatgggtttataacagtacttaaacctcaaggtactaagaacagtaaagagt
taatggtaCATGTGAGCAAAAGGCCAGCAAAAGGCCAGGAACCGTAAAAAGGCCGCGTTGCT
GGCGTTTTTCCATAGGCTCCGCCCCCCTGACGAGCATCACAAAAATCGACGCTCAAGTCAGA
GGTGGCGAAACCCGACAGGACTATAAAGATACCAGGCGTTTCCCCCTGGAAGCTCCCTCGTG
CGCTCTCCTGTTCCGACCCTGCCGCTTACCGGATACCTGTCCGCCTTTCTCCCTTCGGGAAG
CGTGGCGCTTTCTCATAGCTCACGCTGTAGGTATCTCAGTTCGGTGTAGGTCGTTCGCTCCA
AGCTGGGCTGTGTGCACGAACCCCCCGTTCAGCCCGACCGCTGCGCCTTATCCGGTAACTAT
CGTCTTGAGTCCAACCCGGTAAGACACGACTTATCGCCACTGGCAGCAGCCACTGGTAACAG
GATTAGCAGAGCGAGGTATGTAGGCGGTGCTACAGAGTTCTTGAAGTGGTGGCCTAACTACG
GCTACACTAGAAGAACAGTATTTGGTATCTGCGCTCTGCTGAAGCCAGTTACCTTCGGAAAA
AGAGTTGGTAGCTCTTGATCCGGCAAACAAACCACCGCTGGTAGCGGTGGTTTTTTTGTTTG
CAAGCAGCAGATTACGCGCAGAAAAAAAGGATCTCAAGAAGATCCTTTGATCTTTTCTACGG
GGTCTGACGCTCAGTGGAACGAAAACTCACGTTAAGGGATTTTGGTCATGAGATTATCAAAA
AGGATCTTCACCTAGATCCTTTTAAATTAAAAATGAAGTTTTAAATCAATCTAAAGTATATA
TGAGTAAACTTGGTCTGACAGTTACCAATGCTTAATCAGTGAGGCACCTATCTCAGCGATCT
GTCTATTTCGTTCATCCATAGTTGCCTGACTCCCCGTCGTGTAGATAACTACGATACGGGAG
GGCTTACCATCTGGCCCCAGTGCTGCAATGATACCGCGAGACCCACGCTCACCGGCTCCAGA
TTTATCAGCAATAAACCAGCCAGCCGGAAGGGCCGAGCGCAGAAGTGGTCCTGCAACTTTAT
CCGCCTCCATCCAGTCTATTAATTGTTGCCGGGAAGCTAGAGTAAGTAGTTCGCCAGTTAAT
AGTTTGCGCAACGTTGTTGCCATTGCTACAGGCATCGTGGTGTCACGCTCGTCGTTTGGTAT
GGCTTCATTCAGCTCCGGTTCCCAACGATCAAGGCGAGTTACATGATCCCCCATGTTGTGCA
AAAAAGCGGTTAGCTCCTTCGGTCCTCCGATCGTTGTCAGAAGTAAGTTGGCCGCAGTGTTA
TCACTCATGGTTATGGCAGCACTGCATAATTCTCTTACTGTCATGCCATCCGTAAGATGCTT
TTCTGTGACTGGTGAGTACTCAACCAAGTCATTCTGAGAATAGTGTATGCGGCGACCGAGTT
GCTCTTGCCCGGCGTCAATACGGGATAATACCGCGCCACATAGCAGAACTTTAAAAGTGCTC
ATCATTGGAAAACGTTCTTCGGGGCGAAAACTCTCAAGGATCTTACCGCTGTTGAGATCCAG
TTCGATGTAACCCACTCGTGCACCCAACTGATCTTCAGCATCTTTTACTTTCACCAGCGTTT
CTGGGTGAGCAAAAACAGGAAGGCAAAATGCCGCAAAAAAGGGAATAAGGGCGACACGGAAA
TGTTGAATACTCATACTCTTCCTTTTTCAATATTATTGAAGCATTTATCAGGGTTATTGTCT
CATGAGCGGATACATATTTGAATGTATTTAGAAAAATAAACAAATAGGGGTTCCGCGCACAT
TTCCCCGAAAAGTGCCACCTGACGTCTAAGAAACCATTATTATCATGACATTAACCTATAAA
AATAGGCGTATCACGAGGCCCTTTCGTCTTCAAGAATTcgaaaaccagaaagtattctcagt
aatgatagtatggataaagcaggtttctatgaccctttattacagaatctgtgagtttttca
caattaaaaagtaataaaaagtagtgacaacattcactgaactcttattctatgccaacttg
ttccggtatgcccttacacccacaaaagccctatgcataaggtggcattattccagcatgta
ttgcattgtacacacaaagaggtcaagcactccaccacggccctaagcatggtggctgaggt
gggaaggccagaggtaggtgggcccgcgcccttttccactctgaaccatgcctccaagatag
gagggtgggaaagtgctcaagacacattagaaattccccataaaagacaagattgttgaaca
cctgcaagtgaataaagataaactgatctcagaggggaaaaagacgcagggttaggaaacag
caccctgctcgaggacgttctttccaaacagcctgctcatcacccgttcgAATTCCTCGAGT
TTACTCCCTATCAGTGATAGAGAACGTATGAAGAGTTTACTCCCTATCAGTGATAGAGAACG
TATGCAGACTTTACTCCCTATCAGTGATAGAGAACGTATAAGGAGTTTACTCCCTATCAGTG
ATAGAGAACGTATGACCAGTTTACTCCCTATCAGTGATAGAGAACGTATCTACAGTTTACTC
CCTATCAGTGATAGAGAACGTATATCCAGTTTACTCCCTATCAGTGATAGAGAACGTATAAG
CTTTAGGCGTGTACGGTGGGCGCCTATAAAAGCAGAGCTCGTTTAGTGAACCGTCAGATCGC
CTGGAGCAATTCCACAACACTTTTGTCTTATACCAACTTTCCGTACCACTTCCTACCCTCGT
AAAGTCGACACCGGGGCCCAGATCTGGTACCGAGCTCGGATCCACTAGTCCAGTGTGGTGGA
ATTCTGCAGAgaatttttctttggaatcatttttggttgacatctctgttttttgtggatca
gttttttactcttccactctcttttctatattttgcccatcggggctgcggatacctggttt
tattattttttctttgcccaacggggccgtggatacctgccttttaattcttttttattcgc
ccatcggggccgcggatacctgctttttatttttttttccttagcccatcggggtatcggat
acctgctgattcccttcccctctgaacccccaacactctggcccatcggggtgacggatatc
tgctttttaaaaattttctttttttggcccatcggggcttcggatacctgcttttttttttt
ttatttttccttgcccatcggggcctcggatacctgctttaatttttgtttttctggcccat
cggggccgcggatacctgctttgatttttttttttcatcgcccatcggtgctttttatggat
gaaaaaatgttTCGATCGGCCGGATATCACGCGTCATATGGCTAGCCTGCAGGGATCCAATG
TAACTGTATTCAGCGATGACGAAATTCTTAGCTATTGTAATACTCTAGAGGATCTTTGTGAA
GGAACCTTACTTCTGTGGTGTGACATAATTGGACAAACTACCTACAGAGATTTAAAGCTCTA
AGGTAAATATAAAATTTTTAAGTGTATAATGTGTTAAACTACTGATTCTAATTGTTTGTGTA
TTTTAGATTCCAACCTATGGAACTGATGAATGGGAGCAGTGGTGGAATGCCTTTAATGAGGA
AAACCTGTTTTGCTCAGAAGAAATGCCATCTAGTGATGATGAGGCTACTGCTGACTCTCAAC
ATTCTACTCCTCCAAAAAAGAAGAGAAAGGTAGAAGACCCCAAGGACTTTCCTTCAGAATTG
CTAAGTTTTTTGAGTCATGCTGTGTTTAGTAATAGAACTCTTGCTTGCTTTGCTATTTACAC
CACAAAGGAAAAAGCTGCACTGCTATACAAGAAAATTATGGAAAAATATTCTGTAACCTTTA
TAAGTAGGCATAACAGTTATAATCATAACATACTGTTTTTTCTTACTCCACACAGGCATAGA
GTGTCTGCTATTAATAACTATGCTCAAAAATTGTGTACCTTTAGCTTTTTAATTTGTAAAGG
GGTTAATAAGGAATATTTGATGTATAGTGCCTTGACTAGAGATCATAATCAGCCATACCACA
TTTGTAGAGGTTTTACTTGCTTTAAAAAACCTCCCACACCTCCCCCTGAACCTGAAACATAA
AATGAATGCAATTGTTGTTGTTAACTTGTTTATTGCAGCTTATAATGGTTACAAATAAAGCA
ATAGCATCACAAATTTCACAAATAAAGCATTTTTTTCACTGCATTCTAGTTGTGGTTTGTCC
AAACTCATCAATGTATCTTAGAGGGACAGCCCCCCCCCAAAGCCCCCAGGGATGTAATTACG
TCCCTCCCCCGCTAGGGGGCAGCAGCGAGCCGCCCGGGGCTCCGCTCCGGTCCGGCGCTCCC
CCCGCATCCCCGAGCCGGCAGCGTGCGGGGACAGCCCGGGCACGGGGAAGGTGGCACGGGAT
CGCTTTCCTCTGAACGCTTCTCGCTGCTCTTTGAGCCTGCAGACACCTGGGGGGATACGGGG
AAAAGCTAGCAAGGATCTGCGATCGCTCCGGTGCCCGTCAGTGGGCAGAGCGCACATCGCCC
ACAGTCCCCGAGAAGTTGGGGGGAGGGGTCGGCAATTGAACGGGTGCCTAGAGAAGGTGGCG
CGGGGTAAACTGGGAAAGTGATGTCGTGTACTGGCTCCGCCTTTTTCCCGAGGGTGGGGGAG
AACCGTATATAAGTGCAGTAGTCGCCGTGAACGTTCTTTTTCGCAACGGGTTTGCCGCCAGA
ACACAGCTGAAGCTTCGAGGGGCTCGCATCTCTCCTTCACGCGCCCGCCGCCCTACCTGAGG
CCGCCATCCACGCCGGTTGAGTCGCGTTCTGCCGCCTCCCGCCTGTGGTGCCTCCTGAACTG
CGTCCGCCGTCTAGGTAAGTTTAAAGCTCAGGTCGAGTCCGGGCCTTTGTCCGGCGCTCCCT
TGGAGCCTACCTAGACTCAGCCGGCTCTCCACGCTTTGCCTGACCCTGCTTGCTCAACTCTA
CGTCTTTGTTTCGTTTTCTGTTCTGCGCCGTTACAGATCCAAGCTGTGACCGGCGCCTACGA
ATTCGCCACCatggccagctccgaggatgtcatcaaagagtttatgagatttaaggtcaaga
tggagggaagcgtcaacggacacgagttcgagattgagggagaaggagaaggccggccttac
gagggcacacaaaccgctaagctcaaggtcacaaaaggaggacccctccccttctcctggga
tattctgagccctcagttccagtacggaagcaaagcctatgttaaacaccctgccgacatcc
ctgactatctgaagctctccttccctgaaggcttcaagtgggagagattcatgaacttcgag
gacggaggcgtggtgacagtcacacaagatagcaccctccaggacggagagtttatttataa
ggtgaaactcagaggaaccaacttcccctccgatggccctgtcatgcaaaaaaaaacaatgg
gatgggaagcctccaccgagagaatgtatcctgaggatggcgctctgaaaggcgaaattaaa
atgagactgaaactcaaagacggaggacactacgatgccgaggtcaaaacaacctacaaggc
caagaaacaagtgcagctgcctggcgcctacatgactgatattaaactcgacattatcagcc
ataatggggactacaccatcgtggaacaatatgagagagctgagggcagacatagcacaggc
gctggaAGTACTTGAGGATCCtgatcgagtctagagggcccccgctgatcagcctcgactgt
gccttctagttgccagccatctgttgtttgcccctcccccgtgccttccttgaccctggaag
gtgccactcccactgtcctttcctaataaaatgaggaaattgcatcgcattgtctgagtagg
tgtcattctattctggggggtggggggggcaggacagcaagggggaggattgggaagagaa
tagcaggcatgctggggaagatctTCATGTCTGCGGCTCTAGAGCTGCATTAATGAATCGGC
CAACGCGCGGGGAGAGGCGGTTTGCGTATTGGGCGCTCTTCCGCTTCCTCGCTCACTGACTC
GCTGCGCTCGGTCGTTCGGCTGCGGCGAGCGGTATCAGCTCACTCAAAGGCGGTAATACGGT
TATCCACAGAATCAGGGGATAACGCAGGAAAGAACATG
1 KB XIST-minigene/DYRK1A donor plasmid sequence
aaaccagaaagtattctcagtaatgatagtatggataaagcaggtttctatgaccctttatt
acagaatctgtgagtttttcacaattaaaaagtaataaaaagtagtgacaacattcactgaa
ctcttattctatgccaacttgttccggtatgcccttacacccacaaaagccctatgcataag
gtggcattattccagcatgtattgcattgtacacacaaagaggtcaagcactccaccacggc
cctaagcatggtggctgaggtgggaaggccagaggtaggtgggcccgcgcccttttccactc
tgaaccatgcctccaagataggagggtgggaaagtgctcaagacacattagaaattccccat
aaaagacaagattgttgaacacctgcaagtgaataaagataaactgatctcagaggggaaaa
agacgcagggttaggaaacagcaccctgctcgaggacgttctttccaaacagcctgctcatc
acccgttcgAATTCCTCGAGTTTACTCCCTATCAGTGATAGAGAACGTATGAAGAGTTTACT
CCCTATCAGTGATAGAGAACGTATGCAGACTTTACTCCCTATCAGTGATAGAGAACGTATAA
GGAGTTTACTCCCTATCAGTGATAGAGAACGTATGACCAGTTTACTCCCTATCAGTGATAGA
GAACGTATCTACAGTTTACTCCCTATCAGTGATAGAGAACGTATATCCAGTTTACTCCCTAT
CAGTGATAGAGAACGTATAAGCTTTAGGCGTGTACGGTGGGCGCCTATAAAAGCAGAGCTCG
TTTAGTGAACCGTCAGATCGCCTGGAGCAATTCCACAACACTTTTGTCTTATACCAACTTTC
CGTACCACTTCCTACCCTCGTAAAGTCGACACCGGGGCCCAGATCTGGTACCGAGCTCGGAT
CCACTAGTCCAGTGTGGTGGAATTCTGCAGAGAATTTTTCTTTGGAATCATTTTTGGTGACA
TCTCTGTTTTTTGTGGATCAGTTTTTTACTCTTCCACTCTCTTTTCTATATTTTGCCCATCG
GGGCTGCGGATACCTGGTTTTATTATTTTTTCTTTGCCCAACGGGGCCGTGGATACCTGCCT
TTTAATTCTTTTTTATTCGCCCATCGGGGCCGCGGATACCTGCTTTTTATTTTTTTTTCCTT
AGCCCATCGGGGTATCGGATACCTGCTGATTCCCTTCCCCTCTGAACCCCCAACACTCTGGC
CCATCGGGGTGACGGATATCTGCTTTTTAAAAATTTTCTTTTTTTGGCCCATCGGGGCTTCG
GATACCTGCTTTTTTTTTTTTTATTTTTCCTTGCCCATCGGGGCCTCGGATACCTGCTTTAA
TTTTTGTTTTTCTGGCCCATCGGGGCCGCGGATACCTGCTTTGATTTTTTTTTTTCATCGCC
CATCGGTGCTTTTTATGGATGAAAAAATGTTTCTTTGCTGTGTGCTTTTCGTGTTGGGTTTT
GCCGCAGGGACAATATGGCAGGCGTTGTCATATGTATATCATGGCTTTTGTCACGTGGACAT
CATGGCGGGCTTGCCGCATTGTTAAAGATGGCGGGTTTTGCCGCCTAGTCTATTTGTTAGGC
TATAGTGTTTTGTAAACTTCTGTTTCTATTCACATCTTCTCCACTTGAGAGAGACACCAAAA
TCCAGTCAGTATCTAATCTGGCTTTTGTTAACTTCCCTCAGGAGCAGACATTCATATAGGTG
ATACTGTATTTCAGTCCTTTCTTTTGACCCCAGAAGCCCTAGACTGAGAAGATAAAATGGTC
AGGTTGTTGGGGAAAAAAAAAGTGCCAGGCTCTCTAGAGAAAAATGTGAAGAGATGCTCCAG
GCCAATGAGAAGAATTAGACAAGAAATACACAGATGTGCCAGACTTCTGAGAAGCACCTGCC
AGCAACAGCTTCCTTCTTTGAGCTTAGTCCATCCCTCATGAAAAATGACTGACCACTGCTGG
GCAGCAGGAGGGATGATGACCAACTAATTCCCAAACCCCAGTCTCATTGGTACCATCGATCG
GCCGGATATCACGCGTCATATGGCTAGCCTGCAGGGATCCAATGTAACTGTATTCAGCGATG
ACGAAATTCTTAGCTATTGTAATACTCTAGAGGATCTTTGTGAAGGAACCTTACTTCTGTGG
TGTGACATAATTGGACAAACTACCTACAGAGATTTAAAGCTCTAAGGTAAATATAAAATTTT
TAAGTGTATAATGTGTTAAACTACTGATTCTAATTGTTTGTGTATTTTAGATTCCAACCTAT
GGAACTGATGAATGGGAGCAGTGGTGGAATGCCTTTAATGAGGAAAACCTGTTTTGCTCAGA
AGAAATGCCATCTAGTGATGATGAGGCTACTGCTGACTCTCAACATTCTACTCCTCCAAAAA
AGAAGAGAAAGGTAGAAGACCCCAAGGACTTTCCTTCAGAATTGCTAAGTTTTTTGAGTCAT
GCTGTGTTTAGTAATAGAACTCTTGCTTGCTTTGCTATTTACACCACAAAGGAAAAAGCTGC
ACTGCTATACAAGAAAATTATGGAAAAATATTCTGTAACCTTTATAAGTAGGCATAACAGTT
ATAATCATAACATACTGTTTTTTCTTACTCCACACAGGCATAGAGTGTCTGCTATTAATAAC
TATGCTCAAAAATTGTGTACCTTTAGCTTTTTAATTTGTAAAGGGGTTAATAAGGAATATTT
GATGTATAGTGCCTTGACTAGAGATCATAATCAGCCATACCACATTTGTAGAGGTTTTACTT
GCTTTAAAAAACCTCCCACACCTCCCCCTGAACCTGAAACATAAAATGAATGCAATTGTTGT
TGTTAACTTGTTTATTGCAGCTTATAATGGTTACAAATAAAGCAATAGCATCACAAATTTCA
CAAATAAAGCATTTTTTTCACTGCATTCTAGTTGTGGTTTGTCCAAACTCATCAATGTATCT
TAGAGGGACAGCCCCCCCCCAAAGCCCCCAGGGATGTAATTACGTCCCTCCCCCGCTAGGGG
GCAGCAGCGAGCCGCCCGGGGCTCCGCTCCGGTCCGGCGCTCCCCCCGCATCCCCGAGCCGG
CAGCGTGCGGGGACAGCCCGGGCACGGGGAAGGTGGCACGGGATCGCTTTCCTCTGAACGCT
TCTCGCTGCTCTTTGAGCCTGCAGACACCTGGGGGGATACGGGGAAAAGCTAGCAAGGATCT
GCGATCGCTCCGGTGCCCGTCAGTGGGCAGAGCGCACATCGCCCACAGTCCCCGAGAAGTTG
GGGGGAGGGGTCGGCAATTGAACGGGTGCCTAGAGAAGGTGGCGCGGGGTAAACTGGGAAAG
TGATGTCGTGTACTGGCTCCGCCTTTTTCCCGAGGGTGGGGGAGAACCGTATATAAGTGCAG
TAGTCGCCGTGAACGTTCTTTTTCGCAACGGGTTTGCCGCCAGAACACAGCTGAAGCTTCGA
GGGGCTCGCATCTCTCCTTCACGCGCCCGCCGCCCTACCTGAGGCCGCCATCCACGCCGGTT
GAGTCGCGTTCTGCCGCCTCCCGCCTGTGGTGCCTCCTGAACTGCGTCCGCCGTCTAGGTAA
GTTTAAAGCTCAGGTCGAGTCCGGGCCTTTGTCCGGCGCTCCCTTGGAGCCTACCTAGACTC
AGCCGGCTCTCCACGCTTTGCCTGACCCTGCTTGCTCAACTCTACGTCTTTGTTTCGTTTTC
TGTTCTGCGCCGTTACAGATCCAAGCTGTGACCGGCGCCTACGAATTCGCCACCatggccag
ctccgaggatgtcatcaaagagtttatgagatttaaggtcaagatggagggaagcgtcaacg
gacacgagttcgagattgagggagaaggagaaggccggccttacgagggcacacaaaccgct
aagctcaaggtcacaaaaggaggacccctccccttctcctgggatattctgagccctcagtt
ccagtacggaagcaaagcctatgttaaacaccctgccgacatccctgactatctgaagctct
ccttccctgaaggcttcaagtgggagagattcatgaacttcgaggacggaggcgtggtgaca
gtcacacaagatagcaccctccaggacggagagtttatttataaggtgaaactcagaggaac
caacttcccctccgatggccctgtcatgcaaaaaaaaacaatgggatgggaagcctccaccg
agagaatgtatcctgaggatggcgctctgaaaggcgaaattaaaatgagactgaaactcaaa
gacggaggacactacgatgccgaggtcaaaacaacctacaaggccaagaaacaagtgcagct
gcctggcgcctacatgactgatattaaactcgacattatcagccataatggggactacacca
tcgtggaacaatatgagagagctgagggcagacatagcacaggcgctggaAGTACTTGAGGA
TCCtgatcgagtctagagggcccccgctgatcagcctcgactgtgccttctagttgccagcc
atctgttgtttgcccctcccccgtgccttccttgaccctggaaggtgccactcccactgtcc
tttcctaataaaatgaggaaattgcatcgcattgtctgagtaggtgtcattctattctgggg
ggtggggtggggcaggacagcaagggggaggattgggaagagaatagcaggcatgctgggga
agatctTCATGTCTGCGGCTCTAGAGCTGCATTAATGAATCGGCCAACGCGCGGGGAGAGGC
GGTTTGCGTATTGGGCGCTCTTCCGCTTCCTCGCTCACTGACTCGCTGCGCTCGGTCGTTCG
GCTGCGGCGAGCGGTATCAGCTCACTCAAAGGCGGTAATACGGTTATCCACAGAATCAGGGG
ATAACGCAGGAAAGAACATGTacgtaaactggcaaaggggtggctgggccaaaagacagagg
aattaagtaagaagtccaggaaaaatgaacttcacatcaaattttagagcacggtagccatg
aatcttgtgaatagctcccaaaaatgtcctgtggaagacaactagaaagcattctacaatca
ggcacccacctccacctgcagcctcctgtgttgttctcatggggcacctctgggctccagct
cctccaaggcacctccacactctctcaagtacactcttcactcttccccaaacatgattccc
ctactgctctgcctaactcccacttctctttcaagtagcagcttaaacgtcacctcatattt
ggctggaaaatagaatatagacagaggggtaagttaaggctagaaaggcaggctgggtcaac
agaatggcaagctaaaacatgggattttctaaaacagcctaagagggtgccagataaaagtg
tgcaaggagtggcacaactccagtttcatctttagctatagcaattaacaccataaggagtc
tggattcaattttgccatttactagctagctaccaacttctgtgtcgctttgggcaaatcaa
ttaaatccatacctccctttccatctgcagaatgggtttataacagtacttaaacctcaagg
tactaagaacagtaaagagttaatggtaCATGTGAGCAAAAGGCCAGCAAAAGGCCAGGAAC
CGTAAAAAGGCCGCGTTGCTGGCGTTTTTCCATAGGCTCCGCCCCCCTGACGAGCATCACAA
AAATCGACGCTCAAGTCAGAGGTGGCGAAACCCGACAGGACTATAAAGATACCAGGCGTTTC
CCCCTGGAAGCTCCCTCGTGCGCTCTCCTGTTCCGACCCTGCCGCTTACCGGATACCTGTCC
GCCTTTCTCCCTTCGGGAAGCGTGGCGCTTTCTCATAGCTCACGCTGTAGGTATCTCAGTTC
GGTGTAGGTCGTTCGCTCCAAGCTGGGCTGTGTGCACGAACCCCCCGTTCAGCCCGACCGCT
GCGCCTTATCCGGTAACTATCGTCTTGAGTCCAACCCGGTAAGACACGACTTATCGCCACTG
GCAGCAGCCACTGGTAACAGGATTAGCAGAGCGAGGTATGTAGGCGGTGCTACAGAGTTCTT
GAAGTGGTGGCCTAACTACGGCTACACTAGAAGAACAGTATTTGGTATCTGCGCTCTGCTGA
AGCCAGTTACCTTCGGAAAAAGAGTTGGTAGCTCTTGATCCGGCAAACAAACCACCGCTGGT
AGCGGTGGTTTTTTTGTTTGCAAGCAGCAGATTACGCGCAGAAAAAAAGGATCTCAAGAAGA
TCCTTTGATCTTTTCTACGGGGTCTGACGCTCAGTGGAACGAAAACTCACGTTAAGGGATTT
TGGTCATGAGATTATCAAAAAGGATCTTCACCTAGATCCTTTTAAATTAAAAATGAAGTTTT
AAATCAATCTAAAGTATATATGAGTAAACTTGGTCTGACAGTTACCAATGCTTAATCAGTGA
GGCACCTATCTCAGCGATCTGTCTATTTCGTTCATCCATAGTTGCCTGACTCCCCGTCGTGT
AGATAACTACGATACGGGAGGGCTTACCATCTGGCCCCAGTGCTGCAATGATACCGCGAGAC
CCACGCTCACCGGCTCCAGATTTATCAGCAATAAACCAGCCAGCCGGAAGGGCCGAGCGCAG
AAGTGGTCCTGCAACTTTATCCGCCTCCATCCAGTCTATTAATTGTTGCCGGGAAGCTAGAG
TAAGTAGTTCGCCAGTTAATAGTTTGCGCAACGTTGTTGCCATTGCTACAGGCATCGTGGTG
TCACGCTCGTCGTTTGGTATGGCTTCATTCAGCTCCGGTTCCCAACGATCAAGGCGAGTTAC
ATGATCCCCCATGTTGTGCAAAAAAGCGGTTAGCTCCTTCGGTCCTCCGATCGTTGTCAGAA
GTAAGTTGGCCGCAGTGTTATCACTCATGGTTATGGCAGCACTGCATAATTCTCTTACTGTC
ATGCCATCCGTAAGATGCTTTTCTGTGACTGGTGAGTACTCAACCAAGTCATTCTGAGAATA
GTGTATGCGGCGACCGAGTTGCTCTTGCCCGGCGTCAATACGGGATAATACCGCGCCACATA
GCAGAACTTTAAAAGTGCTCATCATTGGAAAACGTTCTTCGGGGCGAAAACTCTCAAGGATC
TTACCGCTGTTGAGATCCAGTTCGATGTAACCCACTCGTGCACCCAACTGATCTTCAGCATC
TTTTACTTTCACCAGCGTTTCTGGGTGAGCAAAAACAGGAAGGCAAAATGCCGCAAAAAAGG
GAATAAGGGCGACACGGAAATGTTGAATACTCATACTCTTCCTTTTTCAATATTATTGAAGC
ATTTATCAGGGTTATTGTCTCATGAGCGGATACATATTTGAATGTATTTAGAAAAATAAACA
AATAGGGGTTCCGCGCACATTTCCCCGAAAAGTGCCACCTGACGTCTAAGAAACCATTATTA
TCATGACATTAACCTATAAAAATAGGCGTATCACGAGGCCCTTTCGTCTTCAAGAATTcga
2.5 KB XIST-minigene/DYRK1A donor plasmid sequence
aaaccagaaagtattctcagtaatgatagtatggataaagcaggtttctatgaccctttatt
acagaatctgtgagtttttcacaattaaaaagtaataaaaagtagtgacaacattcactgaa
ctcttattctatgccaacttgttccggtatgcccttacacccacaaaagccctatgcataag
gtggcattattccagcatgtattgcattgtacacacaaagaggtcaagcactccaccacggc
cctaagcatggtggctgaggtgggaaggccagaggtaggtgggcccgcgcccttttccactc
tgaaccatgcctccaagataggagggtgggaaagtgctcaagacacattagaaattccccat
aaaagacaagattgttgaacacctgcaagtgaataaagataaactgatctcagaggggaaaa
agacgcagggttaggaaacagcaccctgctcgaggacgttctttccaaacagcctgctcatc
acccgttcgAATTCCTCGAGTTTACTCCCTATCAGTGATAGAGAACGTATGAAGAGTTTACT
CCCTATCAGTGATAGAGAACGTATGCAGACTTTACTCCCTATCAGTGATAGAGAACGTATAA
GGAGTTTACTCCCTATCAGTGATAGAGAACGTATGACCAGTTTACTCCCTATCAGTGATAGA
GAACGTATCTACAGTTTACTCCCTATCAGTGATAGAGAACGTATATCCAGTTTACTCCCTAT
CAGTGATAGAGAACGTATAAGCTTTAGGCGTGTACGGTGGGCGCCTATAAAAGCAGAGCTCG
TTTAGTGAACCGTCAGATCGCCTGGAGCAATTCCACAACACTTTTGTCTTATACCAACTTTC
CGTACCACTTCCTACCCTCGTAAAGTCGACACCGGGGCCCAGATCTGGTACCGAGCTCGGAT
CCACTAGTCCAGTGTGGTGGAATTCTGCAGAGAATTTTTCTTTggaatcatttttggtGACA
TCTCTGTTTTTTGTGGATCAGTTTTTTACTCTTCCACTCTCTTTTCTATATTTTGCCCATCG
GGGCTGCGGATACCTGGTTTTATTATTTTTTCTTTGCCCAACGGGGCCGTGGATACCTGCCT
TTTAATTCTTTTTTATTCGCCCATCGGGGCCGCGGATACCTGCTTTTTATTTTTTTTTCCTT
AGCCCATCGGGGTATCGGATACCTGCTGATTCCCTTCCCCTCTGAACCCCCAACACTCTGGC
CCATCGGGGTGACGGATATCTGCTTTTTAAAAATTTTCTTTTTTTGGCCCATCGGGGCTTCG
GATACCTGCTTTTTTTTTTTTTATTTTTCCTTGCCCATCGGGGCCTCGGATACCTGCTTTAA
TTTTTGTTTTTCTGGCCCATCGGGGCCGCGGATACCTGCTTTGATTTTTTTTTTTCATCGCC
CATCGGTGCTTTTTATGGATGAAAAAATGTTGGTTTTGTGGGTTGTTGCACTCTCTGGAATA
TCTACACTTTTTTTTGCTGCTGATCATTTGGTGGTGTGTGAGTGTACCTACCGCTTTGGCAG
AGAATGACTCTGCAGTTAAGCTAAGGGCGTGTTCAGATTGTGGAGGAAAAGTGGCCGCCATT
TTAACTTGCCGCATAACTCGGCTTAGGGCTAGTCGTTTGTGCTAAGTTAAACTAGGGAGGCA
AGATGGATGATAGCAGGTCAGGCAGAGGAAGTCATGTGCATTGCATGAGCTAAACCTATCTG
AATGAATTGATTTGGGGCTTGTTAGGAGCTTTGCGTgattgttgtatcgggaggcagtaaga
atcatcttttatcagtacaagggactagttaaaaatggaaggttaggaaagactaaggtgca
gggcttaaaatGGCGATTTTGACATTGCGGCATTGCTCAGCATGGCGGGCTGTGCTTTGTTA
GGTTGTCCAAAATGGCGGATCCAGTTCTGTCGCAGTGTTCAAGTGGCGGGAAGGCCACATCA
TGATGGGCGAGGCTTTGTTAAGTGGTTAGCATGGTGGTGGACATGTGCGGTCACACAGGAAA
AGATGGCGGCTGAAGGTCTTGCCGCAGTGTAAAACATGGCGGGCCTCTTTGTCTTTGCTGTG
TGCTTTTCGTGTTGGGTTTTGCCGCAGGGACAATATGGCAGGCGTTGTCATATGTATATCAT
GGCTTTTGTCACGTGGACATCATGGCGGGCTTGCCGCATTGTTAAAGATGGCGGGTTTTGCC
GCCTAGTGCCACGCAGAGCGGGAGAAAAGGTGGGATGGACAGTGCTGGATTGCTGCATAACC
CAACCAATTAGAAATGGGGGTGGAATTGATCACAGCCAATTAGAGCAGAAGATGGAATTAGA
CTGATGACACACTGTCCAGCTACTCAGCGAAGACCTGGGTGAATTAGCATGGCACTTCGCAG
CTGTCTTTAGCCAGTCAGGAGAAAGAAGTGGAGGGGCCACGTGTATGTCTCCCAGTGGGCGG
TACACCAGGTGTTTTCAAGGTCTTTTCAAGGACATTTAGCCTTTCCACCTCTGTCCCCTCTT
ATTTGTCCCCTCCTGTCCAGTGCTGCCTCTTGCAGTGCTGGATATCTGGCTGTGTGGTCTGA
ACCTCCCTCCATTCCTCTGTATTGGTGCCTCACCTAAGGCTAAGTATACCTCCCCCCCACCC
CCCAACCCCCCCAACTCCCCCTCTGGTCTGCCCTGCACTGCACTGTTGCCATGGGCAGTGCT
CCAGGCCTGCTTGGTGTGGACATGGTGGTGAGCCGTGGCAAGGACCAGAATGGATCACAGAT
GATCGTTGGCCAACAGGTGGCAGAAGAGGAATTCCTGCCTTCCTCAAGAGGAACACCTACCC
CTTGGCTAATGCTGGGGTCGGATTTTGATTTATATTTATCTTTTGGATGTCAGTCATacagt
ctgattttgtggtttgctagtgtttgaatttaagtcttaagtgactattatagaaatgtact
atttgttaggctatagtgttttgtaaacttctgtttctattcacatcttctccacttgagag
agacaccaaaatccagtcagtatctaatctggcttttgttaacttccctcaggagcagacat
tcatataggtgatactgtatttcagtcctttcttttgaccccagaagccctagactgagaag
ataaaatggtcaggttgttggggaaaaaaaaagtgccaggctctctagagaaaaatgtgaag
agatgctccaggccaatgagaagaattagacaagaaatacacagatgtgccagacttctgag
aagcacctgccagcaacagcttccttctttgagcttagTCCATCCCTCATGAAAAATGACTG
ACCACTGCTGGGCAGCAGGAGGGATGATGACCAACTAATTCCCAAACCCCAGTCTCATTGGT
ACCATCGATCGGCCGGATATCACGCGTCATATGGCTAGCCTGCAGGGATCCAATGTAACTGT
ATTCAGCGATGACGAAATTCTTAGCTATTGTAATACTCTAGAGGATCTTTGTGAAGGAACCT
TACTTCTGTGGTGTGACATAATTGGACAAACTACCTACAGAGATTTAAAGCTCTAAGGTAAA
TATAAAATTTTTAAGTGTATAATGTGTTAAACTACTGATTCTAATTGTTTGTGTATTTTAGA
TTCCAACCTATGGAACTGATGAATGGGAGCAGTGGTGGAATGCCTTTAATGAGGAAAACCTG
TTTTGCTCAGAAGAAATGCCATCTAGTGATGATGAGGCTACTGCTGACTCTCAACATTCTAC
TCCTCCAAAAAAGAAGAGAAAGGTAGAAGACCCCAAGGACTTTCCTTCAGAATTGCTAAGTT
TTTTGAGTCATGCTGTGTTTAGTAATAGAACTCTTGCTTGCTTTGCTATTTACACCACAAAG
GAAAAAGCTGCACTGCTATACAAGAAAATTATGGAAAAATATTCTGTAACCTTTATAAGTAG
GCATAACAGTTATAATCATAACATACTGTTTTTTCTTACTCCACACAGGCATAGAGTGTCTG
CTATTAATAACTATGCTCAAAAATTGTGTACCTTTAGCTTTTTAATTTGTAAAGGGGTTAAT
AAGGAATATTTGATGTATAGTGCCTTGACTAGAGATCATAATCAGCCATACCACATTTGTAG
AGGTTTTACTTGCTTTAAAAAACCTCCCACACCTCCCCCTGAACCTGAAACATAAAATGAAT
GCAATTGTTGTTGTTAACTTGTTTATTGCAGCTTATAATGGTTACAAATAAAGCAATAGCAT
CACAAATTTCACAAATAAAGCATTTTTTTCACTGCATTCTAGTTGTGGTTTGTCCAAACTCA
TCAATGTATCTTAGAGGGACAGCCCCCCCCCAAAGCCCCCAGGGATGTAATTACGTCCCTCC
CCCGCTAGGGGGCAGCAGCGAGCCGCCCGGGGCTCCGCTCCGGTCCGGCGCTCCCCCCGCAT
CCCCGAGCCGGCAGCGTGCGGGGACAGCCCGGGCACGGGGAAGGTGGCACGGGATCGCTTTC
CTCTGAACGCTTCTCGCTGCTCTTTGAGCCTGCAGACACCTGGGGGGATACGGGGAAAAGCT
AGCAAGGATCTGCGATCGCTCCGGTGCCCGTCAGTGGGCAGAGCGCACATCGCCCACAGTCC
CCGAGAAGTTGGGGGGAGGGGTCGGCAATTGAACGGGTGCCTAGAGAAGGTGGCGCGGGGTA
AACTGGGAAAGTGATGTCGTGTACTGGCTCCGCCTTTTTCCCGAGGGTGGGGGAGAACCGTA
TATAAGTGCAGTAGTCGCCGTGAACGTTCTTTTTCGCAACGGGTTTGCCGCCAGAACACAGC
TGAAGCTTCGAGGGGCTCGCATCTCTCCTTCACGCGCCCGCCGCCCTACCTGAGGCCGCCAT
CCACGCCGGTTGAGTCGCGTTCTGCCGCCTCCCGCCTGTGGTGCCTCCTGAACTGCGTCCGC
CGTCTAGGTAAGTTTAAAGCTCAGGTCGAGTCCGGGCCTTTGTCCGGCGCTCCCTTGGAGCC
TACCTAGACTCAGCCGGCTCTCCACGCTTTGCCTGACCCTGCTTGCTCAACTCTACGTCTTT
GTTTCGTTTTCTGTTCTGCGCCGTTACAGATCCAAGCTGTGACCGGCGCCTACGAATTCGCC
ACCatggccagctccgaggatgtcatcaaagagtttatgagatttaaggtcaagatggaggg
aagcgtcaacggacacgagttcgagattgagggagaaggagaaggccggccttacgagggca
cacaaaccgctaagctcaaggtcacaaaaggaggacccctccccttctcctgggatattctg
agccctcagttccagtacggaagcaaagcctatgttaaacaccctgccgacatccctgacta
tctgaagctctccttccctgaaggcttcaagtgggagagattcatgaacttcgaggacggag
gcgtggtgacagtcacacaagatagcaccctccaggacggagagtttatttataaggtgaaa
ctcagaggaaccaacttcccctccgatggccctgtcatgcaaaaaaaaacaatgggatggga
agcctccaccgagagaatgtatcctgaggatggcgctctgaaaggcgaaattaaaatgagac
tgaaactcaaagacggaggacactacgatgccgaggtcaaaacaacctacaaggccaagaaa
caagtgcagctgcctggcgcctacatgactgatattaaactcgacattatcagccataatgg
ggactacaccatcgtggaacaatatgagagagctgagggcagacatagcacaggcgctggaA
GTACTTGAGGATCCtgatcgagtctagagggcccccgctgatcagcctcgactgtgccttct
agttgccagccatctgttgtttgcccctcccccgtgccttccttgaccctggaaggtgccac
tcccactgtcctttcctaataaaatgaggaaattgcatcgcattgtctgagtaggtgtcatt
ctattctggggggtggggtggggcaggacagcaagggggaggattgggaagagaatagcagg
catgctggggaagatctTCATGTCTGCGGCTCTAGAGCTGCATTAATGAATCGGCCAACGCG
CGGGGAGAGGCGGTTTGCGTATTGGGCGCTCTTCCGCTTCCTCGCTCACTGACTCGCTGCGC
TCGGTCGTTCGGCTGCGGCGAGCGGTATCAGCTCACTCAAAGGCGGTAATACGGTTATCCAC
AGAATCAGGGGATAACGCAGGAAAGAACATGTacgtaaactggcaaaggggtggctgggcca
aaagacagaggaattaagtaagaagtccaggaaaaatgaacttcacatcaaattttagagca
cggtagccatgaatcttgtgaatagctcccaaaaatgtcctgtggaagacaactagaaagca
ttctacaatcaggcacccacctccacctgcagcctcctgtgttgttctcatggggcacctct
gggctccagctcctccaaggcacctccacactctctcaagtacactcttcactcttccccaa
acatgattcccctactgctctgcctaactcccacttctctttcaagtagcagcttaaacgtc
acctcatatttggctggaaaatagaatatagacagaggggtaagttaaggctagaaaggcag
gctgggtcaacagaatggcaagctaaaacatgggattttctaaaacagcctaagagggtgcc
agataaaagtgtgcaaggagtggcacaactccagtttcatctttagctatagcaattaacac
cataaggagtctggattcaattttgccatttactagctagctaccaacttctgtgtcgcttt
gggcaaatcaattaaatccatacctccctttccatctgcagaatgggtttataacagtactt
aaacctcaaggtactaagaacagtaaagagttaatggtaCATGTGAGCAAAAGGCCAGCAAA
AGGCCAGGAACCGTAAAAAGGCCGCGTTGCTGGCGTTTTTCCATAGGCTCCGCCCCCCTGAC
GAGCATCACAAAAATCGACGCTCAAGTCAGAGGTGGCGAAACCCGACAGGACTATAAAGATA
CCAGGCGTTTCCCCCTGGAAGCTCCCTCGTGCGCTCTCCTGTTCCGACCCTGCCGCTTACCG
GATACCTGTCCGCCTTTCTCCCTTCGGGAAGCGTGGCGCTTTCTCATAGCTCACGCTGTAGG
TATCTCAGTTCGGTGTAGGTCGTTCGCTCCAAGCTGGGCTGTGTGCACGAACCCCCCGTTCA
GCCCGACCGCTGCGCCTTATCCGGTAACTATCGTCTTGAGTCCAACCCGGTAAGACACGACT
TATCGCCACTGGCAGCAGCCACTGGTAACAGGATTAGCAGAGCGAGGTATGTAGGCGGTGCT
ACAGAGTTCTTGAAGTGGTGGCCTAACTACGGCTACACTAGAAGAACAGTATTTGGTATCTG
CGCTCTGCTGAAGCCAGTTACCTTCGGAAAAAGAGTTGGTAGCTCTTGATCCGGCAAACAAA
CCACCGCTGGTAGCGGTGGTTTTTTTGTTTGCAAGCAGCAGATTACGCGCAGAAAAAAAGGA
TCTCAAGAAGATCCTTTGATCTTTTCTACGGGGTCTGACGCTCAGTGGAACGAAAACTCACG
TTAAGGGATTTTGGTCATGAGATTATCAAAAAGGATCTTCACCTAGATCCTTTTAAATTAAA
AATGAAGTTTTAAATCAATCTAAAGTATATATGAGTAAACTTGGTCTGACAGTTACCAATGC
TTAATCAGTGAGGCACCTATCTCAGCGATCTGTCTATTTCGTTCATCCATAGTTGCCTGACT
CCCCGTCGTGTAGATAACTACGATACGGGAGGGCTTACCATCTGGCCCCAGTGCTGCAATGA
TACCGCGAGACCCACGCTCACCGGCTCCAGATTTATCAGCAATAAACCAGCCAGCCGGAAGG
GCCGAGCGCAGAAGTGGTCCTGCAACTTTATCCGCCTCCATCCAGTCTATTAATTGTTGCCG
GGAAGCTAGAGTAAGTAGTTCGCCAGTTAATAGTTTGCGCAACGTTGTTGCCATTGCTACAG
GCATCGTGGTGTCACGCTCGTCGTTTGGTATGGCTTCATTCAGCTCCGGTTCCCAACGATCA
AGGCGAGTTACATGATCCCCCATGTTGTGCAAAAAAGCGGTTAGCTCCTTCGGTCCTCCGAT
CGTTGTCAGAAGTAAGTTGGCCGCAGTGTTATCACTCATGGTTATGGCAGCACTGCATAATT
CTCTTACTGTCATGCCATCCGTAAGATGCTTTTCTGTGACTGGTGAGTACTCAACCAAGTCA
TTCTGAGAATAGTGTATGCGGCGACCGAGTTGCTCTTGCCCGGCGTCAATACGGGATAATAC
CGCGCCACATAGCAGAACTTTAAAAGTGCTCATCATTGGAAAACGTTCTTCGGGGCGAAAAC
TCTCAAGGATCTTACCGCTGTTGAGATCCAGTTCGATGTAACCCACTCGTGCACCCAACTGA
TCTTCAGCATCTTTTACTTTCACCAGCGTTTCTGGGTGAGCAAAAACAGGAAGGCAAAATGC
CGCAAAAAAGGGAATAAGGGCGACACGGAAATGTTGAATACTCATACTCTTCCTTTTTCAAT
ATTATTGAAGCATTTATCAGGGTTATTGTCTCATGAGCGGATACATATTTGAATGTATTTAG
AAAAATAAACAAATAGGGGTTCCGCGCACATTTCCCCGAAAAGTGCCACCTGACGTCTAAGA
AACCATTATTATCATGACATTAACCTATAAAAATAGGCGTATCACGAGGCCCTTTCGTCTTC
AAGAATTcga
494 bp XIST-A-repeat/APP donor plasmid sequence
GTGATAGAGAACGTATGAAGAGTTTACTCCCTATCAGTGATAGAGAACGTATGCAGACTTTA
CTCCCTATCAGTGATAGAGAACGTATAAGGAGTTTACTCCCTATCAGTGATAGAGAACGTAT
GACCAGTTTACTCCCTATCAGTGATAGAGAACGTATCTACAGTTTACTCCCTATCAGTGATA
GAGAACGTATATCCAGTTTACTCCCTATCAGTGATAGAGAACGTATAAGCTTTAGGCGTGTA
CGGTGGGCGCCTATAAAAGCAGAGCTCGTTTAGTGAACCGTCAGATCGCCTGGAGCAATTCC
ACAACACTTTTGTCTTATACCAACTTTCCGTACCACTTCCTACCCTCGTAAAGTCGACACCG
GGGCCCAGATCTGGTACCGAGCTCGGATCCACTAGTCCAGTGTGGTGGAATTCTGCAGAGAA
TTTTTCTTTGGAATCATTTTTGGTTGACATCTCTGTTTTTTGTGGATCAGTTTTTTACTCTT
CCACTCTCTTTTCTATATTTTGCCCATCGGGGCTGCGGATACCTGGTTTTATTATTTTTTCT
TTGCCCAACGGGGCCGTGGATACCTGCCTTTTAATTCTTTTTTATTCGCCCATCGGGGCCGC
GGATACCTGCTTTTTATTTTTTTTTCCTTAGCCCATCGGGGTATCGGATACCTGCTGATTCC
CTTCCCCTCTGAACCCCCAACACTCTGGCCCATCGGGGTGACGGATATCTGCTTTTTAAAAA
TTTTCTTTTTTTGGCCCATCGGGGCTTCGGATACCTGCTTTTTTTTTTTTTATTTTTCCTTG
CCCATCGGGGCCTCGGATACCTGCTTTAATTTTTGTTTTTCTGGCCCATCGGGGCCGCGGAT
ACCTGCTTTGATTTTTTTTTTTCATCGCCCATCGGTGCTTTTTATGGATGAAAAAATGTTTC
GATCGGCCGGATATCACGCGTCATATGGCTAGCCTGCAGGGATCCAATGTAACTGTATTCAG
CGATGACGAAATTCTTAGCTATTGTAATACTCTAGAGGATCTTTGTGAAGGAACCTTACTTC
TGTGGTGTGACATAATTGGACAAACTACCTACAGAGATTTAAAGCTCTAAGGTAAATATAAA
ATTTTTAAGTGTATAATGTGTTAAACTACTGATTCTAATTGTTTGTGTATTTTAGATTCCAA
CCTATGGAACTGATGAATGGGAGCAGTGGTGGAATGCCTTTAATGAGGAAAACCTGTTTTGC
TCAGAAGAAATGCCATCTAGTGATGATGAGGCTACTGCTGACTCTCAACATTCTACTCCTCC
AAAAAAGAAGAGAAAGGTAGAAGACCCCAAGGACTTTCCTTCAGAATTGCTAAGTTTTTTGA
GTCATGCTGTGTTTAGTAATAGAACTCTTGCTTGCTTTGCTATTTACACCACAAAGGAAAAA
GCTGCACTGCTATACAAGAAAATTATGGAAAAATATTCTGTAACCTTTATAAGTAGGCATAA
CAGTTATAATCATAACATACTGTTTTTTCTTACTCCACACAGGCATAGAGTGTCTGCTATTA
ATAACTATGCTCAAAAATTGTGTACCTTTAGCTTTTTAATTTGTAAAGGGGTTAATAAGGAA
TATTTGATGTATAGTGCCTTGACTAGAGATCATAATCAGCCATACCACATTTGTAGAGGTTT
TACTTGCTTTAAAAAACCTCCCACACCTCCCCCTGAACCTGAAACATAAAATGAATGCAATT
GTTGTTGTTAACTTGTTTATTGCAGCTTATAATGGTTACAAATAAAGCAATAGCATCACAAA
TTTCACAAATAAAGCATTTTTTTCACTGCATTCTAGTTGTGGTTTGTCCAAACTCATCAATG
TATCTTAGAGGGACAGCCCCCCCCCAAAGCCCCCAGGGATGTAATTACGTCCCTCCCCCGCT
AGGGGGCAGCAGCGAGCCGCCCGGGGCTCCGCTCCGGTCCGGCGCTCCCCCCGCATCCCCGA
GCCGGCAGCGTGCGGGGACAGCCCGGGCACGGGGAAGGTGGCACGGGATCGCTTTCCTCTGA
ACGCTTCTCGCTGCTCTTTGAGCCTGCAGACACCTGGGGGGATACGGGGAAAAGCTAGCAAG
GATCTGCGATCGCTCCGGTGCCCGTCAGTGGGCAGAGCGCACATCGCCCACAGTCCCCGAGA
AGTTGGGGGGAGGGGTCGGCAATTGAACGGGTGCCTAGAGAAGGTGGCGCGGGGTAAACTGG
GAAAGTGATGTCGTGTACTGGCTCCGCCTTTTTCCCGAGGGTGGGGGAGAACCGTATATAAG
TGCAGTAGTCGCCGTGAACGTTCTTTTTCGCAACGGGTTTGCCGCCAGAACACAGCTGAAGC
TTCGAGGGGCTCGCATCTCTCCTTCACGCGCCCGCCGCCCTACCTGAGGCCGCCATCCACGC
CGGTTGAGTCGCGTTCTGCCGCCTCCCGCCTGTGGTGCCTCCTGAACTGCGTCCGCCGTCTA
GGTAAGTTTAAAGCTCAGGTCGAGTCCGGGCCTTTGTCCGGCGCTCCCTTGGAGCCTACCTA
GACTCAGCCGGCTCTCCACGCTTTGCCTGACCCTGCTTGCTCAACTCTACGTCTTTGTTTCG
TTTTCTGTTCTGCGCCGTTACAGATCCAAGCTGTGACCGGCGCCTACGAATTCGCCACCATG
GCCAGCTCCGAGGATGTCATCAAAGAGTTTATGAGATTTAAGGTCAAGATGGAGGGAAGCGT
CAACGGACACGAGTTCGAGATTGAGGGAGAAGGAGAAGGCCGGCCTTACGAGGGCACACAAA
CCGCTAAGCTCAAGGTCACAAAAGGAGGACCCCTCCCCTTCTCCTGGGATATTCTGAGCCCT
CAGTTCCAGTACGGAAGCAAAGCCTATGTTAAACACCCTGCCGACATCCCTGACTATCTGAA
GCTCTCCTTCCCTGAAGGCTTCAAGTGGGAGAGATTCATGAACTTCGAGGACGGAGGCGTGG
TGACAGTCACACAAGATAGCACCCTCCAGGACGGAGAGTTTATTTATAAGGTGAAACTCAGA
GGAACCAACTTCCCCTCCGATGGCCCTGTCATGCAAAAAAAAACAATGGGATGGGAAGCCTC
CACCGAGAGAATGTATCCTGAGGATGGCGCTCTGAAAGGCGAAATTAAAATGAGACTGAAAC
TCAAAGACGGAGGACACTACGATGCCGAGGTCAAAACAACCTACAAGGCCAAGAAACAAGTG
CAGCTGCCTGGCGCCTACATGACTGATATTAAACTCGACATTATCAGCCATAATGGGGACTA
CACCATCGTGGAACAATATGAGAGAGCTGAGGGCAGACATAGCACAGGCGCTGGAAGTACTT
GAGGATCCTGATCGAGTCTAGAGGGCCCCCGCTGATCAGCCTCGACTGTGCCTTCTAGTTGC
CAGCCATCTGTTGTTTGCCCCTCCCCCGTGCCTTCCTTGACCCTGGAAGGTGCCACTCCCAC
TGTCCTTTCCTAATAAAATGAGGAAATTGCATCGCATTGTCTGAGTAGGTGTCATTCTATTC
TGGGGGGTGGGGTGGGGCAGGACAGCAAGGGGGAGGATTGGGAAGAGAATAGCAGGCATGCT
GGGGAAGATCTTCATGTCTGCGGCTCTAGAGCTGCATTAATGAATCGGCCAACGCGCGGGGA
GAGGCGGTTTGCGTATTGGGCGCTCTTCCGCTTCCTCGCTCACTGACTCGCTGCGCTCGGTC
GTTCGGCTGGATAACGCAGGAAAGAACATGTGGACTTCAGGCTCCTCCTAGAAAACAAGAAG
GCTTAAAAAGCAAGACAACCTATTAGCTATGTTGCTCAATAAATTTCAATGTTAATTTCCTA
ATTCTTTGATAACACATTCCTGAACTCCTTTGTAATTCAGGCAACATTTTAAAAATCAGATG
ATCTAACTGGACTCCAAGAATTACATAAACAGTGTGAGCAGTGAGACTTCGAATGTGCAATC
AACTATTTTTTTAATCCTGCCGCTAAAAGCCTTCAGTAGCACAGAACTGGTTGTCAGAGATT
TCAGTGTAACCTAACACCTAACGTGAATGACTGCTGTACAAAAATACAGCTGAAAATACTAT
GAGGAAGAGATAGAAACGGATTGTTGATGGCTAAGCAAAACAGCAAATGCTGATGGTTATCA
AACACATGCCGTTCCCACTCTACACAGATAAGATTTCAAAGCTGTTTGAGTCCTAGGTTAAG
TTTTGGGCAAGTTCTGGCTTGATGCCTTATATTCAGATAAACATTTTCCAGGCAGTGAACAT
ATTCAAAGTTGGGGACAGTGGGGTAACCCGAAAACATTCCTGACCTTGATGGACAGATAATC
CAATGGTGGAGACACAAATAAGGCCAAATTGGCTAACCAAAGGTGCTGGAGTGTTAATGCAG
AGGTCTGGCAAGGGTACACCGAAGAGCTCTCAGGAAAGAGTGACTGGTTTGACTTGGAGAAG
GAGAAGGAAGGCAGACCAAAGATCAGAGCAGAAAACATGCCCAGGCTGAGTATGGGTCGGAC
AGGAAGCAaCATGTGAGCAAAAGGCCAGTGCTGGCGTTTTTCCATAGGCTCCGCCCCCCTGA
CGAGCATCACAAAAATCGACGCTCAAGTCAGAGGTGGCGAAACCCGACAGGACTATAAAGAT
ACCAGGCGTTTCCCCCTGGAAGCTCCCTCGTGCGCTCTCCTGTTCCGACCCTGCCGCTTACC
GGATACCTGTCCGCCTTTCTCCCTTCGGGAAGCGTGGCGCTTTCTCATAGCTCACGCTGTAG
GTATCTCAGTTCGGTGTAGGTCGTTCGCTCCAAGCTGGGCTGTGTGCACGAACCCCCCGTTC
AGCCCGACCGCTGCGCCTTATCCGGTAACTATCGTCTTGAGTCCAACCCGGTAAGACACGAC
TTATCGCCACTGGCAGCAGCCACTGGTAACAGGATTAGCAGAGCGAGGTATGTAGGCGGTGC
TACAGAGTTCTTGAAGTGGTGGCCTAACTACGGCTACACTAGAAGAACAGTATTTGGTATCT
GCGCTCTGCTGAAGCCAGTTACCTTCGGAAAAAGAGTTGGTAGCTCTTGATCCGGCAAACAA
ACCACCGCTGGTAGCGGTGGTTTTTTTGTTTGCAAGCAGCAGATTACGCGCAGAAAAAAAGG
ATCTCAAGAAGATCCTTTGATCTTTTCTACGGGGTCTGACGCTCAGTGGAACGAAAACTCAC
GTTAAGGGATTTTGGTCATGAGATTATCAAAAAGGATCTTCACCTAGATCCTTTTAAATTAA
AAATGAAGTTTTAAATCAATCTAAAGTATATATGAGTAAACTTGGTCTGACAGTTACCAATG
CTTAATCAGTGAGGCACCTATCTCAGCGATCTGTCTATTTCGTTCATCCATAGTTGCCTGAC
TCCCCGTCGTGTAGATAACTACGATACGGGAGGGCTTACCATCTGGCCCCAGTGCTGCAATG
ATACCGCGAGACCCACGCTCACCGGCTCCAGATTTATCAGCAATAAACCAGCCAGCCGGAAG
GGCCGAGCGCAGAAGTGGTCCTGCAACTTTATCCGCCTCCATCCAGTCTATTAATTGTTGCC
GGGAAGCTAGAGTAAGTAGTTCGCCAGTTAATAGTTTGCGCAACGTTGTTGCCATTGCTACA
GGCATCGTGGTGTCACGCTCGTCGTTTGGTATGGCTTCATTCAGCTCCGGTTCCCAACGATC
AAGGCGAGTTACATGATCCCCCATGTTGTGCAAAAAAGCGGTTAGCTCCTTCGGTCCTCCGA
TCGTTGTCAGAAGTAAGTTGGCCGCAGTGTTATCACTCATGGTTATGGCAGCACTGCATAAT
TCTCTTACTGTCATGCCATCCGTAAGATGCTTTTCTGTGACTGGTGAGTACTCAACCAAGTC
ATTCTGAGAATAGTGTATGCGGCGACCGAGTTGCTCTTGCCCGGCGTCAATACGGGATAATA
CCGCGCCACATAGCAGAACTTTAAAAGTGCTCATCATTGGAAAACGTTCTTCGGGGCGAAAA
CTCTCAAGGATCTTACCGCTGTTGAGATCCAGTTCGATGTAACCCACTCGTGCACCCAACTG
ATCTTCAGCATCTTTTACTTTCACCAGCGTTTCTGGGTGAGCAAAAACAGGAAGGCAAAATG
CCGCAAAAAAGGGAATAAGGGCGACACGGAAATGTTGAATACTCATACTCTTCCTTTTTCAA
TATTATTGAAGCATTTATCAGGGTTATTGTCTCATGAGCGGATACATATTTGAATGTATTTA
GAAAAATAAACAAATAGGGGTTCCGCGCACATTTCCCCGAAAAGTGCCACCTGACGTCTAAG
AAACCATTATTATCATGACATTAACCTATAAAAATAGGCGTATCACGAGGCCCTTTCGTCTT
CAAGAATTcgaaTTGCTTGATAAGCCTGGGTAGAttgtccttcttaaataacttacaccaaa
gcactatcaaagaagctctccctttgtagagccctcagattcacagaaggaatttttcgaat
tgccatttctataggaataaaggctatcaggaaccacaatactaggagaaaataattctgaa
ggggaggtagcagtgaagactcttatttctattttcttgactatatttataaaaaattgttg
ccgaaagaacagaagaccaagagccaaaaatcacactgcagtcttactcttggatgtaatgt
tactatcagtttcagtgacacaacatcagaggcaatctgatattgacaatggtaaaaatatg
cagacgccaaAATTAGGAGGGTGCAGTTAGTTGACAGTAGAGCCTATAAGTATTCATCTAAG
TTTTCTCTGTTTGAAGAGAAGAAAAGAGAACATCAAACATTTCTAGTATATAGAGCAAGGAT
TCCTAATCTTTTTTTTTTTTTAATGTGCAAACAAGACAAAAGTTACTTCACTGTCTAGGGGT
TATTCCCTACTCTTTAGCACTGGCCTGACTCTCTCACTCCCACATATAAGCATTCAATAAAT
ACTTATGCCAACAATTAAGAAACACCTGGCATGAAAAATAATAACAGTAATAAACTAGAAGG
TAGAAAATGTAATTATTGACAAAGGTGTGGAACAATACAAAATAAATTCCACTTTAGATACC
TGTGAAATTTTTTATTGTGGCACCAGGAGAGTAATTTCCTAACAGAGAAAAGAAAACATAGC
ATCCACACCTCTGTCTTTACCAATTCCTAAATACCAGCTACAGTTCAACTTGTCCACTTCGA
ATTCCTCGAGTTTACTCCCTATCA
APP Arm 1 783 bp
GGACTTCAGGCTCCTCCTAGAAAACAAGAAGGCTTAAAAAGCAAGACAACCTATTAGCTATG
TTGCTCAATAAATTTCAATGTTAATTTCCTAATTCTTTGATAACACATTCCTGAACTCCTTT
GTAATTCAGGCAACATTTTAAAAATCAGATGATCTAACTGGACTCCAAGAATTACATAAACA
GTGTGAGCAGTGAGACTTCGAATGTGCAATCAACTATTTTTTTAATCCTGCCGCTAAAAGCC
TTCAGTAGCACAGAACTGGTTGTCAGAGATTTCAGTGTAACCTAACACCTAACGTGAATGAC
TGCTGTACAAAAATACAGCTGAAAATACTATGAGGAAGAGATAGAAACGGATTGTTGATGGC
TAAGCAAAACAGCAAATGCTGATGGTTATCAAACACATGCCGTTCCCACTCTACACAGATAA
GATTTCAAAGCTGTTTGAGTCCTAGGTTAAGTTTTGGGCAAGTTCTGGCTTGATGCCTTATA
TTCAGATAAACATTTTCCAGGCAGTGAACATATTCAAAGTTGGGGACAGTGGGGTAACCCGA
AAACATTCCTGACCTTGATGGACAGATAATCCAATGGTGGAGACACAAATAAGGCCAAATTG
GCTAACCAAAGGTGCTGGAGTGTTAATGCAGAGGTCTGGCAAGGGTACACCGAAGAGCTCTC
AGGAAAGAGTGACTGGTTTGACTTGGAGAAGGAGAAGGAAGGCAGACCAAAGATCAGAGCAG
AAAACATGCCCAGGCTGAGTATGGGTCGGACAGGAAGCA
APP Arm 2 851 bp
TTGCTTGATAAGCCTGGGTAGAttgtccttcttaaataacttacaccaaagcactatcaaag
aagctctccctttgtagagccctcagattcacagaaggaatttttcgaattgccatttctat
aggaataaaggctatcaggaaccacaatactaggagaaaataattctgaaggggaggtagca
gtgaagactcttatttctattttcttgactatatttataaaaaattgttgccgaaagaacag
aagaccaagagccaaaaatcacactgcagtcttactcttggatgtaatgttactatcagttt
cagtgacacaacatcagaggcaatctgatattgacaatggtaaaaatatgcagacgccaaAA
TTAGGAGGGTGCAGTTAGTTGACAGTAGAGCCTATAAGTATTCATCTAAGTTTTCTCTGTTT
GAAGAGAAGAAAAGAGAACATCAAACATTTCTAGTATATAGAGCAAGGATTCCTAATCTTTT
TTTTTTTTTAATGTGCAAACAAGACAAAAGTTACTTCACTGTCTAGGGGTTATTCCCTACTC
TTTAGCACTGGCCTGACTCTCTCACTCCCACATATAAGCATTCAATAAATACTTATGCCAAC
AATTAAGAAACACCTGGCATGAAAAATAATAACAGTAATAAACTAGAAGGTAGAAAATGTAA
TTATTGACAAAGGTGTGGAACAATACAAAATAAATTCCACTTTAGATACCTGTGAAATTTTT
TATTGTGGCACCAGGAGAGTAATTTCCTAACAGAGAAAAGAAAACATAGCATCCACACCTCT
GTCTTTACCAATTCCTAAATACCAGCTACAGTTCAACTTGTCCAC
sgRNA rs2830068 SNP-T
TAGGAGGAGCCTGAaGTCCG
CRISPR/Cas9/rs2830068 SNP-T APP plasmid sequence
gagggcctatttcccatgattccttcatatttgcatatacgatacaaggctgttagagagat
aattggaattaatttgactgtaaacacaaagatattagtacaaaatacgtgacgtagaaagt
aataatttcttgggtagtttgcagttttaaaattatgttttaaaatggactatcatatgctt
accgtaacttgaaagtatttcgatttcttggctttatatatcttgtggaaaggacgaaacac
CTAGGAGGAGCCTGAAGTCCGgttttagagctagaaatagcaagttaaaataaggctagtcc
gttatcaacttgaaaaagtggcaccgagtcggtgcttttttgttttagagctagaaatagca
agttaaaataaggctagtccgtttttagcgcgtgcgccaattctgcagacaaatggctctag
aggtacccgttacataacttacggtaaatggcccgcctggctgaccgcccaacgacccccgc
ccattgacgtcaatagtaacgccaatagggactttccattgacgtcaatgggtggagtattt
acggtaaactgcccacttggcagtacatcaagtgtatcatatgccaagtacgccccctattg
acgtcaatgacggtaaatggcccgcctggcattgtgcccagtacatgaccttatgggacttt
cctacttggcagtacatctacgtattagtcatcgctattaccatggtcgaggtgagccccac
gttctgcttcactctccccatctcccccccctccccacccccaattttgtatttatttattt
tttaattattttgtgcagcgatgggggcggggggggggggggggcgcgcgccgggggggggg
gggggggggggggggggggggggggcgaggcggagaggtgcggcggcagccaatcagagcgg
cgcgctccgaaagtttccttttatggcgaggcggcggcggcggcggccctataaaaagcgaa
gcgcgcggcgggcgggagtcgctgcgcgctgccttcgccccgtgccccgctccgccgccgcc
tcgcgccgcccgccccggctctgactgaccgcgttactcccacaggtgagcgggcgggacgg
cccttctcctccgggctgtaattagctgagcaagaggtaagggtttaagggatggttggttg
gtggggtattaatgtttaattacctggagcacctgcctgaaatcactttttttcaggttgga
ccggtgccaccatggactataaggaccacgacggagactacaaggatcatgatattgattac
aaagacgatgacgataagatggccccaaagaagaagcggaaggtcggtatccacggagtccc
agcagccgacaagaagtacagcatcggcctggacatcggcaccaactctgtgggctgggccg
tgatcaccgacgagtacaaggtgcccagcaagaaattcaaggtgctgggcaacaccgaccgg
cacagcatcaagaagaacctgatcggagccctgctgttcgacagcggcgaaacagccgaggc
cacccggctgaagagaaccgccagaagaagatacaccagacggaagaaccggatctgctatc
tgcaagagatcttcagcaacgagatggccaaggtggacgacagcttcttccacagactggaa
gagtccttcctggtggaagaggataagaagcacgagcggcaccccatcttcggcaacatcgt
ggacgaggtggcctaccacgagaagtaccccaccatctaccacctgagaaagaaactggtgg
acagcaccgacaaggccgacctgcggctgatctatctggccctggcccacatgatcaagttc
cggggccacttcctgatcgagggcgacctgaaccccgacaacagcgacgtggacaagctgtt
catccagctggtgcagacctacaaccagctgttcgaggaaaaccccatcaacgccagcggcg
tggacgccaaggccatcctgtctgccagactgagcaagagcagacggctggaaaatctgatc
gcccagctgcccggcgagaagaagaatggcctgttcggaaacctgattgccctgagcctggg
cctgacccccaacttcaagagcaacttcgacctggccgaggatgccaaactgcagctgagca
aggacacctacgacgacgacctggacaacctgctggcccagatcggcgaccagtacgccgac
ctgtttctggccgccaagaacctgtccgacgccatcctgctgagcgacatcctgagagtgaa
caccgagatcaccaaggcccccctgagcgcctctatgatcaagagatacgacgagcaccacc
aggacctgaccctgctgaaagctctcgtgcggcagcagctgcctgagaagtacaaagagatt
ttcttcgaccagagcaagaacggctacgccggctacattgacggcggagccagccaggaaga
gttctacaagttcatcaagcccatcctggaaaagatggacggcaccgaggaactgctcgtga
agctgaacagagaggacctgctgcggaagcagcggaccttcgacaacggcagcatcccccac
cagatccacctgggagagctgcacgccattctgcggcggcaggaagatttttacccattcct
gaaggacaaccgggaaaagatcgagaagatcctgaccttccgcatcccctactacgtgggcc
ctctggccaggggaaacagcagattcgcctggatgaccagaaagagcgaggaaaccatcacc
ccctggaacttcgaggaagtggtggacaagggcgcttccgcccagagcttcatcgagcggat
gaccaacttcgataagaacctgcccaacgagaaggtgctgcccaagcacagcctgctgtacg
agtacttcaccgtgtataacgagctgaccaaagtgaaatacgtgaccgagggaatgagaaag
cccgccttcctgagcggcgagcagaaaaaggccatcgtggacctgctgttcaagaccaaccg
gaaagtgaccgtgaagcagctgaaagaggactacttcaagaaaatcgagtgcttcgactccg
tggaaatctccggcgtggaagatcggttcaacgcctccctgggcacataccacgatctgctg
aaaattatcaaggacaaggacttcctggacaatgaggaaaacgaggacattctggaagatat
cgtgctgaccctgacactgtttgaggacagagagatgatcgaggaacggctgaaaacctatg
cccacctgttcgacgacaaagtgatgaagcagctgaagcggcggagatacaccggctggggc
aggctgagccggaagctgatcaacggcatccgggacaagcagtccggcaagacaatcctgga
tttcctgaagtccgacggcttcgccaacagaaacttcatgcagctgatccacgacgacagcc
tgacctttaaagaggacatccagaaagcccaggtgtccggccagggcgatagcctgcacgag
cacattgccaatctggccggcagccccgccattaagaagggcatcctgcagacagtgaaggt
ggtggacgagctcgtgaaagtgatgggccggcacaagcccgagaacatcgtgatcgaaatgg
ccagagagaaccagaccacccagaagggacagaagaacagccgcgagagaatgaagcggatc
gaagagggcatcaaagagctgggcagccagatcctgaaagaacaccccgtggaaaacaccca
gctgcagaacgagaagctgtacctgtactacctgcagaatgggcgggatatgtacgtggacc
aggaactggacatcaaccggctgtccgactacgatgtggaccatatcgtgcctcagagcttt
ctgaaggacgactccatcgacaacaaggtgctgaccagaagcgacaagaaccggggcaagag
cgacaacgtgccctccgaagaggtcgtgaagaagatgaagaactactggcggcagctgctga
acgccaagctgattacccagagaaagttcgacaatctgaccaaggccgagagaggcggcctg
agcgaactggataaggccggcttcatcaagagacagctggtggaaacccggcagatcacaaa
gcacgtggcacagatcctggactcccggatgaacactaagtacgacgagaatgacaagctga
tccgggaagtgaaagtgatcaccctgaagtccaagctggtgtccgatttccggaaggatttc
cagttttacaaagtgcgcgagatcaacaactaccaccacgcccacgacgcctacctgaacgc
cgtcgtgggaaccgccctgatcaaaaagtaccctaagctggaaagcgagttcgtgtacggcg
actacaaggtgtacgacgtgcggaagatgatcgccaagagcgagcaggaaatcggcaaggct
accgccaagtacttcttctacagcaacatcatgaactttttcaagaccgagattaccctggc
caacggcgagatccggaagcggcctctgatcgagacaaacggcgaaaccggggagatcgtgt
gggataagggccgggattttgccaccgtgcggaaagtgctgagcatgccccaagtgaatatc
gtgaaaaagaccgaggtgcagacaggcggcttcagcaaagagtctatcctgcccaagaggaa
cagcgataagctgatcgccagaaagaaggactgggaccctaagaagtacggcggcttcgaca
gccccaccgtggcctattctgtgctggtggtggccaaagtggaaaagggcaagtccaagaaa
ctgaagagtgtgaaagagctgctggggatcaccatcatggaaagaagcagcttcgagaagaa
tcccatcgactttctggaagccaagggctacaaagaagtgaaaaaggacctgatcatcaagc
tgcctaagtactccctgttcgagctggaaaacggccggaagagaatgctggcctctgccggc
gaactgcagaagggaaacgaactggccctgccctccaaatatgtgaacttcctgtacctggc
cagccactatgagaagctgaagggctcccccgaggataatgagcagaaacagctgtttgtgg
aacagcacaagcactacctggacgagatcatcgagcagatcagcgagttctccaagagagtg
atcctggccgacgctaatctggacaaagtgctgtccgcctacaacaagcaccgggataagcc
catcagagagcaggccgagaatatcatccacctgtttaccctgaccaatctgggagcccctg
ccgccttcaagtactttgacaccaccatcgaccggaagaggtacaccagcaccaaagaggtg
ctggacgccaccctgatccaccagagcatcaccggcctgtacgagacacggatcgacctgtc
tcagctgggaggcgacaaaaggccggcggccacgaaaaaggccggccaggcaaaaaagaaaa
aggaattcggcagtggagagggcagaggaagtctgctaacatgcggtgacgtcgaggagaat
cctggcccagtgagcaagggcgaggagctgttcaccggggtggtgcccatcctggtcgagct
ggacggcgacgtaaacggccacaagttcagcgtgtccggcgagggcgagggcgatgccacct
acggcaagctgaccctgaagttcatctgcaccaccggcaagctgcccgtgccctggcccacc
ctcgtgaccaccctgacctacggcgtgcagtgcttcagccgctaccccgaccacatgaagca
gcacgacttcttcaagtccgccatgcccgaaggctacgtccaggagcgcaccatcttcttca
aggacgacggcaactacaagacccgcgccgaggtgaagttcgagggcgacaccctggtgaac
cgcatcgagctgaagggcatcgacttcaaggaggacggcaacatcctggggcacaagctgga
gtacaactacaacagccacaacgtctatatcatggccgacaagcagaagaacggcatcaagg
tgaacttcaagatccgccacaacatcgaggacggcagcgtgcagctcgccgaccactaccag
cagaacacccccatcggcgacggccccgtgctgctgcccgacaaccactacctgagcaccca
gtccgccctgagcaaagaccccaacgagaagcgcgatcacatggtcctgctggagttcgtga
ccgccgccgggatcactctcggcatggacgagctgtacaaggaattctaactagagctcgct
gatcagcctcgactgtgccttctagttgccagccatctgttgtttgcccctcccccgtgcct
tccttgaccctggaaggtgccactcccactgtcctttcctaataaaatgaggaaattgcatc
gcattgtctgagtaggtgtcattctattctggggggtggggtggggcaggacagcaaggggg
aggattgggaagagaatagcaggcatgctggggagcggccgcaggaacccctagtgatggag
ttggccactccctctctgcgcgctcgctcgctcactgaggccgggcgaccaaaggtcgcccg
acgcccgggctttgcccgggcggcctcagtgagcgagcgagcgcgcagctgcctgcaggggc
gcctgatgcggtattttctccttacgcatctgtgcggtatttcacaccgcatacgtcaaagc
aaccatagtacgcgccctgtagcggcgcattaagcgcggcgggtgtggtggttacgcgcagc
gtgaccgctacacttgccagcgccttagcgcccgctcctttcgctttcttcccttcctttct
cgccacgttcgccggctttccccgtcaagctctaaatcgggggctccctttagggttccgat
ttagtgctttacggcacctcgaccccaaaaaacttgatttgggtgatggttcacgtagtggg
ccatcgccctgatagacggtttttcgccctttgacgttggagtccacgttctttaatagtgg
actcttgttccaaactggaacaacactcaactctatctcgggctattcttttgatttataag
ggattttgccgatttcggtctattggttaaaaaatgagctgatttaacaaaaatttaacgcg
aattttaacaaaatattaacgtttacaattttatggtgcactctcagtacaatctgctctga
tgccgcatagttaagccagccccgacacccgccaacacccgctgacgcgccctgacgggctt
gtctgctcccggcatccgcttacagacaagctgtgaccgtctccgggagctgcatgtgtcag
aggttttcaccgtcatcaccgaaacgcgcgagacgaaagggcctcgtgatacgcctattttt
ataggttaatgtcatgataataatggtttcttagacgtcaggtggcacttttcggggaaatg
tgcgcggaacccctatttgtttatttttctaaatacattcaaatatgtatccgctcatgaga
caataaccctgataaatgcttcaataatattgaaaaaggaagagtatgagtattcaacattt
ccgtgtcgcccttattcccttttttgcggcattttgccttcctgtttttgctcacccagaaa
cgctggtgaaagtaaaagatgctgaagatcagttgggtgcacgagtgggttacatcgaactg
gatctcaacagcggtaagatccttgagagttttcgccccgaagaacgttttccaatgatgag
cacttttaaagttctgctatgtggcgcggtattatcccgtattgacgccgggcaagagcaac
tcggtcgccgcatacactattctcagaatgacttggttgagtactcaccagtcacagaaaag
catcttacggatggcatgacagtaagagaattatgcagtgctgccataaccatgagtgataa
cactgcggccaacttacttctgacaacgatcggaggaccgaaggagctaaccgcttttttgc
acaacatgggggatcatgtaactcgccttgatcgttgggaaccggagctgaatgaagccata
ccaaacgacgagcgtgacaccacgatgcctgtagcaatggcaacaacgttgcgcaaactatt
aactggcgaactacttactctagcttcccggcaacaattaatagactggatggaggcggata
aagttgcaggaccacttctgcgctcggcccttccggctggctggtttattgctgataaatct
ggagccggtgagcgtggaagccgcggtatcattgcagcactggggccagatggtaagccctc
ccgtatcgtagttatctacacgacggggagtcaggcaactatggatgaacgaaatagacaga
tcgctgagataggtgcctcactgattaagcattggtaactgtcagaccaagtttactcatat
atactttagattgatttaaaacttcatttttaatttaaaaggatctaggtgaagatcctttt
tgataatctcatgaccaaaatcccttaacgtgagttttcgttccactgagcgtcagaccccg
tagaaaagatcaaaggatcttcttgagatcctttttttctgcgcgtaatctgctgcttgcaa
acaaaaaaaccaccgctaccagcggtggtttgtttgccggatcaagagctaccaactctttt
tccgaaggtaactggcttcagcagagcgcagataccaaatactgttcttctagtgtagccgt
agttaggccaccacttcaagaactctgtagcaccgcctacatacctcgctctgctaatcctg
ttaccagtggctgctgccagtggcgataagtcgtgtcttaccgggttggactcaagacgata
gttaccggataaggcgcagcggtcgggctgaacggggggttcgtgcacacagcccagcttgg
agcgaacgacctacaccgaactgagatacctacagcgtgagctatgagaaagcgccacgctt
cccgaagggagaaaggcggacaggtatccggtaagcggcagggtcggaacaggagagcgcac
gagggagcttccagggggaaacgcctggtatctttatagtcctgtcgggtttcgccacctct
gacttgagcgtcgatttttgtgatgctcgtcaggggggcggagcctatggaaaaacgccagc
aacgcggcctttttacggttcctggccttttgctggccttttgctcacatgt
sgRNA rs2830068 SNP-C
TAGGAGGAGCCTGAgGTCCG
CRISPR/Cas9/rs2830068 SNP-C APP plasmid sequence
gagggcctatttcccatgattccttcatatttgcatatacgatacaaggctgttagagagat
aattggaattaatttgactgtaaacacaaagatattagtacaaaatacgtgacgtagaaagt
aataatttcttgggtagtttgcagttttaaaattatgttttaaaatggactatcatatgctt
accgtaacttgaaagtatttcgatttcttggctttatatatcttgtggaaaggacgaaacac
cTAGGAGGAGCCTGAgGTCCGgttttagagctagaaatagcaagttaaaataaggctagtcc
gttatcaacttgaaaaagtggcaccgagtcggtgcttttttgttttagagctagaaatagca
agttaaaataaggctagtccgtttttagcgcgtgcgccaattctgcagacaaatggctctag
aggtacccgttacataacttacggtaaatggcccgcctggctgaccgcccaacgacccccgc
ccattgacgtcaatagtaacgccaatagggactttccattgacgtcaatgggtggagtattt
acggtaaactgcccacttggcagtacatcaagtgtatcatatgccaagtacgccccctattg
acgtcaatgacggtaaatggcccgcctggcattgtgcccagtacatgaccttatgggacttt
cctacttggcagtacatctacgtattagtcatcgctattaccatggtcgaggtgagccccac
gttctgcttcactctccccatctcccccccctccccacccccaattttgtatttatttattt
tttaattattttgtgcagcgatgggggcggggggggggggggggcgcgcgccgggggggggg
gggggggggggggggggggggggggcgaggcggagaggtgcggcggcagccaatcagagcgg
cgcgctccgaaagtttccttttatggcgaggcggcggcggcggcggccctataaaaagcgaa
gcgcgcggcgggcgggagtcgctgcgcgctgccttcgccccgtgccccgctccgccgccgcc
tcgcgccgcccgccccggctctgactgaccgcgttactcccacaggtgagcgggcgggacgg
cccttctcctccgggctgtaattagctgagcaagaggtaagggtttaagggatggttggttg
gtggggtattaatgtttaattacctggagcacctgcctgaaatcactttttttcaggttgga
ccggtgccaccatggactataaggaccacgacggagactacaaggatcatgatattgattac
aaagacgatgacgataagatggccccaaagaagaagcggaaggtcggtatccacggagtccc
agcagccgacaagaagtacagcatcggcctggacatcggcaccaactctgtgggctgggccg
tgatcaccgacgagtacaaggtgcccagcaagaaattcaaggtgctgggcaacaccgaccgg
cacagcatcaagaagaacctgatcggagccctgctgttcgacagcggcgaaacagccgaggc
cacccggctgaagagaaccgccagaagaagatacaccagacggaagaaccggatctgctatc
tgcaagagatcttcagcaacgagatggccaaggtggacgacagcttcttccacagactggaa
gagtccttcctggtggaagaggataagaagcacgagcggcaccccatcttcggcaacatcgt
ggacgaggtggcctaccacgagaagtaccccaccatctaccacctgagaaagaaactggtgg
acagcaccgacaaggccgacctgcggctgatctatctggccctggcccacatgatcaagttc
cggggccacttcctgatcgagggcgacctgaaccccgacaacagcgacgtggacaagctgtt
catccagctggtgcagacctacaaccagctgttcgaggaaaaccccatcaacgccagcggcg
tggacgccaaggccatcctgtctgccagactgagcaagagcagacggctggaaaatctgatc
gcccagctgcccggcgagaagaagaatggcctgttcggaaacctgattgccctgagcctggg
cctgacccccaacttcaagagcaacttcgacctggccgaggatgccaaactgcagctgagca
aggacacctacgacgacgacctggacaacctgctggcccagatcggcgaccagtacgccgac
ctgtttctggccgccaagaacctgtccgacgccatcctgctgagcgacatcctgagagtgaa
caccgagatcaccaaggcccccctgagcgcctctatgatcaagagatacgacgagcaccacc
aggacctgaccctgctgaaagctctcgtgcggcagcagctgcctgagaagtacaaagagatt
ttcttcgaccagagcaagaacggctacgccggctacattgacggcggagccagccaggaaga
gttctacaagttcatcaagcccatcctggaaaagatggacggcaccgaggaactgctcgtga
agctgaacagagaggacctgctgcggaagcagcggaccttcgacaacggcagcatcccccac
cagatccacctgggagagctgcacgccattctgcggcggcaggaagatttttacccattcct
gaaggacaaccgggaaaagatcgagaagatcctgaccttccgcatcccctactacgtgggcc
ctctggccaggggaaacagcagattcgcctggatgaccagaaagagcgaggaaaccatcacc
ccctggaacttcgaggaagtggtggacaagggcgcttccgcccagagcttcatcgagcggat
gaccaacttcgataagaacctgcccaacgagaaggtgctgcccaagcacagcctgctgtacg
agtacttcaccgtgtataacgagctgaccaaagtgaaatacgtgaccgagggaatgagaaag
cccgccttcctgagcggcgagcagaaaaaggccatcgtggacctgctgttcaagaccaaccg
gaaagtgaccgtgaagcagctgaaagaggactacttcaagaaaatcgagtgcttcgactccg
tggaaatctccggcgtggaagatcggttcaacgcctccctgggcacataccacgatctgctg
aaaattatcaaggacaaggacttcctggacaatgaggaaaacgaggacattctggaagatat
cgtgctgaccctgacactgtttgaggacagagagatgatcgaggaacggctgaaaacctatg
cccacctgttcgacgacaaagtgatgaagcagctgaagcggcggagatacaccggctggggc
aggctgagccggaagctgatcaacggcatccgggacaagcagtccggcaagacaatcctgga
tttcctgaagtccgacggcttcgccaacagaaacttcatgcagctgatccacgacgacagcc
tgacctttaaagaggacatccagaaagcccaggtgtccggccagggcgatagcctgcacgag
cacattgccaatctggccggcagccccgccattaagaagggcatcctgcagacagtgaaggt
ggtggacgagctcgtgaaagtgatgggccggcacaagcccgagaacatcgtgatcgaaatgg
ccagagagaaccagaccacccagaagggacagaagaacagccgcgagagaatgaagcggatc
gaagagggcatcaaagagctgggcagccagatcctgaaagaacaccccgtggaaaacaccca
gctgcagaacgagaagctgtacctgtactacctgcagaatgggcgggatatgtacgtggacc
aggaactggacatcaaccggctgtccgactacgatgtggaccatatcgtgcctcagagcttt
ctgaaggacgactccatcgacaacaaggtgctgaccagaagcgacaagaaccggggcaagag
cgacaacgtgccctccgaagaggtcgtgaagaagatgaagaactactggcggcagctgctga
acgccaagctgattacccagagaaagttcgacaatctgaccaaggccgagagaggcggcctg
agcgaactggataaggccggcttcatcaagagacagctggtggaaacccggcagatcacaaa
gcacgtggcacagatcctggactcccggatgaacactaagtacgacgagaatgacaagctga
tccgggaagtgaaagtgatcaccctgaagtccaagctggtgtccgatttccggaaggatttc
cagttttacaaagtgcgcgagatcaacaactaccaccacgcccacgacgcctacctgaacgc
cgtcgtgggaaccgccctgatcaaaaagtaccctaagctggaaagcgagttcgtgtacggcg
actacaaggtgtacgacgtgcggaagatgatcgccaagagcgagcaggaaatcggcaaggct
accgccaagtacttcttctacagcaacatcatgaactttttcaagaccgagattaccctggc
caacggcgagatccggaagcggcctctgatcgagacaaacggcgaaaccggggagatcgtgt
gggataagggccgggattttgccaccgtgcggaaagtgctgagcatgccccaagtgaatatc
gtgaaaaagaccgaggtgcagacaggcggcttcagcaaagagtctatcctgcccaagaggaa
cagcgataagctgatcgccagaaagaaggactgggaccctaagaagtacggcggcttcgaca
gccccaccgtggcctattctgtgctggtggtggccaaagtggaaaagggcaagtccaagaaa
ctgaagagtgtgaaagagctgctggggatcaccatcatggaaagaagcagcttcgagaagaa
tcccatcgactttctggaagccaagggctacaaagaagtgaaaaaggacctgatcatcaagc
tgcctaagtactccctgttcgagctggaaaacggccggaagagaatgctggcctctgccggc
gaactgcagaagggaaacgaactggccctgccctccaaatatgtgaacttcctgtacctggc
cagccactatgagaagctgaagggctcccccgaggataatgagcagaaacagctgtttgtgg
aacagcacaagcactacctggacgagatcatcgagcagatcagcgagttctccaagagagtg
atcctggccgacgctaatctggacaaagtgctgtccgcctacaacaagcaccgggataagcc
catcagagagcaggccgagaatatcatccacctgtttaccctgaccaatctgggagcccctg
ccgccttcaagtactttgacaccaccatcgaccggaagaggtacaccagcaccaaagaggtg
ctggacgccaccctgatccaccagagcatcaccggcctgtacgagacacggatcgacctgtc
tcagctgggaggcgacaaaaggccggcggccacgaaaaaggccggccaggcaaaaaagaaaa
aggaattcggcagtggagagggcagaggaagtctgctaacatgcggtgacgtcgaggagaat
cctggcccagtgagcaagggcgaggagctgttcaccggggtggtgcccatcctggtcgagct
ggacggcgacgtaaacggccacaagttcagcgtgtccggcgagggcgagggcgatgccacct
acggcaagctgaccctgaagttcatctgcaccaccggcaagctgcccgtgccctggcccacc
ctcgtgaccaccctgacctacggcgtgcagtgcttcagccgctaccccgaccacatgaagca
gcacgacttcttcaagtccgccatgcccgaaggctacgtccaggagcgcaccatcttcttca
aggacgacggcaactacaagacccgcgccgaggtgaagttcgagggcgacaccctggtgaac
cgcatcgagctgaagggcatcgacttcaaggaggacggcaacatcctggggcacaagctgga
gtacaactacaacagccacaacgtctatatcatggccgacaagcagaagaacggcatcaagg
tgaacttcaagatccgccacaacatcgaggacggcagcgtgcagctcgccgaccactaccag
cagaacacccccatcggcgacggccccgtgctgctgcccgacaaccactacctgagcaccca
gtccgccctgagcaaagaccccaacgagaagcgcgatcacatggtcctgctggagttcgtga
ccgccgccgggatcactctcggcatggacgagctgtacaaggaattctaactagagctcgct
gatcagcctcgactgtgccttctagttgccagccatctgttgtttgcccctcccccgtgcct
tccttgaccctggaaggtgccactcccactgtcctttcctaataaaatgaggaaattgcatc
gcattgtctgagtaggtgtcattctattctggggggtggggtggggcaggacagcaaggggg
aggattgggaagagaatagcaggcatgctggggagcggccgcaggaacccctagtgatggag
ttggccactccctctctgcgcgctcgctcgctcactgaggccgggcgaccaaaggtcgcccg
acgcccgggctttgcccgggcggcctcagtgagcgagcgagcgcgcagctgcctgcaggggc
gcctgatgcggtattttctccttacgcatctgtgcggtatttcacaccgcatacgtcaaagc
aaccatagtacgcgccctgtagcggcgcattaagcgcggcgggtgtggtggttacgcgcagc
gtgaccgctacacttgccagcgccttagcgcccgctcctttcgctttcttcccttcctttct
cgccacgttcgccggctttccccgtcaagctctaaatcgggggctccctttagggttccgat
ttagtgctttacggcacctcgaccccaaaaaacttgatttgggtgatggttcacgtagtggg
ccatcgccctgatagacggtttttcgccctttgacgttggagtccacgttctttaatagtgg
actcttgttccaaactggaacaacactcaactctatctcgggctattcttttgatttataag
ggattttgccgatttcggtctattggttaaaaaatgagctgatttaacaaaaatttaacgcg
aattttaacaaaatattaacgtttacaattttatggtgcactctcagtacaatctgctctga
tgccgcatagttaagccagccccgacacccgccaacacccgctgacgcgccctgacgggctt
gtctgctcccggcatccgcttacagacaagctgtgaccgtctccgggagctgcatgtgtcag
aggttttcaccgtcatcaccgaaacgcgcgagacgaaagggcctcgtgatacgcctattttt
ataggttaatgtcatgataataatggtttcttagacgtcaggtggcacttttcggggaaatg
tgcgcggaacccctatttgtttatttttctaaatacattcaaatatgtatccgctcatgaga
caataaccctgataaatgcttcaataatattgaaaaaggaagagtatgagtattcaacattt
ccgtgtcgcccttattcccttttttgcggcattttgccttcctgtttttgctcacccagaaa
cgctggtgaaagtaaaagatgctgaagatcagttgggtgcacgagtgggttacatcgaactg
gatctcaacagcggtaagatccttgagagttttcgccccgaagaacgttttccaatgatgag
cacttttaaagttctgctatgtggcgcggtattatcccgtattgacgccgggcaagagcaac
tcggtcgccgcatacactattctcagaatgacttggttgagtactcaccagtcacagaaaag
catcttacggatggcatgacagtaagagaattatgcagtgctgccataaccatgagtgataa
cactgcggccaacttacttctgacaacgatcggaggaccgaaggagctaaccgcttttttgc
acaacatgggggatcatgtaactcgccttgatcgttgggaaccggagctgaatgaagccata
ccaaacgacgagcgtgacaccacgatgcctgtagcaatggcaacaacgttgcgcaaactatt
aactggcgaactacttactctagcttcccggcaacaattaatagactggatggaggcggata
aagttgcaggaccacttctgcgctcggcccttccggctggctggtttattgctgataaatct
ggagccggtgagcgtggaagccgcggtatcattgcagcactggggccagatggtaagccctc
ccgtatcgtagttatctacacgacggggagtcaggcaactatggatgaacgaaatagacaga
tcgctgagataggtgcctcactgattaagcattggtaactgtcagaccaagtttactcatat
atactttagattgatttaaaacttcatttttaatttaaaaggatctaggtgaagatcctttt
tgataatctcatgaccaaaatcccttaacgtgagttttcgttccactgagcgtcagaccccg
tagaaaagatcaaaggatcttcttgagatcctttttttctgcgcgtaatctgctgcttgcaa
acaaaaaaaccaccgctaccagcggtggtttgtttgccggatcaagagctaccaactctttt
tccgaaggtaactggcttcagcagagcgcagataccaaatactgttcttctagtgtagccgt
agttaggccaccacttcaagaactctgtagcaccgcctacatacctcgctctgctaatcctg
ttaccagtggctgctgccagtggcgataagtcgtgtcttaccgggttggactcaagacgata
gttaccggataaggcgcagcggtcgggctgaacggggggttcgtgcacacagcccagcttgg
agcgaacgacctacaccgaactgagatacctacagcgtgagctatgagaaagcgccacgctt
cccgaagggagaaaggcggacaggtatccggtaagcggcagggtcggaacaggagagcgcac
gagggagcttccagggggaaacgcctggtatctttatagtcctgtcgggtttcgccacctct
gacttgagcgtcgatttttgtgatgctcgtcaggggggcggagcctatggaaaaacgccagc
aacgcggcctttttacggttcctggccttttgctggccttttgctcacatgt
pTRE3G-A-Repeat-EF1a-RFP: DYRK1A Sequence:
CTCGAGTTTACTCCCTATCAGTGATAGAGAACGTATGAAGAGTTTACTCCCTATCAGTGATA
GAGAACGTATGCAGACTTTACTCCCTATCAGTGATAGAGAACGTATAAGGAGTTTACTCCCT
ATCAGTGATAGAGAACGTATGACCAGTTTACTCCCTATCAGTGATAGAGAACGTATCTACAG
TTTACTCCCTATCAGTGATAGAGAACGTATATCCAGTTTACTCCCTATCAGTGATAGAGAAC
GTATAAGCTTTAGGCGTGTACGGTGGGCGCCTATAAAAGCAGAGCTCGTTTAGTGAACCGTC
AGATCGCCTGGAGCAATTCCACAACACTTTTGTCTTATACCAACTTTCCGTACCACTTCCTA
CCCTCGTAAAGTCGACACCGGGGCCCAGATCTGGTACCGAGCTCGGATCCACTAGTCCAGTG
TGGTGGAATTCTGCAGAGAATTTTTCTTTGGAATCATTTTTGGTTGACATCTCTGTTTTTTG
TGGATCAGTTTTTTACTCTTCCACTCTCTTTTCTATATTTTGCCCATCGGGGCTGCGGATAC
CTGGTTTTATTATTTTTTCTTTGCCCAACGGGGCCGTGGATACCTGCCTTTTAATTCTTTTT
TATTCGCCCATCGGGGCCGCGGATACCTGCTTTTTATTTTTTTTTCCTTAGCCCATCGGGGT
ATCGGATACCTGCTGATTCCCTTCCCCTCTGAACCCCCAACACTCTGGCCCATCGGGGTGAC
GGATATCTGCTTTTTAAAAATTTTCTTTTTTTGGCCCATCGGGGCTTCGGATACCTGCTTTT
TTTTTTTTTATTTTTCCTTGCCCATCGGGGCCTCGGATACCTGCTTTAATTTTTGTTTTTCT
GGCCCATCGGGGCCGCGGATACCTGCTTTGATTTTTTTTTTTCATCGCCCATCGGTGCTTTT
TATGGATGAAAAAATGTTTCGATCGGCCGGATATCACGCGTCATATGGCTAGCCTGCAGGGA
TCCAATGTAACTGTATTCAGCGATGACGAAATTCTTAGCTATTGTAATACTCTAGAGGATCT
TTGTGAAGGAACCTTACTTCTGTGGTGTGACATAATTGGACAAACTACCTACAGAGATTTAA
AGCTCTAAGGTAAATATAAAATTTTTAAGTGTATAATGTGTTAAACTACTGATTCTAATTGT
TTGTGTATTTTAGATTCCAACCTATGGAACTGATGAATGGGAGCAGTGGTGGAATGCCTTTA
ATGAGGAAAACCTGTTTTGCTCAGAAGAAATGCCATCTAGTGATGATGAGGCTACTGCTGAC
TCTCAACATTCTACTCCTCCAAAAAAGAAGAGAAAGGTAGAAGACCCCAAGGACTTTCCTTC
AGAATTGCTAAGTTTTTTGAGTCATGCTGTGTTTAGTAATAGAACTCTTGCTTGCTTTGCTA
TTTACACCACAAAGGAAAAAGCTGCACTGCTATACAAGAAAATTATGGAAAAATATTCTGTA
ACCTTTATAAGTAGGCATAACAGTTATAATCATAACATACTGTTTTTTCTTACTCCACACAG
GCATAGAGTGTCTGCTATTAATAACTATGCTCAAAAATTGTGTACCTTTAGCTTTTTAATTT
GTAAAGGGGTTAATAAGGAATATTTGATGTATAGTGCCTTGACTAGAGATCATAATCAGCCA
TACCACATTTGTAGAGGTTTTACTTGCTTTAAAAAACCTCCCACACCTCCCCCTGAACCTGA
AACATAAAATGAATGCAATTGTTGTTGTTAACTTGTTTATTGCAGCTTATAATGGTTACAAA
TAAAGCAATAGCATCACAAATTTCACAAATAAAGCATTTTTTTCACTGCATTCTAGTTGTGG
TTTGTCCAAACTCATCAATGTATCTTAGAGGGACAGCCCCCCCCCAAAGCCCCCAGGGATGT
AATTACGTCCCTCCCCCGCTAGGGGGCAGCAGCGAGCCGCCCGGGGCTCCGCTCCGGTCCGG
CGCTCCCCCCGCATCCCCGAGCCGGCAGCGTGCGGGGACAGCCCGGGCACGGGGAAGGTGGC
ACGGGATCGCTTTCCTCTGAACGCTTCTCGCTGCTCTTTGAGCCTGCAGACACCTGGGGGGA
TACGGGGAAAAGCTAGCAAGGATCTGCGATCGCTCCGGTGCCCGTCAGTGGGCAGAGCGCAC
ATCGCCCACAGTCCCCGAGAAGTTGGGGGGAGGGGTCGGCAATTGAACGGGTGCCTAGAGAA
GGTGGCGCGGGGTAAACTGGGAAAGTGATGTCGTGTACTGGCTCCGCCTTTTTCCCGAGGGT
GGGGGAGAACCGTATATAAGTGCAGTAGTCGCCGTGAACGTTCTTTTTCGCAACGGGTTTGC
CGCCAGAACACAGCTGAAGCTTCGAGGGGCTCGCATCTCTCCTTCACGCGCCCGCCGCCCTA
CCTGAGGCCGCCATCCACGCCGGTTGAGTCGCGTTCTGCCGCCTCCCGCCTGTGGTGCCTCC
TGAACTGCGTCCGCCGTCTAGGTAAGTTTAAAGCTCAGGTCGAGTCCGGGCCTTTGTCCGGC
GCTCCCTTGGAGCCTACCTAGACTCAGCCGGCTCTCCACGCTTTGCCTGACCCTGCTTGCTC
AACTCTACGTCTTTGTTTCGTTTTCTGTTCTGCGCCGTTACAGATCCAAGCTGTGACCGGCG
CCTACGAATTCGCCACCATGGCCAGCTCCGAGGATGTCATCAAAGAGTTTATGAGATTTAAG
GTCAAGATGGAGGGAAGCGTCAACGGACACGAGTTCGAGATTGAGGGAGAAGGAGAAGGCCG
GCCTTACGAGGGCACACAAACCGCTAAGCTCAAGGTCACAAAAGGAGGACCCCTCCCCTTCT
CCTGGGATATTCTGAGCCCTCAGTTCCAGTACGGAAGCAAAGCCTATGTTAAACACCCTGCC
GACATCCCTGACTATCTGAAGCTCTCCTTCCCTGAAGGCTTCAAGTGGGAGAGATTCATGAA
CTTCGAGGACGGAGGCGTGGTGACAGTCACACAAGATAGCACCCTCCAGGACGGAGAGTTTA
TTTATAAGGTGAAACTCAGAGGAACCAACTTCCCCTCCGATGGCCCTGTCATGCAAAAAAAA
ACAATGGGATGGGAAGCCTCCACCGAGAGAATGTATCCTGAGGATGGCGCTCTGAAAGGCGA
AATTAAAATGAGACTGAAACTCAAAGACGGAGGACACTACGATGCCGAGGTCAAAACAACCT
ACAAGGCCAAGAAACAAGTGCAGCTGCCTGGCGCCTACATGACTGATATTAAACTCGACATT
ATCAGCCATAATGGGGACTACACCATCGTGGAACAATATGAGAGAGCTGAGGGCAGACATAG
CACAGGCGCTGGAAGTACTTGAGGATCCTGATCGAGTCTAGAGGGCCCCCGCTGATCAGCCT
CGACTGTGCCTTCTAGTTGCCAGCCATCTGTTGTTTGCCCCTCCCCCGTGCCTTCCTTGACC
CTGGAAGGTGCCACTCCCACTGTCCTTTCCTAATAAAATGAGGAAATTGCATCGCATTGTCT
GAGTAGGTGTCATTCTATTCTGGGGGGTGGGGTGGGGCAGGACAGCAAGGGGGAGGATTGGG
AAGAGAATAGCAGGCATGCTGGGGAAGATCTTCATGTCTGCGGCTCTAGAGCTGCATTAATG
AATCGGCCAACGCGCGGGGAGAGGCGGTTTGCGTATTGGGCGCTCTTCCGCTTCCTCGCTCA
CTGACTCGCTGCGCTCGGTCGTTCGGCTGCGGCGAGCGGTATCAGCTCACTCAAAGGCGGTA
ATACGGTTATCCACAGAATCAGGGGATAACGCAGGAAAGAACATGTACGTAAACTGGCAAAG
GGGTGGCTGGGCCAAAAGACAGAGGAATTAAGTAAGAAGTCCAGGAAAAATGAACTTCACAT
CAAATTTTAGAGCACGGTAGCCATGAATCTTGTGAATAGCTCCCAAAAATGTCCTGTGGAAG
ACAACTAGAAAGCATTCTACAATCAGGCACCCACCTCCACCTGCAGCCTCCTGTGTTGTTCT
CATGGGGCACCTCTGGGCTCCAGCTCCTCCAAGGCACCTCCACACTCTCTCAAGTACACTCT
TCACTCTTCCCCAAACATGATTCCCCTACTGCTCTGCCTAACTCCCACTTCTCTTTCAAGTA
GCAGCTTAAACGTCACCTCATATTTGGCTGGAAAATAGAATATAGACAGAGGGGTAAGTTAA
GGCTAGAAAGGCAGGCTGGGTCAACAGAATGGCAAGCTAAAACATGGGATTTTCTAAAACAG
CCTAAGAGGGTGACAGATAAAAGTGTGCAAGGAGTGGCACAACTCCAGTTTCATCTTTAGCT
ATAGCAATTAACACCATAAGGAGTCTGGATTCAATTTTGCCATTTACTAGCTAGCTACCAAC
TTCTGTGTCGCTTTGGGCAAATCAATTAAATCCATACCTCCCTTTCCATCTGCAGAATGGGT
TTATAACAGTACTTAAACCTCAAGGTACTAAGAACAGTAAAGAGTTAATGGTACATGTGAGC
AAAAGGCCAGCAAAAGGCCAGGAACCGTAAAAAGGCCGCGTTGCTGGCGTTTTTCCATAGGC
TCCGCCCCCCTGACGAGCATCACAAAAATCGACGCTCAAGTCAGAGGTGGCGAAACCCGACA
GGACTATAAAGATACCAGGCGTTTCCCCCTGGAAGCTCCCTCGTGCGCTCTCCTGTTCCGAC
CCTGCCGCTTACCGGATACCTGTCCGCCTTTCTCCCTTCGGGAAGCGTGGCGCTTTCTCATA
GCTCACGCTGTAGGTATCTCAGTTCGGTGTAGGTCGTTCGCTCCAAGCTGGGCTGTGTGCAC
GAACCCCCCGTTCAGCCCGACCGCTGCGCCTTATCCGGTAACTATCGTCTTGAGTCCAACCC
GGTAAGACACGACTTATCGCCACTGGCAGCAGCCACTGGTAACAGGATTAGCAGAGCGAGGT
ATGTAGGCGGTGCTACAGAGTTCTTGAAGTGGTGGCCTAACTACGGCTACACTAGAAGAACA
GTATTTGGTATCTGCGCTCTGCTGAAGCCAGTTACCTTCGGAAAAAGAGTTGGTAGCTCTTG
ATCCGGCAAACAAACCACCGCTGGTAGCGGTGGTTTTTTTGTTTGCAAGCAGCAGATTACGC
GCAGAAAAAAAGGATCTCAAGAAGATCCTTTGATCTTTTCTACGGGGTCTGACGCTCAGTGG
AACGAAAACTCACGTTAAGGGATTTTGGTCATGAGATTATCAAAAAGGATCTTCACCTAGAT
CCTTTTAAATTAAAAATGAAGTTTTAAATCAATCTAAAGTATATATGAGTAAACTTGGTCTG
ACAGTTACCAATGCTTAATCAGTGAGGCACCTATCTCAGCGATCTGTCTATTTCGTTCATCC
ATAGTTGCCTGACTCCCCGTCGTGTAGATAACTACGATACGGGAGGGCTTACCATCTGGCCC
CAGTGCTGCAATGATACCGCGAGACCCACGCTCACCGGCTCCAGATTTATCAGCAATAAACC
AGCCAGCCGGAAGGGCCGAGCGCAGAAGTGGTCCTGCAACTTTATCCGCCTCCATCCAGTCT
ATTAATTGTTGCCGGGAAGCTAGAGTAAGTAGTTCGCCAGTTAATAGTTTGCGCAACGTTGT
TGCCATTGCTACAGGCATCGTGGTGTCACGCTCGTCGTTTGGTATGGCTTCATTCAGCTCCG
GTTCCCAACGATCAAGGCGAGTTACATGATCCCCCATGTTGTGCAAAAAAGCGGTTAGCTCC
TTCGGTCCTCCGATCGTTGTCAGAAGTAAGTTGGCCGCAGTGTTATCACTCATGGTTATGGC
AGCACTGCATAATTCTCTTACTGTCATGCCATCCGTAAGATGCTTTTCTGTGACTGGTGAGT
ACTCAACCAAGTCATTCTGAGAATAGTGTATGCGGCGACCGAGTTGCTCTTGCCCGGCGTCA
ATACGGGATAATACCGCGCCACATAGCAGAACTTTAAAAGTGCTCATCATTGGAAAACGTTC
TTCGGGGCGAAAACTCTCAAGGATCTTACCGCTGTTGAGATCCAGTTCGATGTAACCCACTC
GTGCACCCAACTGATCTTCAGCATCTTTTACTTTCACCAGCGTTTCTGGGTGAGCAAAAACA
GGAAGGCAAAATGCCGCAAAAAAGGGAATAAGGGCGACACGGAAATGTTGAATACTCATACT
CTTCCTTTTTCAATATTATTGAAGCATTTATCAGGGTTATTGTCTCATGAGCGGATACATAT
TTGAATGTATTTAGAAAAATAAACAAATAGGGGTTCCGCGCACATTTCCCCGAAAAGTGCCA
CCTGACGTCTAAGAAACCATTATTATCATGACATTAACCTATAAAAATAGGCGTATCACGAG
GCCCTTTCGTCTTCAAGAATTCGAAAACCAGAAAGTATTCTCAGTAATGATAGTATGGATAA
AGCAGGTTTCTATGACCCTTTATTACAGAATCTGTGAGTTTTTCACAATTAAAAAGTAATAA
AAAGTAGTGACAACATTCACTGAACTCTTATTCTATGCCAACTTGTTCCGGTATGCCCTTAC
ACCCACAAAAGCCCTATGCATAAGGTGGCATTATTCCAGCATGTATTGCATTGTACACACAA
AGAGGTCAAGCACTCCACCACGGCCCTAAGCATGGTGGCTGAGGTGGGAAGGCCAGAGGTAG
GTGGGCCCGCGCCCTTTTCCACTCTGAACCATGCCTCCAAGATAGGAGGGTGGGAAAGTGCT
CAAGACACATTAGAAATTCCCCATAAAAGACAAGATTGTTGAACACCTGCAAGTGAATAAAG
ATAAACTGATCTCAGAGGGGAAAAAGACGCAGGGTTAGGAAACAGCACCCTGCTCGAGGACG
TTCTTTCCAAACAGCCTGCTCATCACCCGTTCGAATTC

### REFERENCE LIST

Almeida, M., Pintacuda, G., Masui, O., Koseki, Y., Gdula, M., Cerase, A., . . . Brockdorff, N. (2017). PCGF3/5-PRC1 initiates Polycomb recruitment in X chromosome inactivation. Science, 356(6342), 1081-1084. doi:10.1126/science.aal2512
Bao, X., Lian, X., & Palecek, S. P. (2016). Directed Endothelial Progenitor Differentiation from Human Pluripotent Stem Cells Via Wnt Activation Under Defined Conditions. Methods in Molecular Biology, 1481, 183-196. doi:10.1007/978-1-4939-6393-5_17
Bickmore, W. A. (2013). The spatial organization of the human genome. Annu Rev Genomics Hum Genet, 14, 67-84. doi:10.1146/annurev-genom-091212-153515
Bickmore, W. A., & Teague, P. (2002). Influences of chromosome size, gene density and nuclear position on the frequency of constitutional translocations in the human population. Chromosome Research, 10(8), 707-715. doi:10.1023/a:1021589031769
Brockdorff, N. (2018). Local Tandem Repeat Expansion in Xist RNA as a Model for the Functionalisation of ncRNA. Noncoding RNA, 4(4). doi:10.3390/ncrna4040028
Brockdorff, N., Bowness, J. S., & Wei, G. (2020). Progress toward understanding chromosome silencing by Xist RNA. Genes and Development, 34(11-12), 733-744. doi:10.1101/gad.337196.120
Butler, J. T., Hall, L. L., Smith, K. P., & Lawrence, J. B. (2009). Changing nuclear landscape and unique PML structures during early epigenetic transitions of human embryonic stem cells. Journal of Cellular Biochemistry, 107(4), 609-621. doi:10.1002/jcb.22183
Byron, M., Hall, L. L., & Lawrence, J. B. (2013). A multifaceted FISH approach to study endogenous RNAs and DNAs in native nuclear and cell structures. Curr Protoc Hum Genet, Chapter 4, Unit 4 15. doi:10.1002/0471142905.hg0415s76
Chaumeil, J., Le Baccon, P., Wutz, A., & Heard, E. (2006). A novel role for Xist RNA in the formation of a repressive nuclear compartment into which genes are recruited when silenced. Genes and Development, 20(16), 2223-2237. doi:10.1101/gad.380906
Chen, C. K., Blanco, M., Jackson, C., Aznauryan, E., Ollikainen, N., Surka, C., . . . Guttman, M. (2016). Xist recruits the X chromosome to the nuclear lamina to enable chromosome-wide silencing. Science, 354(6311), 468-472. doi:10.1126/science.aae0047
Chu, C., Zhang, Q. C., da Rocha, S. T., Flynn, R. A., Bharadwaj, M., Calabrese, J. M., . . . Chang, H. Y. (2015). Systematic discovery of Xist RNA binding proteins. Cell, 161(2), 404-416. doi:10.1016/j.cell.2015.03.025
Clemson, C. M., Chow, J. C., Brown, C. J., & Lawrence, J. B. (1998). Stabilization and localization of Xist RNA are controlled by separate mechanisms and are not sufficient for X inactivation. Journal of Cell Biology, 142(1), 13-23. Retrieved from ncbi.nlm.nih.gov/pubmed/9660859
Clemson, C. M., Hall, L. L., Byron, M., McNeil, J., & Lawrence, J. B. (2006). The X chromosome is organized into a gene-rich outer rim and an internal core containing silenced nongenic sequences. Proceedings of the National Academy of Sciences of the United States of America, 103(20), 7688-7693. doi:10.1073/pnas.0601069103
Clemson, C. M., McNeil, J. A., Willard, H. F., & Lawrence, J. B. (1996). XIST RNA paints the inactive X chromosome at interphase: evidence for a novel RNA involved in nuclear/chromosome structure. Journal of Cell Biology, 132(3), 259-275. doi:10.1083/jcb.132.3.259
Colognori, D., Sunwoo, H., Wang, D., Wang, C. Y., & Lee, J. T. (2020). Xist Repeats A and B Account for Two Distinct Phases of X Inactivation Establishment. Developmental Cell, 54(1), 21-32 e25. doi:10.1016/j.devcel.2020.05.021
Czerminski, J. T., & Lawrence, J. B. (2020). Silencing Trisomy 21 with XIST in Neural Stem Cells Promotes Neuronal Differentiation. Developmental Cell, 52(3), 294-308 e293. doi:10.1016/j.devcel.2019.12.015
Davidovich, C., Goodrich, K. J., Gooding, A. R., & Cech, T. R. (2014). A dimeric state for PRC2. Nucleic Acids Research, 42(14), 9236-9248. doi:10.1093/nar/gku540
DeKelver, R. C., Choi, V. M., Moehle, E. A., Paschon, D. E., Hockemeyer, D., Meijsing, S. H., . . . Urnov, F. D. (2010). Functional genomics, proteomics, and regulatory DNA analysis in isogenic settings using zinc finger nuclease-driven transgenesis into a safe harbor locus in the human genome. Genome Research, 20(8), 1133-1142. doi:10.1101/gr.106773.110
Eltoukhy, A. A., Siegwart, D. J., Alabi, C. A., Rajan, J. S., Langer, R., & Anderson, D. G. (2012). Effect of molecular weight of amine end-modified poly(beta-amino ester)s on gene delivery efficiency and toxicity. Biomaterials, 33(13), 3594-3603. doi:10.1016/j.biomaterials.2012.01.046
Engreitz, J. M., Pandya-Jones, A., McDonel, P., Shishkin, A., Sirokman, K., Surka, C., . . . Guttman, M. (2013). The Xist lncRNA exploits three-dimensional genome architecture to spread across the X chromosome. Science, 341(6147), 1237973. doi:10.1126/science.1237973
Ha, N., Lai, L. T., Chelliah, R., Zhen, Y., Yi Vanessa, S. P., Lai, S. K., . . . Zhang, L. F. (2018). Live-Cell Imaging and Functional Dissection of Xist RNA Reveal Mechanisms of X Chromosome Inactivation and Reactivation. iScience, 8, 1-14. doi:10.1016/j.isci.2018.09.007
Hall, L. L., & Lawrence, J. B. (2010). XIST RNA and architecture of the inactive X chromosome: implications for the repeat genome. Cold Spring Harbor Symposia on Quantitative Biology, 75, 345-356. doi:10.1101/sqb.2010.75.030
Hall, L. L., Byron, M., Butler, J., Becker, K. A., Nelson, A., Amit, M., . . . Lawrence, J. B. (2008). X-inactivation reveals epigenetic anomalies in most hESC but identifies sublines that initiate as expected. Journal of Cellular Physiology, 216(2), 445-452. doi:10.1002/jcp.21411
Hall, L. L., Byron, M., Pageau, G., & Lawrence, J. B. (2009). AURKB-mediated effects on chromatin regulate binding versus release of XIST RNA to the inactive chromosome. Journal of Cell Biology, 186(4), 491-507. doi:10.1083/jcb.200811143
Hall, L. L., Byron, M., Sakai, K., Carrel, L., Willard, H. F., & Lawrence, J. B. (2002). An ectopic human XIST gene can induce chromosome inactivation in postdifferentiation human HT-1080 cells. Proceedings of the National Academy of Sciences of the United States of America, 99(13), 8677-8682. doi:10.1073/pnas.132468999
Hasegawa, Y., Brockdorff, N., Kawano, S., Tsutui, K., Tsutui, K., & Nakagawa, S. (2010). The matrix protein hnRNP U is required for chromosomal localization of Xist RNA. Developmental Cell, 19(3), 469-476. doi:10.1016/j.devcel.2010.08.006
Helbig, R., & Fackelmayer, F. O. (2003). Scaffold attachment factor A (SAF-A) is concentrated in inactive X chromosome territories through its RGG domain. Chromosoma, 112(4), 173-182. doi:10.1007/s00412-003-0258-0
Jiang, J., Jing, Y., Cost, G. J., Chiang, J. C., Kolpa, H. J., Cotton, A. M., . . . Lawrence, J. B. (2013). Translating dosage compensation to trisomy 21. Nature, 500(7462), 296-300. doi:10.1038/nature12394
Kolpa, H. J., Fackelmayer, F. O., & Lawrence, J. B. (2016). SAF-A Requirement in Anchoring XIST RNA to Chromatin Varies in Transformed and Primary Cells. Developmental Cell, 39(1), 9-10. doi:10.1016/j.devcel.2016.09.021
Mahy, N. L., Perry, P. E., & Bickmore, W. A. (2002). Gene density and transcription influence the localization of chromatin outside of chromosome territories detectable by FISH. Journal of Cell Biology, 159(5), 753-763. doi:10.1083/jcb.200207115
McCarthy, D. J., Chen, Y., & Smyth, G. K. (2012). Differential expression analysis of multifactor RNA-Seq experiments with respect to biological variation. Nucleic Acids Research, 40(10), 4288-4297. doi:10.1093/nar/gks042
McHugh, C. A., Chen, C. K., Chow, A., Surka, C. F., Tran, C., McDonel, P., . . . Guttman, M. (2015). The Xist lncRNA interacts directly with SHARP to silence transcription through HDAC3. Nature, 521(7551), 232-236. doi:10.1038/nature14443
Minks, J., Baldry, S. E., Yang, C., Cotton, A. M., & Brown, C. J. (2013). XIST-induced silencing of flanking genes is achieved by additive action of repeat a monomers in human somatic cells. Epigenetics Chromatin, 6(1), 23. doi:10.1186/1756-8935-6-23
Nesterova, T. B., Wei, G., Coker, H., Pintacuda, G., Bowness, J. S., Zhang, T., . . . Brockdorff, N. (2019). Systematic allelic analysis defines the interplay of key pathways in X chromosome inactivation. Nat Commun, 10(1), 3129. doi:10.1038/s41467-019-11171-3
Park, I. H., Arora, N., Huo, H., Maherali, N., Ahfeldt, T., Shimamura, A., . . . Daley, G. Q. (2008). Disease-specific induced pluripotent stem cells. Cell, 134(5), 877-886. doi:10.1016/j.cell.2008.07.041
Ridings-Figueroa, R., Stewart, E. R., Nesterova, T. B., Coker, H., Pintacuda, G., Godwin, J., . . . Coverley, D. (2017). The nuclear matrix protein CIZ1 facilitates localization of Xist RNA to the inactive X-chromosome territory. Genes and Development, 31(9), 876-888. doi:10.1101/gad.295907.117
Shin, J., Bossenz, M., Chung, Y., Ma, H., Byron, M., Taniguchi-Ishigaki, N., . . . Bach, I. (2010). Maternal Rnf12/RLIM is required for imprinted X-chromosome inactivation in mice. Nature, 467(7318), 977-981. doi:10.1038/nature09457
Stamoulis, G., Garieri, M., Makrythanasis, P., Letourneau, A., Guipponi, M., Panousis, N., . . . Antonarakis, S. E. (2019). Single cell transcriptome in aneuploidies reveals mechanisms of gene dosage imbalance. Nat Commun, 10(1), 4495. doi:10.1038/s41467-019-12273-8
Sunwoo, H., Colognori, D., Froberg, J. E., Jeon, Y., & Lee, J. T. (2017). Repeat E anchors Xist RNA to the inactive X chromosomal compartment through CDKN1A-interacting protein (CIZ1). Proceedings of the National Academy of Sciences, 114(40), 10654-10659. doi:10.1073/pnas.1711206114
Swarts, D. R. A., Stewart, E. R., Higgins, G. S., & Coverley, D. (2018). CIZ1-F, an alternatively spliced variant of the DNA replication protein CIZ1 with distinct expression and localisation, is overrepresented in early stage common solid tumours. Cell Cycle, 17(18), 2268-2283. doi:10.1080/15384101.2018.1526600
Wang, C. Y., Jegu, T., Chu, H. P., Oh, H. J., & Lee, J. T. (2018). SMCHD1 Merges Chromosome Compartments and Assists Formation of Super-Structures on the Inactive X. Cell, 174(2), 406-421 e425. doi:10.1016/j.cell.2018.05.007
Warder, D. E., & Keherly, M. J. (2003). Ciz1, Cip1 interacting zinc finger protein 1 binds the consensus DNA sequence ARYSR(0-2)YYAC. Journal of Biomedical Science, 10(4), 406-417. doi:10.1007/bf02256432
Wutz, A., Rasmussen, T. P., & Jaenisch, R. (2002). Chromosomal silencing and localization are mediated by different domains of Xist RNA. Nature Genetics, 30(2), 167-174. doi:10.1038/ng820
Xing, Y., Johnson, C. V., Dobner, P. R., & Lawrence, J. B. (1993). Higher level organization of individual gene transcription and RNA splicing. Science, 259(5099), 1326-1330. Retrieved from ncbi.nlm.nih.gov/pubmed/8446901
Zhao, J., Sun, B. K., Erwin, J. A., Song, J. J., & Lee, J. T. (2008). Polycomb proteins targeted by a short repeat RNA to the mouse X chromosome. Science, 322(5902), 750-756. doi:10.1126/science.1163045
Zugates, G. T., Peng, W., Zumbuehl, A., Jhunjhunwala, S., Huang, Y. H., Langer, R., . . . Anderson, D. G. (2007). Rapid optimization of gene delivery by parallel end-modification of poly(beta-amino ester)s. Molecular Therapy, 15(7), 1306-1312. doi:10.1038/mt.sj.6300132
Zylicz, J. J., Bousard, A., Zumer, K., Dossin, F., Mohammad, E., da Rocha, S. T., . . . Heard, E. (2019). The Implication of Early Chromatin Changes in X Chromosome Inactivation. Cell, 176(1-2), 182-197.e123. doi:10.1016/j.cell.2018.11.041
Cabrejo, L., Guyant-Marechal, L., Laquerriere, A., Vercelletto, M., De la Fourniere, F., Thomas-Anterion, C., . . . Hannequin, D. (2006). Phenotype associated with APP duplication in five families. Brain, 129(Pt 11), 2966-2976. doi:10.1093/brain/awl237
Kasuga, K., Shimohata, T., Nishimura, A., Shiga, A., Mizuguchi, T., Tokunaga, J., . . . Ikeuchi, T. (2009). Identification of independent APP locus duplication in Japanese patients with early-onset Alzheimer disease. J Neurol Neurosurg Psychiatry, 80(9), 1050-1052. doi:10.1136/jnnp.2008.161703
Mann, D. M., & Esiri, M. M. (1989). The pattern of acquisition of plaques and tangles in the brains of patients under 50 years of age with Down's syndrome. J Neurol Sci, 89(2-3), 169-179. doi:10.1016/0022-510x(89)90019-1
Rovelet-Lecrux, A., Hannequin, D., Raux, G., Le Meur, N., Laquerriere, A., Vital, A., . . . Campion, D. (2006). APP locus duplication causes autosomal dominant early-onset Alzheimer disease with cerebral amyloid angiopathy. Nat Genet, 38(1), 24-26. doi:10.1038/ng1718
Sleegers, K., Brouwers, N., Gijselinck, I., Theuns, J., Goossens, D., Wauters, J., . . . Van Broeckhoven, C. (2006). APP duplication is sufficient to cause early onset Alzheimer's dementia with cerebral amyloid angiopathy. Brain, 129(Pt 11), 2977-2983. doi:10.1093/brain/awl203
Wisniewski, K. E., Wisniewski, H. M., & Wen, G. Y. (1985). Occurrence of neuropathological changes and dementia of Alzheimer's disease in Down's syndrome. Ann Neurol, 17(3), 278-282. doi:10.1002/ana.410170310
Zigman, W. B., Schupf, N., Sersen, E., & Silverman, W. (1996). Prevalence of dementia in adults with and without Down syndrome. Am J Ment Retard, 100(4), 403-412.

1. A method of silencing one or more alleles of a target gene in a cell, the method comprising inserting a silencing sequence of up to 5 kB comprising a promoter sequence and 6-50 A-repeats, wherein each A-repeat comprises a sequence that is at least 80% identical to the sequence GCCCA[T/A]CGGGG[C/T]N[G/T/A][C/T]GGATA[C/T]CTG, wherein N is any nucleotide, optionally with T-rich flanking regions in between each repeat, into the genome of the cell, wherein the silencing sequence is inserted into a site that is up to 5 Mb away from the target gene promoter.

2. The method of claim 1, wherein genomic insertion of the silencing sequence is directed by zinc-finger nucleases or TALENs that specifically target the genomic insertion site.

3. The method of claim 1, wherein genomic insertion of the nucleotide sequence is directed by Cas9 complexed with a guide RNA that specifically target the genomic insertion site.

4. The method of claim 1, wherein the silencing sequence is inserted at a copy number variation or single-nucleotide polymorphism (SNP) that is located within a 5′ UTR, intron, or exon of one or more alleles of the target gene.

5. The method of claim 1, wherein the silencing sequence is inserted at a sequence that is present on just one homologous chromosome, optionally a single-nucleotide polymorphism (SNP) or copy number variation (CNV), that is present within a 5′ UTR, intron, or exon of one allele of the target gene but absent in other alleles of the target gene.

6. The method of claim 5, wherein the target gene is present in two or more copies in the cell, and the presence of two or more copies of the target gene is associated with a disease.

7. The method of claim 6, wherein the disease is selected from the group of Down Syndrome, Alzheimer's, Chromosomal imbalance disorders, and microduplication disorders.

8. The method of claim 6, wherein the disease is Down Syndrome or Alzheimer's Disease and the target gene is amyloid precursor protein (APP), DYRK1A, DSCR3 (VPS26C), TTC3, PIGP, HLCS, RCAN1, CBR1, DONSON, ETS2, PSMG1, MX1, BACE2, IFNAR1, IFNGR2, IFNAR2, and/or ILL.

9. The method of claim 1, wherein the cell is a cell in or from a living subject, preferably a mammal, preferably a human, who has a disease.

10. The method of claim 8, wherein the disease is selected from the group of Down Syndrome, Alzheimer's disease, Chromosomal imbalance disorders, and microduplication disorders.

11. The method of claim 1, wherein the target gene is APP or DYRK1A.

12. The method of claim 1, wherein the method results in silencing of a plurality of genes that have promoters within up to 5 Mb, preferably up to 100-500 kb, of the insertion site.

13. A silencing sequence of up to 5 kB comprising a promoter sequence and 6-50, preferably 6-20, 8-20, 8-50, 9-20, 9-50, or 20-50, A-repeats, wherein each A-repeat comprises a sequence that is at least 80% identical to the sequence GCCCA[T/A]CGGGG[C/T]N[G/T/A][C/T]GGATA[C/T]CTG, wherein N is any nucleotide, optionally with T-rich flanking regions in between each repeat.

14.-24. (canceled)

25. The method of claim 1, wherein the silencing sequence comprises a promoter 6-20, 8-20, 8-50, 9-20, 9-50, or 20-50 A-repeats.

26. The method of claim 1, wherein the silencing sequence is inserted into a site that is 100-500 kb, away from the target gene promoter.


SEQUENCE LISTING (93 sequences)

SEQ ID NO: 1 - DNA, 24 bp, synthetic construct
  source 1..24: mol_type = other DNA
gcccawcggggyndyggatayctg

SEQ ID NO: 2 - DNA, 11 bp, synthetic construct
  source 1..11: mol_type = other DNA
ggcgccatntt

SEQ ID NO: 3 - DNA, 497 bp, synthetic construct
  source 1..497: mol_type = other DNA
gaatttttctttggaatcatttttggttgacatctctgttttttgtggatcagttttttactcttccactctcttttctatattttgcccatcggggctgcggatacctggttttattattttttctttgcccaacggggccgtggatacctgccttttaattcttttttattcgcccatcggggccgcggatacctgctttttatttttttttccttagcccatcggggtatcggatacctgctgattcccttcccctctgaacccccaacactctggcccatcggggtgacggatatctgctttttaaaaattttctttttttggcccatcggggcttcggatacctgctttttttttttttatttttccttgcccatcggggcctcggatacctgctttaatttttgtttttctggcccatcggggccgcggatacctgctttgatttttttttttcatcgcccatcggtgctttttatggatgaaaaaatgtt

SEQ ID NO: 4 - DNA, 43 bp, synthetic construct
  source 1..43: mol_type = other DNA
ttttctatattttgcccatcggggctgcggatacctggtttta

SEQ ID NO: 5 - DNA, 45 bp, synthetic construct
  source 1..45: mol_type = other DNA
ttattttttctttgcccaacggggccgtggatacctgccttttaa

SEQ ID NO: 6 - DNA, 45 bp, synthetic construct
  source 1..45: mol_type = other DNA
ttcttttttattcgcccatcggggccgcggatacctgctttttat

SEQ ID NO: 7 - DNA, 59 bp, synthetic construct
  source 1..59: mol_type = other DNA
ttttttttccttagcccatcggggtatcggatacctgctgattcccttcccctctgaac

SEQ ID NO: 8 - DNA, 48 bp, synthetic construct
  source 1..48: mol_type = other DNA
ccccaacactctggcccatcggggtgacggatatctgctttttaaaaa

SEQ ID NO: 9 - DNA, 47 bp, synthetic construct
  source 1..47: mol_type = other DNA
ttttctttttttggcccatcggggcttcggatacctgcttttttttt

SEQ ID NO: 10 - DNA, 44 bp, synthetic construct
  source 1..44: mol_type = other DNA
ttttatttttccttgcccatcggggcctcggatacctgctttaa

SEQ ID NO: 11 - DNA, 46 bp, synthetic construct
  source 1..46: mol_type = other DNA
tttttgtttttctggcccatcggggccgcggatacctgctttgatt

SEQ ID NO: 12 - DNA, 25 bp, synthetic construct
  source 1..25: mol_type = other DNA
tttttttttcatcgcccatcggtgc

SEQ ID NO: 13 - DNA, 44 bp, synthetic construct
  source 1..44: mol_type = other DNA
ttcgcccatcggggccgcggatacctgctttttatttttttttc

SEQ ID NO: 14 - DNA, 24 bp, synthetic construct
  source 1..24: mol_type = other DNA
gcccatcggggctgcggatacctg

SEQ ID NO: 15 - DNA, 24 bp, synthetic construct
  source 1..24: mol_type = other DNA
gcccaacggggccgtggatacctg

SEQ ID NO: 16 - DNA, 24 bp, synthetic construct
  source 1..24: mol_type = other DNA
gcccatcggggccgcggatacctg

SEQ ID NO: 17 - DNA, 24 bp, synthetic construct
  source 1..24: mol_type = other DNA
gcccatcggggtatcggatacctg

SEQ ID NO: 18 - DNA, 24 bp, synthetic construct
  source 1..24: mol_type = other DNA
gcccatcggggtgacggatatctg

SEQ ID NO: 19 - DNA, 24 bp, synthetic construct
  source 1..24: mol_type = other DNA
gcccatcggggcttcggatacctg

SEQ ID NO: 20 - DNA, 24 bp, synthetic construct
  source 1..24: mol_type = other DNA
gcccatcggggcctcggatacctg

SEQ ID NO: 21 - DNA, 43 bp, synthetic construct
  source 1..43: mol_type = other DNA
ttattttttctttgcccaacggggccgtggatacctgcctttt

SEQ ID NO: 22 - DNA, 43 bp, synthetic construct
  source 1..43: mol_type = other DNA
ttcttttttattcgcccatcggggccgcggatacctgcttttt

SEQ ID NO: 23 - DNA, 43 bp, synthetic construct
  source 1..43: mol_type = other DNA
ttttttttccttagcccatcggggtatcggatacctgctgatt

SEQ ID NO: 24 - DNA, 43 bp, synthetic construct
  source 1..43: mol_type = other DNA
ccccaacactctggcccatcggggtgacggatatctgcttttt

SEQ ID NO: 25 - DNA, 43 bp, synthetic construct
  source 1..43: mol_type = other DNA
ttttctttttttggcccatcggggcttcggatacctgcttttt

SEQ ID NO: 26 - DNA, 43 bp, synthetic construct
  source 1..43: mol_type = other DNA
tttatttttccttgcccatcggggcctcggatacctgctttaa

SEQ ID NO: 27 - DNA, 43 bp, synthetic construct
  source 1..43: mol_type = other DNA
ttttgtttttctggcccatcggggccgcggatacctgctttga

SEQ ID NO: 28 - DNA, 31 bp, synthetic construct
  source 1..31: mol_type = other DNA
ggaagatcttcatgtctgcggctctagagct

SEQ ID NO: 29 - DNA, 36 bp, synthetic construct
  source 1..36: mol_type = other DNA
aaagaaaaattctctgcagaattccaccacactgga

SEQ ID NO: 30 - DNA, 43 bp, synthetic construct
  source 1..43: mol_type = other DNA
aattctgcagagaatttttctttggaatcatttttggttgaca

SEQ ID NO: 31 - DNA, 37 bp, synthetic construct
  source 1..37: mol_type = other DNA
ccgatcgaaacattttttcatccataaaaagcaccga

SEQ ID NO: 32 - DNA, 34 bp, synthetic construct
  source 1..34: mol_type = other DNA
ggatgaaaaaatgtttcgatcggccggatatcac

SEQ ID NO: 33 - DNA, 41 bp, synthetic construct
  source 1..41: mol_type = other DNA
gctgtccctctaagatacattgatgagtttggacaaaccac

SEQ ID NO: 34 - DNA, 30 bp, synthetic construct
  source 1..30: mol_type = other DNA
tgtatcttagagggacagcccccccccaaa

SEQ ID NO: 35 - DNA, 29 bp, synthetic construct
  source 1..29: mol_type = other DNA
aggatcctcaagtacttccagcgcctgtg

SEQ ID NO: 36 - DNA, 33 bp, synthetic construct
  source 1..33: mol_type = other DNA
aggcgctggaagtacttgaggatcctgatcgag

SEQ ID NO: 37 - DNA, 32 bp, synthetic construct
  source 1..32: mol_type = other DNA
acatgaagatcttccccagcatgcctgctatt

SEQ ID NO: 38 - DNA, 497 bp, synthetic construct; identical to SEQ ID NO: 3
  source 1..497: mol_type = other DNA

SEQ ID NO: 39 - DNA, 43 bp, synthetic construct; identical to SEQ ID NO: 4
  source 1..43: mol_type = other DNA

SEQ ID NO: 40 - DNA, 45 bp, synthetic construct; identical to SEQ ID NO: 5
  source 1..45: mol_type = other DNA

SEQ ID NO: 41 - DNA, 47 bp, synthetic construct; identical to SEQ ID NO: 9
  source 1..47: mol_type = other DNA

SEQ ID NO: 42 - DNA, 51 bp, synthetic construct
  source 1..51: mol_type = other DNA
aggtatccgcggccccgatgggcagaaaaacaaaaattaaagcaggtatcc

SEQ ID NO: 43 - DNA, 53 bp, synthetic construct
  source 1..53: mol_type = other DNA
aggtatccgcggccccgatgggcgaataaaaaagaattaaaaggcaggtatcc

SEQ ID NO: 44 - DNA, 51 bp, synthetic construct
  source 1..51: mol_type = other DNA
aggtatccacggccccgttgggcaaagaaaaaataataaaaccaggtatcc

SEQ ID NO: 45 - DNA, 53 bp, synthetic construct
  source 1..53: mol_type = other DNA
aggtatccgataccccgatgggctaaggaaaaaaaaataaaaagcaggtatcc

SEQ ID NO: 46 - DNA, 56 bp, synthetic construct
  source 1..56: mol_type = other DNA
aggtatccgaagccccgatgggccaaaaaaagaaaatttttaaaaagcagatatcc

SEQ ID NO: 47 - DNA, 356 bp, synthetic construct
  source 1..356: mol_type = other DNA
gacattgcggcattgctcagcatggcgggctgtgctttgttaggttgtccaaaatggcggatccagttctgtcgcagtgttcaagtggcgggaaggccacatcatgatgggcgaggctttgttaagtggttagcatggtggtggacatgtgcggtcacacaggaaaagatggcggctgaaggtcttgccgcagtgtaaaacatggcgggcctctttgtctttgctgtgtgcttttcgtgttgggttttgccgcagggacaatatggcaggcgttgtcatatgtatatcatggcttttgtcacgtggacatcatggcgggcttgccgcattgttaaagatggcgggttttgccgcct

SEQ ID NO: 48 - DNA, 40 bp, synthetic construct
  source 1..40: mol_type = other DNA
gacattgcggcattgctcagcatggcgggctgtgcttttg

SEQ ID NO: 49 - DNA, 40 bp, synthetic construct
  source 1..40: mol_type = other DNA
ggtcttgccgcagtgtaaaacatggcgggcctctttgtct

SEQ ID NO: 50 - DNA, 40 bp, synthetic construct
  source 1..40: mol_type = other DNA
gggcttgccgcattgttaaagatggcgggttttgccgcct

SEQ ID NO: 51 - DNA, 40 bp, synthetic construct
  source 1..40: mol_type = other DNA
ggncttgccgcattgttaaacatggcgggctttgctgtct

SEQ ID NO: 52 - DNA, 97 bp, synthetic construct
  source 1..97: mol_type = other DNA
atacctccccccccaccccccaacccccccaactccccacccccaccccccaccccccacctccccacccccctacccccctacccccctacccccc

SEQ ID NO: 53 - DNA, 119 bp, synthetic construct
  source 1..119: mol_type = other DNA
cctccccagccctgctcccagcaaacccctagtctagccccagccctactcccaccccgccccagccctgccccagccccagtcccctaaccccccagccctagccccagtcccagtcc

SEQ ID NO: 54 - DNA, 120 bp, synthetic construct
  source 1..120: mol_type = other DNA
tgctcaaaataagttgtccattgcttatcctattatactgggatattccgtttacccttggcattgctgatcttcagtactgactccttgaccattttcagttaatgcatacaatcccat

SEQ ID NO: 55 - DNA, 2634 bp, synthetic construct
  source 1..2634: mol_type = other DNA
ccttttgaaggacagcatggttggtgacacctaaggccccatttcttggcctcccaatatgtgtgattgtatttgtcgaggttgctatgcactagagaaggaaagtgctcccctcatccccacttttcccttccagcaggaagtgcccaccccataagacccttttatttggagagtctaggtgcacaattgtaagtgaccacaagcatgcatcttggacatttatgtgcgtaatcgcacactgctcattccatgtgaataaggtcctactctccgaccccttttgcaatacagaagggttgctgataacgcagtccccttttcttggcatgttgtgtgtgattataatcgtctgggatcctatgcactagaaaaggagggtcctctccacatacctcagtctcacctttcccttccagcagggagtgcccactccataagactctcacatttggacagtcaaggtgcgtaattgttaagtgaacacaaccatgcaccttagacatggatttgcataactacacacagctcaacctatctgaataaaatcctactctcagaccccttttgcagtacagcaggggtgctgatcaccaaggccctttttcctggcctggtatgcgtgtgattatgtttgtcccggttcctgtgtattagacatggaagcctcccctgccacactccacccccaatcttcctttcccttccggcagggagtgccctctccataagacgcttacgtttggacaatcaaggtgcacagttgtaagtgaccacaggcatacaccttggacattaatgtgcataaccactttgcccattccatctgaataaggtcctactctcagaccccttttgcagtacagcaggggtgctgatcaccaaggccccttttcttggcctgttatgtgcgtgattatatttgtctgggttcctgtgtattagacaaggaagccttccccccgcccccacccccactcccagtcttcctttcccttccagcagggagtgccccctccataagatcattacatttggacaatcaaggtgcacaattataagtgaccacagccatgcaccttggacattattggacattaatgtgcgtaactgcacatggcccatcccatctgaataaggtcctactctcagatgccctttgcagtacagcaggggtactgaatcaccaaggccctttttcttggcctgttatgtgtgtgattatatttatcccagtttctgtgtaatagacatgaaagcctcccctgccacaccccacctccaatcttcctttcccttccaccagggagtgtccactccatatacccttacatttggacaatcaaggtgcacaattgtaagtgagcataggcactcaccttggacatgaatgtgcataactgcacatggcccatcccatctgaataaggtcctactctcagaccctttttgcagtacagcaggggtgctgatcaccaaggccccttttcctggcctgttatgtgtgtgattatatttgttccagttcctgtgtaatagacatggaagcctcccctgccacactccacccccaatcttcctttcccttctggcaggaagtacccgctccataagacccttacatttggacagtcaaggtgcacaattgtatgtgaccacaaccatgcaccttggacataaatgtgtgtaactgcacatggcccatcccatctgaataaggtcctactctcagaccccttttgcagtacagtaggtgtgctgataaccaaggcccctcttcctggcctgttaacgtatgtgattatatttgtctgggttccagtgtataagacatggaagcctcccctgccccaccccaccctcaatcttcctttcccttctggcagggagtgccagctccataagaaccttacatttggacagtcaaggtgcacaattctaagtgaccgcagccatgcaccttggtcaataatgtgtgtaactgcacacggcctatctcatctgaataaggccttactctcagaccccttttgcagtacagcaggggtgctgataaccaaggcccattttcctggcctgttatgtgtgtgattatatttgtccaggtttctgtgtactagacaaggaagcctcctctgccccatcccatctacgcataatctttcttttcctcccagcagggagtgctcactccataagacccttacatttggacaatcaaggtgcacaattgtaagtgaccacaaccatgcatcttggaaatttatgtgcataactgcacatggcttatcctatttgaataaagtcctactctcagaccccctttgcagtatagctggggtgctgatcactgaggcctctttgcttggcttgtctatattcttgtgtactagataagggcaccttctcatggactccctttgcttttcaacaaggagtacccactactttttaagattcttatatttgtccaaagtacatggttttaattgaccacaacaatgtcccttggacattaatgtatgtaatcaccacatggttcatcctaattaaacaaagttctaccttctcaccctccatttgcagtataccagggttgctgaccccctaagtccccttttcttggcttgttga

SEQ ID NO: 56 - DNA, 279 bp, synthetic construct
  source 1..279: mol_type = other DNA
ccttttgaaggacagcatggttggtgacacctaaggccccatttcttggcctcccaatatgtgtgattgtatttgtcgaggttgctatgcactagagaaggaaagtgctcccctcatccccacttttcccttccagcaggaagtgcccaccccataagacccttttatttggagagtctaggtgcacaattgtaagtgaccacaagcatgcatcttggacatttatgtgcgtaatcgcacactgctcattccatgtgaataaggtcctactctccgacc

SEQ ID NO: 57 - DNA, 284 bp, synthetic construct
  source 1..284: mol_type = other DNA
ccttttgcaatacagaagggttgctgataacgcagtccccttttcttggcatgttgtgtgtgattataatcgtctgggatcctatgcactagaaaaggagggtcctctccacatacctcagtctcacctttcccttccagcagggagtgcccactccataagactctcacatttggacagtcaaggtgcgtaattgttaagtgaacacaaccatgcaccttagacatggatttgcataactacacacagctcaacctatctgaataaaatcctactctcagacc

SEQ ID NO: 58 - DNA, 288 bp, synthetic construct
  source 1..288: mol_type = other DNA
ccttttgcagtacagcaggggtgctgatcaccaaggccctttttcctggcctggtatgcgtgtgattatgtttgtcccggttcctgtgtattagacatggaagcctcccctgccacactccacccccaatcttcctttcccttccggcagggagtgccctctccataagacgcttacgtttggacaatcaaggtgcacagttgtaagtgaccacaggcatacaccttggacattaatgtgcataaccactttgcccattccatctgaataaggtcctactctcagacc

SEQ ID NO: 59 - DNA, 305 bp, synthetic construct
  source 1..305: mol_type = other DNA
ccttttgcagtacagcaggggtgctgatcaccaaggccccttttcttggcctgttatgtgcgtgattatatttgtctgggttcctgtgtattagacaaggaagccttccccccgcccccacccccactcccagtcttcctttcccttccagcagggagtgccccctccataagatcattacatttggacaatcaaggtgcacaattataagtgaccacagccatgcaccttggacattattggacattaatgtgcgtaactgcacatggcccatcccatctgaataaggtcctactctcagatgc

SEQ ID NO: 60 - DNA, 289 bp, synthetic construct
  source 1..289: mol_type = other DNA
cctttgcagtacagcaggggtactgaatcaccaaggccctttttcttggcctgttatgtgtgtgattatatttatcccagtttctgtgtaatagacatgaaagcctcccctgccacaccccacctccaatcttcctttcccttccaccagggagtgtccactccatatacccttacatttggacaatcaaggtgcacaattgtaagtgagcataggcactcaccttggacatgaatgtgcataactgcacatggcccatcccatctgaataaggtcctactctcagacc

SEQ ID NO: 61 - DNA, 290 bp, synthetic construct
  source 1..290: mol_type = other DNA
ctttttgcagtacagcaggggtgctgatcaccaaggccccttttcctggcctgttatgtgtgtgattatatttgttccagttcctgtgtaatagacatggaagcctcccctgccacactccacccccaatcttcctttcccttctggcaggaagtacccgctccataagacccttacatttggacagtcaaggtgcacaattgtatgtgaccacaaccatgcaccttggacataaatgtgtgtaactgcacatggcccatcccatctgaataaggtcctactctcagacc

SEQ ID NO: 62 - DNA, 291 bp, synthetic construct
  source 1..291: mol_type = other DNA
ccttttgcagtacagtaggtgtgctgataaccaaggcccctcttcctggcctgttaacgtatgtgattatatttgtctgggttccagtgtataagacatggaagcctcccctgccccaccccaccctcaatcttcctttcccttctggcagggagtgccagctccataagaaccttacatttggacagtcaaggtgcacaattctaagtgaccgcagccatgcaccttggtcaataatgtgtgtaactgcacacggcctatctcatctgaataaggccttactctcagacc

SEQ ID NO: 63 - DNA, 294 bp, synthetic construct
  source 1..294: mol_type = other DNA
ccttttgcagtacagcaggggtgctgataaccaaggcccattttcctggcctgttatgtgtgtgattatatttgtccaggtttctgtgtactagacaaggaagcctcctctgccccatcccatctacgcataatctttcttttcctcccagcagggagtgctcactccataagacccttacatttggacaatcaaggtgcacaattgtaagtgaccacaaccatgcatcttggaaatttatgtgcataactgcacatggcttatcctatttgaataaagtcctactctcagacc

SEQ ID NO: 64 - DNA, 295 bp, synthetic construct
  source 1..295: mol_type = other DNA
ccctttgcagtatagctggggtgctgatcactgaggcctctttgcttggcttgtctatattcttgtgtactagataagggcaccttctcatggactccctttgcttttcaacaaggagtacccactactttttaagattcttatatttgtccaaagtacatggttttaattgaccacaacaatgtcccttggacattaatgtatgtaatcaccacatggttcatcctaattaaacaaagttctaccttctcaccctccatttgcagtataccagggttgctgaccccctaagtcc

SEQ ID NO: 65 - DNA, 19 bp, synthetic construct
  source 1..19: mol_type = other DNA
ccttttcttggcttgttga

SEQ ID NO: 66 - DNA, 497 bp, synthetic construct; identical to SEQ ID NO: 3
  source 1..497: mol_type = other DNA

SEQ ID NO: 67 - DNA, 120 bp, synthetic construct; identical to SEQ ID NO: 54
  source 1..120: mol_type = other DNA

SEQ ID NO: 68 - DNA, 2634 bp, synthetic construct; identical to SEQ ID NO: 55
  source 1..2634: mol_type = other DNA

SEQ ID NO: 69 - DNA, 209 bp, synthetic construct
  source 1..209: mol_type = other DNA
atcttcctcagaagaataggcttgttgttttacagtgttagtgatccattccctttgacgatccctaggtggagatggggcatgaggatcctccaggggaaaagctcactaccactgggcaacaaccctaggtcaggaggttctgtcaagatactttcctggtcccagataggaagataaagtctcaaaaacaaccaccacacgtcaag

SEQ ID NO: 70 - DNA, 1756 bp, synthetic construct
  source 1..1756: mol_type = other DNA
tgtgtatttctttgtctctttctttcttgtctttgctctttgttctctatctaaagtgtgtcttacccatttccatgtttctcttgctaatttctttcgtgtgtgcctttgcctcattttctctttttgttcacaagagtggtctgtgtcttgtcttagacatatctctcatttttcattttgttgctatttctctttgctctcctagatgtggctcttctttcacgctttatttcatgtctcctttttgggtcacatgctgtgtgctttttgtccttttcttgttctgtctacctctcctttctctgcctacctctcttttctctttgtgaactgtgattatttgttaccccttccccttctcgttcgttttaaatttcaccttttttctgagtctggcctcctttctgctgtttctactttttatctcacatttctcatttctgcatttcctttctgcctctcttgggctattctctctctcctcccctgcgtgcctcagcatctcttgctgtttgtgattttctatttcagtattaatctctgttggcttgtatttgttctctgcttcttccctttctactcacctttgagtatttcagcctcttcatgaatctatctccctctctttgatttcatgtaatctctccttaaatatttctttgcatatgtgggcaagtgtacgtgtgtgtgtgtcatgtgtggcagaggggcttcctaacccctgcctgataggtgcagaacgtcggctatcagagcaagcattgtggagcggttccttatgccaggctgccatgtgagatgatccaagaccaaaacaaggccctagactgcagtaaaacccagaactcaagtagggcagaaggtggaaggctcatatggatagaaggcccaaagtataagacagatggtttgagacttgagacccgaggactaagatggaaagcccatgttccaagatagatagaagcctcaggcctgaaaccaacaaaagcctcaagagccaagaaaacagagggtggcctgaattggaccgaaggcctgagttggatggaagtctcaaggcttgagttagaagtcttaagacctgggacaggacacatggaaggcctaagaactgagacttgtgacacaaggccaacgacctaagattagcccagggttgtagctggaagacctacaacccaaggatggaaggcccctgtcacaaagcctacctagatggatagaggacccaagcgaaaaaggtatctcaagactaacggccggaatctggaggcccatgacccagaacccaggaaggatagaagcttgaagacctggggaaatcccaagatgagaaccctaaaccctacctcttttctattgtttacacttcttactcttagatatttccagttctcctgtttatctttaagcctgattcttttgagatgtactttttgatgttgccggttacctttagattgacagtattatgcctgggccagtcttgagccagctttaaatcacagcttttacctatttgttaggctatagtgttttgtaaacttctgtttctattcacatcttctccacttgagagagacaccaaaatccagtcagtatctaatctggcttttgttaacttccctcaggagcagacattcatataggtgatactgtatttcagtcctttcttttgaccccagaagccctagactgagaagataaaatggtcaggttgttggggaaaaaaaagtgccaggctctctag

SEQ ID NO: 71 - DNA, 713 bp, synthetic construct
  source 1..713: mol_type = other DNA
gaatttttctttggaatcatttttggttgacatctctgttttttgtggatcagttttttactcttccactctcttttctatattttgcccatcggggctgcggatacctggttttattattttttctttgcccaacggggccgtggatacctgccttttaattcttttttattcgcccatcggggccgcggatacctgctttttatttttttttccttagcccatcggggtatcggatacctgctgattcccttcccctctgaacccccaacactctggcccatcggggtgacggatatctgctttttaaaaattttctttttttggcccatcggggcttcggatacctgctttttttttttttatttttccttgcccatcggggcctcggatacctgctttaatttttgtttttctggcccatcggggccgcggatacctgctttgatttttttttttcatcgcccatcggtgctttttatggatgaaaaaatgttatacctccccccccaccccccaacccccccaactccccacccccaccccccaccccccacctccccacccccctacccccctacccccctacccccccctccccagccctgctcccagcaaacccctagtctagccccagccctactcccaccccgccccagccctgccccagccccagtcccctaaccccccagccctagccccagtcccagtcc

SEQ ID NO: 72 - DNA, 1069 bp, synthetic construct
  source 1..1069: mol_type = other DNA
gaatttttctttggaatcatttttggttgacatctctgttttttgtggatcagttttttactcttccactctcttttctatattttgcccatcggggctgcggatacctggttttattattttttctttgcccaacggggccgtggatacctgccttttaattcttttttattcgcccatcggggccgcggatacctgctttttatttttttttccttagcccatcggggtatcggatacctgctgattcccttcccctctgaacccccaacactctggcccatcggggtgacggatatctgctttttaaaaattttctttttttggcccatcggggcttcggatacctgctttttttttttttatttttccttgcccatcggggcctcggatacctgctttaatttttgtttttctggcccatcggggccgcggatacctgctttgatttttttttttcatcgcccatcggtgctttttatggatgaaaaaatgttgacattgcggcattgctcagcatggcgggctgtgctttgttaggttgtccaaaatggcggatccagttctgtcgcagtgttcaagtggcgggaaggccacatcatgatgggcgaggctttgttaagtggttagcatggtggtggacatgtgcggtcacacaggaaaagatggcggctgaaggtcttgccgcagtgtaaaacatggcgggcctctttgtctttgctgtgtgcttttcgtgttgggttttgccgcagggacaatatggcaggcgttgtcatatgtatatcatggcttttgtcacgtggacatcatggcgggcttgccgcattgttaaagatggcgggttttgccgcctatacctccccccccaccccccaacccccccaactccccacccccaccccccaccccccacctccccacccccctacccccctacccccctacccccccctccccagccctgctcccagcaaacccctagtctagccccagccctactcccaccccgccccagccctgccccagccccagtcccctaaccccccagccctagccccagtcccagtcc

SEQ ID NO: 73 - DNA, 1189 bp, synthetic construct
  source 1..1189: mol_type = other DNA
gaatttttctttggaatcatttttggttgacatctctgttttttgtggatcagttttttactcttccactctcttttctatattttgcccatcggggctgcggatacctggttttattattttttctttgcccaacggggccgtggatacctgccttttaattcttttttattcgcccatcggggccgcggatacctgctttttatttttttttccttagcccatcggggtatcggatacctgctgattcccttcccctctgaacccccaacactctggcccatcggggtgacggatatctgctttttaaaaattttctttttttggcccatcggggcttcggatacctgctttttttttttttatttttccttgcccatcggggcctcggatacctgctttaatttttgtttttctggcccatcggggccgcggatacctgctttgatttttttttttcatcgcccatcggtgctttttatggatgaaaaaatgttgacattgcggcattgctcagcatggcgggctgtgctttgttaggttgtccaaaatggcggatccagttctgtcgcagtgttcaagtggcgggaaggccacatcatgatgggcgaggctttgttaagtggttagcatggtggtggacatgtgcggtcacacaggaaaagatggcggctgaaggtcttgccgcagtgtaaaacatggcgggcctctttgtctttgctgtgtgcttttcgtgttgggttttgccgcagggacaatatggcaggcgttgtcatatgtatatcatggcttttgtcacgtggacatcatggcgggcttgccgcattgttaaagatggcgggttttgccgcctatacctccccccccaccccccaacccccccaactccccacccccaccccccaccccccacctccccacccccctacccccctacccccctacccccccctccccagccctgctcccagcaaacccctagtctagccccagccctactcccaccccgccccagccctgccccagccccagtcccctaaccccccagccctagccccagtcccagtcctgctcaaaataagttgtccattgcttatcctattatactgggatattccgtttacccttggcattgctgatcttcagtactgactccttgaccattttcagttaatgcatacaatcccat

SEQ ID NO: 74 - DNA, 3823 bp, synthetic construct
  source 1..3823: mol_type = other DNA
gaatttttctttggaatcatttttggttgacatctctgttttttgtggatcagttttttactcttccactctcttttctatattttgcccatcggggctgcggatacctggttttattattttttctttgcccaacggggccgtggatacctgccttttaattcttttttattcgcccatcggggccgcggatacctgctttttatttttttttccttagcccatcggggtatcggatacctgctgattcccttcccctctgaacccccaacactctggcccatcggggtgacggatatctgctttttaaaaattttctttttttggcccatcggggcttcggatacctgctttttttttttttatttttccttgcccatcggggcctcggatacctgctttaatttttgtttttctggcccatcggggccgcggatacctgctttgatttttttttttcatcgcccatcggtgctttttatggatgaaaaaatgttgacattgcggcattgctcagcatggcgggctgtgctttgttaggttgtccaaaatggcggatccagttctgtcgcagtgttcaagtggcgggaaggccacatcatgatgggcgaggctttgttaagtggttagcatggtggtggacatgtgcggtcacacaggaaaagatggcggctgaaggtcttgccgcagtgtaaaacatggcgggcctctttgtctttgctgtgtgcttttcgtgttgggttttgccgcagggacaatatggcaggcgttgtcatatgtatatcatggcttttgtcacgtggacatcatggcgggcttgccgcattgttaaagatggcgggttttgccgcctatacctccccccccaccccccaacccccccaactccccacccccaccccccaccccccacctccccacccccctacccccctacccccctacccccccctccccagccctgctcccagcaaacccctagtctagccccagccctactcccaccccgccccagccctgccccagccccagtcccctaaccccccagccctagccccagtcccagtcctgctcaaaataagttgtccattgcttatcctattatactgggatattccgtttacccttggcattgctgatcttcagtactgactccttgaccattttcagttaatgcatacaatcccatccttttgaaggacagcatggttggtgacacctaaggccccatttcttggcctcccaatatgtgtgattgtatttgtcgaggttgctatgcactagagaaggaaagtgctcccctcatccccacttttcccttccagcaggaagtgcccaccccataagacccttttatttggagagtctaggtgcacaattgtaagtgaccacaagcatgcatcttggacatttatgtgcgtaatcgcacactgctcattccatgtgaataaggtcctactctccgaccccttttgcaatacagaagggttgctgataacgcagtccccttttcttggcatgttgtgtgtgattataatcgtctgggatcctatgcactagaaaaggagggtcctctccacatacctcagtctcacctttcccttccagcagggagtgcccactccataagactctcacatttggacagtcaaggtgcgtaattgttaagtgaacacaaccatgcaccttagacatggatttgcataactacacacagctcaacctatctgaataaaatcctactctcagaccccttttgcagtacagcaggggtgctgatcaccaaggccctttttcctggcctggtatgcgtgtgattatgtttgtcccggttcctgtgtattagacatggaagcctcccctgccacactccacccccaatcttcctttcccttccggcagggagtgccctctccataagacgcttacgtttggacaatcaaggtgcacagttgtaagtgaccacaggcatacaccttggacattaatgtgcataaccactttgcccattccatctgaataaggtcctactctcagaccccttttgcagtacagcaggggtgctgatcaccaaggccccttttcttggcctgttatgtgcgtgattatatttgtctgggttcctgtgtattagacaaggaagccttccccccgcccccacccccactcccagtcttcctttcccttccagcagggagtgccccctccataagatcattacatttggacaatcaaggtgcacaattataagtgaccacagccatgcaccttggacattattggacattaatgtgcgtaactgcacatggcccatcccatctgaataaggtcctactctcagatgccctttgcagtacagcaggggtactgaatcaccaaggccctttttcttggcctgttatgtgtgtgattatatttatcccagtttctgtgtaatagacatgaaagcctcccctgccacaccccacctccaatcttcctttcccttccaccagggagtgtccactccatatacccttacatttggacaatcaaggtgcacaattgtaagtgagcataggcactcaccttggacatgaatgtgcataactgcacatggcccatcccatctgaataaggtcctactctcagaccctttttgcagtacagcaggggtgctgatcaccaaggccccttttcctggcctgttatgtgtgtgattatatttgttccagttcctgtgtaatagacatggaagcctcccctgccacactccacccccaatcttcctttcccttctggcaggaagtacccgctccataagacccttacatttggacagtcaaggtgcacaattgtatgtgaccacaaccatgcaccttggacataaatgtgtgtaactgcacatggcccatcccatctgaataaggtcctactctcagaccccttttgcagtacagtaggtgtgctgataaccaaggcccctcttcctggcctgttaacgtatgtgattatatttgtctgggttccagtgtataagacatggaagcctcccctgccccaccccaccctcaatcttcctttcccttctggcagggagtgccagctccataagaaccttacatttggacagtcaaggtgcacaattctaagtgaccgcagccatgcaccttggtcaataatgtgtgtaactgcacacggcctatctcatctgaataaggccttactctcagaccccttttgcagtacagcaggggtgctgataaccaaggcccattttcctggcctgttatgtgtgtgattatatttgtccaggtttctgtgtactagacaaggaagcctcctctgccccatcccatctacgcataatctttcttttcctcccagcagggagtgctcactccataagacccttacatttggacaatcaaggtgcacaattgtaagtgaccacaaccatgcatcttggaaatttatgtgcataactgcacatggcttatcctatttgaataaagtcctactctcagaccccctttgcagtatagctggggtgctgatcactgaggcctctttgcttggcttgtctatattcttgtgtactagataagggcaccttctcatggactccctttgcttttcaacaaggagtacccactactttttaagattcttatatttgtccaaagtacatggttttaattgaccacaacaatgtcccttggacattaatgtatgtaatcaccacatggttcatcctaattaaacaaagttctaccttctcaccctccatttgcagtataccagggttgctgaccccctaagtccccttttcttggcttgttga

SEQ ID NO: 75 - DNA, 2945 bp, synthetic construct
  source 1..2945: mol_type = other DNA
gaatttttctttggaatcatttttggttgacatctctgttttttgtggatcagttttttactcttccactctcttttctatattttgcccatcggggctgcggatacctggttttattattttttctttgcccaacggggccgtggatacctgccttttaattcttttttattcgcccatcggggccgcggatacctgctttttatttttttttccttagcccatcggggtatcggatacctgctgattcccttcccctctgaacccccaacactctggcccatcggggtgacggatatctgctttttaaaaattttctttttttggcccatcggggcttcggatacctgctttttttttttttatttttccttgcccatcggggcctcggatacctgctttaatttttgtttttctggcccatcggggccgcggatacctgctttgatttttttttttcatcgcccatcggtgctttttatggatgaaaaaatgttgacattgcggcattgctcagcatggcgggctgtgctttgttaggttgtccaaaatggcggatccagttctgtcgcagtgttcaagtggcgggaaggccacatcatgatgggcgaggctttgttaagtggttagcatggtggtggacatgtgcggtcacacaggaaaagatggcggctgaaggtcttgccgcagtgtaaaacatggcgggcctctttgtctttgctgtgtgcttttcgtgttgggttttgccgcagggacaatatggcaggcgttgtcatatgtatatcatggcttttgtcacgtggacatcatggcgggcttgccgcattgttaaagatggcgggttttgccgcctatacctccccccccaccccccaacccccccaactccccacccccaccccccaccccccacctccccacccccctacccccctacccccctacccccccctccccagccctgctcccagcaaacccctagtctagccccagccctactcccaccccgccccagccctgccccagccccagtcccctaaccccccagccctagccccagtcccagtcctgctcaaaataagttgtccattgcttatcctattatactgggatattccgtttacccttggcattgctgatcttcagtactgactccttgaccattttcagttaatgcatacaatcccattgtgtatttctttgtctctttctttcttgtctttgctctttgttctctatctaaagtgtgtcttacccatttccatgtttctcttgctaatttctttcgtgtgtgcctttgcctcattttctctttttgttcacaagagtggtctgtgtcttgtcttagacatatctctcatttttcattttgttgctatttctctttgctctcctagatgtggctcttctttcacgctttatttcatgtctcctttttgggtcacatgctgtgtgctttttgtccttttcttgttctgtctacctctcctttctctgcctacctctcttttctctttgtgaactgtgattatttgttaccccttccccttctcgttcgttttaaatttcaccttttttctgagtctggcctcctttctgctgtttctactttttatctcacatttctcatttctgcatttcctttctgcctctcttgggctattctctctctcctcccctgcgtgcctcagcatctcttgctgtttgtgattttctatttcagtattaatctctgttggcttgtatttgttctctgcttcttccctttctactcacctttgagtatttcagcctcttcatgaatctatctccctctctttgatttcatgtaatctctccttaaatatttctttgcatatgtgggcaagtgtacgtgtgtgtgtgtcatgtgtggcagaggggcttcctaacccctgcctgataggtgcagaacgtcggctatcagagcaagcattgtggagcggttccttatgccaggctgccatgtgagatgatccaagaccaaaacaaggccctagactgcagtaaaacccagaactcaagtagggcagaaggtggaaggctcatatggatagaaggcccaaagtataagacagatggtttgagacttgagacccgaggactaagatggaaagcccatgttccaagatagatagaagcctcaggcctgaaaccaacaaaagcctcaagagccaagaaaacagagggtggcctgaattggaccgaaggcctgagttggatggaagtctcaaggcttgagttagaagtcttaagacctgggacaggacacatggaaggcctaagaactgagacttgtgacacaaggccaacgacctaagattagcccagggttgtagctggaagacctacaacccaaggatggaaggcccctgtcacaaagcctacctagatggatagaggacccaagcgaaaaaggtatctcaagactaacggccggaatctggaggcccatgacccagaacccaggaaggatagaagcttgaagacctggggaaatcccaagatgagaaccctaaaccctacctcttttctattgtttacacttcttactcttagatatttccagttctcctgtttatctttaagcctgattcttttgagatgtactttttgatgttgccggttacctttagattgacagtattatgcctgggccagtcttgagccagctttaaatcacagcttttacctatttgttaggctatagtgttttgtaaacttctgtttctattcacatcttctccacttgagagagacaccaaaatccagtcagtatctaatctggcttttgttaacttccctcaggagcagacattcatataggtgatactgtatttcagtcctttcttttgaccccagaagccctagactgagaagataaaatggtcaggttgttggggaaaaaaaagtgccaggctctctag

SEQ ID NO: 76 - DNA, 5459 bp, synthetic construct
  source 1..5459: mol_type = other DNA
gaatttttctttggaatcatttttggttgacatctctgttttttgtggatcagttttttactcttccactctcttttctatattttgcccatcggggctgcggatacctggttttattattttttctttgcccaacggggccgtggatacctgccttttaattcttttttattcgcccatcggggccgcggatacctgctttttatttttttttccttagcccatcggggtatcggatacctgctgattcccttcccctctgaacccccaacactctggcccatcggggtgacggatatctgctttttaaaaattttctttttttggcccatcggggcttcggatacctgctttttttttttttatttttccttgcccatcggggcctcggatacctgctttaatttttgtttttctggcccatcggggccgcggatacctgctttgatttttttttttcatcgcccatcggtgctttttatggatgaaaaaatgttgacattgcggcattgctcagcatggcgggctgtgctttgttaggttgtccaaaatggcggatccagttctgtcgcagtgttcaagtggcgggaaggccacatcatgatgggcgaggctttgttaagtggttagcatggtggtggacatgtgcggtcacacaggaaaagatggcggctgaaggtcttgccgcagtgtaaaacatggcgggcctctttgtctttgctgtgtgcttttcgtgttgggttttgccgcagggacaatatggcaggcgttgtcatatgtatatcatggcttttgtcacgtggacatcatggcgggcttgccgcattgttaaagatggcgggttttgccgcctatacctccccccccaccccccaacccccccaactccccacccccaccccccaccccccacctccccacccccctacccccctacccccctacccccccctccccagccctgctcccagcaaacccctagtctagccccagccctactcccaccccgccccagccctgccccagccccagtcccctaaccccccagccctagccccagtcccagtccccttttgaaggacagcatggttggtgacacctaaggccccatttcttggcctcccaatatgtgtgattgtatttgtcgaggttgctatgcactagagaaggaaagtgctcccctcatccccacttttcccttccagcaggaagtgcccaccccataagacccttttatttggagagtctaggtgcacaattgtaagtgaccacaagcatgcatcttggacatttatgtgcgtaatcgcacactgctcattccatgtgaataaggtcctactctccgaccccttttgcaatacagaagggttgctgataacgcagtccccttttcttggcatgttgtgtgtgattataatcgtctgggatcctatgcactagaaaaggagggtcctctccacatacctcagtctcacctttcccttccagcagggagtgcccactccataagactctcacatttggacagtcaaggtgcgtaattgttaagtgaacacaaccatgcaccttagacatggatttgcataactacacacagctcaacctatctgaataaaatcctactctcagaccccttttgcagtacagcaggggtgctgatcaccaaggccctttttcctggcctggtatgcgtgtgattatgtttgtcccggttcctgtgtattagacatggaagcctcccctgccacactccacccccaatcttcctttcccttccggcagggagtgccctctccataagacgcttacgtttggacaatcaaggtgcacagttgtaagtgaccacaggcatacaccttggacattaatgtgcataaccactttgcccattccatctgaataaggtcctactctcagaccccttttgcagtacagcaggggtgctgatcaccaaggccccttttcttggcctgttatgtgcgtgattatatttgtctgggttcctgtgtattagacaaggaagccttccccccgcccccacccccactcccagtcttcctttcccttccagcagggagtgccccctccataagatcattacatttggacaatcaaggtgcacaattataagtgaccacagccatgcaccttggacattattggacattaatgtgcgtaactgcacatggcccatcccatctgaataaggtcctactctcagatgccctttgcagtacagcaggggtactgaatcaccaaggccctttttcttggcctgttatgtgtgtgattatatttatcccagtttctgtgtaatagacatgaaagcctcccctgccacaccccacctccaatcttcctttcccttccaccagggagtgtccactccatatacccttacatttggacaatcaaggtgcacaattgtaagtgagcataggcactcaccttggacatgaatgtgcataactgcacatggcccatcccatctgaataaggtcctactctcagaccctttttgcagtacagcaggggtgctgatcaccaaggccccttttcctggcctgttatgtgtgtgattatatttgttccagttcctgtgtaatagacatggaagcctcccctgccacactccacccccaatcttcctttcccttctggcaggaagtacccgctccataagacccttacatttggacagtcaaggtgcacaattgtatgtgaccacaaccatgcaccttggacataaatgtgtgtaactgcacatggcccatcccatctgaataaggtcctactctcagaccccttttgcagtacagtaggtgtgctgataaccaaggcccctcttcctggcctgttaacgtatgtgattatatttgtctgggttccagtgtataagacatggaagcctcccctgccccaccccaccctcaatcttcctttcccttctggcagggagtgccagctccataagaaccttacatttggacagtcaaggtgcacaattctaagtgaccgcagccatgcaccttggtcaataatgtgtgtaactgcacacggcctatctcatctgaataaggccttactctcagaccccttttgcagtacagcaggggtgctgataaccaaggcccattttcctggcctgttatgtgtgtgattatatttgtccaggtttctgtgtactagacaaggaagcctcctctgccccatcccatctacgcataatctttcttttcctcccagcagggagtgctcactccataagacccttacatttggacaatcaaggtgcacaattgtaagtgaccacaaccatgcatcttggaaatttatgtgcataactgcacatggcttatcctatttgaataaagtcctactctcagaccccctttgcagtatagctggggtgctgatcactgaggcctctttgcttggcttgtctatattcttgtgtactagataagggcaccttctcatggactccctttgcttttcaacaaggagtacccactactttttaagattcttatatttgtccaaagtacatggttttaattgaccacaacaatgtcccttggacattaatgtatgtaatcaccacatggttcatcctaattaaacaaagttctaccttctcaccctccatttgcagtataccagggttgctgaccccctaagtccccttttcttggcttgttgatgtgtatttctttgtctctttctttcttgtctttgctctttgttctctatctaaagtgtgtcttacccatttccatgtttctcttgctaatttctttcgtgtgtgcctttgcctcattttctctttttgttcacaagagtggtctgtgtcttgtcttagacatatctctcatttttcattttgttgctatttctctttgctctcctagatgtggctcttctttcacgctttatttcatgtctcctttttgggtcacatgctgtgtgctttttgtccttttcttgttctgtctacctctcctttctctgcctacctctcttttctctttgtgaactgtgattatttgttaccccttccccttctcgttcgttttaaatttcaccttttttctgagtctggcctcctttctgctgtttctactttttatctcacatttctcatttctgcatttcctttctgcctctcttgggctattctctctctcctcccctgcgtgcctcagcatctcttgctgtttgtgattttctatttcagtattaatctctgttggcttgtatttgttctctgcttcttccctttctactcacctttgagtatttcagcctcttcatgaatctatctccctctctttgatttcatgtaatctctccttaaatatttctttgcatatgtgggcaagtgtacgtgtgtgtgtgtcatgtgtggcagaggggcttcctaacccctgcctgataggtgcagaacgtcggctatcagagcaagcattgtggagcggttccttatgccaggctgccatgtgagatgatccaagaccaaaacaaggccctagactgcagtaaaacccagaactcaagtagggcagaaggtggaaggctcatatggatagaaggcccaaagtataagacagatggtttgagacttgagacccgaggactaagatggaaagcccatgttccaagatagatagaagcctcaggcctgaaaccaacaaaagcctcaagagccaagaaaacagagggtggcctgaattggaccgaaggcctgagttggatggaagtctcaaggcttgagttagaagtcttaagacctgggacaggacacatggaaggcctaagaactgagacttgtgacacaaggccaacgacctaagattagcccagggttgtagctggaagacctacaacccaaggatggaaggcccctgtcacaaagcctacctagatggatagaggacccaagcgaaaaaggtatctcaagactaacggccggaatctggaggcccatgacccagaacccaggaaggatagaagcttgaagacctggggaaatcccaagatgagaaccctaaaccctacctcttttctattgtttacacttcttactcttagatatttccagttctcctgtttatctttaagcctgattcttttgagatgtactttttgatgttgccggttacctttagattgacagtattatgcctgggccagtcttgagccagctttaaatcacagcttttacctatttgttaggctatagtgttttgtaaacttctgtttctattcacatcttctccacttgagagagacaccaaaatccagtcagtatctaatctggcttttgttaacttccctcaggagcagacattcatataggtgatactgtatttcagtcctttcttttgaccccagaagccctagactgagaagataaaatggtcaggttgttggggaaaaaaaagtgccaggctctctag

SEQ ID NO: 77 - DNA, 5579 bp, synthetic construct
  source 1..5579: mol_type = other DNA
gaatttttctttggaatcatttttggttgacatctctgttttttgtggatcagttttttactcttccactctcttttctatattttgcccatcggggctgcggatacctggttttattattttttctttgcccaacggggccgtggatacctgccttttaattcttttttattcgcccatcggggccgcggatacctgctttttatttttttttccttagcccatcggggtatcggatacctgctgattcccttcccctctgaacccccaacactctggcccatcggggtgacggatatctgctttttaaaaattttctttttttggcccatcggggcttcggatacctgctttttttttttttatttttccttgcccatcggggcctcggatacctgctttaatttttgtttttctggcccatcggggccgcggatacctgctttgatttttttttttcatcgcccatcggtgctttttatggatgaaaaaatgttgacattgcggcattgctcagcatggcgggctgtgctttgttaggttgtccaaaatggcggatccagttctgtcgcagtgttcaagtggcgggaaggccacatcatgatgggcgaggctttgttaagtggttagcatggtggtggacatgtgcggtcacacaggaaaagatggcggctgaaggtcttgccgcagtgtaaaacatggcgggcctctttgtctttgctgtgtgcttttcgtgttgggttttgccgcagggacaatatggcaggcgttgtcatatgtatatcatggcttttgtcacgtggacatcatggcgggcttgccgcattgttaaagatggcgggttttgccgcctatacctccccccccaccccccaacccccccaactccccacccccaccccccaccccccacctccccacccccctacccccctacccccctacccccccctccccagccctgctcccagcaaacccctagtctagccccagccctactcccaccccgccccagccctgccccagccccagtcccctaaccccccagccctagccccagtcccagtcctgctcaaaataagttgtccattgcttatcctattatactgggatattccgtttacccttggcattgctgatcttcagtactgactccttgaccattttcagttaatgcatacaatcccatccttttgaaggacagcatggttggtgacacctaaggccccatttcttggcctcccaatatgtgtgattgtatttgtcgaggttgctatgcactagagaaggaaagtgctcccctcatccccacttttcccttccagcaggaagtgcccaccccataagacccttttatttggagagtctaggtgcacaattgtaagtgaccacaagcatgcatcttggacatttatgtgcgtaatcgcacactgctcattccatgtgaataaggtcctactctccgaccccttttgcaatacagaagggttgctgataacgcagtccccttttcttggcatgttgtgtgtgattataatcgtctgggatcctatgcactagaaaaggagggtcctctccacatacctcagtctcacctttcccttccagcagggagtgcccactccataagactctcacatttggacagtcaaggtgcgtaattgttaagtgaacacaaccatgcaccttagacatggatttgcataactacacacagctcaacctatctgaataaaatcctactctcagaccccttttgcagtacagcaggggtgctgatcaccaaggccctttttcctggcctggtatgcgtgtgattatgtttgtcccggttcctgtgtattagacatggaagcctcccctgccacactccacccccaatcttcctttcccttccggcagggagtgccctctccataagacgcttacgtttggacaatcaaggtgcacagttgtaagtgaccacaggcatacaccttggacattaatgtgcataaccactttgcccattccatctgaataaggtcctactctcagaccccttttgcagtacagcaggggtgctgatcaccaaggccccttttcttggcctgttatgtgcgtgattatatttgtctgggttcctgtgtattagacaaggaagccttccccccgcccccacccccactcccagtcttcctttcccttccagcagggagtgccccctccataagatcattacatttggacaatcaaggtgcacaattataagtgaccacagccatgcaccttggacattattggacattaatgtgcgtaactgcacatggcccatcccatctgaataaggtcctactctcagatgccctttgcagtacagcaggggtactgaatcaccaaggccctttttcttggcctgttatgtgtgtgattatatttatcccagtttctgtgtaatagacatgaaagcctcccctgccacaccccacctccaatcttcctttcccttccaccagggagtgtccactccatatacccttacatttggacaatcaaggtgcacaattgtaagtgagcataggcactcaccttggacatgaatgtgcataactgcacatggcccatcccatctgaataaggtcctactctcagaccctttttgcagtacagcaggggtgctgatcaccaaggccccttttcctggcctgttatgtgtgtgattatatttgttccagttcctgtgtaatagacatggaagcctcccctgccacactccacccccaatcttcctttcccttctggcaggaagtacccgctccataagacccttacatttggacagtcaaggtgcacaattgtatgtgaccacaaccatgcaccttggacataaatgtgtgtaactgcacatggcccatcccatctgaataaggtcctactctcagaccccttttgcagtacagtaggtgtgctgataaccaaggcccctcttcctggcctgttaacgtatgtgattatatttgtctgggttccagtgtataagacatggaagcctcccctgccccaccccaccctcaatcttcctttcccttctggcagggagtgccagctccataagaaccttacatttggacagtcaaggtgcacaattctaagtgaccgcagccatgcaccttggtcaataatgtgtgtaactgcacacggcctatctcatctgaataaggccttactctcagaccccttttgcagtacagcaggggtgctgataaccaaggcccattttcctggcctgttatgtgtgtgattatatttgtccaggtttctgtgtactagacaaggaagcctcctctgccccatcccatctacgcataatctttcttttcctcccagcagggagtgctcactccataagacccttacatttggacaatcaaggtgcacaattgtaagtgaccacaaccatgcatcttggaaatttatgtgcataactgcacatggcttatcctatttgaataaagtcctactctcagaccccctttgcagtatagctggggtgctgatcactgaggcctctttgcttggcttgtctatattcttgtgtactagataagggcaccttctcatggactccctttgcttttcaacaaggagtacccactactttttaagattcttatatttgtccaaagtacatggttttaattgaccacaacaatgtcccttggacattaatgtatgtaatcaccacatggttcatcctaattaaacaaagttctaccttctcaccctccatttgcagtataccagggttgctgaccccctaagtccccttttcttggcttgttgatgtgtatttctttgtctctttctttcttgtctttgctctttgttctctatctaaagtgtgtcttacccatttccatgtttctcttgctaatttctttcgtgtgtgcctttgcctcattttctctttttgttcacaagagtggtctgtgtcttgtcttagacatatctctcatttttcattttgttgctatttctctttgctctcctagatgtggctcttctttcacgctttatttcatgtctcctttttgggtcacatgctgtgtgctttttgtccttttcttgttctgtctacctctcctttctctgcctacctctcttttctctttgtgaactgtgattatttgttaccccttccccttctcgttcgttttaaatttcaccttttttctgagtctggcctcctttctgctgtttctactttttatctcacatttctcatttctgcatttcctttctgcctctcttgggctattctctctctcctcccctgcgtgcctcagcatctcttgctgtttgtgattttctatttcagtattaatctctgttggcttgtatttgttctctgcttcttccctttctactcacctttgagtatttcagcctcttcatgaatctatctccctctctttgatttcatgtaatctctccttaaatatttctttgcatatgtgggcaagtgtacgtgtgtgtgtgtcatgtgtggcagaggggcttcctaacccctgcctgataggtgcagaacgtcggctatcagagcaagcattgtggagcggttccttatgccaggctgccatgtgagatgatccaagaccaaaacaaggccctagactgcagtaaaacccagaactcaagtagggcagaaggtggaaggctcatatggatagaaggcccaaagtataagacagatggtttgagacttgagacccgaggactaagatggaaagcccatgttccaagatagatagaagcctcaggcctgaaaccaacaaaagcctcaagagccaagaaaacagagggtggcctgaattggaccgaaggcctgagttggatggaagtctcaaggcttgagttagaagtcttaagacctgggacaggacacatggaaggcctaagaactgagacttgtgacacaaggccaacgacctaagattagcccagggttgtagctggaagacctacaacccaaggatggaaggcccctgtcacaaagcctacctagatggatagaggacccaagcgaaaaaggtatctcaagactaacggccggaatctggaggcccatgacccagaacccaggaaggatagaagcttgaagacctggggaaatcccaagatgagaaccctaaaccctacctcttttctattgtttacacttcttactcttagatatttccagttctcctgtttatctttaagcctgattcttttgagatgtactttttgatgttgccggttacctttagattgacagtattatgcctgggccagtcttgagccagctttaaatcacagcttttacctatttgttaggctatagtgttttgtaaacttctgtttctattcacatcttctccacttgagagagacaccaaaatccagtcagtatctaatctggcttttgttaacttccctcaggagcagacattcatataggtgatactgtatttcagtcctttcttttgaccccagaagccctagactgagaagataaaatggtcaggttgttggggaaaaaaaagtgccaggctctctag

SEQ ID NO: 78 - DNA, 3154 bp, synthetic construct
  source 1..3154: mol_type = other DNA
gaatttttctttggaatcatttttggttgacatctctgttttttgtggatcagttttttactcttccactctcttttctatattttgcccatcggggctgcggatacctggttttattattttttctttgcccaacggggccgtggatacctgccttttaattcttttttattcgcccatcggggccgcggatacctgctttttatttttttttccttagcccatcggggtatcggatacctgctgattcccttcccctctgaacccccaacactctggcccatcggggtgacggatatctgctttttaaaaattttctttttttggcccatcggggcttcggatacctgctttttttttttttatttttccttgcccatcggggcctcggatacctgctttaatttttgtttttctggcccatcggggccgcggatacctgctttgatttttttttttcatcgcccatcggtgctttttatggatgaaaaaatgttgacattgcggcattgctcagcatggcgggctgtgctttgttaggttgtccaaaatggcggatccagttctgtcgcagtgttcaagtggcgggaaggccacatcatgatgggcgaggctttgttaagtggttagcatggtggtggacatgtgcggtcacacaggaaaagatggcggctgaaggtcttgccgcagtgtaaaacatggcgggcctctttgtctttgctgtgtgcttttcgtgttgggttttgccgcagggacaatatggcaggcgttgtcatatgtatatcatggcttttgtcacgtggacatcatggcgggcttgccgcattgttaaagatggcgggttttgccgcctatacctccccccccaccccccaacccccccaactccccacccccaccccccaccccccacctccccacccccctacccccctacccccctacccccccctccccagccctgctcccagcaaacccctagtctagccccagccctactcccaccccgccccagccctgccccagccccagtcccctaaccccccagccctagccccagtcccagtcctgctcaaaataagttgtccattgcttatcctattatactgggatattccgtttacccttggcattgctgatcttcagtactgactccttgaccattttcagttaatgcatacaatcccatatcttcctcagaagaataggcttgttgttttacagtgttagtgatccattccctttgacgatccctaggtggagatggggcatgaggatcctccaggggaaaagctcactaccactgggcaacaaccctaggtcaggaggttctgtcaagatactttcctggtcccagataggaagataaagtctcaaaaacaaccaccacacgtcaagtgtgtatttctttgtctctttctttcttgtctttgctctttgttctctatctaaagtgtgtcttacccatttccatgtttctcttgctaatttctttcgtgtgtgcctttgcctcattttctctttttgttcacaagagtggtctgtgtcttgtcttagacatatctctcatttttcattttgttgctatttctctttgctctcctagatgtggctcttctttcacgctttatttcatgtctcctttttgggtcacatgctgtgtgctttttgtccttttcttgttctgtctacctctcctttctctgcctacctctcttttctctttgtgaactgtgattatttgttaccccttccccttctcgttcgttttaaatttcaccttttttctgagtctggcctcctttctgctgtttctactttttatctcacatttctcatttctgcatttcctttctgcctctcttgggctattctctctctcctcccctgcgtgcctcagcatctcttgctgtttgtgattttctatttcagtattaatctctgttggcttgtatttgttctctgcttcttccctttctactcacctttgagtatttcagcctcttcatgaatctatctccctctctttgatttcatgtaatctctccttaaatatttctttgcatatgtgggcaagtgtacgtgtgtgtgtgtcatgtgtggcagaggggcttcctaacccctgcctgataggtgcagaacgtcggctatcagagcaagcattgtggagcggttccttatgccaggctgccatgtgagatgatccaagaccaaaacaaggccctagactgcagtaaaacccagaactcaagtagggcagaaggtggaaggctcatatggatagaaggcccaaagtataagacagatggtttgagacttgagacccgaggactaagatggaaagcccatgttccaagatagatagaagcctcaggcctgaaaccaacaaaagcctcaagagccaagaaaacagagggtggcctgaattggaccgaaggcctgagttggatggaagtctcaaggcttgagttagaagtcttaagacctgggacaggacacatggaaggcctaagaactgagacttgtgacacaaggccaacgacctaagattagcccagggttgtagctggaagacctacaacccaaggatggaaggcccctgtcacaaagcctacctagatggatagaggacccaagcgaaaaaggtatctcaagactaacggccggaatctggaggcccatgacccagaacccaggaaggatagaagcttgaagacctggggaaatcccaagatgagaaccctaaaccctacctcttttctattgtttacacttcttactcttagatatttccagttctcctgtttatctttaagcctgattcttttgagatgtactttttgatgttgccggttacctttagattgacagtattatgcctgggccagtcttgagccagctttaaatcacagcttttacctatttgttaggctatagtgttttgtaaacttctgtttctattcacatcttctccacttgagagagacaccaaaatccagtcagtatctaatctggcttttgttaacttccctcaggagcagacattcatataggtgatactgtatttcagtcctttcttttgaccccagaagccctagactgagaagataaaatggtcaggttgttggggaaaaaaaagtgccaggctctctag

SEQ ID NO: 79 - DNA, 988 bp, synthetic construct
  source 1..988: mol_type = other DNA
gaatttttctttggaatcatttttggtgacatctctgttttttgtggatcagttttttactcttccactctcttttctatattttgcccatcggggctgcggatacctggttttattattttttctttgcccaacggggccgtggatacctgccttttaattcttttttattcgcccatcggggccgcggatacctgctttttatttttttttccttagcccatcggggtatcggatacctgctgattcccttcccctctgaacccccaacactctggcccatcggggtgacggatatctgctttttaaaaattttctttttttggcccatcggggcttcggatacctgctttttttttttttatttttccttgcccatcggggcctcggatacctgctttaatttttgtttttctggcccatcggggccgcggatacctgctttgatttttttttttcatcgcccatcggtgctttttatggatgaaaaaatgtttctttgctgtgtgcttttcgtgttgggttttgccgcagggacaatatggcaggcgttgtcatatgtatatcatggcttttgtcacgtggacatcatggcgggcttgccgcattgttaaagatggcgggttttgccgcctagtctatttgttaggctatagtgttttgtaaacttctgtttctattcacatcttctccacttgagagagacaccaaaatccagtcagtatctaatctggcttttgttaacttccctcaggagcagacattcatataggtgatactgtatttcagtcctttcttttgaccccagaagccctagactgagaagataaaatggtcaggttgttggggaaaaaaaaagtgccaggctctctagagaaaaatgtgaagagatgctccaggccaatgagaagaattagacaagaaatacacagatgtgccagacttctgagaagcacctgccagcaacagcttccttctttgagcttag

SEQ ID NO: 80 - DNA, 2363 bp, synthetic construct
  source 1..2363: mol_type = other DNA
gaatttttctttggaatcatttttggtgacatctctgttttttgtggatcagttttttactcttccactctcttttctatattttgcccatcggggctgcggatacctggttttattattttttctttgcccaacggggccgtggatacctgccttttaattcttttttattcgcccatcggggccgcggatacctgctttttatttttttttccttagcccatcggggtatcggatacctgctgattcccttcccctctgaacccccaacactctggcccatcggggtgacggatatctgctttttaaaaattttctttttttggcccatcggggcttcggatacctgctttttttttttttatttttccttgcccatcggggcctcggatacctgctttaatttttgtttttctggcccatcggggccgcggatacctgctttgatttttttttttcatcgcccatcggtgctttttatggatgaaaaaatgttggttttgtgggttgttgcactctctggaatatctacacttttttttgctgctgatcatttggtggtgtgtgagtgtacctaccgctttggcagagaatgactctgcagttaagctaagggcgtgttcagattgtggaggaaaagtggccgccattttaacttgccgcataactcggcttagggctagtcgtttgtgctaagttaaactagggaggcaagatggatgatagcaggtcaggcagaggaagtcatgtgcattgcatgagctaaacctatctgaatgaattgatttggggcttgttaggagctttgcgtgattgttgtatcgggaggcagtaagaatcatcttttatcagtacaagggactagttaaaaatggaaggttaggaaagactaaggtgcagggcttaaaatggcgattttgacattgcggcattgctcagcatggcgggctgtgctttgttaggttgtccaaaatggcggatccagttctgtcgcagtgttcaagtggcgggaaggccacatcatgatgggcgaggctttgttaagtggttagcatggtggtggacatgtgcggtcacacaggaaaagatggcggctgaaggtcttgccgcagtgtaaaacatggcgggcctctttgtctttgctgtgtgcttttcgtgttgggttttgccgcagggacaatatggcaggcgttgtcatatgtatatcatggcttttgtcacgtggacatcatggcgggcttgccgcattgttaaagatggcgggttttgccgcctagtgccacgcagagcgggagaaaaggtgggatggacagtgctggattgctgcataacccaaccaattagaaatgggggtggaattgatcacagccaattagagcagaagatggaattagactgatgacacactgtccagctactcagcgaagacctgggtgaattagcatggcacttcgcagctgtctttagccagtcaggagaaagaagtggaggggccacgtgtatgtctcccagtgggcggtacaccaggtgttttcaaggtcttttcaaggacatttagcctttccacctctgtcccctcttatttgtcccctcctgtccagtgctgcctcttgcagtgctggatatctggctgtgtggtctgaacctccctccattcctctgtattggtgcctcacctaaggctaagtatacctcccccccaccccccaacccccccaactccccctctggtctgccctgcactgcactgttgccatgggcagtgctccaggcctgcttggtgtggacatggtggtgagccgtggcaaggaccagaatggatcacagatgatcgttggccaacaggtggcagaagaggaattcctgccttcctcaagaggaacacctaccccttggctaatgctggggtcggattttgatttatatttatcttttggatgtcagtcatacagtctgattttgtggtttgctagtgtttgaatttaagtcttaagtgactattatagaaatgtactatttgttaggctatagtgttttgtaaacttctgtttctattcacatcttctccacttgagagagacaccaaaatccagtcagtatctaatctggcttttgttaacttccctcaggagcagacattcatataggtgatactgtatttcagtcctttcttttgaccccagaagccctagactgagaagataaaatggtcaggttgttggggaaaaaaaaagtgccaggctctctagagaaaaatgtgaagagatgctccaggccaatgagaagaattagacaagaaatacacagatgtgccagacttctgagaagcacctgccagcaacagcttccttctttgagcttag

SEQ ID NO: 81 - DNA, 684 bp, synthetic construct
  source 1..684: mol_type = other DNA
gtaaactggcaaaggggtggctgggccaaaagacagaggaattaagtaagaagtccaggaaaaatgaacttcacatcaaattttagagcacggtagccatgaatcttgtgaatagctcccaaaaatgtcctgtggaagacaactagaaagcattctacaatcaggcacccacctccacctgcagcctcctgtgttgttctcatggggcacctctgggctccagctcctccaaggcacctccacactctctcaagtacactcttcactcttccccaaacatgattcccctactgctctgcctaactcccacttctctttcaagtagcagcttaaacgtcacctcatatttggctggaaaatagaatatagacagaggggtaagttaaggctagaaaggcaggctgggtcaacagaatggcaagctaaaacatgggattttctaaaacagcctaagagggtgccagataaaagtgtgcaaggagtggcacaactccagtttcatctttagctatagcaattaacaccataaggagtctggattcaattttgccatttactagctagctaccaacttctgtgtcgctttgggcaaatcaattaaatccatacctccctttccatctgcagaatgggtttataacagtacttaaacctcaaggtactaagaacagtaaagagttaatg

SEQ ID NO: 82 - DNA, 502 bp, synthetic construct
  source 1..502: mol_type = other DNA
aaaccagaaagtattctcagtaatgatagtatggataaagcaggtttctatgaccctttattacagaatctgtgagtttttcacaattaaaaagtaataaaaagtagtgacaacattcactgaactcttattctatgccaacttgttccggtatgcccttacacccacaaaagccctatgcataaggtggcattattccagcatgtattgcattgtacacacaaagaggtcaagcactccaccacggccctaagcatggtggctgaggtgggaaggccagaggtaggtgggcccgcgcccttttccactctgaaccatgcctccaagataggagggtgggaaagtgctcaagacacattagaaattccccataaaagacaagattgttgaacacctgcaagtgaataaagataaactgatctcagaggggaaaaagacgcagggttaggaaacagcaccctgctcgaggacgttctttccaaacagcctgctcatcacccgt

SEQ ID NO: 83 - DNA, 6920 bp, synthetic construct
  source 1..6920: mol_type = other DNA
tacgtaaactggcaaaggggtggctgggccaaaagacagaggaattaagtaagaagtccaggaaaaatgaacttcacatcaaattttagagcacggtagccatgaatcttgtgaatagctcccaaaaatgtcctgtggaagacaactagaaagcattctacaatcaggcacccacctccacctgcagcctcctgtgttgttctcatggggcacctctgggctccagctcctccaaggcacctccacactctctcaagtacactcttcactcttccccaaacatgattcccctactgctctgcctaactcccacttctctttcaagtagcagcttaaacgtcacctcatatttggctggaaaatagaatatagacagaggggtaagttaaggctagaaaggcaggctgggtcaacagaatggcaagctaaaacatgggattttctaaaacagcctaagagggtgccagataaaagtgtgcaaggagtggcacaactccagtttcatctttagctatagcaattaacaccataaggagtctggattcaattttgccatttactagctagctaccaacttctgtgtcgctttgggcaaatcaattaaatccatacctccctttccatctgcagaatgggtttataacagtacttaaacctcaaggtactaagaacagtaaagagttaatggtacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcgaaaaccagaaagtattctcagtaatgatagtatggataaagcaggtttctatgaccctttattacagaatctgtgagtttttcacaattaaaaagtaataaaaagtagtgacaacattcactgaactcttattctatgccaacttgttccggtatgcccttacacccacaaaagccctatgcataaggtggcattattccagcatgtattgcattgtacacacaaagaggtcaagcactccaccacggccctaagcatggtggctgaggtgggaaggccagaggtaggtgggcccgcgcccttttccactctgaaccatgcctccaagataggagggtgggaaagtgctcaagacacattagaaattccccataaaagacaagattgttgaacacctgcaagtgaataaagataaactgatctcagaggggaaaaagacgcagggttaggaaacagcaccctgctcgaggacgttctttccaaacagcctgctcatcacccgttcgaattcctcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaagtcgacaccggggcccagatctggtaccgagctcggatccactagtccagtgtggtggaattctgcagagaatttttctttggaatcatttttggttgacatctctgttttttgtggatcagttttttactcttccactctcttttctatattttgcccatcggggctgcggatacctggttttattattttttctttgcccaacggggccgtggatacctgccttttaattcttttttattcgcccatcggggccgcggatacctgctttttatttttttttccttagcccatcggggtatcggatacctgctgattcccttcccctctgaacccccaacactctggcccatcggggtgacggatatctgctttttaaaaattttctttttttggcccatcggggcttcggatacctgctttttttttttttatttttccttgcccatcggggcctcggatacctgctttaatttttgtttttctggcccatcggggccgcggatacctgctttgatttttttttttcatcgcccatcggtgctttttatggatgaaaaaatgtttcgatcggccggatatcacgcgtcatatggctagcctgcagggatccaatgtaactgtattcagcgatgacgaaattcttagctattgtaatactctagaggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagctagcaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagtccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaattcgccaccatggccagctccgaggatgtcatcaaagagtttatgagatttaaggtcaagatggagggaagcgtcaacggacacgagttcgagattgagggagaaggagaaggccggccttacgagggcacacaaaccgctaagctcaaggtcacaaaaggaggacccctccccttctcctgggatattctgagccctcagttccagtacggaagcaaagcctatgttaaacaccctgccgacatccctgactatctgaagctctccttccctgaaggcttcaagtgggagagattcatgaacttcgaggacggaggcgtggtgacagtcacacaagatagcaccctccaggacggagagtttatttataaggtgaaactcagaggaaccaacttcccctccgatggccctgtcatgcaaaaaaaaacaatgggatgggaagcctccaccgagagaatgtatcctgaggatggcgctctgaaaggcgaaattaaaatgagactgaaactcaaagacggaggacactacgatgccgaggtcaaaacaacctacaaggccaagaaacaagtgcagctgcctggcgcctacatgactgatattaaactcgacattatcagccataatggggactacaccatcgtggaacaatatgagagagctgagggcagacatagcacaggcgctggaagtacttgaggatcctgatcgagtctagagggcccccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaagatcttcatgtctgcggctctagagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatg

SEQ ID NO: 84 - DNA, 7501 bp, synthetic construct
  source 1..7501: mol_type = other DNA
aaaccagaaagtattctcagtaatgatagtatggataaagcaggtttctatgaccctttattacagaatctgtgagtttttcacaattaaaaagtaataaaaagtagtgacaacattcactgaactcttattctatgccaacttgttccggtatgcccttacacccacaaaagccctatgcataaggtggcattattccagcatgtattgcattgtacacacaaagaggtcaagcactccaccacggccctaagcatggtggctgaggtgggaaggccagaggtaggtgggcccgcgcccttttccactctgaaccatgcctccaagataggagggtgggaaagtgctcaagacacattagaaattccccataaaagacaagattgttgaacacctgcaagtgaataaagataaactgatctcagaggggaaaaagacgcagggttaggaaacagcaccctgctcgaggacgttctttccaaacagcctgctcatcacccgttcgaattcctcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaagtcgacaccggggcccagatctggtaccgagctcggatccactagtccagtgtggtggaattctgcagagaatttttctttggaatcatttttggtgacatctctgttttttgtggatcagttttttactcttccactctcttttctatattttgcccatcggggctgcggatacctggttttattattttttctttgcccaacggggccgtggatacctgccttttaattcttttttattcgcccatcggggccgcggatacctgctttttatttttttttccttagcccatcggggtatcggatacctgctgattcccttcccctctgaacccccaacactctggcccatcggggtgacggatatctgctttttaaaaattttctttttttggcccatcggggcttcggatacctgctttttttttttttatttttccttgcccatcggggcctcggatacctgctttaatttttgtttttctggcccatcggggccgcggatacctgctttgatttttttttttcatcgcccatcggtgctttttatggatgaaaaaatgtttctttgctgtgtgcttttcgtgttgggttttgccgcagggacaatatggcaggcgttgtcatatgtatatcatggcttttgtcacgtggacatcatggcgggcttgccgcattgttaaagatggcgggttttgccgcctagtctatttgttaggctatagtgttttgtaaacttctgtttctattcacatcttctccacttgagagagacaccaaaatccagtcagtatctaatctggcttttgttaacttccctcaggagcagacattcatataggtgatactgtatttcagtcctttcttttgaccccagaagccctagactgagaagataaaatggtcaggttgttggggaaaaaaaaagtgccaggctctctagagaaaaatgtgaagagatgctccaggccaatgagaagaattagacaagaaatacacagatgtgccagacttctgagaagcacctgccagcaacagcttccttctttgagcttagtccatccctcatgaaaaatgactgaccactgctgggcagcaggagggatgatgaccaactaattcccaaaccccagtctcattggtaccatcgatcggccggatatcacgcgtcatatggctagcctgcagggatccaatgtaactgtattcagcgatgacgaaattcttagctattgtaatactctagaggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagctagcaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagtccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaattcgccaccatggccagctccgaggatgtcatcaaagagtttatgagatttaaggtcaagatggagggaagcgtcaacggacacgagttcgagattgagggagaaggagaaggccggccttacgagggcacacaaaccgctaagctcaaggtcacaaaaggaggacccctccccttctcctgggatattctgagccctcagttccagtacggaagcaaagcctatgttaaacaccctgccgacatccctgactatctgaagctctccttccctgaaggcttcaagtgggagagattcatgaacttcgaggacggaggcgtggtgacagtcacacaagatagcaccctccaggacggagagtttatttataaggtgaaactcagaggaaccaacttcccctccgatggccctgtcatgcaaaaaaaaacaatgggatgggaagcctccaccgagagaatgtatcctgaggatggcgctctgaaaggcgaaattaaaatgagactgaaactcaaagacggaggacactacgatgccgaggtcaaaacaacctacaaggccaagaaacaagtgcagctgcctggcgcctacatgactgatattaaactcgacattatcagccataatggggactacaccatcgtggaacaatatgagagagctgagggcagacatagcacaggcgctggaagtacttgaggatcctgatcgagtctagagggcccccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaagatcttcatgtctgcggctctagagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtacgtaaactggcaaaggggtggctgggccaaaagacagaggaattaagtaagaagtccaggaaaaatgaacttcacatcaaattttagagcacggtagccatgaatcttgtgaatagctcccaaaaatgtcctgtggaagacaactagaaagcattctacaatcaggcacccacctccacctgcagcctcctgtgttgttctcatggggcacctctgggctccagctcctccaaggcacctccacactctctcaagtacactcttcactcttccccaaacatgattcccctactgctctgcctaactcccacttctctttcaagtagcagcttaaacgtcacctcatatttggctggaaaatagaatatagacagaggggtaagttaaggctagaaaggcaggctgggtcaacagaatggcaagctaaaacatgggattttctaaaacagcctaagagggtgccagataaaagtgtgcaaggagtggcacaactccagtttcatctttagctatagcaattaacaccataaggagtctggattcaattttgccatttactagctagctaccaacttctgtgtcgctttgggcaaatcaattaaatccatacctccctttccatctgcagaatgggtttataacagtacttaaacctcaaggtactaagaacagtaaagagttaatggtacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcga

SEQ ID NO: 85 - DNA, 8876 bp, synthetic construct
  source 1..8876: mol_type = other DNA
aaaccagaaagtattctcagtaatgatagtatggataaagcaggtttctatgaccctttattacagaatctgtgagtttttcacaattaaaaagtaataaaaagtagtgacaacattcactgaactcttattctatgccaacttgttccggtatgcccttacacccacaaaagccctatgcataaggtggcattattccagcatgtattgcattgtacacacaaagaggtcaagcactccaccacggccctaagcatggtggctgaggtgggaaggccagaggtaggtgggcccgcgcccttttccactctgaaccatgcctccaagataggagggtgggaaagtgctcaagacacattagaaattccccataaaagacaagattgttgaacacctgcaagtgaataaagataaactgatctcagaggggaaaaagacgcagggttaggaaacagcaccctgctcgaggacgttctttccaaacagcctgctcatcacccgttcgaattcctcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaagtcgacaccggggcccagatctggtaccgagctcggatccactagtccagtgtggtggaattctgcagagaatttttctttggaatcatttttggtgacatctctgttttttgtggatcagttttttactcttccactctcttttctatattttgcccatcggggctgcggatacctggttttattattttttctttgcccaacggggccgtggatacctgccttttaattcttttttattcgcccatcggggccgcggatacctgctttttatttttttttccttagcccatcggggtatcggatacctgctgattcccttcccctctgaacccccaacactctggcccatcggggtgacggatatctgctttttaaaaattttctttttttggcccatcggggcttcggatacctgctttttttttttttatttttccttgcccatcggggcctcggatacctgctttaatttttgtttttctggcccatcggggccgcggatacctgctttgatttttttttttcatcgcccatcggtgctttttatggatgaaaaaatgttggttttgtgggttgttgcactctctggaatatctacacttttttttgctgctgatcatttggtggtgtgtgagtgtacctaccgctttggcagagaatgactctgcagttaagctaagggcgtgttcagattgtggaggaaaagtggccgccattttaacttgccgcataactcggcttagggctagtcgtttgtgctaagttaaactagggaggcaagatggatgatagcaggtcaggcagaggaagtcatgtgcattgcatgagctaaacctatctgaatgaattgatttggggcttgttaggagctttgcgtgattgttgtatcgggaggcagtaagaatcatcttttatcagtacaagggactagttaaaaatggaaggttaggaaagactaaggtgcagggcttaaaatggcgattttgacattgcggcattgctcagcatggcgggctgtgctttgttaggttgtccaaaatggcggatccagttctgtcgcagtgttcaagtggcgggaaggccacatcatgatgggcgaggctttgttaagtggttagcatggtggtggacatgtgcggtcacacaggaaaagatggcggctgaaggtcttgccgcagtgtaaaacatggcgggcctctttgtctttgctgtgtgcttttcgtgttgggttttgccgcagggacaatatggcaggcgttgtcatatgtatatcatggcttttgtcacgtggacatcatggcgggcttgccgcattgttaaagatggcgggttttgccgcctagtgccacgcagagcgggagaaaaggtgggatggacagtgctggattgctgcataacccaaccaattagaaatgggggtggaattgatcacagccaattagagcagaagatggaattagactgatgacacactgtccagctactcagcgaagacctgggtgaattagcatggcacttcgcagctgtctttagccagtcaggagaaagaagtggaggggccacgtgtatgtctcccagtgggcggtacaccaggtgttttcaaggtcttttcaaggacatttagcctttccacctctgtcccctcttatttgtcccctcctgtccagtgctgcctcttgcagtgctggatatctggctgtgtggtctgaacctccctccattcctctgtattggtgcctcacctaaggctaagtatacctcccccccaccccccaacccccccaactccccctctggtctgccctgcactgcactgttgccatgggcagtgctccaggcctgcttggtgtggacatggtggtgagccgtggcaaggaccagaatggatcacagatgatcgttggccaacaggtggcagaagaggaattcctgccttcctcaagaggaacacctaccccttggctaatgctggggtcggattttgatttatatttatcttttggatgtcagtcatacagtctgattttgtggtttgctagtgtttgaatttaagtcttaagtgactattatagaaatgtactatttgttaggctatagtgttttgtaaacttctgtttctattcacatcttctccacttgagagagacaccaaaatccagtcagtatctaatctggcttttgttaacttccctcaggagcagacattcatataggtgatactgtatttcagtcctttcttttgaccccagaagccctagactgagaagataaaatggtcaggttgttggggaaaaaaaaagtgccaggctctctagagaaaaatgtgaagagatgctccaggccaatgagaagaattagacaagaaatacacagatgtgccagacttctgagaagcacctgccagcaacagcttccttctttgagcttagtccatccctcatgaaaaatgactgaccactgctgggcagcaggagggatgatgaccaactaattcccaaaccccagtctcattggtaccatcgatcggccggatatcacgcgtcatatggctagcctgcagggatccaatgtaactgtattcagcgatgacgaaattcttagctattgtaatactctagaggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagctagcaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagtccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaattcgccaccatggccagctccgaggatgtcatcaaagagtttatgagatttaaggtcaagatggagggaagcgtcaacggacacgagttcgagattgagggagaaggagaaggccggccttacgagggcacacaaaccgctaagctcaaggtcacaaaaggaggacccctccccttctcctgggatattctgagccctcagttccagtacggaagcaaagcctatgttaaacaccctgccgacatccctgactatctgaagctctccttccctgaaggcttcaagtgggagagattcatgaacttcgaggacggaggcgtggtgacagtcacacaagatagcaccctccaggacggagagtttatttataaggtgaaactcagaggaaccaacttcccctccgatggccctgtcatgcaaaaaaaaacaatgggatgggaagcctccaccgagagaatgtatcctgaggatggcgctctgaaaggcgaaattaaaatgagactgaaactcaaagacggaggacactacgatgccgaggtcaaaacaacctacaaggccaagaaacaagtgcagctgcctggcgcctacatgactgatattaaactcgacattatcagccataatggggactacaccatcgtggaacaatatgagagagctgagggcagacatagcacaggcgctggaagtacttgaggatcctgatcgagtctagagggcccccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaagatcttcatgtctgcggctctagagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtacgtaaactggcaaaggggtggctgggccaaaagacagaggaattaagtaagaagtccaggaaaaatgaacttcacatcaaattttagagcacggtagccatgaatcttgtgaatagctcccaaaaatgtcctgtggaagacaactagaaagcattctacaatcaggcacccacctccacctgcagcctcctgtgttgttctcatggggcacctctgggctccagctcctccaaggcacctccacactctctcaagtacactcttcactcttccccaaacatgattcccctactgctctgcctaactcccacttctctttcaagtagcagcttaaacgtcacctcatatttggctggaaaatagaatatagacagaggggtaagttaaggctagaaaggcaggctgggtcaacagaatggcaagctaaaacatgggattttctaaaacagcctaagagggtgccagataaaagtgtgcaaggagtggcacaactccagtttcatctttagctatagcaattaacaccataaggagtctggattcaattttgccatttactagctagctaccaacttctgtgtcgctttgggcaaatcaattaaatccatacctccctttccatctgcagaatgggtttataacagtacttaaacctcaaggtactaagaacagtaaagagttaatggtacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcga

SEQ ID NO: 86 - DNA, 7278 bp, synthetic construct
  source 1..7278: mol_type = other DNA
gtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaagtcgacaccggggcccagatctggtaccgagctcggatccactagtccagtgtggtggaattctgcagagaatttttctttggaatcatttttggttgacatctctgttttttgtggatcagttttttactcttccactctcttttctatattttgcccatcggggctgcggatacctggttttattattttttctttgcccaacggggccgtggatacctgccttttaattcttttttattcgcccatcggggccgcggatacctgctttttatttttttttccttagcccatcggggtatcggatacctgctgattcccttcccctctgaacccccaacactctggcccatcggggtgacggatatctgctttttaaaaattttctttttttggcccatcggggcttcggatacctgctttttttttttttatttttccttgcccatcggggcctcggatacctgctttaatttttgtttttctggcccatcggggccgcggatacctgctttgatttttttttttcatcgcccatcggtgctttttatggatgaaaaaatgtttcgatcggccggatatcacgcgtcatatggctagcctgcagggatccaatgtaactgtattcagcgatgacgaaattcttagctattgtaatactctagaggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagctagcaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagtccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaattcgccaccatggccagctccgaggatgtcatcaaagagtttatgagatttaaggtcaagatggagggaagcgtcaacggacacgagttcgagattgagggagaaggagaaggccggccttacgagggcacacaaaccgctaagctcaaggtcacaaaaggaggacccctccccttctcctgggatattctgagccctcagttccagtacggaagcaaagcctatgttaaacaccctgccgacatccctgactatctgaagctctccttccctgaaggcttcaagtgggagagattcatgaacttcgaggacggaggcgtggtgacagtcacacaagatagcaccctccaggacggagagtttatttataaggtgaaactcagaggaaccaacttcccctccgatggccctgtcatgcaaaaaaaaacaatgggatgggaagcctccaccgagagaatgtatcctgaggatggcgctctgaaaggcgaaattaaaatgagactgaaactcaaagacggaggacactacgatgccgaggtcaaaacaacctacaaggccaagaaacaagtgcagctgcctggcgcctacatgactgatattaaactcgacattatcagccataatggggactacaccatcgtggaacaatatgagagagctgagggcagacatagcacaggcgctggaagtacttgaggatcctgatcgagtctagagggcccccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaagatcttcatgtctgcggctctagagctgcattaatgaatcggccaacgcgcggggagaggcggtttgcgtattgggcgctcttccgcttcctcgctcactgactcgctgcgctcggtcgttcggctggataacgcaggaaagaacatgtggacttcaggctcctcctagaaaacaagaaggcttaaaaagcaagacaacctattagctatgttgctcaataaatttcaatgttaatttcctaattctttgataacacattcctgaactcctttgtaattcaggcaacattttaaaaatcagatgatctaactggactccaagaattacataaacagtgtgagcagtgagacttcgaatgtgcaatcaactatttttttaatcctgccgctaaaagccttcagtagcacagaactggttgtcagagatttcagtgtaacctaacacctaacgtgaatgactgctgtacaaaaatacagctgaaaatactatgaggaagagatagaaacggattgttgatggctaagcaaaacagcaaatgctgatggttatcaaacacatgccgttcccactctacacagataagatttcaaagctgtttgagtcctaggttaagttttgggcaagttctggcttgatgccttatattcagataaacattttccaggcagtgaacatattcaaagttggggacagtggggtaacccgaaaacattcctgaccttgatggacagataatccaatggtggagacacaaataaggccaaattggctaaccaaaggtgctggagtgttaatgcagaggtctggcaagggtacaccgaagagctctcaggaaagagtgactggtttgacttggagaaggagaaggaaggcagaccaaagatcagagcagaaaacatgcccaggctgagtatgggtcggacaggaagcaacatgtgagcaaaaggccagtgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcgaattgcttgataagcctgggtagattgtccttcttaaataacttacaccaaagcactatcaaagaagctctccctttgtagagccctcagattcacagaaggaatttttcgaattgccatttctataggaataaaggctatcaggaaccacaatactaggagaaaataattctgaaggggaggtagcagtgaagactcttatttctattttcttgactatatttataaaaaattgttgccgaaagaacagaagaccaagagccaaaaatcacactgcagtcttactcttggatgtaatgttactatcagtttcagtgacacaacatcagaggcaatctgatattgacaatggtaaaaatatgcagacgccaaaattaggagggtgcagttagttgacagtagagcctataagtattcatctaagttttctctgtttgaagagaagaaaagagaacatcaaacatttctagtatatagagcaaggattcctaatctttttttttttttaatgtgcaaacaagacaaaagttacttcactgtctaggggttattccctactctttagcactggcctgactctctcactcccacatataagcattcaataaatacttatgccaacaattaagaaacacctggcatgaaaaataataacagtaataaactagaaggtagaaaatgtaattattgacaaaggtgtggaacaatacaaaataaattccactttagatacctgtgaaattttttattgtggcaccaggagagtaatttcctaacagagaaaagaaaacatagcatccacacctctgtctttaccaattcctaaataccagctacagttcaacttgtccacttcgaattcctcgagtttactccctatca

SEQ ID NO: 87 - DNA, 783 bp, synthetic construct
  source 1..783: mol_type = other DNA
ggacttcaggctcctcctagaaaacaagaaggcttaaaaagcaagacaacctattagctatgttgctcaataaatttcaatgttaatttcctaattctttgataacacattcctgaactcctttgtaattcaggcaacattttaaaaatcagatgatctaactggactccaagaattacataaacagtgtgagcagtgagacttcgaatgtgcaatcaactatttttttaatcctgccgctaaaagccttcagtagcacagaactggttgtcagagatttcagtgtaacctaacacctaacgtgaatgactgctgtacaaaaatacagctgaaaatactatgaggaagagatagaaacggattgttgatggctaagcaaaacagcaaatgctgatggttatcaaacacatgccgttcccactctacacagataagatttcaaagctgtttgagtcctaggttaagttttgggcaagttctggcttgatgccttatattcagataaacattttccaggcagtgaacatattcaaagttggggacagtggggtaacccgaaaacattcctgaccttgatggacagataatccaatggtggagacacaaataaggccaaattggctaaccaaaggtgctggagtgttaatgcagaggtctggcaagggtacaccgaagagctctcaggaaagagtgactggtttgacttggagaaggagaaggaaggcagaccaaagatcagagcagaaaacatgcccaggctgagtatgggtcggacaggaagca

SEQ ID NO: 88 - DNA, 851 bp, synthetic construct
  source 1..851: mol_type = other DNA
ttgcttgataagcctgggtagattgtccttcttaaataacttacaccaaagcactatcaaagaagctctccctttgtagagccctcagattcacagaaggaatttttcgaattgccatttctataggaataaaggctatcaggaaccacaatactaggagaaaataattctgaaggggaggtagcagtgaagactcttatttctattttcttgactatatttataaaaaattgttgccgaaagaacagaagaccaagagccaaaaatcacactgcagtcttactcttggatgtaatgttactatcagtttcagtgacacaacatcagaggcaatctgatattgacaatggtaaaaatatgcagacgccaaaattaggagggtgcagttagttgacagtagagcctataagtattcatctaagttttctctgtttgaagagaagaaaagagaacatcaaacatttctagtatatagagcaaggattcctaatctttttttttttttaatgtgcaaacaagacaaaagttacttcactgtctaggggttattccctactctttagcactggcctgactctctcactcccacatataagcattcaataaatacttatgccaacaattaagaaacacctggcatgaaaaataataacagtaataaactagaaggtagaaaatgtaattattgacaaaggtgtggaacaatacaaaataaattccactttagatacctgtgaaattttttattgtggcaccaggagagtaatttcctaacagagaaaagaaaacatagcatccacacctctgtctttaccaattcctaaataccagctacagttcaacttgtccac

SEQ ID NO: 89 - DNA, 20 bp, synthetic construct
  source 1..20: mol_type = other DNA
taggaggagcctgaagtccg

SEQ ID NO: 90 - DNA, 9290 bp, synthetic construct
  source 1..9290: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacctaggaggagcctgaagtccggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccgggggggggggggggggggggggggggggggggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaaggaattcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 91 - DNA, 20 bp, synthetic construct
  source 1..20: mol_type = other DNA
taggaggagcctgaggtccg

SEQ ID NO: 92 - DNA, 9290 bp, synthetic construct
  source 1..9290: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacctaggaggagcctgaggtccggttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccgggggggggggggggggggggggggggggggggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaaggaattcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 93 - DNA, 6920 bp, synthetic construct
  source 1..6920: mol_type = other DNA
ctcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaagtcgacaccggggcccagatctggtaccgagctcggatccactagtccagtgtggtggaattctgcagagaatttttctttggaatcatttttggttgacatctctgttttttgtggatcagttttttactcttccactctcttttctatattttgcccatcggggctgcggatacctggttttattattttttctttgcccaacggggccgtggatacctgccttttaattcttttttattcgcccatcggggccgcggatacctgctttttatttttttttccttagcccatcggggtatcggatacctgctgattcccttcccctctgaacccccaacactctggcccatcggggtgacggatatctgctttttaaaaattttctttttttggcccatcggggcttcggatacctgctttttttttttttatttttccttgcccatcggggcctcggatacctgctttaatttttgtttttctggcccatcggggccgcggatacctgctttgatttttttttttcatcgcccatcggtgctttttatggatgaaaaaatgtttcgatcggccggatatcacgcgtcatatggctagcctgcagggatccaatgtaactgtattcagcgatgacgaaattcttagctattgtaatactctagaggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttagagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaagctagcaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagtccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgaattcgccaccatggccagctccgaggatgtcatcaaagagtttatgagatttaaggtcaagatggagggaagcgtcaacggacacgagttcgagattgagggagaaggagaaggccggccttacgagggcacacaaaccgctaagctcaaggtcacaaaaggaggacccctccccttctcctgggatattctgagccctcagttccagtacggaagcaaagcctatgttaaacaccctgccgacatccctgactatctgaagctctccttccctgaaggcttcaagtgggagagattcatgaacttcgaggacggaggcgtggtgacagtcacacaagatagcaccctccaggacggagagtttatttataaggtgaaactcagaggaaccaacttcccctccgatggccctgtcatgcaaaaaaaaacaatgggatgggaagcctccaccgagagaatgtatcctgaggatggcgctctgaaaggcgaaattaaaatgagactgaaactcaaagacggaggacactacgatgccgaggtcaaaacaacctacaaggccaagaaacaagtgcagctgcctggcgcctacatgactgatattaaactcgacattatcagccataatggggactacaccatcgtggaacaatatgagagagctgagggcagacatagcacaggcgctggaagtacttgaggatcctgatcgagtctagagggcccccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaagatcttcatgtctgcggctctagagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtacgtaaactggcaaaggggtggctgggccaaaagacagaggaattaagtaagaagtccaggaaaaatgaacttcacatcaaattttagagcacggtagccatgaatcttgtgaatagctcccaaaaatgtcctgtggaagacaactagaaagcattctacaatcaggcacccacctccacctgcagcctcctgtgttgttctcatggggcacctctgggctccagctcctccaaggcacctccacactctctcaagtacactcttcactcttccccaaacatgattcccctactgctctgcctaactcccacttctctttcaagtagcagcttaaacgtcacctcatatttggctggaaaatagaatatagacagaggggtaagttaaggctagaaaggcaggctgggtcaacagaatggcaagctaaaacatgggattttctaaaacagcctaagagggtgacagataaaagtgtgcaaggagtggcacaactccagtttcatctttagctatagcaattaacaccataaggagtctggattcaattttgccatttactagctagctaccaacttctgtgtcgctttgggcaaatcaattaaatccatacctccctttccatctgcagaatgggtttataacagtacttaaacctcaaggtactaagaacagtaaagagttaatggtacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcgaaaaccagaaagtattctcagtaatgatagtatggataaagcaggtttctatgaccctttattacagaatctgtgagtttttcacaattaaaaagtaataaaaagtagtgacaacattcactgaactcttattctatgccaacttgttccggtatgcccttacacccacaaaagccctatgcataaggtggcattattccagcatgtattgcattgtacacacaaagaggtcaagcactccaccacggccctaagcatggtggctgaggtgggaaggccagaggtaggtgggcccgcgcccttttccactctgaaccatgcctccaagataggagggtgggaaagtgctcaagacacattagaaattccccataaaagacaagattgttgaacacctgcaagtgaataaagataaactgatctcagaggggaaaaagacgcagggttaggaaacagcaccctgctcgaggacgttctttccaaacagcctgctcatcacccgttcgaattc